(19) United States
(12) Patent Application Publication
(10) Pub. No.: US 2021/0115513 A1
(43) Pub. Date: Apr. 22, 2021

(54) METHODS OF USING GENETIC MARKERS ASSOCIATED WITH ENDOMETRIOSIS

(71) Applicant: JUNEAU BIOSCIENCES, L.L.C., Salt Lake City, UT (US)

(72) Inventors: Kenneth WARD, Salt Lake City, UT (US); Rakesh N. CHETTIER, Salt Lake City, UT (US); Hans M. ALBERTSEN, Salt Lake City, UT (US)

(21) Appl. No.: 16/493,944

(22) PCT Filed: Mar. 15, 2018
(86) PCT No.: PCT/US18/22743

§ 371 Date: Sep. 13, 2019

Related U.S. Application Data
(60) Provisional application No. 62/471,448, filed on Mar. 15, 2017.
(60) Provisional application No. 62/471,457, filed on Mar. 15, 2017.
(60) Provisional application No. 62/471,462, filed on Mar. 15, 2017.
(60) Provisional application No. 62/508,379, filed on May 18, 2017.
(60) Provisional application No. 62/588,265, filed on Nov. 17, 2017.
(60) Provisional application No. 62/588,268, filed on Nov. 17, 2017.
(60) Provisional application No. 62/639,711, filed on Mar. 7, 2018.
(60) Provisional application No. 62/639,730, filed on Mar. 7, 2018.

(51) Int. Cl. C12Q 1/6883; C12Q 1/686

(52) U.S. Cl. CPC C12Q 1/6883; C12Q 2600/156; C12Q 1/686

(57) ABSTRACT

Disclosed herein are methods of using genetic markers associated with endometriosis, for example via a computer-implemented program to predict risk of developing endometriosis, and methods of preventing or treating endometriosis or a symptom thereof.

### CROSS-REFERENCE

This application claims the benefit of U.S. Provisional Application No. 62/471,448, filed Mar. 15, 2017, U.S. Provisional Application No. 62/471,457, filed Mar. 15, 2017, U.S. Provisional Application No. 62/471,462, filed Mar. 15, 2017, U.S. Provisional Application No. 62/508,379, filed May 18, 2017, U.S. Provisional Application No. 62/588,265, filed Nov. 17, 2017, U.S. Provisional Application No. 62/588,268, filed Nov. 17, 2017, U.S. Provisional Application No. 62/639,711, filed Mar. 7, 2018, and U.S. Provisional Application No. 62/639,730, filed Mar. 7, 2018, which are hereby incorporated by reference in their entireties.

### BRIEF SUMMARY

The inventive embodiments provided in this Brief Summary are meant to be illustrative only and to provide an overview of selective embodiments disclosed herein. The Brief Summary, being illustrative and selective, does not limit the scope of any claim, does not provide the entire scope of inventive embodiments disclosed or contemplated herein, and should not be construed as limiting or constraining the scope of this disclosure or any claimed inventive embodiment.

In one of many aspects, provided herein is a method comprising: (a) hybridizing a nucleic acid probe to a nucleic acid sample from a human subject suspected of having or developing endometriosis; and (b) detecting a genetic variant in a panel comprising two or more genetic variants defining a minor allele listed in Table 1.

In another aspect, provided herein is a method comprising detecting one or more genetic variants defining a minor allele listed in Table 1 in genetic material from a human subject suspected of having or developing endometriosis.

In another aspect, provided herein is a method comprising: sequencing one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof to identify one or more protein damaging or loss of function variants in a human subject suspected of having or developing endometriosis; and administering an endometriosis therapy to the human subject.

In another aspect, provided herein is a method of preventing endometriosis comprising administering a hormonal therapy to a human subject having at least one genetic variant defining a minor allele listed in Table 1.

In another aspect, provided herein is a method of treating endometriosis associated infertility comprising administering an assisted reproductive therapy to a human subject having at least one genetic variant defining a minor allele listed in Table 2.

In another aspect, provided herein is a method comprising administering a pain medication to a human subject having at least one genetic variant defining a minor allele listed in Table 3.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE DRAWINGS

FIG. 1 is a set of bar charts showing distribution of predictive score using 775 rare variants among 917 endometriosis subjects and 917 controls generated through simulation using the ExAc published frequencies (All rare variants are assumed to be independent).

FIG. 2 is a boxplot of the predictive score across the clinical subtypes of endometriosis. Endoscore is uniform across the severity of endometriosis.

FIG. 3 is a pie chart showing diverse pathways implicated by these 729 genes. No pathway reaches statistical significance, but multiple genes implicated in the Wnt, cadherin, integrin, and inflammation medicated by cytokine signaling pathways.

FIG. 4 is a diagram showing three experimental design strategies. Sequencing nuclear families can help identify Mendelian segregation, whereas relative pairs can help uncover distant relationships with IBD. Unrelated individuals are typically studied to identify common variants with small effects.

FIG. 5 is a diagram showing a nuclear family with an IGF2 mutation on the left and an extended pedigree with a LONP1 mutation to the right.

FIG. 6 is a diagram of mutation patterns cis/trans/haplotypes.

FIG. 7 is a bar chart showing example of results: genes implicated in GWAS (genome-wide association studies) meta-analyses.

FIG. 8 is a set of diagrams showing striking excess of pathogenic mutations (p<10⁻¹⁶).

FIG. 9 is a set of charts showing examples of FN1 and GREB1 in which multiple damaging mutations were found.

FIG. 10 is a diagram showing a computer-based system that may be programmed or otherwise configured to implement methods provided herein.

FIG. 11 is a diagram showing a method and system as disclosed herein.

### DETAILED DESCRIPTION

Unless defined otherwise, all technical and scientific terms used herein have the same meaning as commonly understood by one of the ordinary skill in the art to which this invention belongs. Although any methods and materials similar or equivalent to those described herein can be used in the practice or testing of the compositions or unit doses herein, some methods and materials are now described. Unless mentioned otherwise, the techniques employed or contemplated herein are standard methodologies. The materials, methods and examples are illustrative only and not limiting.

The details of one or more inventive instances are set forth in the accompanying drawings, the claims, and the description herein. Other features, objects, and advantages of the inventive instances disclosed and contemplated herein can be combined with any other instance unless explicitly excluded.

In some of many aspects, the present disclosure provides methods of using genetic markers associated with endometriosis, for example via a computer-implemented program to predict risk of developing endometriosis, and methods of preventing or treating endometriosis or a symptom thereof. The methods disclosed herein can prevent or cancel an invasive procedure, such as a laparoscopy, that would otherwise have been performed on a subject but for the results, for example a (negative) diagnosis/prognosis, from the methods disclosed herein performed on the subject.

In some cases, genetic markers disclosed herein can be used for early diagnosis and prognosis of endometriosis, as well as early clinical intervention to mitigate progression of the disease. The use of these genetic markers can allow selection of subjects for clinical trials involving novel treatment methods. In some instances, genetic markers disclosed herein can be used to predict endometriosis and endometriosis progression, for example in treatment decisions for individuals who are recognized as having endometriosis. In some instances, genetic markers disclosed herein can enable prognosis of endometriosis in much larger populations compared with the populations which can currently be evaluated by using existing risk factors and biomarkers.

In some cases, disclosed herein is a method for endometriosis diagnosis/prognosis that can utilize detection of endometriosis associated biomarkers such as single nucleotide polymorphisms (SNPs), insertion deletion polymorphisms (indels), damaging mutation variants, loss of function variants, synonymous mutation variants, nonsynonymous mutation variants, nonsense mutations, recessive markers, splicing/splice-site variants, frameshift mutations, insertions, deletions, genomic rearrangements, stop-gain, stop-loss, Rare Variants (RVs), some of which are identified in Tables 1-4 (or diagnostically and predicatively functionally comparable biomarkers). In some instances, the method can comprise using a statistical assessment method such as Multi Dimensional Scaling analysis (MDS), logistic regression, or Bayesian analysis.

Some of the variants listed in Table 1 can be splicing variants, for example TMED3 (NM_007364:exon1:c.168+1G>A), NM_001276480:c.-160+1G>A, KCNK6 (NM_004823:exon2:c.323-1G>A), RGPD4 (NM_182588:exon19:c.2606-1G>T), NM_001001891:exon18:c.1988+1G>A, NM_001882:exon3:c.176-2->C. The NM number indicates that a particular GenBank cDNA reference sequence was used for reference. The “c” indicates that the nucleotide number which follows is based on coding DNA sequence. The numbers provide the position of the mutation in the DNA. For instance, 168+1G>A means one base after (+1) the 168th coding nucleotide at the end of the exon is mutated form a G to an A. Likewise for NM_182588:exon19:c.2606-1G>T, one base before (−1) the 2606th coding nucleotide. NM_001882:exon3:c.176-2→C involves an insertion of a C.

In some cases, disclosed herein is a treatment method to a subject determined to have or be predisposed to endometriosis. In some instances, the method can comprise administering to the subject a hormone therapy or an assisted reproductive therapy. In some instances, the method can comprise administering to the subject a therapy that at least partially compensates for endometriosis, prevents or reduces the severity of endometriosis that the subject would otherwise develop, or prevents endometriosis related complications, cancers, or associated disorders.

In some cases, provided herein is identification of new variants such as SNPs or indels, unique combinations of such variants, and haplotypes of variants that are associated with endometriosis and related pathologies. In some instances, the polymorphisms disclosed herein can be directly useful as targets for the design of diagnostic reagents and the development of therapeutic agents for use in the diagnosis and treatment of endometriosis and related pathologies. Based on the identification of variants associated with endometriosis, the present disclosure can provide methods of detecting these variants as well as the design and preparation of detection reagents needed to accomplish this task. Provided herein are novel variants in genetic sequences involved in endometriosis, methods of detecting these variants in a test sample, methods of identifying individuals who have an altered risk of developing endometriosis and for suggesting treatment options for endometriosis based on the presence of a variant(s) disclosed herein or its encoded product and methods of identifying individuals who are more or less likely to respond to a treatment.

In some cases, provided herein are variants such as SNPs and indels associated with endometriosis, nucleic acid molecules containing variants, methods and reagents for the detection of the variants disclosed herein, uses of these variants for the development of detection reagents, and assays or kits that utilize such reagents. In some instances, the variants disclosed herein can be useful for diagnosing, screening for, and evaluating predisposition to endometriosis and progression of endometriosis. In some instances, the variants can be useful in the determining individual subject treatment plans and design of clinical trials of devices for possible use in the treatment of endometriosis. In some instances, the variants and their encoded products can be useful targets for the development of therapeutic agents. In some instances, the variants combined with other non-genetic clinical factors can be useful for diagnosing, screening, evaluating predisposition to endometriosis, assessing risk of progression of endometriosis, determining individual subject treatment plans and design of clinical trials of devices for possible use in the treatment of endometriosis. In some instances, the variants can be useful in the selection of recipients for an oral contraceptive type therapeutic.

### Definitions

Unless otherwise indicated, open terms for example “contain,” “containing,” “include,” “including,” and the like mean comprising.

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein, following or not following the term “about,” can be at 85-115% (i.e., plus or minus 15%) of the numerical ranges and/or values.

As used herein, “endometriosis” refers to any nonmalignant disorder in which functioning endometrial tissue is present in a location in the body other than the endometrium of the uterus, i.e. outside the uterine cavity or is present within the myometrium of the uterus. For purposes herein it also includes conditions, such as adenomyosis/adenomyoma, that exhibit myometrial tissue in the lesions. Endometriosis can include endometriosis externa, endometrioma, adenomyosis, adenomyomas, adenomyotic nodules of the uterosacral ligaments, endometriotic nodules other than of the uterosacral ligaments, autoimmune endometriosis, mild endometriosis, moderate endometriosis, severe endometriosis, superficial (peritoneal) endometriosis, deep (invasive) endometriosis, ovarian endometriosis, endometriosis-related cancers, and/or “endometriosis-associated conditions”. Unless stated otherwise, the term endometriosis is used herein to describe any of these conditions.

As used herein, “treatment” includes one or more of: reducing the frequency and/or severity of symptoms, elimination of symptoms and/or their underlying cause, and improvement or remediation of damage. For example, treatment of endometriosis includes, for example, relieving the pain experienced by a woman suffering from endometriosis, and/or causing the regression or disappearance of endometriotic lesions.

“Haplotype” can mean a combination of genotypes on the same chromosome occurring in a linkage disequilibrium block. Haplotypes serve as markers for linkage disequilibrium blocks, and at the same time provide information about the arrangement of genotypes within the blocks. Typing of only certain variants which serve as tags can, therefore, reveal all genotypes for variants located within a block. Thus, the use of haplotypes greatly facilitates identification of candidate genes associated with diseases and drug sensitivity.

“Linkage disequilibrium” or “LD” can mean that a particular combination of alleles (alternative nucleotides) or genetic variants for example at two or more different SNP (or RV) sites are non-randomly co-inherited (i.e., the combination of alleles at the different SNP (or RV) sites occurs more or less frequently in a population than the separate frequencies of occurrence of each allele or the frequency of a random formation of haplotypes from alleles in a given population). The term “LD” can differ from “linkage,” which describes the association of two or more loci on a chromosome with limited recombination between them. LD can also be used to refer to any non-random genetic association between allele(s) at two or more different SNP (or RV) sites. In some instances, when a genetic marker (e.g. SNP or RV) is identified as the genetic marker associated with a disease (in this instance endometriosis), it can be the minor allele (MA) of the particular genetic marker that is associated with the disease. In some instances, if the Odds Ratio (OR) of the MA is greater than 1.0, the MA of the genetic marker (in this instance the endometriosis associated genetic marker) can be correlated with an increased risk of endometriosis in a case subject as compared to a control subject and can be considered a causative marker (C), and if the OR of the MA less than 1.0, the MA of the genetic marker can be correlated with a decreased risk of endometriosis in a case subject as compared to a control subject and can be considered a protective marker (P). “Linkage disequilibrium block” or “LD block” can mean a region of the genome that contains multiple variants located in proximity to each other and that are transmitted as a block.

Biological samples obtained from individuals (e.g., human subjects) may be any sample from which a genetic material (e.g., nucleic acid sample) may be derived. Samples/Genetic materials may be from buccal swabs, saliva, blood, hair, nail, skin, cell, or any other type of tissue sample. In some instances, the genetic material (e.g., nucleic acid sample) comprises mRNA, cDNA, genomic DNA, or PCR amplified products produced therefrom, or any combination thereof. In some instances, the genetic material (e.g., nucleic acid sample) comprises PCR amplified nucleic acids produced from cDNA or mRNA. In some instances, the genetic material (e.g., nucleic acid sample) comprises PCR amplified nucleic acids produced from genomic DNA.

Analysis of Rare and Private Mutations in Sequenced Endometriosis Genes

In some cases, the present disclosure provides an analysis to evaluate a coding region of a gene as a component of a genetic diagnostic or predictive test for endometriosis. In some instances, the analysis can comprise one or more of the approaches disclosed herein.

In some instances, the analysis can comprise performing DNA variant search on the next generation sequencing output file using a standard software designed for this purpose, for example Life Technologies TMAP algorithm with their default parameter settings, and Life Technologies Torrent Variant Caller software. ANNOVAR can be used to classify coding variants as synonymous, missense, frameshift, splicing, stop-gain, or stop-loss. Variants can be considered “loss-of-function” if the variant causes a stop-loss, stop-gain, splicing, or frame-shift insertion or deletion).

In some instances, the analysis can comprise evaluating prediction of an effect of each variant on protein function in silico using a variety of different software algorithms: Polyphen 2, Sift, Mutation Accessor, Mutation Taster, FATHMM, LRT, MetaLR, or any combination thereof. Missense variants can be deemed “damaging” if they are predicted to be damaging by at least one of the seven algorithms tested.

In some instances, the analysis can comprise searching population databases (e.g., gnomAD) and proprietary endometriosis allele frequency databases for the prevalence of any loss of function or damaging mutations identified by these analyses. The log of the odds ratio can be used to weight the marker when the variant has been previously observed in the reference databases. When a damaging variant or loss of function variant has never been reported in the reference databases, a default odds ratio of 10 can be used to weight the finding.

In some instances, the analysis can comprise incorporating findings into the Risk Score as with the other low-frequency alleles. Risk Score=Summation [log(OR)×Count], where count equals the number of low frequency alleles detected at each endometriosis associated locus. Risk scores can be converted to probability using a nomogram based on confirmed diagnoses.

In some instances, the methods of the present disclosure can provide a high sensitivity of detecting gene mutations and diagnosing endometriosis that is greater than 60%, 65%, 70%, 75%, 80%, 85%, 90%, 91%, 92%, 93%, 94%, 95%, 95.5%, 96%, 96.5%, 97%, 97.5%, 98%, 98.5%, 99%, 99.5% or more. In some instances, the methods disclosed herein can provide a high specificity of detecting and classifying gene mutations and endometriosis, for example, greater than 80%, 85%, 90%, 91%, 92%, 93%, 94%, 95%, 95.5%, 96%, 96.5%, 97%, 97.5%, 98%, 98.5%, 99%, 99.5% or more. In some instances, a nominal specificity for the method disclosed herein can be greater than or equal to 70%. In some instances, a nominal Negative Predictive Value (NPV) for the method disclosed herein can be greater than or equal to 95%. In some instances, a NPV for the method disclosed herein can be about 95%, 95.5%, 96%, 96.5%, 97%, 97.5%, 98%, 98.5%, 99%, 99.5% or more. In some instances, a nominal Positive Predictive Value (PPV) for the method disclosed herein can be greater than or equal to 95%. In some instances, a PPV for the method disclosed herein can be about 95%, 95.5%, 96%, 96.5%, 97%, 97.5%, 98%, 98.5%, 99%, 99.5% or more. In some instances, the accuracy of the methods disclosed herein in diagnosing endometriosis can be greater than 70%, 75%, 80%, 85%, 90%, 91%, 92%, 93%, 94%, 95%, 95.5%, 96%, 96.5%, 97%, 97.5%, 98%, 98.5%, 99%, 99.5% or more.

Computer Implemented Methods

In some aspects, the present disclosure provides methods for analysis of gene sequence data associated software and computer systems. The method, for example being computer implemented, can enable a clinical geneticist or other healthcare technician to sift through vast amounts of gene sequence data, to identify potential disease-causing genomic variants. In some cases, the gene sequence data is from a patient who may be suspected of having a genetic disorder such as endometriosis.

In some cases, provided herein is a method for identifying a genetic disorder such as endometriosis or predicting a risk thereof in an individual, or identifying a genetic variant that is causative of a phenotype in an individual. In some instances, the method can comprise determining gene sequence for a patient suspected of having a genetic disorder, identifying sequence variants, annotating the identified variants based on one or more criteria, and filtering or searching the variants at least partially based on the annotations, to thereby identify potential disease-causing variants.

In some instances, the gene sequence is obtained by use of a sequencing instrument, or alternatively, gene sequence data is obtained from another source, such as for example, a commercial sequencing service provider. Gene sequence can be chromosomal sequence, cDNA sequence, or any nucleotide sequence information that allows for detection of genetic disease. Generally, the amount of sequence information is such that computational tools are required for data analysis. For example, the sequence data may represent at least half of the individual's genomic or cDNA sequence (e.g., of a representative cell population or tissue), or the individuals entire genomic or cDNA sequence. In various embodiments, the sequence data comprises the nucleotide sequence for at least 1 million base pairs, at least 10 million base pairs, or at least 50 million base pairs. In certain embodiments, the DNA sequence is the individual's exome sequence or full exonic sequence component (i.e., the exome; sequence for each of the exons in each of the known genes in the entire genome). In some embodiments, the source of genomic DNA or cDNA may be any suitable source, and may be a sample particularly indicative of a disease or phenotype of interest, including blood cells (e.g, PBMCs, or a T-cell or B-cell population). In certain embodiments, the source of the sample is a tissue or sample that is potentially malignant.

In some instances, whole genome sequence can comprise the entire sequence (including all chromosomes) of an individual's germline genome. In some embodiments, the concatenated length for a whole genome sequence is approximately 3.2 Gbases or 3.2 billion nucleotides.

In some instances, the gene sequence may be determined by any suitable method. For example, the gene sequence may be a cDNA sequence determined by clonal amplification (e.g., emulsion PCR) and sequencing. Base calling may be conducted based on any available method, including Sanger sequencing (chain termination), pH sequencing, pyrosequencing, sequencing-by-hybridization, sequencing-by-ligation, etc. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system. 10052 In some instances, the gene sequence may be mapped with one or more reference sequences to identify sequence variants. For example, the base reads are mapped against a reference sequence, which in various embodiments is presumed to be a “normal” non-disease sequence. The DNS sequence derived from the Human Genome Project is generally used as a “premier” reference sequence. A number of mapping applications are known, and include TMAP, BWA, GSMAPPER, ELAND, MOSAIK, and MAQ. Various other alignment tools are known, and could also be implemented to map the base reads.

In some cases, based on the sequence alignments, and mapping results, sequence variants can be identified. Types of variants may include insertions, deletions, indels (a colocalized insertion and deletion), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions. While the type of variants analyzed is not limited, the most numerous of the variant types will be single nucleotide substitutions, for which a wealth of data is currently available. In various embodiments, comparison of the test sequence with the reference sequence will produce at least 500 variants, at least 1000 variants, at least 3,000 variants, at least 5,000 variants, at least 10,000 variants, at least 20,000 variants, or at least 50,000 variants, but in some embodiments, will produce at least 1 million variants, at least 2 million variants, at least 3 million variants, at least 4 million variants, or at least 10 million variants. The tools provided herein enable the user to navigate the vast amounts of genetic data to identify potentially disease-causing variants.

In some cases, a wealth of data can be extracted for the identified variants, including one or more of conservation scores, genic/genomic location, zygosity, SNP ID, Polyphen, FATHMM, LRT, Mutation Accessor, and SIFT predictions, splice site predictions, amino acid properties, disease associations, annotations for known variants, variant or allele frequency data, and gene annotations. Data may be calculated and/or extracted from one or more internal or external databases. Since certain categories of annotations (e.g., amino acid properties/PolyPhen and SIFT data) are dependent on a nature of the region of the genome in which they are contained (e.g., whether a variant is contained within a region translated to give rise to an amino acid sequence in a resultant protein), these annotations can be carried out for each known transcript. Exemplary external databases include OMIM (Online Mendelian Inheritance in Man), HGMD (The Human Gene Mutation Databse), PubMed, PolyPhen, SIFT, SpliceSite, reference genome databases, the University of California Santa Cruz (UCSC) genome database, CLINVAR database, the BioBase biological databases, the dbSNP Short Genetic Variations database, the Rat Genome Database (RGD), and/or the like. Various other databases may be employed for extracting data on identified variants. Variant information may be further stored in a central data repository, and the data extracted for future sequence analyses.

In some instances, variants may be tagged by the user with additional descriptive information to aid subsequent analysis. For example, confidence in the existence of the variant can be recorded as confirmed, preliminary, or sequence artifact. Certain sequencing technologies have a tendency to produce certain types of sequence artifacts, and the method herein can allow such suspected artifacts to be recorded. The variants may be further tagged in basic categories of benign, pathogenic, or unknown, or as potentially of interest.

In some instances, queries can be run to identify variants meeting certain criteria, or variant report pages can be browsed by chromosomal position or by gene, the latter allowing researchers to focus on only those variations that exist in a particular set of genes of interest. In some embodiments, the user selects only variants with well-documented and published disease associations (e.g., by filtering based on HGMD or other disease annotation). Alternatively, the user can filter for variants not previously associated with disease, but of a type likely to be deleterious, such as those introducing frameshifts, non-synonymous substitutions (predicted by Polyphen or SIFT), or premature terminations. Further, the user can exclude from analysis those variants believed to be neutral (based on their frequency of occurrence in studies populations), for example, through exclusion of variants in dbSNP. Additional exclusion criteria include mode of inheritance (e.g., heterozygosity), depth of coverage, and quality score.

In certain embodiments, base calling is carried out to extract the sequence of the sequencing reads from an image file produced by an instrument scanner. Following base calling and base quality trimming/filtering, the reads are mapped against a reference sequence (assumed to be normal for the phenotype under analysis) to identify variations (variants) between the two with the assumption that one or more of these differences will be associated with phenotype of the individual whose DNA is under analysis. Subsequently, each variant is annotated with data that can be used to determine the likelihood that that particular variant is associated with the phenotype under analysis. The analysis may be fully or partially automated as described in detail below, and may include use of a central repository for data storage and analysis, and to present the data to analysts and clinical geneticists in a format that makes identification of variants with a high likelihood of being associated with the phenotypic difference more efficient and effective.

In some embodiments, a user can be provided with the ability to run cross sample queries where the variants from multiple samples are interrogated simultaneously. In such embodiments, for example, a user can build a query to return data on only those variants that are exactly shared across a user defined group of samples. This can be useful for family based analyses where the same variant is believed to be associated with disease in each of the affected family members. For another example, the user can also build a query to return only those variants that are present in genes where the gene contains at least one, but not necessarily the same, variant. This can be useful where a group of individuals with disease are not related (the variants associated with the disease are not necessary exactly the same, but result in a common alteration in normal function). For yet another example, the user can specify to ignore genes containing variants in a user defined group of samples. This can be useful to exclude polymorphisms (variants believed or confirmed not to be associated with disease) where the user has access to a user defined group of control individuals who are believed to not have the disease associated variant. For each of these queries a user can additionally filter the variants by specifying any or all of the previously discussed filters on top of the cross sample analyses. This allows a user to identify variants matching these criteria, which are shared between or segregated amongst samples.

For example, a variant analysis system can be implemented locally, or implemented using a host device and a network or cloud computing. For example, the variant analysis system can be software stored in memory of a personal computing device (PC) and implemented by a processor of the PC. In such embodiments, for example, the PC can download the software from a host device and/or install the software using any suitable device such as a compact disc (CD).

The method may employ a computer-readable medium, or non-transitory processor-readable medium. Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code can include, but are not limited to, micro-code or micro-instructions, machine instructions, such as produced by a compiler, code used to produce a web service, and files containing higher-level instructions that are executed by a computer using an interpreter. For example, embodiments may be implemented using Python, Java, C++, or other programming languages (e.g., object-oriented programming languages) and development tools. Additional examples of computer code can include, but are not limited to, control signals, encrypted code, and compressed code.

In some cases, variants provided herein may be “provided” in a variety of mediums to facilitate use thereof. As used in this section, “provided” refers to a manufacture, other than an isolated nucleic acid molecule, that contains variant information of the present disclosure. Such a manufacture provides the variant information in a form that allows a skilled artisan to examine the manufacture using means not directly applicable to examining the variants or a subset thereof as they exist in nature or in purified form. The variant information that may be provided in such a form includes any of the variant information provided by the present disclosure such as, for example, polymorphic nucleic acid and/or amino acid sequence information, information about observed variant alleles, alternative codons, populations, allele frequencies, variant types, and/or affected proteins, or any other information provided herein.

In some instances, the variants can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence of the present disclosure. One such medium is provided with the present application, namely, the present application contains computer readable medium (CD-R) that has nucleic acid sequences (and encoded protein sequences) containing variants provided/recorded thereon in ASCII text format in a Sequence Listing along with accompanying Tables that contain detailed variant and sequence information.

As used herein, “recorded” can refer to a process for storing information on computer readable medium. A skilled artisan can readily adopt any of the presently known methods for recording information on computer readable medium to generate manufactures comprising the variant information of the present disclosure. A variety of data storage structures are available to a skilled artisan for creating a computer readable medium having recorded thereon a nucleotide or amino acid sequence of the present disclosure. The choice of the data storage structure will generally be based on the means chosen to access the stored information. In addition, a variety of data processor programs and formats can be used to store the nucleotide/amino acid sequence information of the present disclosure on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the variant information of the present disclosure.

By providing the variants in computer readable form, a skilled artisan can access the variant information for a variety of purposes. Computer software is publicly available which allows a skilled artisan to access sequence information provided in a computer readable medium. Examples of publicly available computer software include BLAST and BLAZE search algorithms.

In some cases, the present disclosure can provide systems, particularly computer-based systems, which contain the variant information described herein. Such systems may be designed to store and/or analyze information on, for example, a large number of variant positions, or information on variant genotypes from a large number of individuals. The variant information of the present disclosure represents a valuable information source. The variant information of the present disclosure stored/analyzed in a computer-based system may be used for such computer-intensive applications as determining or analyzing variant allele frequencies in a population, mapping endometriosis genes, genotype-phenotype association studies, grouping variants into haplotypes, correlating variant haplotypes with response to particular treatments or for various other bioinformatic, pharmacogenomic or drug development.

As used herein, “a computer-based system” can refer to the hardware means, software means, and data storage means used to analyze the variant information of the present disclosure. The minimum hardware means of the computer-based systems of the present disclosure typically comprises a central processing unit (CPU), input means, output means, and data storage means. A skilled artisan can readily appreciate that any one of the currently available computer-based systems are suitable for use in the present disclosure. Such a system can be changed into a system of the present disclosure by utilizing the variant information provided on the CD-R, or a subset thereof, without any experimentation.

As stated above, the computer-based systems can comprise a data storage means having stored therein variants of the present disclosure and the necessary hardware means and software means for supporting and implementing a search means. As used herein, “data storage means” refers to memory which can store variant information of the present disclosure, or a memory access means which can access manufactures having recorded thereon the variant information of the present disclosure.

As used herein, “search means” can refer to one or more programs or algorithms that are implemented on the computer-based system to identify or analyze variants in a target sequence based on the variant information stored within the data storage means. Search means can be used to determine which nucleotide is present at a particular variant position in the target sequence. As used herein, a “target sequence” can be any DNA sequence containing the variant position(s) to be searched or queried.

A variety of structural formats for the input and output means can be used to input and output the information in the computer-based systems of the present disclosure. An exemplary format for an output means is a display that depicts the presence or absence of specified nucleotides (alleles) at particular variant positions of interest. Such presentation can provide a rapid, binary scoring system for many variants simultaneously.

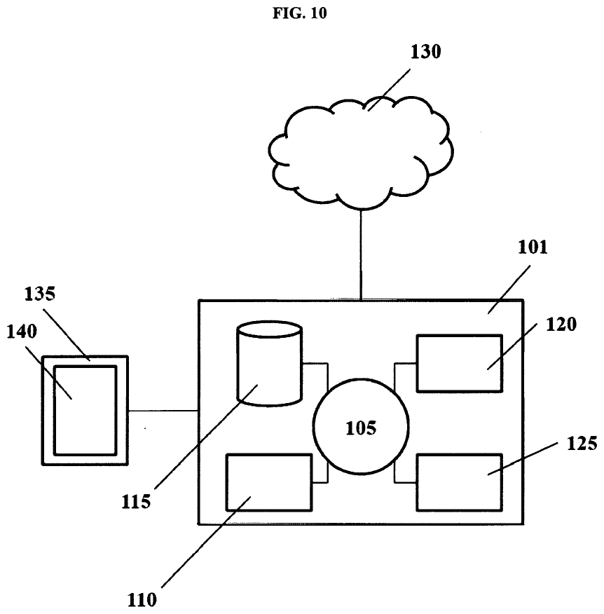

In some cases, the present disclosure provides computer-based systems that are programmed to implement methods of the disclosure. FIG. 10 shows a computer system 101 that can be programmed or configured for endometriosis diagnosis. The computer system 101 can regulate various aspects of detection of genetic variants associated with endometriosis of the present disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a monitor. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, Polyphen 2, Sift, Mutation Accessor, Mutation Taster, FATHMM, LRT, MetaLR, or any combination thereof.

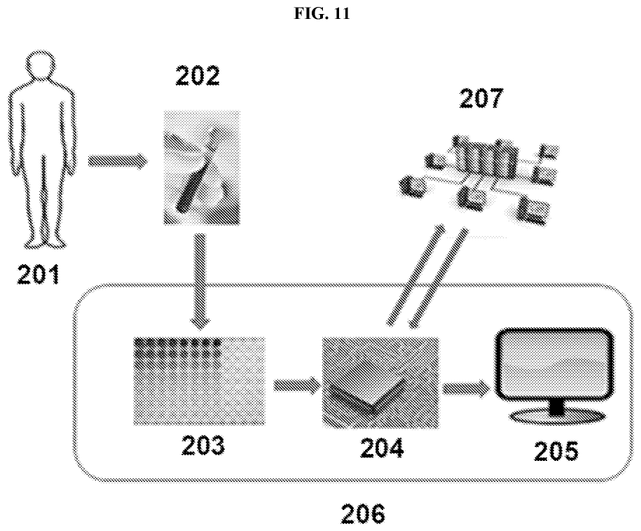

In some cases, as shown in FIG. 11, a sample 202 containing a genetic material may be obtained from a subject 201, such as a human subject. A sample 202 may be subjected to one or more methods as described herein, such as performing an assay. In some cases, an assay may comprise hybridization, amplification, sequencing, labeling, epigenetically modifying a base, or any combination thereof. One or more results from a method may be input into a processor 204. One or more input parameters such as a sample identification, subject identification, sample type, a reference, or other information may be input into a processor 204. One or more metrics from an assay may be input into a processor 204 such that the processor may produce a result, such as a diagnosis of endometriosis or a recommendation for a treatment. A processor may send a result, an input parameter, a metric, a reference, or any combination thereof to a display 205, such as a visual display or graphical user interface. A processor 204 may (i) send a result, an input parameter, a metric, or any combination thereof to a server 207, (ii) receive a result, an input parameter, a metric, or any combination thereof from a server 207, (iii) or a combination thereof.

Methods of Detection of Variants

In some aspects, the present disclosure provides methods to detect variants, e.g, detecting a genetic variant in a panel comprising two or more genetic variants defining a minor allele disclosed herein (e.g., in Table 1). In some instances, the detecting comprises, DNA sequencing, hybridization with a complementary probe, an oligonucleotide ligation assay, a PCR-based assay, or any combination thereof. In some instances, the panel comprises at least: 2, 3, 4, 5, 6, 7, 8, 9, 10, 15, 20, 25, 30, 35, 40, 45, 50, 60, 70, 75, 80, 90, 100, 150, 200, 250, 300, 350, 400, 450, 500, or more genetic variants defining minor alleles disclosed herein (e.g., in Table 1). In some instances, the genetic variant to detect or detected has an odds ratio (OR) of at least: 0.1, 1, 1.5, 2, 5, 10, 20, 50, 100, 127, 130, 140, 150, 200, 300, 400, 500, 600, 700, 800, 900, 1000, 1500, 2000, 2500, 3000, 3500, 4000, 4500, 5000, or more. In some embodiments, the OR is at least 127. In some instances, the panel to detect further comprises one or more protein damaging or loss of function variants in one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof. In some instances, the panel further comprises one or more additional variants defining a minor allele listed in Table 4.

In some cases, variants of the present disclosure may include single nucleotide polymorphisms (SNPs), insertion deletion polymorphisms (indels), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions.

Variants for example SNPs are usually preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP is an amino acid “coding” sequence. A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa.

A synonymous codon change, or silent mutation is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. An indel that occur in a coding DNA segment gives rise to a frameshift mutation.

Causative variants are those that produce alterations in gene expression or in the structure and/or function of a gene product, and therefore are predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic endometriosis.

An association study of a variant and a specific disorder involves determining the presence or frequency of the variant allele in biological samples from individuals with the disorder of interest, such as endometriosis, and comparing the information to that of controls (i.e., individuals who do not have the disorder; controls may be also referred to as “healthy” or “normal” individuals) who are for example of similar age and race. The appropriate selection of patients and controls is important to the success of variant association studies. Therefore, a pool of individuals with well-characterized phenotypes is extremely desirable.

A variant may be screened in tissue samples or any biological sample obtained from an affected individual, and compared to control samples, and selected for its increased (or decreased) occurrence in a specific pathological condition, such as pathologies related to endometriosis. Once a statistically significant association is established between one or more variant(s) and a pathological condition (or other phenotype) of interest, then the region around the variant can optionally be thoroughly screened to identify the causative genetic locus/sequence(s) (e.g., causative variant/mutation, gene, regulatory region, etc.) that influences the pathological condition or phenotype. Association studies may be conducted within the general population and are not limited to studies performed on related individuals in affected families (linkage studies). For diagnostic and prognostic purposes, if a particular variant site is found to be useful for diagnosing a disease, such as endometriosis, other variant sites which are in LD with this variant site would also be expected to be useful for diagnosing the condition. Linkage disequilibrium is described in the human genome as blocks of variants along a chromosome segment that do not segregate independently (i.e., that are non-randomly co-inherited). The starting (5′ end) and ending (3′ end) of these blocks can vary depending on the criteria used for linkage disequilibrium in a given database, such as the value of D′ or r² used to determine linkage disequilibrium.

In some instances, variants can be identified in a study using a whole-genome case-control approach to identify single nucleotide polymorphisms that were closely associated with the development of endometriosis, as well as variants found to be in linkage disequilibrium with (i.e., within the same linkage disequilibrium block as) the endometriosis-associated variants, which can provide haplotypes (i.e., groups of variants that are co-inherited) to be readily inferred. Thus, the present disclosure provides individual variants associated with endometriosis, as well as combinations of variants and haplotypes in genetic regions associated with endometriosis, methods of detecting these polymorphisms in a test sample, methods of determining the risk of an individual of having or developing endometriosis and for clinical sub-classification of endometriosis.

In some cases, the present disclosure provides variants associated with endometriosis, as well as variants that were previously known in the art, but were not previously known to be associated with endometriosis. Accordingly, the present disclosure provides novel compositions and methods based on the variants disclosed herein, and also provides novel methods of using the known but previously unassociated variants in methods relating to endometriosis (e.g., for diagnosing endometriosis. etc.).

In some instances, particular variant alleles of the present disclosure can be associated with either an increased risk of having or developing endometriosis, or a decreased risk of having or developing endometriosis. Variant alleles that are associated with a decreased risk may be referred to as “protective” alleles, and variant alleles that are associated with an increased risk may be referred to as “susceptibility” alleles, “risk factors”, or “high-risk” alleles. Thus, whereas certain variants can be assayed to determine whether an individual possesses a variant allele that is indicative of an increased risk of having or developing endometriosis (i.e., a susceptibility allele), other variants can be assayed to determine whether an individual possesses a variant allele that is indicative of a decreased risk of having or developing endometriosis (i.e., a protective allele). Similarly, particular variant alleles of the present disclosure can be associated with either an increased or decreased likelihood of responding to a particular treatment. The term “altered” may be used herein to encompass either of these two possibilities (e.g., an increased or a decreased risk/likelihood).

In some instances, nucleic acid molecules may be double-stranded molecules and that reference to a particular site on one strand refers, as well, to the corresponding site on a complementary strand. In defining a variant position, variant allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular variant position, variant allele, or nucleotide sequence. Probes and primers may be designed to hybridize to either strand and variant genotyping methods disclosed herein may generally target either strand. Throughout the specification, in identifying a variant position, reference is generally made to the forward or “sense” strand, solely for the purpose of convenience. Since endogenous nucleic acid sequences exist in the form of a double helix (a duplex comprising two complementary nucleic acid strands), it is understood that the variants disclosed herein will have counterpart nucleic acid sequences and variants associated with the complementary “reverse” or “antisense” nucleic acid strand. Such complementary nucleic acid sequences, and the complementary variants present in those sequences, are also included within the scope of the present disclosure.

Genotyping Methods

In some cases, the process of determining which specific nucleotide (i.e., allele) is present at each of one or more variant positions, such as a variant position in a nucleic acid molecule characterized by a variant, is referred to as variant genotyping. The present disclosure provides methods of variant genotyping, such as for use in screening for endometriosis or related pathologies, or determining predisposition thereto, or determining responsiveness to a form of treatment, or in genome mapping or variant association analysis, etc.

Nucleic acid samples can be genotyped to determine which allele(s) is/are present at any given genetic region (e.g., variant position) of interest by methods well known in the art. The neighboring sequence can be used to design variant detection reagents such as oligonucleotide probes, which may optionally be implemented in a kit format. Common variant genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms can include, but are not limited to, methods in which protection from cleavage agents is used to detect mismatched bases in RNA/RNA or RNA/DNA duplexes, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis (DGGE). Sequence variations at specific locations can also be assessed by nuclease protection assays such as RNase and SI protection or chemical cleavage methods.

In some instances, a variant genotyping can be performed using the TaqMan assay, which is also known as the 5′ nuclease assay. The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced. During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target variant-containing template which is amplified during PCR, and the probe is designed to hybridize to the target variant site only if a particular variant allele is present. TaqMan primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the present disclosure are useful in diagnostic assays for endometriosis and related pathologies, and can be readily incorporated into a kit format. The present disclosure also includes modifications of the Taqman assay well known in the art such as the use of Molecular Beacon probes and other variant formats.

In some instances, a method for genotyping the variants can be the use of two oligonucleotide probes in an OLA. In this method, one probe hybridizes to a segment of a target nucleic acid with its 3′ most end aligned with the variant site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the variant site. If there is a mismatch, ligation would not occur. After the reaction, the ligated probes are separated from the target nucleic acid molecule, and detected as indicators of the presence of a variant.

In some instances, a method for variant genotyping is based on mass spectrometry. Mass spectrometry takes advantage of the unique mass of each of the four nucleotides of DNA. variants can be unambiguously genotyped by mass spectrometry by measuring the differences in the mass of nucleic acids having alternative variant alleles. MALDI-TOF (Matrix Assisted Laser Desorption Ionization-Time of Flight) mass spectrometry technology is exemplary for extremely precise determinations of molecular mass, such as variants. Numerous approaches to variant analysis have been developed based on mass spectrometry. Exemplary mass spectrometry-based methods of variant genotyping include primer extension assays, which can also be utilized in combination with other approaches, such as traditional gel-based formats and microarrays.

In some instances, a method for genotyping the variants of the present disclosure is the use of electrospray mass spectrometry for direct analysis of an amplified nucleic acid. In this method, in one aspect, an amplified nucleic acid product may be isotopically enriched in an isotope of oxygen (O), carbon (C), nitrogen (N) or any combination of those elements. In an exemplary embodiment the amplified nucleic acid is isotopically enriched to a level of greater than 99.9% in the elements of O¹⁶, C¹² and N¹⁴ The amplified isotopically enriched product can then be analyzed by electrospray mass spectrometry to determine the nucleic acid composition and the corresponding variant genotyping. Isotopically enriched amplified products result in a corresponding increase in sensitivity and accuracy in the mass spectrum. In another aspect of this method an amplified nucleic acid that is not isotopically enriched can also have composition and variant genotype determined by electrospray mass spectrometry.

In some instances, variants can be scored by direct DNA sequencing. The nucleic acid sequences of the present disclosure enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), is commonly used in the art for automated sequencing.

Variant genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target variant under conditions such that hybridization and amplification of the target nucleic acid region occurs, and determining the nucleotide present at the variant position of interest, or, in some assays, detecting the presence or absence of an amplification product (assays can be designed so that hybridization and/or amplification will only occur if a particular variant allele is present or absent). In some assays, the size of the amplification product is detected and compared to the length of a control sample; for example, deletions and insertions can be detected by a change in size of the amplified product compared to a normal genotype.

In some instances, a variant genotyping can be used in applications that include, but are not limited to, variant-endometriosis association analysis, endometriosis predisposition screening, endometriosis diagnosis, endometriosis prognosis, endometriosis progression monitoring, determining therapeutic strategies based on an individual's genotype, and stratifying a patient population for clinical trials for a treatment such as minimally invasive device for the treatment of endometriosis.

Analysis of Genetic Association Between Variants and Phenotypic Traits

In some cases, genotyping for endometriosis diagnosis, endometriosis predisposition screening, endometriosis prognosis and endometriosis treatment and other uses described herein, can rely on initially establishing a genetic association between one or more specific variants and the particular phenotypic traits of interest.

In some instances, in a genetic association study, the cause of interest to be tested is a certain allele or a variant or a combination of alleles or a haplotype from several variants. Thus, tissue specimens (e.g., saliva) from the sampled individuals may be collected and genomic DNA genotyped for the variant(s) of interest. In addition to the phenotypic trait of interest, other information such as demographic (e.g., age, gender, ethnicity, etc.), clinical, and environmental information that may influence the outcome of the trait can be collected to further characterize and define the sample set. Specifically, in an endometriosis genetic association study, clinical information such as body mass index, age and diet may be collected. In many cases, these factors are known to be associated with diseases and/or variant allele frequencies. There are likely gene-environment and/or gene-gene interactions as well. Analysis methods to address gene-environment and gene-gene interactions (for example, the effects of the presence of both susceptibility alleles at two different genes can be greater than the effects of the individual alleles at two genes combined) are discussed below.

In some instances, after all the relevant phenotypic and genotypic information has been obtained, statistical analyses are carried out to determine if there is any significant correlation between the presence of an allele or a genotype with the phenotypic characteristics of an individual. For example, data inspection and cleaning are first performed before carrying out statistical tests for genetic association. Epidemiological and clinical data of the samples can be summarized by descriptive statistics with tables and graphs. Data validation is for example performed to check for data completion, inconsistent entries, and outliers. Chi-squared tests may then be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium (HWE) in both cases and controls for individual markers can be indicative of genotyping errors. If HWE is violated in a majority of markers, it is indicative of population substructure that should be further investigated. Moreover, Hardy-Weinberg disequilibrium in cases only can indicate genetic association of the markers with the disease of interest.

In some instances, to test whether an allele of a single variant is associated with the case or control status of a phenotypic trait, one skilled in the art can compare allele frequencies in cases and controls. Standard chi-squared tests and Fisher exact tests can be carried out on a 2.times.2 table (2 variant alleles.times.2 outcomes in the categorical trait of interest). To test whether genotypes of a variant are associated, chi-squared tests can be carried out on a 3.times.2 table (3 genotypes.times.2 outcomes). Score tests are also carried out for genotypic association to contrast the three genotypic frequencies (major homozygotes, heterozygotes and minor homozygotes) in cases and controls, and to look for trends using 3 different modes of inheritance, namely dominant (with contrast coefficients 2, −1, −1), additive (with contrast coefficients 1, 0, −1) and recessive (with contrast coefficients 1, 1, −2). Odds ratios for minor versus major alleles, and odds ratios for heterozygote and homozygote variants versus the wild type genotypes are calculated with the desired confidence limits, usually 95%. In the present study a software algorithm, PLINK, has been applied to automate the calculation of Hardy-Weinberg equilibrium, chi-square, p-values and odds-ratios for very large numbers of variants and Case-Control individuals simultaneously.

In some instances, in order to control for confounding effects and to test for interactions a stepwise multiple logistic regression analysis using statistical packages such as SAS or R may be performed. Logistic regression is a model-building technique in which the best fitting and most parsimonious model is built to describe the relation between the dichotomous outcome (for instance, getting a certain endometriosis or not) and a set of independent variables (for instance, genotypes of different associated genes, and the associated demographic and environmental factors). The most common model is one in which the logit transformation of the odds ratios is expressed as a linear combination of the variables (main effects) and their cross-product terms (interactions). To test whether a certain variable or interaction is significantly associated with the outcome, coefficients in the model are first estimated and then tested for statistical significance of their departure from zero.

In some instances, in addition to performing association tests one marker at a time, haplotype association analysis may also be performed to study a number of markers that are closely linked together. Haplotype association tests can have better power than genotypic or allelic association tests when the tested markers are not the disease-causing mutations themselves but are in linkage disequilibrium with such mutations. The test will even be more powerful if the endometriosis is indeed caused by a combination of alleles on a haplotype. In order to perform haplotype association effectively, marker-marker linkage disequilibrium measures, both D′ and r², are typically calculated for the markers within a gene to elucidate the haplotype structure. Variants within a gene can be organized in block pattern, and a high degree of linkage disequilibrium exists within blocks and very little linkage disequilibrium exists between blocks. Haplotype association with the endometriosis status can be performed using such blocks once they have been elucidated.

Haplotype association tests can be carried out in a similar fashion as the allelic and genotypic association tests. Each haplotype in a gene is analogous to an allele in a multi-allelic marker. One skilled in the art can either compare the haplotype frequencies in cases and controls or test genetic association with different pairs of haplotypes. Score tests can be done on haplotypes using the program “haplo.score”. In that method, haplotypes are first inferred by EM algorithm and score tests are carried out with a generalized linear model (GLM) framework that allows the adjustment of other factors.

In some instances, an important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0.1 (a significance level on the lenient side) may be used for generating hypotheses for significant association of a variant with certain phenotypic characteristics of a endometriosis. It is exemplary that a p-value <0.05 (a significance level traditionally used in the art) is achieved in order for a variant to be considered to have an association with a endometriosis. It is more exemplary that a p-value <0.01 (a significance level on the stringent side) is achieved for an association to be declared. Permutation tests to control for the false discovery rates, FDR, can further be employed. Such methods to control for multiplicity would be exemplary when the tests are dependent and controlling for false discovery rates is sufficient as opposed to controlling for the experiment-wise error rates.

In some instances, since both genotyping and endometriosis status classification can involve errors, sensitivity analyses may be performed to see how odds ratios and p-values would change upon various estimates on genotyping and endometriosis classification error rates.

Once individual risk factors, genetic or non-genetic, have been found for the predisposition to endometriosis, the next step can be to set up a classification/prediction scheme to predict the category (for instance, endometriosis or no endometriosis) that an individual will be in depending on his genotypes of associated variants and other non-genetic risk factors. Logistic regression for discrete trait and linear regression for continuous trait are standard techniques for such tasks. Moreover, other techniques can also be used for setting up classification. Such techniques include, but are not limited to, MART, CART, neural network, and discriminant analyses that are suitable for use in comparing the performance of different methods.

Endometriosis Diagnosis and Predisposition Screening

In some cases, information on association/correlation between genotypes and endometriosis-related phenotypes can be exploited in several ways. For example, in the case of a highly statistically significant association between one or more variants with predisposition to a disease for which treatment is available, detection of such a genotype pattern in an individual may justify particular treatment, or at least the institution of regular monitoring of the individual. In the case of a weaker but still statistically significant association between a variant and a human disease, immediate therapeutic intervention or monitoring may not be justified after detecting the susceptibility allele or variant.

The variants disclosed herein may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single variant may affect more than one phenotypic trait. Likewise, a single phenotypic trait may be affected by multiple variants in different genes.

The variants disclosed herein may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single variant may affect more than one phenotypic trait. Likewise, a single phenotypic trait may be affected by multiple variants in different genes.

Haplotypes can be particularly useful in that, for example, fewer variants can be genotyped to determine if a particular genomic region harbors a locus that influences a particular phenotype, such as in linkage disequilibrium-based variant association analysis.

Linkage disequilibrium (LD) can refer to the co-inheritance of alleles (e.g., alternative nucleotides) at two or more different variant sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different variant sites, which is generally due to the physical proximity of the two loci along a chromosome. LD can occur when two or more variants sites are in close physical proximity to each other on a given chromosome and therefore alleles at these variant sites will tend to remain unseparated for multiple generations with the consequence that a particular nucleotide (allele) at one variant site will show a non-random association with a particular nucleotide (allele) at a different variant site located nearby. Hence, genotyping one of the variant sites will give almost the same information as genotyping the other variant site that is in LD.

For diagnostic purposes, if a particular variant site is found to be useful for diagnosing endometriosis, then the skilled artisan would recognize that other variant sites which are in LD with this variant site would also be useful for diagnosing the condition. Various degrees of LD can be encountered between two or more variants with the result being that some variants are more closely associated (i.e., in stronger LD) than others. Furthermore, the physical distance over which LD extends along a chromosome differs between different regions of the genome, and therefore the degree of physical separation between two or more variant sites necessary for LD to occur can differ between different regions of the genome.

For diagnostic applications, polymorphisms (e.g., variants and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., endometriosis) that is influenced by the causative variant(s). Thus, polymorphic markers that are in LD with causative polymorphisms are useful as diagnostic markers, and are particularly useful when the actual causative polymorphism(s) is/are unknown.

The contribution or association of particular variants and/or variant haplotypes with endometriosis phenotypes, such as endometriosis, can enable the variants of the present disclosure to be used to develop superior diagnostic tests capable of identifying individuals who express a detectable trait, such as endometriosis. as the result of a specific genotype, or individuals whose genotype places them at an increased or decreased risk of developing a detectable trait at a subsequent time as compared to individuals who do not have that genotype. As described herein, diagnostics may be based on a single variant or a group of variants. In some instances, combined detection of a plurality of variations, for example about 2, 3, 4, 5, 6, 7, 8, 9, 10, 11, 12, 13, 14, 15, 16, 17, 18, 19, 20, 24, 25, 30, 32, 35, 40, 45, 48, 50, 55, 60, 64, 70, 75, 80, 85, 80, 96, 100, or any other number in-between, or more, of the variants provided herein can increase the probability of an accurate diagnosis. To further increase the accuracy of diagnosis or predisposition screening, analysis of the variants of the present disclosure can be combined with that of other polymorphisms or other risk factors of endometriosis, such as gender and age.

In some instances, the method herein can indicate a certain increased (or decreased) degree or likelihood of developing the endometriosis based on statistically significant association results. This information can be valuable to initiate earlier preventive treatments or to allow an individual carrying one or more significant variants or variant haplotypes to regularly scheduled physical exams to monitor for the appearance or change of their endometriosis in order to identify and begin treatment of the endometriosis at an early stage.

The diagnostic techniques herein may employ a variety of methodologies to determine whether a test subject has a variant or a variant pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the disclosure may be any detectable trait that is commonly observed in pathologies and disorders related to endometriosis.

Another aspect of the present disclosure relates to a method of determining whether an individual is at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more variant positions, wherein the assayed nucleotide(s) is/are indicative of an increased or decreased risk of developing the trait or indicative that the individual expresses the trait as a result of possessing a particular trait-causing or trait-influencing allele.

The variants herein can be used to identify novel therapeutic targets for endometriosis. For example, genes containing the disease-associated variants (“variant genes”) or their products, as well as genes or their products that are directly or indirectly regulated by or interacting with these variant genes or their products, can be targeted for the development of therapeutics that, for example, treat the endometriosis or prevent or delay endometriosis onset. The therapeutics may be composed of, for example, small molecules, proteins, protein fragments or peptides, antibodies, nucleic acids, or their derivatives or mimetics which modulate the functions or levels of the target genes or gene products.

The variants/haplotypes herein can be useful for improving many different aspects of the drug development process. For example, individuals can be selected for clinical trials based on their variant genotype. Individuals with variant genotypes that indicate that they are most likely to respond to or most likely to benefit from a device or a drug can be included in the trials and those individuals whose variant genotypes indicate that they are less likely to or would not respond to a device or a drug, or suffer adverse reactions, can be eliminated from the clinical trials. This not only improves the safety of clinical trials, but also will enhance the chances that the trial will demonstrate statistically significant efficacy. Furthermore, the variants of the present disclosure may explain why certain previously developed devices or drugs performed poorly in clinical trials and may help identify a subset of the population that would benefit from a drug that had previously performed poorly in clinical trials, thereby “rescuing” previously developed therapeutic treatment methods or drugs, and enabling the methods or drug to be made available to a particular endometriosis patient population that can benefit from it.

Detection Kits and Systems

In some instances, based on a variant such as SNP or indels and associated sequence information disclosed herein, detection reagents can be developed and used to assay any variant of the present disclosure individually or in combination, and such detection reagents can be readily incorporated into one of the established kit or system formats which are well known in the art. The terms “kits” and “systems” can refer to such things as combinations of multiple variant detection reagents, or one or more variant detection reagents in combination with one or more other types of elements or components (e.g., other types of biochemical reagents, containers, packages such as packaging intended for commercial sale, substrates to which variant detection reagents are attached, electronic hardware components, etc.). Accordingly, the present disclosure further provides variant detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more variants of the present disclosure. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more variant detection reagents (along with, optionally, other biochemical reagents) packaged in one or more containers.

In some instances, provided herein is a kit comprising one or more variant detection agents, and methods for detecting the variants disclosed herein by employing detection reagents and optionally a questionnaire of non-genetic clinical factors. In some instances, provided herein is a method of identifying an individual having an increased or decreased risk of developing endometriosis by detecting the presence or absence of a variant allele disclosed herein. In some instances, provided herein is a method for diagnosis of endometriosis by detecting the presence or absence of a variant allele disclosed herein is provided. In some instances, provided herein is a method for predicting endometriosis sub-classification by detecting the presence or absence of a variant allele. In some instances, the questionnaire would be completed by a medical professional based on medical history physical exam or other clinical findings. In some instances, the questionnaire would include any other non-genetic clinical factors known to be associated with the risk of developing endometriosis. In some instances, a reagent for detecting a variant in the context of its naturally-occurring flanking nucleotide sequences (which can be, e.g., either DNA or mRNA) is provided. In some instances, the reagent may be in the form of a hybridization probe or an amplification primer that is useful in the specific detection of a variant of interest. In some instances, a variant can be a genetic polymorphism having a Minor Allele Frequency (MAF) of at least 1% in a population (such as for instance the Caucasian population or the CEU population) and an RV is understood to be a genetic polymorphism having a Minor Allele Frequency (MAF) of less than 1% in a population (such as for instance the Caucasian population or the CEU population).

In some instances, a detection kit can contain one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a variant-containing nucleic acid molecule. A kit may further contain means for determining the amount of a target nucleic acid, and means for comparing the amount with a standard, and can comprise instructions for using the kit to detect the variant-containing nucleic acid molecule of interest. In one embodiment of the present disclosure, kits are provided which contain the necessary reagents to carry out one or more assays to detect one or more variants disclosed herein. In an exemplary embodiment of the present disclosure, the detection kits/systems can be in the form of nucleic acid arrays, or compartmentalized kits, including microfluidic/lab-on-a-chip systems.

In some instances, variant detection kits/systems may contain, for example, one or more probes, or pairs of probes, that hybridize to a nucleic acid molecule at or near each target variant position. Multiple pairs of allele-specific probes may be included in the kit/system to simultaneously assay large numbers of variants, at least one of which is a variant of the present disclosure. In some kits/systems, the allele-specific probes are immobilized to a substrate such as an array or bead. For example, the same substrate can comprise allele-specific probes for detecting at least 1; 10; 100; 1000; 10,000; 100,000; 500,000 (or any other number in-between) or substantially all of the variants disclosed herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

In some instances, any number of probes, such as allele-specific probes, may be implemented in an array, and each probe or pair of probes can hybridize to a different variant position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). For example, probes are attached to a solid support in an ordered, addressable array.

In some instances, a microarray can be composed of a large number of unique, single-stranded polynucleotides fixed to a solid support. Typical polynucleotides are for example about 6-60 nucleotides in length, more for example about 15-30 nucleotides in length, and most for example about 18-25 nucleotides in length. For certain types of microarrays or other detection kits/systems, it may be suitable to use oligonucleotides that are only about 7-20 nucleotides in length. In other types of arrays, such as arrays used in conjunction with chemiluminescent detection technology, exemplary probe lengths can be, for example, about 15-80 nucleotides in length, for example about 50-70 nucleotides in length, more for example about 55-65 nucleotides in length, and most for example about 60 nucleotides in length. The microarray or detection kit can contain polynucleotides that cover the known 5′ or 3′ sequence of the target variant site, sequential polynucleotides that cover the full-length sequence of a gene/transcript; or unique polynucleotides selected from particular areas along the length of a target gene/transcript sequence, particularly areas corresponding to one or more variants disclosed herein. Polynucleotides used in the microarray or detection kit can be specific to a variant or variants of interest (e.g., specific to a particular SNP allele at a target SNP site, or specific to particular SNP alleles at multiple different SNP sites), or specific to a polymorphic gene/transcript or genes/transcripts of interest.

In some instances, hybridization assays based on polynucleotide arrays rely on the differences in hybridization stability of the probes to perfectly matched and mismatched target sequence variants. For variant genotyping, it is generally suitable that stringency conditions used in hybridization assays are high enough such that nucleic acid molecules that differ from one another at as little as a single variant position can be differentiated (e.g., typical variant hybridization assays are designed so that hybridization will occur only if one particular nucleotide is present at a variant position, but will not occur if an alternative nucleotide is present at that variant position). Such high stringency conditions may be suitable when using, for example, nucleic acid arrays of allele-specific probes for variant detection. In some instances, the arrays are used in conjunction with chemiluminescent detection technology.

In some instances, a nucleic acid array can comprise an array of probes of about 15-25 nucleotides in length. In further embodiments, a nucleic acid array can comprise any number of probes, in which at least one probe is capable of detecting one or more variants disclosed herein and/or at least one probe comprises a fragment of one of the sequences selected from the group consisting of those disclosed herein, and sequences complementary thereto, said fragment comprising at least about 8 consecutive nucleotides, for example 10, 12, 15, 16, 18, 20, more for example 22, 25, 30, 40, 47, 50, 55, 60, 65, 70, 80, 90, 100, or more consecutive nucleotides (or any other number in-between) and containing (or being complementary to) a variant. In some embodiments, the nucleotide complementary to the variant site is within 5, 4, 3, 2, or 1 nucleotide from the center of the probe, more for example at the center of said probe.

In some instances, using such arrays or other kits/systems, the present disclosure provides methods of identifying the variants disclosed herein in a test sample. Such methods typically involve incubating a test sample of nucleic acids with an array comprising one or more probes corresponding to at least one variant position of the present disclosure, and assaying for binding of a nucleic acid from the test sample with one or more of the probes. Conditions for incubating a variant detection reagent (or a kit/system that employs one or more such variant detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the variants disclosed herein.

In some instances, a detection kit/system may include components that are used to prepare nucleic acids from a test sample for the subsequent amplification and/or detection of a variant-containing nucleic acid molecule. Such sample preparation components can be used to produce nucleic acid extracts, including DNA and/or RNA, extracts from any bodily fluids. In a exemplary embodiment of the disclosure, the bodily fluid is blood, saliva or buccal swabs. The test samples used in the above-described methods will vary based on such factors as the assay format, nature of the detection method, and the specific tissues, cells or extracts used as the test sample to be assayed. Methods of preparing nucleic acids are well known in the art and can be readily adapted to obtain a sample that is compatible with the system utilized. In some instances, in addition to reagents for preparation of nucleic acids and reagents for detection of one of the variants of this disclosure, the kit may include a questionnaire inquiring about non-genetic clinical factors such as age, gender, or any other non-genetic clinical factors known to be associated with endometriosis.

In some instances, a form of kit can be a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other variant detection reagent for detecting one or more variants of the present disclosure, one or more containers which contain wash reagents (such as phosphate buffered saline, Tris-buffers, etc.), and one or more containers which contain the reagents used to reveal the presence of the bound probe or other variant detection reagents. The kit can optionally further comprise compartments and/or reagents for, for example, nucleic acid amplification or other enzymatic reactions such as primer extension reactions, hybridization, ligation, electrophoresis (for example capillary electrophoresis), mass spectrometry, and/or laser-induced fluorescent detection. The kit may also include instructions for using the kit. In such microfluidic devices, the containers may be referred to as, for example, microfluidic “compartments”, “chambers”, or “channels”.

In some instances, microfluidic devices, which may also be referred to as “lab-on-a-chip” systems, biomedical micro-electro-mechanical systems (bioMEMs), or multicomponent integrated systems, are exemplary kits/systems of the present disclosure for analyzing variants. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more variants of the present disclosure. One example of a microfluidic system is the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. In some instances, for genotyping variants, a microfluidic system may integrate, for example, nucleic acid amplification, primer extension, capillary electrophoresis, and a detection method such as laser induced fluorescence detection.

Methods of Treatment

In some aspects, disclosed herein is a method of treating a select subject in need thereof. The use of these genetic markers can allow selection of subjects for clinical trials involving novel treatment methods. In some cases, genetic markers disclosed herein can be used for early diagnosis and prognosis of endometriosis, as well as early clinical intervention to mitigate progression of the disease. In some instances, genetic markers disclosed herein can be used to predict endometriosis and endometriosis progression, for example in treatment decisions for individuals who are recognized as having endometriosis.

In some cases, a treatment disclosed herein includes one or more of: reducing the frequency and/or severity of symptoms, elimination of symptoms and/or their underlying cause, and improvement or remediation of damage. For example, treatment of endometriosis includes, relieving the pain experienced by a woman suffering from endometriosis, and/or causing the regression or disappearance of endometriotic lesions.

In some cases, the treatment can be an advanced reproductive therapy such as in vitro in fertilization (IVF); a hormonal treatment; progestogen; progestin; an oral contraceptive; a hormonal contraceptive; danocrine; gentrinone; a gonadotrophin releasing hormone agonist; Lupron; danazol; an aromatase inhibitor; pentoxifylline; surgical treatment; laparoscopy; cauterization; or cystectomy. In some instances, the progestogen can be progesterone, desogestrel, etonogestrel, gestodene, levonorgestrel, medroxyprogesterone, norethisterone, norgestimate, megestrol, megestrol acetate, norgestrel, a pharmaceutically acceptable salt thereof (e.g., acetate), or any combination thereof. In some instances, a therapeutic used herein is selected from progestins, estrogens, antiestrogens, and antiprogestins, for example micronized danazol in a micro- or nanoparticulate formulation.

In some cases, a method of treatment disclosed herein comprises direct administration into or within an endometriotic lesion in a subject suffering from endometriosis of a pharmaceutical composition comprising a therapeutic disclosed herein. In some instances, the therapeutic is micronized in a suspension, e.g., non-oil based suspension. In some embodiments, the suspension comprises water, sodium sulfate, a quaternary ammonium wetting agent, glycerol, propylene glycol, polyethylene glycol, polypropylene glycol, a hydrophilic colloid, or any combination thereof.

The term “effective amount,” as used herein, can refer to a sufficient amount of a therapeutic being administered which relieve to some extent one or more of the symptoms of the disease or condition being treated. The result can be reduction and/or alleviation of the signs, symptoms, or causes of a disease, or any other desired alteration of a biological system. A therapeutic can be administered for prophylactic, enhancing, and/or therapeutic treatments. An appropriate “effective” amount in any individual case can be determined using techniques, such as a dose escalation study.

A treatment can comprise administering a therapeutic to a subject, intralesionally, transvaginally, intravenously, subcutaneously, intramuscularly, by inhalation, dermally, intra-articular injection, orally, intrathecally, transdermally, intranasally, via a peritoneal route, or directly onto or into a lesion/site, e.g., via endoscopically, open surgical administration, or injection route of application. In some instances, intralesional administration can mean administration into or within a pathological area. Administration can be effected by injection into a lesion and/or by instillation into a pre-existing cavity, such as in endometrioma. With reference to treatments for endometriosis provided herein, intralesional administration can refer to treatment within endometriotic tissue or a cyst formed by such tissue, such as by injection into a cyst. In some instances, intralesional administration can include administration into tissue in such close proximity to the endometriotic tissue such that the progestogen acts directly on the endometriotic tissue. In some instances, intralesional administration may or may not include administration to tissue remote from the endometriotic tissue that the progestogen acts on the endometriotic tissue through systemic circulation. In some instances, intralesional administration administration or delivery includes transvaginal, endoscopic or open surgical administration including, but are not limited to, via laparotomy. In some instances, transvaginal administration can refer to all procedures, including drug delivery, performed through the vagina, including intravaginal delivery and transvaginal sonography (ultrasonography through the vagina).

In some instances, administration is by injection into the endometriotic tissue or into a cyst formed by such tissue; or into tissue immediately surrounding the endometriotic tissue in such proximity that the progestogen acts directly on the endometriotic tissue. In some embodiments, the tissue is visualized, for example laparoscopically or by ultrasound, and the progestogen is administered by intralesional (intracystic) injection by, for example direct visualization under ultrasound guidance or by any other suitable methods. A suitable amount of the theraeputic, e.g., progestrogen expressed in terms of progestrone of about 1-2 gm per lesion/cyst, can be applied. Precise quantity generally is determined on case to case basis, depending upon parameters, such as the size of the endometriotic tissue mass, the mode of the administration, and the number and time intervals between treatments.

In some instances, methods herein can comprise intralesional delivery of the medicaments into the lesion. Intralesional delivery includes, for example, transvaginal, endoscopic or open surgical administration including via laparotomy. Delivery can be effected, for example, through a needle or needle like device by injection or a similar injectable or syringe-like device that can be delivered into the lesion, such as transvaginally, endoscopically or by open surgical administration including via laparotomy. In some embodiments, the method includes intravaginal and transvaginal delivery. For intravaginal/transvaginal delivery an ultrasound probe can be used to guide delivery of the needle from the vagina into lesions such as endometriomas and utero sacral nodules. Under ultrasound guidance the needle tip is placed in the lesion, the contents of the lesion aspirated if necessary and the formulation is injected into the lesion. In an exemplary delivery system a 17 to 20 gauge needle can be used for injection of the drug. Such system can be used for intralesional delivery including, but not limited to, transvaginal, endoscopic or open surgical administration including via laparotomy. For treatment of endometrioma 17 or 18 gauge needles are used under ultrasound guidance for aspiration of the thick contents of the lesion and delivery of the formulation. The length of the needle used depends on the depth of the lesion. Pre-loaded syringes and other administration systems, which obviate the need for reloading the drug can be used.

In some cases, a therapeutic (e.g., an active agent) used herein can be a solution, a suspension, liquid, a paste, aqueous, non-aqueous fluid, semi-solids, colloid, gel, lotion, cream, solid (e.g., tablet, powder, pellet, particulate, capsule, packet), or any combination thereof. In some instances, a therapeutic disclosed herein is formulated as a dosage form of tablet, capsule, gel, lollipop, parenteral, intraspinal infusion, inhalation, spray, aerosol, transdermal patch, iontophoresis transport, absorbing gel, liquid, liquid tannate, suppositories, injection, I.V. drip, or a combination thereof to treat subjects. In some instances, the active agents are formulated as single oral dosage form such as a tablet, capsule, cachet, soft gelatin capsule, hard gelatin capsule, extended release capsule, tannate tablet, oral disintegrating tablet, multi-layer tablet, effervescent tablet, bead, liquid, oral suspension, chewable lozenge, oral solution, lozenge, lollipop, oral syrup, sterile packaged powder including pharmaceutically-acceptable excipients, other oral dosage forms, or a combination thereof. In some instances, a therapeutic of the disclosure herein can be administered using one or more different dosage forms which are further disclosed herein. In some instances, therapeutics disclosed herein are provided in modified release dosage forms (such as immediate release, controlled release, or both),

The methods, compositions, and kits of this disclosure can comprise a method to prevent, treat, arrest, reverse, or ameliorate the symptoms of a condition of a subject, e.g., a patient. A subject can be, for example, an elderly adult, adult, adolescent, pre-adolescence, teenager, or child. A subject can be, for example, 10-50 years old, 10-40 years old, 10-30 years old, 10-25 years old, 10-21 years old, 10-18 years old, 10-16 years old, 18-25 years old, or 16-34 years old. The subject can be a female mammal, e.g., a female human being. In some instances, the human subject can be asymptomatic for endometriosis.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial.

A treatment, e.g., administration of a therapeutic, can occur 1, 2, 3, 4, 5, 6, 7, or 8 times daily. A treatment, e.g., administration of a therapeutic, can occur 1, 2, 3, 4, 5, 6, or 7 times weekly. A treatment, e.g., administration of a therapeutic, can occur 1, 2, 3, 4, 5, 6, 7, 8, 9, or 10 times monthly. A treatment, e.g., administration of a therapeutic, can occur 1, 2, 3, 4, 5, 6, 7, 8, 9, 10, 11, or 12 times yearly. In some instances, therapeutics disclosed herein are administered to a subject at about every 4 to about 6 hours, about every 12 hours, about every 24 hours, about every 48 hours, or more often. In some instances, therapeutics disclosed herein can be administered once, twice, three times, four times, five times, six times, seven times, eight times, or more often daily. In some instances, a dosage form disclosed herein provides an effective plasma concentration of an active agent at from about 1 minute to about 20 minutes after administration, such as about: 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 21 min, 22 min, 23 min, 24 min, 25 min. In some instances, a dosage form of the disclosure herein provides an effective plasma concentration of an active agent at from about 20 minutes to about 24 hours after administration, such as about 20 minutes, 30 minutes, 40 minutes, 50 minutes, 1 hr, 1.2 hrs, 1.4 hrs, 1.6 hrs, 1.8 hrs, 2 hrs, 2.2 hrs, 2.4 hrs, 2.6 hrs, 2.8 hrs, 3 hrs, 3.2 hrs, 3.4 hrs, 3.6 hrs, 3.8 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or 24 hrs following administration. In some instances, an active agent can be present in an effective plasma concentration in a subject for about 4 to about 6 hours, about 12 hours, about 24 hour, or 1 day to 30 days, including but not limited to 1, 2, 3, 4, 5, 6, 7, 8, 9, 10, 12, 12, 13, 14, 15, 16, 17, 18, 19, 20, 21, 22, 23, 24, 25, 26, 27, 28, 29 or 30 days.

In some instances, a therapeutic (e.g., an active agent) is administered to a subject in a dosage of about 0.01 mg to about 500 mg per day, e.g., about 1-50 mg/day for an average person. In some embodiments, the daily dosage is from about 0.01 mg to about 5 mg, about 1 to about 10 mg, about 5 mg to about 20 mg, about 10 mg to about 50 mg, about 20 mg to about 100 mg, about 50 mg to about 150 mg, about 100 mg to about 250 mg, about 150 mg to about 300 mg, or about 250 mg to about 500 mg.

In some instances, each administration of a therapeutic (e.g., an active agent) is in an amount of about: 0.1-5 mg, 0.1-10 mg, 1-5 mg, 1-10 mg, 1-20 mg, 10-20 mg, 10-30 mg, 10-40 mg, 10-50 mg, 20-30 mg, 20-40 mg, 20-50 mg, 25-50 mg, 30-40 mg, 30-50 mg, 30-60 mg, 40-50 mg, 40-60 mg, 50-60 mg, 50-75 mg, 60-80 mg, 75-100 mg, or 80-100 mg, for example: about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 55 mg, about 60 mg, about 65 mg, about 70 mg, about 75 mg, about 80 mg, about 85 mg, about 90 mg, about 95 mg, or about 100 mg.

In some instances, a therapeutic (e.g., an active agent) is administered to a subject in a dosage of about 0.01 g to about 100 g per day, e.g., about 1-10 g/day for an average person. In some embodiments, the daily dosage is from about 0.01 g to about 5 g, about 1 to about 10 g, about 5 g to about 20 g, about 10 g to about 50 g, about 20 g to about 100 g, or about 50 g to about 100 g.

In some instances, each administration of a therapeutic (e.g., an active agent) is in an amount of about: 0.01-1 g, 0.1-5 g, 0.1-10 g, 1-5 g, 1-10 g, 1-20 g, 10-20 g, 10-30 g, 10-40 g, 10-50 g, 20-30 g, 20-40 g, 20-50 g, 25-50 g, 30-40 g, 30-50 g, 30-60 g, 40-50 g, 40-60 g, 50-60 g, 50-75 g, 60-80 g, 75-100 g, or 80-100 g, for example: about 0.5 g, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, about 15 g, about 15.5 g, about 16 g, about 16.5 g, about 17 g, about 17.5 g, about 18 g, about 18.5 g, about 19 g, about 19.5 g, about 20 g, about 22.5 g, about 25 g, about 27.5 g, about 30 g, about 32.5 g, about 35 g, about 37.5 g, about 40 g, about 42.5 g, about 45 g, about 47.5 g, about 50 g, about 55 g, about 60 g, about 65 g, about 70 g, about 75 g, about 80 g, about 85 g, about 90 g, about 95 g, or about 100 g.

In some instances, a therapeutic (e.g., in a liquid) administered to a subject having an active agent concentration of about: 0.01-0.1, 0.1-1, 1-10, 1-20, 5-30, 5-40, 5-50, 10-20, 10-25, 10-30, 10-40, 10-50, 15-20, 15-25, 15-30, 15-40, 15-50, 20-30, 20-40, 20-50, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, 50-100, 50-150, 50-200, 50-300, 100-300, 100-400, 100-500, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 μM, or any combination thereof.

In some cases, a therapeutic can comprise one or more active agents, administered to a subject at least about: 0.001 mg, 0.01 mg, 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg, or per kg body weight of a subject in need thereof. The therapeutic may comprise a total dose of one or more active agents administered at about 0.1 to about 10.0 mg, for example, about 0.1-10.0 mg, about 0.1-9.0 mg, about 0.1-8.0 mg, about 0.1-7.0 mg, about 0.1-6.0 mg, about 0.1-5.0 mg, about 0.1-4.0 mg, about 0.1-3.0 mg, about 0.1-2.0 mg, about 0.1-1.0 mg, about 0.1-0.5 mg, about 0.2-10.0 mg, about 0.2-9.0 mg, about 0.2-8.0 mg, about 0.2-7.0 mg, about 0.2-6.0 mg, about 0.2-5.0 mg, about 0.24.0 mg, about 0.2-3.0 mg, about 0.2-2.0 mg, about 0.2-1.0 mg, about 0.2-0.5 mg, about 0.5-10.0 mg, about 0.5-9.0 mg, about 0.5-8.0 mg, about 0.5-7.0 mg, about 0.5-6.0 mg, about 0.5-5.0 mg, about 0.5-4.0 mg, about 0.5-3.0 mg, about 0.5-2.0 mg, about 0.5-1.0 mg, about 1.0-10.0 mg, about 1.0-5.0 mg, about 1.0-4.0 mg, about 1.0-3.0 mg, about 1.0-2.0 mg, about 2.0-10.0 mg, about 2.0-9.0 mg, about 2.0-8.0 mg, about 2.0-7.0 mg, about 2.0-6.0 mg, about 2.0-5.0 mg, about 2.0-4.0 mg, about 2.0-3.0 mg, about 5.0-10.0 mg, about 5.0-9.0 mg, about 5.0-8.0 mg, about 5.0-7.0 mg, about 5.0-6.0 mg, about 6.0-10.0 mg, about 6.0-9.0 mg, about 6.0-8.0 mg, about 6.0-7.0 mg, about 7.0-10.0 mg, about 7.0-9.0 mg, about 7.0-8.0 mg, about 8.0-10.0 mg, about 8.0-9.0 mg, or about 9.0-10.0 mg, or per kg body weight of a subject in need thereof.

In some cases, a method of treatment disclosed herein comprises administering a therapeutic. In some instances, the method comprises administering a therapeutic includes one or more of the following steps: a) obtaining a genetic material sample of a human female subject, b) identifying in the genetic material of the subject a genetic marker having an association with endometriosis, c) assessing the subject's risk of endometriosis or risk of endometriosis progression, d) identifying the subject as having an altered risk of endometriosis or an altered risk of endometriosis progression, e) administering to the subject a therapeutic, or any combination thereof.

In some instances, the subject may be endometriosis presymptomatic or the subject may exhibit endometriosis symptoms. In some instances, the assessment of risk may include non-genetic clinical factors. In some instances, the therapeutic is adapted to the specific subject so as to be a proper and effective amount of therapeutic for the subject. In some instances, the administration of the therapeutic may comprise multiple sequential instances of administration of the therapeutic and that such sequence instances may occur over an extended period of time or may occur on an indefinite on-going basis. In some instances, the therapeutic may be a gene or protein based therapy adapted to the specific needs of a select patient.

Hormonal Therapy

In some cases, a treatment method herein comprises supplementing the body with a hormone thereof such as a steroid hormone, for example a method of preventing endometriosis comprising administering a hormonal therapy to a human subject having at least one genetic variant defining a minor allele disclosed herein, e.g., listed in Table 1. In some instances, the hormone can be progestin, progestogen, progesterone, desogestrel, etonogestrel, gestodene, levonorgestrel, medroxyprogesterone, norethisterone, norgestimate, megestrol, megestrol acetate, norgestrel, a pharmaceutically acceptable salt thereof (e.g., acetate), or any combination thereof. In some instances, a therapeutic used herein is selected from progestins, estrogens, antiestrogens, and antiprogestins, for example micronized danazol in a micro- or nanoparticulate formulation. Methods and therapeutics presented herein can utilize an active agent in a freebase, salt, hydrate, polymorph, isomer, diastereomer, prodrug, metabolite, ion pair complex, or chelate form. An active agent can be formed using a pharmaceutically acceptable non-toxic acid or base, including an inorganic acid or base, or an organic acid or base. In some instances, an active agent that can be utilized in connection with the methods and compositions presented herein is a pharmaceutically acceptable salt derived from acids including, but not limited to, the following: acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid. For further description of pharmaceutically acceptable salts that can be used in the methods described herein see, for example, S. M. Barge et al., “Pharmaceutical Salts,” 1977, J. Pharm. Sci. 66:1-19, which is incorporated herein by reference in its entirety.

In some instances, the therapeutic may take the form of a testosterone or a modified testosterone such as Danazol. In some instances, the therapeutic can be a hormonal treatment therapeutic which may be administered alone or in combination with a gene therapy. For instance, the therapeutic may be an estrogen containing composition, a progesterone containing composition, a progestin containing composition, a gonadotropin releasing-hormone (GnRH) agonist, a gonadotropin releasing-hormone (GnRH) antagonist, or other ovulation suppression composition, or a combination thereof. In some instances, the GnRH agonist may take the form of a GnRH agonist in combination with a patient specific substantially low dose of estrogen, progestin, or tibolone via an add-back administration. In some instances, in such add-back therapy, the dosage of estrogen, progestin, or tibolone is relatively small so as to not reduce the effectiveness of the GnRH agonist. In some instances, the therapeutic is an oral contraceptive (OC). In some instances, the OC is in a pill form that is comprised at least partially of estrogen, progesterone, or a combination thereof. In some instances, the progesterone component may be any of Desogestrel, Drospirenone, Ethynodiol, Levonorgestrel, Norethindrone, Norgestimate, and Norgestrel, and the estrogen component may further be any of Mestranol, Estradiol, and Ethinyl. In some instances, the OC may be any commercially available OC including ALESSE, APRI, ARANELLE, AVIANE, BREVICON, CAMILA, CESIA, CRYSELLE, CYCLESSA, DEMULEN, DESOGEN, ENPRESSE, ERRIN, ESTROSTEP, JOLIVETTE, JUNEL, KARIVA, LEENA, LESSINA, LEVLEN, LEVORA, LOESTRIN, LUTERA, MICROGESTIN, MICRONOR, MIRCETTE, MODICON, MONONESSA, NECON, NORA, NORDETTE, NORINYL, NOR-QD, NORTREL, OGESTREL, ORTHO-CEPT, ORTHO-CYCLEN, ORTHO-NOVUM, ORTHO-TRI-CYCLEN, OVCON, OVRAL, OVRETTE, PORTIA, PREVIFEM, RECLIPSEN, SOLIA, SPRINTEC, TRINESSA, TRI-NORINYL, TRIPHASIL, TRIVORA, VELIVET, YASMIN, AND ZOVIA (the preceding names are the registered trademarks of the respective providers).

Assisted Reproductive Therapy

In some cases, a method herein can comprise administering to a select subject assisted reproductive therapy (ART), for example a method of treating endometriosis associated infertility comprising administering ART to a select human subject having at least one genetic variant defining a minor allele disclosed herein, e.g., listed in Table 2. In some instances, ART can comprise in vitro fertilization (IVF), embryo transfer (ET), fertility medication, intracytoplasmic sperm injection (ICSI), cryopreservation, or any combination thereof. In some instances, ART can comprise surgically removing eggs from a woman's ovaries, combining them with sperm in the laboratory, and returning them to the woman's body or donating them to another woman.

In some instances, the in vitro fertilization (IVF) procedure can provide for a live birth event following the IVF procedure. In some instances, a method herein provides a probability of a live birth event occurring resulting from the first or subsequent in vitro fertilization cycle based at least in part on items of information from the female subjects.

In some instances, the IVF can comprise ovulation induction, utilizing fertility medication can comprise agents that stimulate the development of follicles in the ovary. Examples are gonadotropins and gonadotropin releasing hormone.

In some instances, IVF can comprise transvaginal ovum retrieval (OVR), which can be a process whereby a small needle is inserted through the back of the vagina and guided via ultrasound into the ovarian follicles to collect the fluid that contains the eggs.

In some instances, IVF can comprise embryo transfer, which can be the step in the process whereby one or several embryos are placed into the uterus of the female with the intent to establish a pregnancy.

In some instances, IVF can comprise assisted zona hatching (AZH), which can be performed shortly before the embryo is transferred to the uterus. A small opening can be made in the outer layer surrounding the egg in order to help the embryo hatch out and aid in the implantation process of the growing embryo.

In some instances, IVF can comprise artificial insemination, for example intrauterine insemination, intracervical insemination, intrauterine tuboperitoneal insemination, intratubal insemination, or any combination thereof.

In some instances, IVF can comprise intracytoplasmic sperm injection (ICSI), which can be beneficial in the case of male factor infertility where sperm counts are very low or failed fertilization occurred with previous IVF attempt(s). The ICSI procedure can involve a single sperm carefully injected into the center of an egg using a microneedle. With ICSI, only one sperm per egg is needed. Without ICSI, one may need between 50,000 and 100,000. In some embodiments, this method can be employed when donor sperm is used.

In some instances, IVF can comprise autologous endometrial coculture, which can be a possible treatment for patients who have failed previous IVF attempts or who have poor embryo quality. The patient's fertilized eggs can be placed on top of a layer of cells from the patient's own uterine lining, creating a more natural environment for embryo development.

In some instances, IVF can comprise zygote intrafallopian transfer (ZIFT), in which egg cells can be removed from the woman's ovaries and fertilized in the laboratory; the resulting zygote can be then placed into the fallopian tube.

In some instances, IVF can comprise cytoplasmic transfer, in which the contents of a fertile egg from a donor can be injected into the infertile egg of the patient along with the sperm.

In some instances, IVF can comprise egg donors, which are resources for women with no eggs due to surgery, chemotherapy, or genetic causes; or with poor egg quality, previously unsuccessful IVF cycles or advanced maternal age. In the egg donor process, eggs can be retrieved from a donor's ovaries, fertilized in the laboratory with the sperm from the recipient's partner, and the resulting healthy embryos can be returned to the recipient's uterus.

In some instances, IVF can comprise sperm donation, which may provide the source for the sperm used in IVF procedures where the male partner produces no sperm or has an inheritable disease, or where the woman being treated has no male partner.

In some instances, IVF can comprise preimplantation genetic diagnosis (PGD), which can involve the use of genetic screening mechanisms such as fluorescent in-situ hybridization (FISH) or comparative genomic hybridization (CGH) to help identify genetically abnormal embryos and improve healthy outcomes.

In some instances, IVF can comprise embryo splitting can be used for twinning to increase the number of available embryos.

In some instances, ART can comprise gamete intrafallopian transfer (GIFT), in which a mixture of sperm and eggs can be placed directly into a woman's fallopian tubes using laparoscopy following a transvaginal ovum retrieval.

In some instances, ART can comprise reproductive surgery, treating e.g. fallopian tube obstruction and vas deferens obstruction, or reversing a vasectomy by a reverse vasectomy. In surgical sperm retrieval (SSR) the reproductive urologist can obtain sperm from the vas deferens, epididymis or directly from the testis in a short outpatient procedure. By cryopreservation, eggs, sperm and reproductive tissue can be preserved for later IVF.

In some instances, a subject to treat can be a pre-in vitro fertilization (pre-IVF) procedure patient. In certain embodiments, the items of information relating to preselected patient variables for determining the probability of a live birth event for a pre-IVF procedure patient may include age, diminished ovarian reserve, 3 follicle stimulating hormone (FSH) level, body mass index, polycystic ovarian disease, season, unexplained female infertility, number of spontaneous miscarriages, year, other causes of female infertility, number of previous pregnancies, number of previous term deliveries, endometriosis, tubal disease, tubal ligation, male infertility, uterine fibroids, hydrosalpinx, and male infertility causes.

In some instances, a subject to treat can be a pre-surgical (pre-OR) procedure patient (pre-OR is also referred to herein as pre-oocyte retrieval). In certain embodiments, the items of information relating to preselected patient variables for determining the probability of a live birth event for a pre-OR procedure patient may include age, endometrial thickness, total number of oocytes, total amount of gonatropins administered, number of total motile sperm after wash, number of total motile sperm before wash, day 3 follicle stimulating hormone (FSH) level, body mass index, sperm collection, age of spouse, season number of spontaneous miscarriages, unexplained female infertility, number of previous term deliveries, year, number of previous pregnancies, other causes of female infertility, endometriosis, male infertility, tubal ligation, polycystic ovarian disease, tubal disease, sperm from donor, hydrosalpinx, uterine fibroids, and male infertility causes.

In some instances, a subject to treat can be a post-in vitro fertilization (post-IVF) procedure patient. In certain embodiments, the items of information relating to preselected patient variables for determining the probability of a live birth event for a post-IVF procedure patient may include blastocyst development rate, total number of embryos, total amount of gonatropins administered, endometrial thickness, flare protocol, average number of cells per embryo, type of catheter used, percentage of 8-cell embryos transferred, day 3 follicle stimulating hormone (FSH) level, body mass index, number of motile sperm before wash, number of motile sperm after wash, average grade of embryos, day of embryo transfer, season, number of spontaneous miscarriages, number of previous term deliveries, oral contraceptive pills, sperm collection, percent of unfertilized eggs, number of embryos arrested at 4-cell stage, compaction on day 3 after transfer, percent of normal fertilization, percent of abnormally fertilized eggs, percent of normal and mature oocytes, number of previous pregnancies, year, polycystic ovarian disease, unexplained female infertility, tubal disease, male infertility only, male infertility causes, endometriosis, other causes of female infertility, uterine fibroids, tubal ligation, sperm from donor, hydrosalpinx, performance of ICSI, or assisted hatching.

Pain Managing Medications

In some cases, a method disclosed herein can comprise administering a pain medication to a select subject, for example to a human subject having at least one genetic variant defining a minor allele listed in Table 3. In some instances, the pain medication comprises a nonsteroidal anti-inflammatory drug (NSAID), ibuprofen, naproxen, acetaminophen, an opioid, a cannabis-based therapeutic, or any combination thereof.

In some instances, the pain medication described herein can comprise an NSAID, for example amoxiprin, benorilate, choline magnesium salicylate, diflunisal, faislamine, methyl salicylate, magnesium salicylate, diclofenac, aceclofenac, acemetacin, bromfenac, etodolac, indometacin, nabumetone, sulindac, tolmetin, ibuprofen, carprofen, fenbuprofen, flubiprofen, ketaprofen, ketorolac, loxoprofen, naproxen, suprofen, mefenamic acid, meclofenamic acid, piroxicam, lomoxicam, meloxicam, tenoxicam, phenylbutazone, azapropazone, metamizole, oxyphenbutazone, or sulfinprazone, or a pharmaceutically acceptable salt thereof.

In some instances, the pain medication described herein can comprise an opioid analgesic, for example hydrocodone, oxycodone, morphine, diamorphine, codeine, pethidine, alfentanil, buprenorphine, butorphanol, dezocine, fentanyl, hydromorphone, levomethadyl acetate, levorphanol, meperidine, methadone, morphine sulfate, nalbuphine, oxymorphone, pentazocine, propoxyphene, remifentanil, sufentanil, or tramadol, or a pharmaceutically acceptable salt thereof.

In some instances, the pain medication described herein can comprise a cannabis-based therapeutic such as a cannabinoid for the treatment, reduction or prevention of pain. Exemplary cannabinoid for the treatment of pain include, without limitation, nabilone, dronabinol (THC), cannabidiol (CBD), cannabinol (CBN), cannabichromeme (CBC), cannabigerol (CBG), tetrahydrocannabivarin (THCV), tetrahydrocannabinolic acid (THCA), cannabidivarin (CBDV), cannadidiolic acid (CBDA), ajulemic acid, dexanabinol, cannabinor, HU 308, HU 331, and a pharmaceutically acceptable salt thereof.

### Specific Embodiments

A number of methods and systems are disclosed herein. Specific exemplary embodiments of these methods and systems are disclosed below.

Embodiment 1. A method comprising: hybridizing a nucleic acid probe to a nucleic acid sample from a human subject suspected of having or developing endometriosis; and detecting a genetic variant in a panel comprising two or more genetic variants defining a minor allele listed in Table 1.

Embodiment 2. The method of embodiment 1, wherein the nucleic acid sample comprises mRNA, cDNA, genomic DNA, or PCR amplified products produced therefrom, or any combination thereof.

Embodiment 3. The method of embodiment 1 or 2, wherein the nucleic acid sample comprises PCR amplified nucleic acids produced from cDNA or mRNA.

Embodiment 4. The method of embodiment 1 or 2, wherein the nucleic acid sample comprises PCR amplified nucleic acids produced from genomic DNA.

Embodiment 5. The method of any one of embodiments 1-4, wherein the nucleic acid probe is a sequencing primer.

Embodiment 6. The method of any one of embodiments 1-4, wherein the nucleic acid probe is an allele specific probe.

Embodiment 7. The method of any one of embodiments 1-6, wherein the detecting comprises DNA sequencing, hybridization with a complementary probe, an oligonucleotide ligation assay, a PCR-based assay, or any combination thereof.

Embodiment 8. The method of any one of embodiments 1-7, wherein the panel comprises at least: 5, 10, 15, 20, 25, 50, 75, 100, 150, 200, 250, 500, or more genetic variants defining minor alleles listed in Table 1.

Embodiment 9. The method of any one of embodiments 1-8, wherein the genetic variant has an odds ratio (OR) of at least: 1.5, 2, 5, 10, 20, 50, 100, or more.

Embodiment 10. The method of any one of embodiments 1-9, wherein the genetic variant comprises a synonymous mutation, a non-synonymous mutation, a nonsense mutation, an insertion, a deletion, a splice-site variant, a frameshift mutation, or any combination thereof.

Embodiment 11. The method of any one of embodiments 1-9, wherein the genetic variant comprises a protein damaging mutation.

Embodiment 12. The method of any one of embodiments 1-10, wherein the panel further comprises one or more protein damaging or loss of function variants in one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof.

Embodiment 13. The method of embodiment 12, further comprising sequencing the one or more genes to identify the one or more protein damaging or loss of function variants.

Embodiment 14. The method of embodiment 13, wherein the one or more protein damaging or loss of function variants are identified based on a predictive computer algorithm.

Embodiment 15. The method of embodiment 13 of 14, wherein the one or more protein damaging or loss of function variants are identified based on reference to a database.

Embodiment 16. The method of any one of embodiments 12-15, wherein the one or more protein damaging or loss of function variants comprise a stop-gain mutation, a spice-site mutation, a frameshift mutation, a missense mutation, or any combination thereof.

Embodiment 17. The method of any one of embodiments 1-16, wherein the panel further comprises one or more additional variants defining a minor allele listed in Table 4.

Embodiment 18. The method of any one of embodiments 1-17, wherein the panel is capable of identifying human subjects as having or being at risk of developing endometriosis with a specificity of at least: 80%, 85%, 90%, 95%, 96%, 97%, 98%, or 99%.

Embodiment 19. The method of any one of embodiments 1-18, wherein the panel is capable of identifying human subjects as having or being at risk of developing endometriosis with a sensitivity of at least: 800%, 85%, 90%, 95%, 96%, 97%, 98%, or 99%.

Embodiment 20. The method of any one of embodiments 1-19, wherein the panel is capable of identifying human subjects as having or being at risk of developing endometriosis with an accuracy of at least: 80%, 85%, 90%, 95%, 96%, 97%, 98%, or 99%.

Embodiment 21. The method of any one of embodiments 1-20, further comprising administering a therapeutic to the human subject.

Embodiment 22. The method of embodiment 21, wherein the therapeutic comprises hormonal therapy, an advanced reproductive therapy, a pain managing medication, or any combination thereof.

Embodiment 23. The method of embodiment 21, wherein the therapeutic comprises hormonal contraceptives, gonadotropin-releasing hormone (Gn-RH) agonists, gonadotropin-releasing hormone (Gn-RH) antagonists, progestin, danazol, or any combination thereof.

Embodiment 24. The method of any one of embodiments 1-23, wherein the human subject is asymptomatic for endometriosis.

Embodiment 25. The method of any one of embodiments 1-24, wherein the human subject is a teenager.

Embodiment 26. A method comprising detecting one or more genetic variants defining a minor allele listed in Table 1 in genetic material from a human subject suspected of having or developing endometriosis.

Embodiment 27. The method of embodiment 26, wherein the genetic material comprises mRNA, cDNA, genomic DNA, or PCR amplified products produced therefrom, or any combination thereof.

Embodiment 28. The method of embodiment 26 or 27, wherein the detecting comprises DNA sequencing, hybridization with a complementary probe, an oligonucleotide ligation assay, a PCR-based assay, of any combination thereof.

Embodiment 29. The method of any one of embodiments 26-28, wherein the detecting comprises hybridizing a nucleic acid probe to the genetic material.

Embodiment 30. The method of any one of embodiments 26-29, wherein the detecting comprises testing for the presence or absence of at least: 2, 3, 4, 5, 6, 7, 8, 9, 10, 15, 20, 25, 50, 100, 150, 250, or 500 genetic variants defining a minor allele listed in Table 1.

Embodiment 31. The method of any one of embodiments 26-30, wherein the one or more genetic variants have an odds ratio (OR) of at least: 1.5, 2, 5, 10, 20, 50, 100, or more.

Embodiment 32. The method of any one of embodiments 26-31, further comprising administering a therapeutic to the human subject.

Embodiment 33. A method comprising: sequencing one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof to identify one or more protein damaging or loss of function variants in a human subject suspected of having or developing endometriosis; and administering an endometriosis therapy to the human subject.

Embodiment 34. The method of embodiment 33, wherein the one or more protein damaging or loss of function variants are identified based on a predictive computer algorithm, reference to a database, or a combination thereof.

Embodiment 35. The method of embodiment 33 or 34, wherein the one or more protein damaging or loss of function variants comprise a stop-gain mutation, a spice-site mutation, a frameshift mutation, a missense mutation, or any combination thereof.

Embodiment 36. The method of any one of embodiments 33-35, wherein the endometriosis therapy comprises a hormonal therapy, an assisted reproductive therapy, a pain medication, or any combination thereof.

Embodiment 37. A method of preventing endometriosis comprising administering a hormonal therapy to a human subject having at least one genetic variant defining a minor allele listed in Table 1.

Embodiment 38. The method of embodiment 37, wherein the hormonal therapy comprises administration of hormonal contraceptives, gonadotropin-releasing hormone (Gn-RH) agonists, gonadotropin-releasing hormone (Gn-RH) antagonists, progestin, danazol, or any combination thereof.

Embodiment 39. A method of treating endometriosis associated infertility comprising administering an assisted reproductive therapy to a human subject having at least one genetic variant defining a minor allele listed in Table 2.

Embodiment 40. The method of embodiment 39, wherein the assisted reproductive therapy comprises in vitro fertilization, intrauterine insemination, ovulation induction, gamete intrafallopian transfer, or any combination thereof.

Embodiment 41. A method comprising administering a pain medication to a human subject having at least one genetic variant defining a minor allele listed in Table 3.

Embodiment 42. The method of embodiment 41, wherein the pain medication comprises a nonsteroidal anti-inflammatory drug (NSAID), ibuprofen, naproxen, an opioid, a cannabis-based therapeutic, or any combination thereof.

Embodiment 43. The method of any one of embodiment 37-42, further comprising detecting the at least one genetic variant in a genetic material from the human subject.

Embodiment 44. The method of embodiment 43, wherein the detecting comprises DNA sequencing, hybridization with a complementary probe, an oligonucleotide ligation assay, a PCR-based assay, or any combination thereof.

Embodiment 45. The method of embodiment 43, wherein the detecting comprises hybridizing a nucleic acid probe to the genetic material.

Embodiment 46. The method of embodiment 45, wherein the nucleic acid probe is a sequencing primer or an allele-specific probe.

Embodiment 47. The method of any one of embodiments 37-46, wherein the at least one genetic variant has an odds ratio (OR) of at least: 1.5, 2, 5, 10, 20, 50, 100, or more.

Embodiment 48. The method of any one of embodiments 37-47, wherein the at least one genetic variant comprises a synonymous mutation, a non-synonymous mutation, a nonsense mutation, an insertion, a deletion, a splice-site variant, a frameshift mutation, or any combination thereof.

### EXAMPLES

### Example 1. Low-Frequency, Damaging Mutations in Hundreds of Genes are Risk Factors for Endometriosis

This study performed exome-wide association analysis for rare low frequency mutations in the women with endometriosis. Rare exome variants associated with endometriosis were searched using an exome genotyping array and confirmatory whole exome sequencing (WES).

Consent and Medical Review

All subjects and controls were provided written informed consent in accordance with study protocols approved by Quorum Review IRB (Seattle, Wash. 98101). Trained OB/GYN clinicians performed the medical record review and clinical assessment of each patient.

Methods

Illumina Exome Human BeadChip. 1518 Caucasian patients with surgically confirmed endometriosis were tested for more than 200,000 rare non-synonymous variants (minor allele frequency <0.005). Allele frequencies were compared to the population datasets (genotyping dataset UK Michigan (n=50,000) and publicly available sequencing dataset Exac (n=33,000).

Affymetrix Axiom Custom Chip. 1888 Caucasian patients with surgically confirmed endometriosis were tested for more than 700,000 variants. Allele frequencies were compared to the population sequencing dataset Exac (n=33,000). Replication was performed on 530 endometriosis subjects with whole exome sequencing data. Association testing was performed using Fisher's exact test. Nominal threshold was selected for significance (p<0.05). Panther software was used to test gene ontologies. A predictive score (E) was estimated for each subject as follows: E=Σ log(L95ORj)*Cj, in which C is a count of risk allele, L950R is a lower limit of 95% CI of an odds ratio, and j is 1, 2, 3 . . . n, wherein n is the number of the associated variants.

Results

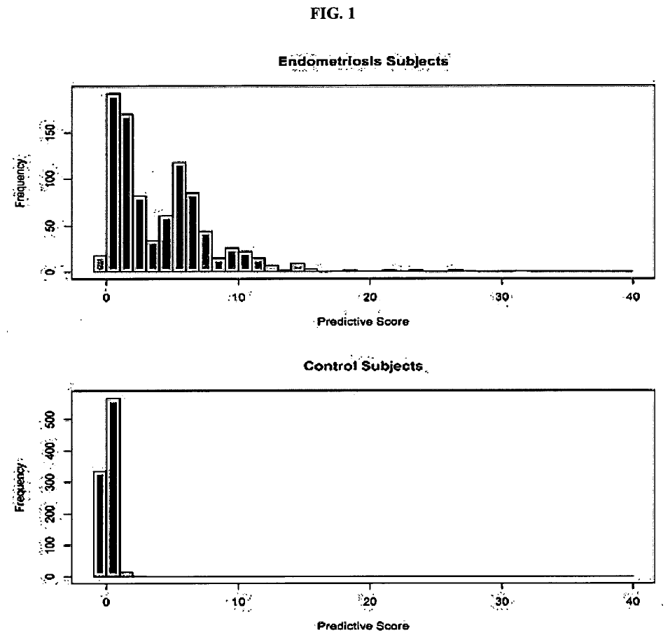

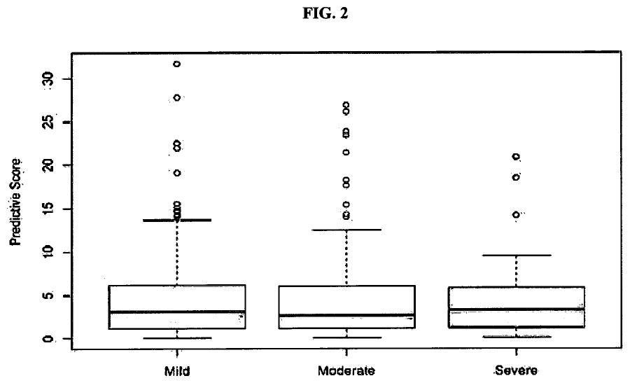

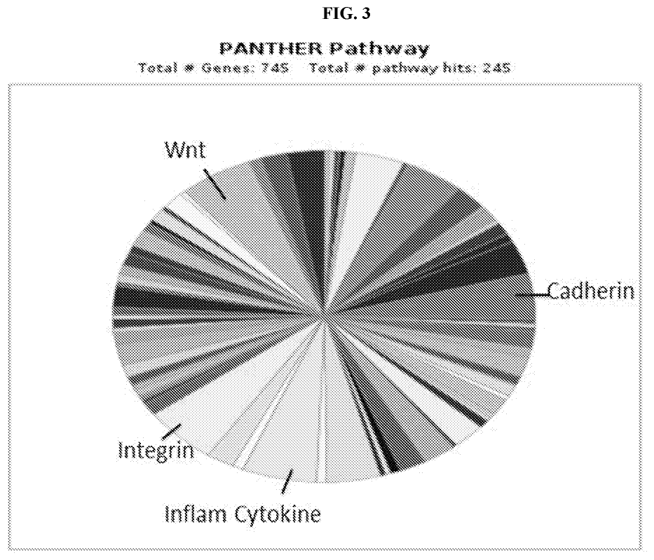

775 rare variants associated with endometriosis were identified, 561 of which were identified using Illumina Exome Beadchip, and 214 of which were identified using Affymetrix Axiom Custom Chip. FIG. 1 to FIG. 3 illustrate the results. Multiple low-frequency coding variants can be important in the genetic architecture of endometriosis. The relative risk of having endometriosis is significantly higher in women with multiple damaging variants, suggesting that they may serve as useful predictive or diagnostic markers. Genes involved with Wnt, cadherin, integrin, and inflammation medicated by cytokine signaling pathways are enriched, but trends did not reach significance.

### Example 2. Genetic Variation Underlying the Clinical Heterogeneity of Endometriosis

The study investigated whether two of the typical symptoms-pain and infertility may be linked to distinct genetic factors. A pool of 2818 non-synonymous SNP markers were selected to classify markers associated with pain or infertility patients. In one group, cases were included that reported pain as their primary symptom but not infertility (n=727), and in the other group, cases were included with infertility as their primary symptom with only minimal or no pain (n=138). SNPs were then evaluated for significant variation between the two groups.

Methods

Genotyping. The samples were genotyped on a custom designed microarray using the Affymetrix Axiom platform per the manufacturer's instructions.

Statistical Analysis. Differences in allele frequencies between the two cohorts were tested for each SNP by a 1-degree-of-freedom Corchran-Armitage Trend test.

Ethnicity. Subjects were confirmed Caucasian ethnicity using principal component analysis.

Population Controls. The marker frequencies were compared to population control dataset of European Ethnicity (n=33,000; ExAc Database) to associate the marker to the respective group.

Consent and Medical Review

All subjects were provided written informed consent in accordance with study protocols approved by Quorum Review IRB (Seattle, Wash. 98101). Trained OB/GYN clinicians performed the medical record review and clinical assessment of each patient. Inclusion criteria in the endometriosis case population in the study were surgically confirmed diagnosis of endometriosis.

Results

The analysis identified nine SNP variants with differential prevalence between pelvic pain patients and infertility patients as shown in Table 5.

| | | | | AA | Allele Frequency | | | CPP vs. INF | |
|---|---|---|---|---|---|---|---|---|---|
| SNP | Gene | Chr | Pos | change | ExAC | GPP | INF | P_{trend} | OR |

Genes associated with chronic pain

| | | | | | | | | | |
|---|---|---|---|---|---|---|---|---|---|
| rs172562 | TBX18 | 6 | 85,473,758 | G48R | 0.5706 | 0.4805 | 0.5766 | 0.0024 | 1.47 |
| rs12339210 | WHRN/ | 9 | 117,170,241 | P562A | 0.1636 | 0.1007 | 0.1606 | 0.0040 | 1.69 |
| | DFNB31 | | | | | | | | |
| rs35471617 | COL21A1 | 6 | 56,033,094 | T343M | 0.1274 | 0.0639 | 0.1159 | 0.0021 | 1.92 |
| rs72899872 | LPR1B | 2 | 141,232,800 | A3178T | 0.0127 | 0 | 0.0109 | 0.0001 | ∞ |

Genes associated with infertility

| | | | | | | | | | |
|---|---|---|---|---|---|---|---|---|---|
| rs8139422 | CRELD2 | 22 | 50,315,363 | D182E | 0.0313 | 0.0282 | 0.0616 | 0.0040 | 2.27 |
| rs78214713 | OR51Q1 | 11 | 5,444,040 | L204F | 0.0066 | 0.0089 | 0.029 | 0.0259 | 3.33 |
| rs7597367 | SCLY | 2 | 238,973,062 | K60E | 0.0006 | 0 | 0.0073 | 0.0011 | ∞ |
| rs35880972 | BIRC8 | 19 | 53,793,162 | A156T | 0.0004 | 0 | 0.0072 | 0.0012 | ∞ |
| rs34505126 | BMP3 | 4 | 81,967,240 | T222M | 0.0006 | 0 | 0.0072 | 0.0012 | ∞ |

Table 5 summarizes the results from a comparison of endometriosis associated variants with significantly different allele frequencies between patients with pelvic pain or infertility. ExAc refers to frequencies reported by the ExAc consortium. CPP refers to chronic pelvic pain and INF to infertility. Italic front indicates frequencies deviant from the general population.

The analysis identified five genes (CRELD2, OR51Q1, SCLY, BIRC8, BMP3) associated with infertility and four genes (TBX18, WHRN, COL21A1, LRP1B) associated with chronic pain. There was a sufficient power (>0.8) to detect markers with OR greater than 1.5 at significance level of 0.05. A review of the function of the genes identified can implicate several of the genes in both the pain and infertility pathways. Both WHRN and TBX18 which show differential allele frequencies in patients with pelvic pain have been shown to be linked to pain-pathways. Mutations in WHRN have been linked to deafness and mechano- and thermo-sensitive deficiencies and can stabilize the paranodal region and axonal cytoskeleton in myelinated axons. TBX18 is an important development regulator of the pericardium, prostate, nephrons, urogenital tubes, and seminiferous tubules and mutations in TBX18 have been linked to pain in the chest, back, and flank. Conversely, CRELD2 which show differential allele frequencies in infertility patients is linked with fertility. CRELD2 is expressed in Oviductal epithelial cells in a manner that is very strongly correlated with the menstrual cycle and suggestive of an important reproductive role.

Pain and infertility can be two common but distinct clinical symptoms of endometriosis. In the present study, 9 non-synonymous variants were identified from a broad group of endometriosis associated variants that show distinct association with only one of the two symptoms and thus are suggestive of genetic classification of clinical subgroups of endometriosis.

### Example 3. Novel High-Risk Damaging Mutations Discovered in Familial Endometriosis

Whole exome sequencing (WES) was used in endometriosis families to determine if inherited, rare, high-risk protein coding variants contribute to endometriosis. Endometriosis is a complex disease with underlying genetic and environmental factors. Array-based genotyping platforms are well suited for GWA studies detecting association with common variants (minor allele frequencies >3-5%), whereas sequencing is required to detect rare and low-frequency protein coding variants. Subjects with familial endometriosis tend to carry a higher burden of genetic variants; families can be less likely to have potentially confounding (population stratification) effects. Studying genetic variants located on the same DNA strand (haplotypes) can help resolve the inheritance pattern of a disease variant by determining if two individuals who carry the same genetic variant have inherited the variant via shared recent ancestry (same haplotype) or whether their variants are derived from two independent mutation events (different haplotypes).

Methods

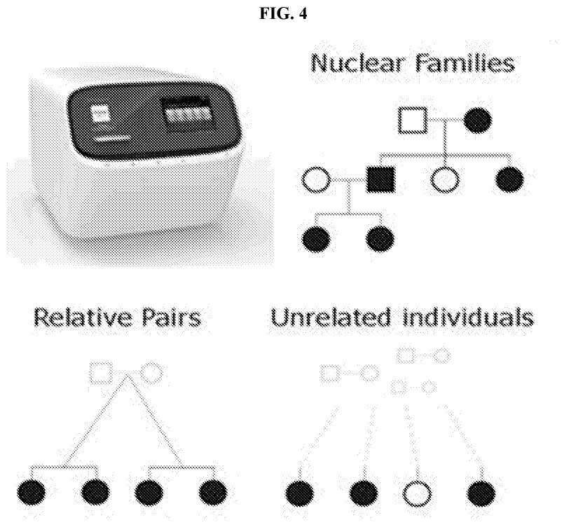

WES was performed on 489 women with familial endometriosis and 530 unrelated women (confirmed with identity-by-descent test) with endometriosis. Wes was also performed using Ion Proton Instrument (FIG. 4) and AmpliSeq Exome Capture kit. All missense and protein truncating variants with a MAF<1% in ExAc databse (Broad Institute) were considered for downstream analysis. Variant frequencies were compared with population frequency in ExAc database (n=33,000) using Fisher's exact test (exac. broadinstitute.org). Several software packages were used to predict whether the identified mutation would damage the encoded protein.

Consent and Medical Review

All subjects were provided written informed consent in accordance with study protocols approved by Quorum Review IRB (Seattle, Wash. 98101). Inclusion criteria were surgically confirmed diagnosis.

Results

This study identified 4 protein damaging variants significantly more prevalent in familial endometriosis. The 4 high-risk variants also pass genome-wide significance as shown in Table 6 below. Association was verified for all but the BRD9 variant in the cohort of unrelated endometriosis patient.

TABLE 6
Four genes with low-frequency damaging mutations showing association to endometriosis.

| | Index mutation | | | | | Gene burden | | | |
|---|---|---|---|---|---|---|---|---|---|
| Gene | AA_{change} | Endo_{Frq} | Exac_{Frq} | P | OR | Endo_{Frq} | Exac_{Frq} | P | OR |
| LONP1 | splice | 0.0028 | Not | 4.2 × 10⁻¹⁹ | Inf | 0.0302 | 0.0199 | 2.6 × 10⁻² | 1.5[1 − 2] |
| | | | seen | | | | | | |
| IGF2 | Q33X | 0.0048 | 0.0009 | 3.0 × 10⁻¹⁰ | 15[8 − 27] | 0.0085 | 0.0014 | 3.0 × 10⁻⁵ | 6[3 − 12] |
| BRD9 | K39R | 0.0009 | 0.0017 | 5.6 × 10⁻⁹ | 10[5 − 21] | 0.0057 | 0.0101 | 2.1 × 10⁻¹ | 0.6[0.3 − 1.3] |
| SNAP91 | T555A | 0.0106 | 0.0050 | 1.1 × 10⁻⁸ | 5[3 − 8] | 0.0179 | 0.0045 | 1.3 × 10⁻⁶ | 4[2 − 6] |

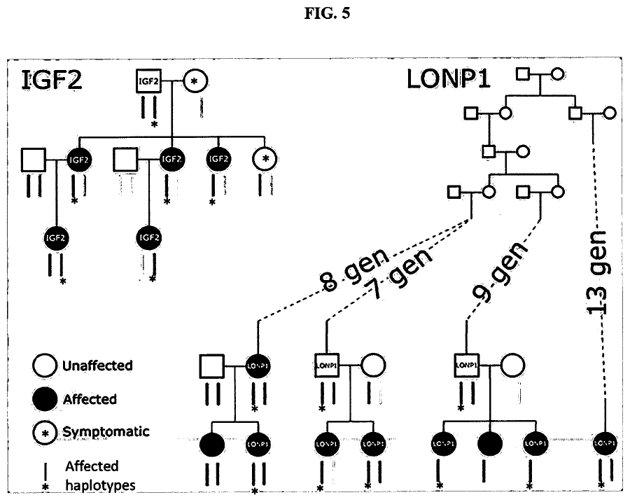

LONP1 (Lon protease) is a nuclear encoded protease in the mitochondria responsible for the degradation of misfolded proteins. LONP1 is expressed in endometrium and endometrial cancer, and affects endothelial mesenchymal transition in a dose dependent manner. Using a Genealogy database (GenDB) a shared ancestor ˜13 generations ago was identified. All affected individuals shown with LONP1 variant in FIG. 5 share identical haplotype of ˜140 kb which is concordant with a single shared ancestor 11-15 generations in the past.

IGF2 (Insulin-like growth factor 2) has previously been implicated in endometriosis in Korean women. The IGF axis has been implicated in growth regulation of endometriosis. In blood, IGF2 is an imprinted gene expressed only from the paternal haplotype.

SNAP91 (Synaptosome Associated Protein 91) and BRD9 (Bromodomain Containing 9) are novel endometriosis candidates but little is known about their function.

This study identified low-frequency damaging protein mutations segregating in families with endometriosis. IGF2 is the second implicated gene identified associated with endometriosis after NLRP2. Only 50 imprinted genes are known in humans to date suggesting imprinting plays a role in endometriosis. LONP1 and IGF2 regulate EMT in the pathogenesis of endometriosis.

### Example 4. CCDC168 and MUC12 Show Recessive Effects in Women with Endometriosis

Compound heterozygosity help identify genes involved in endometriosis. Whole Exome Sequencing (WES) was used on samples from 1,385 participants.

Samples

1019 Endometriosis samples were sequenced, 530 of which were for discovery, 301 of which were for replication, and 188 of which were related (2^{nd} cousin or closer). 366 control samples were sequenced.

Variant and Gene selection

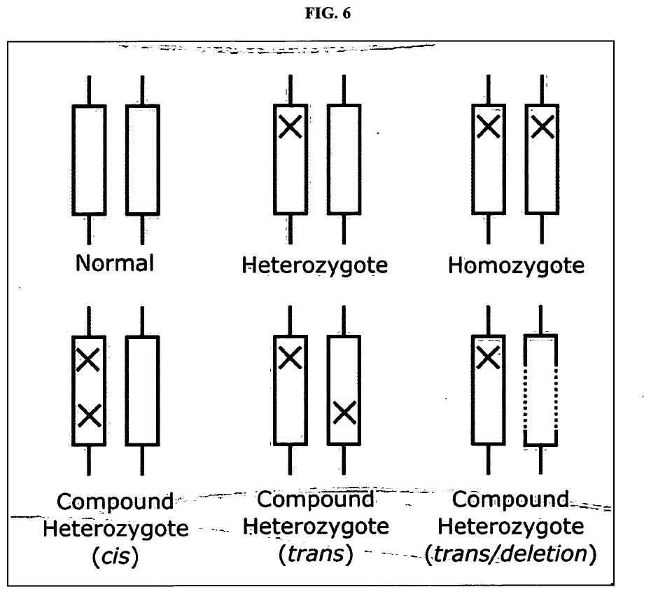

Protein-altering variants in discovery w frequency <1% in ExAC. 3039 genes were found individuals with 2+ variants per gene in the discovery set and thus can possibly be recessive genes. FIG. 6 illustrates mutation patterns cis/trans/haplotypes. Excess burden analysis of samples with 2+ protein-altering variants. Discovery (530 Endo vs 366 Ctl)—two genes with excess burden, P_{Fisher}<0.001. Replication (301 Endo vs 366 Ctl)—both genes replicate, P_{Fisher}<0.05.

Results

CCDC168 and MUC12 show significant excess variant count in endometriosis. Sample counts with rare protein-altering variants (ExAC_{freq}<1%)

TABLE 7
Variant count of CCDC168

| 95 Unique variants | 2+ | 0-1 |
|---|---|---|
| Cases | 31 | 988 |
| Controls | 0 | 366 |
| gnomAD (0.05) | 1 | 365 |

TABLE 8
Variant count of MUC12

| 82 Unique variants | 2+ | 0-1 |
|---|---|---|
| Cases | 47 | 970 |
| Controls | 1 | 365 |
| gnomAD (0.14) | 7 | 359 |

The variant counts of 2+ include all homozygotes, hemizygotes, and compound heterozygotes (cis and trans). Both genes show significant excess in endometriosis samples with 2+ hits also when compared with gnomAD.

The two novel genes, CCDC168 and MUC12, have large recessive effects in endometriosis and can be biologically relevant in endometriosis. 7.6% of endometriosis patients can have compound heterozygote mutations with 4-30 fold excess compared with control populations.

CCDC168 is a coiled-coil domain containing 168. CCDC168 can be differentially expressed in malignancies. Antibody staining can show prominent staining in various epithelial tissues. In some instances, CCDC168 is only present in placental animals (those with endometrium).

MUC12 is a transmembrane mucin expressed across many epithelial tissues including colon, pancreas, prostate or uterus. In some instances, transmembrane mucins are single-stranded proteins undergo proteolytic cleavage splitting TM and EC domains, lubricate epithelial surfaces, bind ligands, regulate epithelial wound healing, and/or extracellular domain detach with excess force (intracellular signaling and EMT). In some instances, a transmembrane mucin disclosed herein is MUC1, MUC4, MUC12, or MUC16. The extra cellular domain of MUC16 can be cancer antigen 125 (CA125), an important marker of ovarian cancer and endometriosis.

### Example 5. Rare Synonymous Mutations Show Strong Association with Endometriosis

The study is to determine if rare synonymous variants might contribute to the genetic risk for developing endometriosis. Synonymous and non-synonymous DNA variants can occur within the protein-coding part of a gene. Synonymous variants do not affect the amino-acid sequence, and non-synonymous variants do affect the amino-acid sequence, due to the redundancy in the genetic code. GWAS intergenic SNP variants may be determined from eQTL fine mapping, and rare non-synonymous variants may be determined from Whole Exome Sequencing.

Methods

Whole exome sequencing was performed on 1,077 study participants with surgically diagnosed endometriosis. Saliva DNA underwent AmpliSeq sequencing on an Ion Proton, and sequence was assembled using the Torrent software. Variant frequencies were compared to frequencies in gnomAD, which was used as reference for population-wide variant frequencies. Synonymous variants with a minor allele frequency <0.01 in the general population were considered. Fisher's Exact test was used to calculate association statistics. PANTHER database was used for GO (Gene Ontology) term enrichment analysis.

Results

114,877 synonymous rare variants were identified among patients. 648 synonymous variants passed the nominal significance threshold (p<0.05) across 617 genes. Table 9 shows five variants strongly associated with endometriosis that pass the genome-wide significance threshold of p≤5×10⁻⁸.

TABLE 9
Five strongly associated synonymous variants

| Gene | Chr | Position | P | OR | Nucl change | Amino Acid |
|---|---|---|---|---|---|---|
| KRTAP5-1 | 11 | 1,606,402 | 2.0 × 10⁻¹¹ | 43 | C78T | S26S |
| GPR137 | 11 | 64,051,889 | 6.7 × 10⁻¹⁵ | 49 | G51A | G17G |
| UBC | 12 | 125,398,297 | 1.5 × 10⁻³³ | 94 | T21C | T7T |
| ADAMTS7 | 15 | 79,058,944 | 2.5 × 10⁻¹¹ | 11 | T3309A | A1103A |
| SYNE1 | 6 | 152,457,795 | 6.7 × 10⁻⁸ | 5 | G25617A | E8539E |

17 genes have 2-or-more rare synonymous disease associated variants were found with only one expected by chance (p<0.001): ABCC5, ANK3, ATP8B4, CCDC147, CELSR1, DNAH3, EML6, HERC2, ITGA2, KIF23, LAMA5, PKD1, SLC22A20, SSPO, TENM2, TUBGCP2, VPS18. GO-term analysis show significant enrichment of a single GO term: “cytoskeletal structure and regulation” (OR=13.4). Rare intronic splice-junction variants were considered among the 17 genes, and 5 variants in CCDC147, LAMA5, and SSPO may affect the risk-burden.

This is the first time that rare synonymous variants may have been implicated in endometriosis. The genes may carry these mutations that are enriched for cytoskeletal function. Go-term and functional analysis implicate cytoskeletal regulation in the genetic predisposition of endometriosis. There variants may prove useful in developing a non-invasive test for endometriosis.

### Example 6. Large Effect Mutations in Endometriosis Genes Implicated by GWAS

Genome-wide association studies (GWAS) implicate several chromosomal regions as genetic risk factors for endometriosis. These regions have been “tagged” by polymorphic markers located between genes or in non-coding introns. Sequenced were the exons of 16 genes in GWAS regions to search for causative mutations, i.e., to find gene mutations responsible for the association observed in 16 genes implicated by endometriosis GWAS.

Methods

AmpliSeq sequencing on Ion Protons was conducted on DNA samples from 1,019 women with confirmed endometriosis. After sequence assembly using Torrent software, variant annotation was performed using ANNOVAR (hg19 reference). Frequencies of coding variants were compared against a large reference dataset (sequence data from 63,369 non-Finnish Europeans in gnomAD). Variants were found using Torrent Variant Caller (UCSC hg19). Association statistics were calculated using Fisher's Exact test; linkage disequilibrium statistics were calculated using LDlink. Cases: n=1,019 European women with confirmed endometriosis. Controls: n=63,369 non-Finnish Europeans in gnomAD).

Results

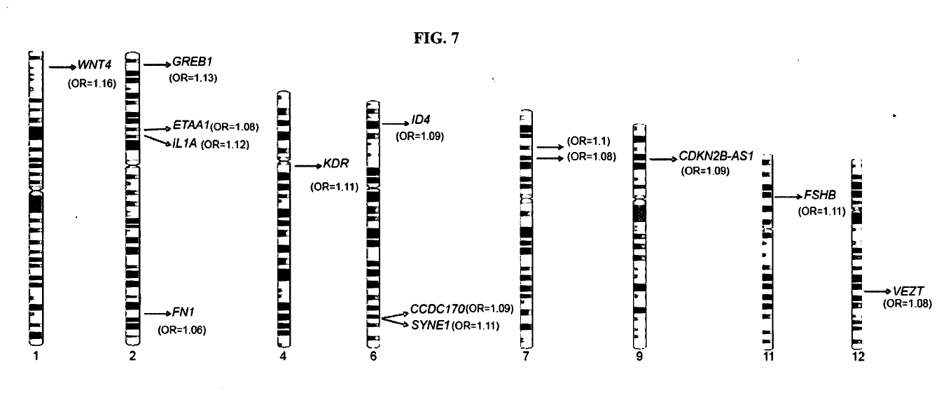

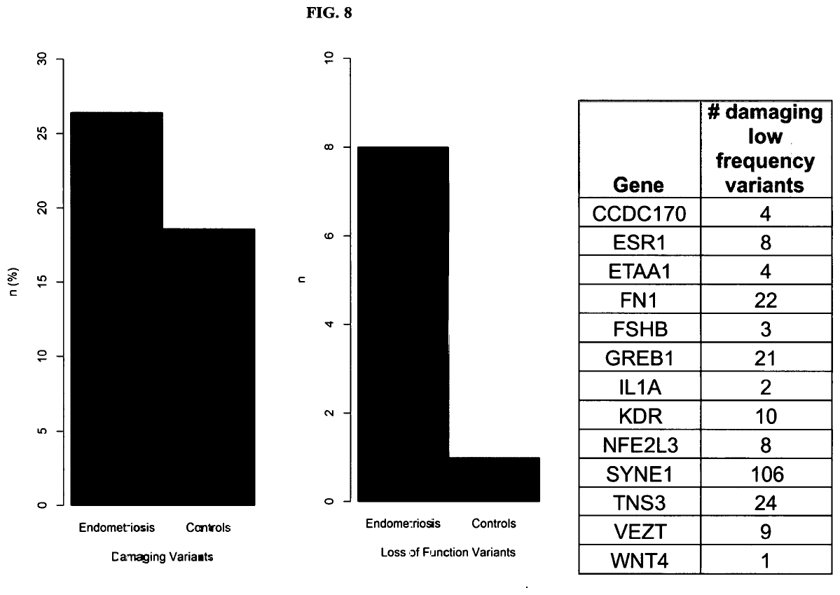

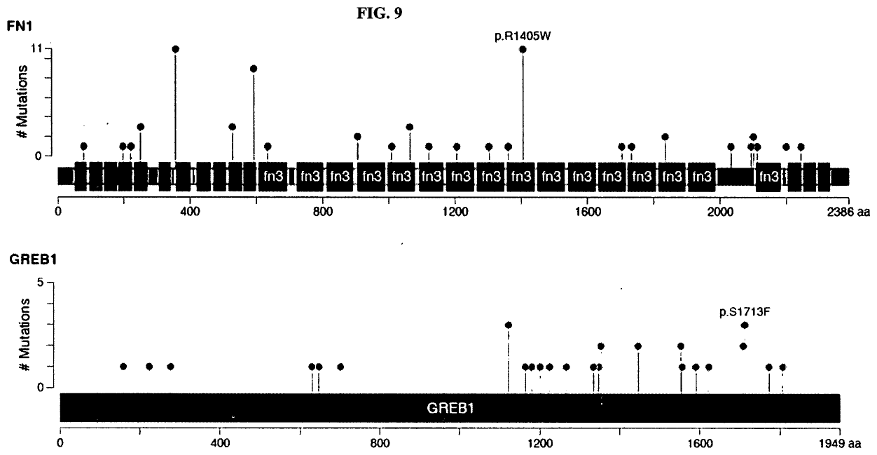

571 variants were detected; 333 of these alter an amino acid in the encoded protein and 234 low-frequency (MAF<1%), missense mutations are predicted to be pathogenic (in-silico). Likely pathologic variants are uncommon in the reference data (which contains women with endometriosis and males carrying risk factors); but the identified variants were often seen in multiple endometriosis patients. The excess of pathogenic mutations in cases was striking (p<10⁻¹⁶). 4 mutations (see Table 10) have high odds ratios for endometriosis with p values well below a multiple testing threshold (p≤9×10⁻⁵). Mutations predicted to shorten the encoded protein (loss of function) were also detected (2 splicing changes, and 7 “stop” mutations). Stop mutations (seen in five genes: GREB1, NFE2L3, FN1, SYNE1 and VEZT) were more prevalent in the endometriosis cohort compared to the population data (p=1.7×10⁻¹³). There is no measureable linkage disequilibrium between any of the new variants and tagging GWAS markers. FIG. 7 to FIG. 9 further illustrate the results.

TABLE 10
Mutations with p values below multiple correction threshold. Inf
means that the variant was not observed in the control cohort.

| | | | Endome- | | |
|---|---|---|---|---|---|
| | Protein | Control | triosis | | Odds Ratio |
| Gene | change | Frequency | Frequency | p(fisher) | [L95-U05] |
| FN1 | p.V527M | Not seen | 0.00147 | 4.03E−06 | Inf. |
| NFE2L3 | p.I233V | Not seen | 0.00147 | 4.03E−06 | Inf. |
| SYNE1 | p.E8539E | 0.00206 | 0.00785 | 1.11E−05 | 3.84 |
| VEZT | p.P712S | 0.00005 | 0.00196 | 1.23E−05 | 41.50 |

This is the first comprehensive study of coding mutations in all 16 GWAS candidate genes. Coding variants may not explain the association observed in GWAS studies, thus regulatory mutations outside of the coding regions are likely to be involved. The mutations having large effects confirm an important role for these genes in the pathogenesis of endometriosis.

### Example 7. Detailed Methods for Detection of Low Frequency Variants Medical Review

The inclusion criteria in the endometriosis case population in the present study were surgically confirmed diagnosis of endometriosis with laparoscopy being the preferred method. Trained OB/GYN clinicians performed the medical record review and clinical assessment of each individual patient. Patients were considered to be affected if they had biopsy-proven lesions or if operative reports revealed unambiguous gross lesions. Patients were further categorized by severity, clinical history of pelvic pain, infertility, dyspareunia or dysmenorrhea and family history. Patients were grouped into one of three classes of severity: mild, moderate or severe, following the general guidelines set forth by ASRM. This analysis compared cases with 100% prevalence of endometriosis to controls with the population prevalence of endometriosis (5-10%).

DNA Extraction.

Saliva samples were collected using the Oragene 300 saliva collection kit (DNA Genotek; Ottawa, Ontario, Canada) and DNA was extracted using an automated extraction instrument, AutoPure LS (Qiagen; Valencia, Calif.), and manufacturer's reagents and protocols. DNA quality was evaluated by calculation absorbance ratio OD260/OD280, and DNA quantification was measured using PicoGreenH (Life Technologies; Grand Island, N.Y.).

Microarray Genotyping.

The discovery set of 2019 endometriosis cases and 25476 population controls were genotyped using the Illumina Human OmniExpress Chip (Illumina; San Diego, Calif.) according to protocols provided by the manufacture. An additional 905 endometriosis cases were genotyped on a custom designed microarray using the Affymetrix GeneTitan platform according to the manufacturer's instructions.

Sample Quality Control.

Samples were excluded from the analysis if they missed any of the following quality thresholds:
a) Evidence of familial relationship closer that 3rd-degree (pi-hat>0.2) using genome-wide Identity-By-State (IBS) estimation implemented in PLINK
b) Samples with missing genotypes >0.02
c) Samples with non-European admixture >0.05 as determined by ADMIXTURE

SNP Quality Control.

SNPS were excluded from the analysis if they missed any of the following quality thresholds:
a) SNPs from copy number variant regions or regions with adjacent SNPs
b) SNPs failing Hardy-Weinberg Equilibrium (HWE) P<=10⁻³
c) SNPs with minor allele frequency (MAF)<=0.01 in the control population
d) SNP call rate <=98%

Admixture.

ADMIXTURE (ver. 1.22) was used to estimate the individual ancestry proportion. The software estimates the relative admixture proportions of a given number of a priori defined ancestral groups contributing to the genome of each individual. The POPRES dataset (Nelson M R et al. 2008) was used as a reference group to create a supervised set of 9 ancestral clusters. Seven of them belong to the European subgroups along with African and Asian groups. Since POPRES dataset utilized Affymetrix 5.0 chip, 105,079 autosomal SNPs that overlapped with the Illumina OmniExpress dataset were used. Among the 105,079 SNPs, a subset of 33,067 SNPs was selected that showed greater genetic variation (absolute difference in frequency) among the 9 reference groups. The pair-wise autosomal genetic distance determined by Fixation Index (FST) using 33,067 SNPs was calculated for the 9 reference groups as listed in POPRES dataset. Subsequently, a conditional test was used to estimate the admixture proportions in the unknown samples as described by Alexander et al. (2009).

Principal Component Analysis (PCA).

PCA was applied to account for population stratification among the European subgroups. The previously identified 33,067 SNPs were selected to infer the axes of variation using EIGENSTRAT. Only the top 10 eigenvectors were analyzed. Most of the variance among the European populations was observed in the first and second eigenvector. The first eigenvector accounts for the east-west European geographical variation while the second accounts for the north-south component. Only the top 10 eigenvectors showed population differences using Anova statistics (p<0.01). The PCA adjusted Armitrage trend P-values were calculated using the top 10 eigenvectors as covariates.

Association Analysis.

After the quality of all data was confirmed for accuracy, genetic association was determined using the whole-genome association analysis toolset, PLINK (ver. 1.07). Differences in allele frequencies between endometriosis patients and population controls were tested for each SNP by a 1 degrees of freedom Cochran-Armitrage Trend test. The allelic odds ratios were calculated with a confidence interval of 95%. SNPs that passed the quality control parameters were prioritized using the PCA adjusted cochran-Armitrage trend test P-values. The combined/metaanalysis of different datasets was performed using Cochran-Mantel-Hanszel method as well as using Cochran-Armitrage Trend test. Breslow Day test was used to determine between-cluster heterogeneity in the odds ratio for the disease/SNP association.

Software Used.

PLINK (version 1.07; http://pngu.mgh.harvard.edu/^{˜}purcell/plink/index.shtml). R (version 2.15.0; http://www.r-project.org/). EIGENSTRAT (version 3.0; http://genepath.med.harvard.edu/^{˜}reich/Software.htm).

### Example 8. Detailed Methods for Gene Sequencing and Detection of Low-Frequency Damaging Variants

DNA Extraction and Genotyping.

DNA used in the present study was extracted from blood or saliva using standard extraction methods. Genotyping was performed using the Illumina HumanExome (Illumina, San Diego, Calif.) according to protocols provided by the manufactures.

Sample and SNP Quality Control

The discovery set of 1518 cases were genotyped using the Illumina Human Exome Chip (Illumina; San Diego, Calif.) per protocols provided by the manufacture.

Samples were excluded from the analysis if they missed any of the following quality thresholds:
a) Evidence of familial relationship closer that 3rd-degree (\hat{π}>0.2) using genome-wide Identity-By-State (IBS) estimation implemented in PLINK.
b) Samples with missing genotypes >0.02
c) Samples with non-European admixture >0.05 as determined by ADMIXTURE

SNPS were excluded from the analysis if they missed any of the following quality thresholds:
a) SNPs with Illumina GenTrain Score <0.65
b) SNPs from copy number variant regions or regions with adjacent SNPs
c) SNP call rate ≤98%

Exome Sequencing and Variant Discovery

Whole exome sequencing (WES) was performed on 2400 endometriosis cohort using Ion Proton Instrument as per the manufacturers protocol (Life Technologies, Carlsbad Calif.) using their AmpliSeq Exome Capture Kit. Sequence alignment and variant calling was performed against the reference human genome (UCSC hg19 version). The variant discovery was performed using Life Technologies TMAP algorithm with their default parameter settings, and Life Technologies Torrent Variant Caller was used to discover variants. The variants identified from the Torrent Variant Caller were taken further for downstream analysis. The variants included were single nucleotide variants, short insertions, or deletions. Variant annotation was performed using ANNOVAR. The coding variants were classified as missense, frameshift, splicing, stop-gain, or stop-loss. Variants were considered “loss-of-function” if they caused a stop-gain, splicing, or frame-shift insertion or deletion. Prediction of protein function was evaluated in silico using seven different algorithms (Polyphen 2, Sift, Mutation Accessor, Mutation Taster, FATHMM, LRT, and MetaLR. Missense variants were deemed “damaging missense” if they were predicted damaging by at least one of the seven algorithms tested. The genes that harbor these variants were also checked against the published “FLAGS” gene list (Shyr C et al. 2014) to understand whether the gene is frequently mutated in humans.

Low Frequency Variants

Variants that pass the population control frequency (gnomAD) of MAF<1% were called “low frequency variants”. These variants were analyzed to test for association using Fisher's Exact Test. The low frequency variants were prioritized based on their Fisher's p value.

Gene Burden

The genetic burden was calculated for each gene by collapsing/combining all low frequency variants identified through WES. Fisher's Exact Test was used to determine excess gene burden in endometriosis subjects compared to the control population counts as observed in gnomAD database by generating 2×2 table per gene for the number of reference and alternative alleles. The genes were then prioritized based on their Fisher's p value.

TABLE 1
Variants associated with endometriosis. Inf means that the variant was not
observed in the control cohort.

| | | | Alter- | | | | | | | | |
|---|---|---|---|---|---|---|---|---|---|---|---|
| | | | nate | | | | | | | | |
| | | Ref- | Al- | | | | | | | | |
| | | er- | lele/ | | Amino | | | | | | |
| | | ence | Minor | | Acid | | Con- | p | OR | | SEQ |
| | Posi- | Al- | Al- | | posi- | Case | trol | val- | L95- | | ID |
| Chr | tion | lele | lele | Gene | tion | MAF | MAF | ue | [U95] | Context Sequence | NO |
| chr | 113921 | G | A | TNFRS | p.R175 | 0.006 | 0.004 | 2.97 | 1.57 | CCTGGGGAGGGGCTGGCTGC | SEQ |
| 1 | 6 | | | F18 | C | 86 | 37 | E−02 | [1.07- | GGTCGGTGGCCCCGGAGGAC | ID |
| | | | | | | | | | 2.31] | [G/A]GCCAGGCTCACACCC | NO: |
| | | | | | | | | | | ACAGGTCTCCCAGCCGCCCC | 1 |
| | | | | | | | | | | TTCTC | |
| chr | 145259 | T | C | ATAD3 | p.W11 | 0.007 | 0.000 | 2.93 | 19.24 | GCTGGAAGCCCTGAGCCTGC | SEQ |
| 1 | 2 | | | A | 0R | 35 | 38 | E−22 | [11.09- | TGCACACACTAGTCTGGGCA | ID |
| | | | | | | | | | 33.38] | [T/C]GGAGTCTCTGCCGTG | NO: |
| | | | | | | | | | | CCGGAGCCGTGCAGACACAG | 2 |
| | | | | | | | | | | GAGCG | |
| chr | 370358 | C | T | LRRC4 | p.V301 | 0.006 | 0.004 | 2.53 | 1.61 | ACGTGCAGGACCCTGAGCAG | SEQ |
| 1 | 9 | | | 7 | M | 62 | 12 | E−02 | [1.09- | CAGCCGGCCGGCATCTCCCA | ID |
| | | | | | | | | | 2.38] | [C/T]GTCCTGCTCCTCCCC | NO: |
| | | | | | | | | | | ATCACCACCTTCCCGCCTCT | 3 |
| | | | | | | | | | | GCTTC | |
| chr | 908311 | G | T | SLC2A7 | p.T59N | 0.006 | 0.003 | 1.69 | 1.7 | GAGCTTCCCGTCCATGAATG | SEQ |
| 1 | 2 | | | | | 13 | 61 | E−02 | [1.14- | TTGCGTGTCGCTCAAAGTAG | ID |
| | | | | | | | | | 2.55] | [G/T]TTTCGTTGTAAAATG | NO: |
| | | | | | | | | | | ACTTGAAGACCTGGAAAACA | 4 |
| | | | | | | | | | | TTGCC | |
| chr | 105293 | A | G | DFFA | p.I69T | 0.007 | 0.005 | 4.67 | 1.46 | ATTGGAAGGTAGACACAGAA | SEQ |
| 1 | 26 | | | | | 60 | 20 | E−02 | [1.02- | AGTAATCGTCATCATCCACT | ID |
| | | | | | | | | | 2.1] | [A/G]TGGTGCCATCCTCTG | NO: |
| | | | | | | | | | | CCAGGACCAGGGTGACTGGT | 5 |
| | | | | | | | | | | GTCAG | |
| chr | 119833 | C | T | KIAA20 | p.E410 | 0.005 | 0.003 | 1.44 | 1.72 | ATCTGCTGGACGGAGGACAG | SEQ |
| 1 | 52 | | | 13 | K | 88 | 42 | E−02 | [1.14- | CCGCCCCGGCCACAGGTTCT | ID |
| | | | | | | | | | 2.61] | [C/T]GGCGTGCATGGTGGC | NO: |
| | | | | | | | | | | GTGCCCGCTGAAGCAGTGAT | 6 |
| | | | | | | | | | | CTTCA | |
| chr | 128559 | A | G | PRAME | p.N42 | 0.005 | 0.003 | 3.74 | 1.63 | TCCTGCCCCTGAGGAGAGTT | SEQ |
| 1 | 96 | | | F1 | 6D | 39 | 31 | E−02 | [1.06- | TGAATTCCTTGGTTCGTGTC | ID |
| | | | | | | | | | 2.52] | [A/G]ATTGGGAGATCTTCA | NO: |
| | | | | | | | | | | CCCCACTTCGGGCTGAGCTG | 7 |
| | | | | | | | | | | ATGTG | |
| chr | 128560 | C | T | PRAME | p.G453 | 0.014 | 0.003 | 6.69 | 4.78 | CACTGAGGGAAGTCAGGCAG | SEQ |
| 1 | 79 | | | F1 | G | 22 | 01 | E−20 | [3.6- | CCCAAGAGGATCTTCATTGG | ID |
| | | | | | | | | | 6.33] | [C/T]CCCACCCCCTGCCCT | NO: |
| | | | | | | | | | | TCCTGTGGCTCATCACCGTC | 8 |
| | | | | | | | | | | TGAGG | |
| chr | 136692 | C | T | PRAME | p.E352 | 0.006 | 0.000 | 5.37 | 201.46 | TGGGAGTAGTGGATCTGACA | SEQ |
| 1 | 76 | | | F14 | K | 86 | 03 | E−35 | [61.22- | GCCCTCCAAGATGAGGGTTT | ID |
| | | | | | | | | | 662.92] | [C/T]GAGAGAGGCAGCAAT | NO: |
| | | | | | | | | | | TTTCTCTAGCAGAGCTCCGA | 9 |
| | | | | | | | | | | GGGGT | |
| chr | 159869 | A | T | RSC1A | p.N20 | 0.005 | 0.002 | 2.92 | 1.78 | AACATAGGGGACCTTGAGCT | SEQ |
| 1 | 77 | | | 1 | 51 | 205 | 931 | E−02 | [1- | TCCTGAAGAAAGGCAACAGA | ID |
| | | | | | | | | | 2.94] | [A/T]TCAACACAAAATTGT | NO: |
| | | | | | | | | | | TGATTTGGAAGCTACGATGA | 10 |
| | | | | | | | | | | AAGGA | |
| chr | 176033 | C | T | PADI3 | p.H508 | 0.009 | 0.006 | 2.64 | 1.47 | CCTGCTTCAAGCTCTTCCAG | SEQ |
| 1 | 40 | | | | H | 07 | 19 | E−02 | [1.05- | GAAAAGCAGAAGTGTGGCCA | ID |
| | | | | | | | | | 2.05] | [C/T]GGGAGGGCCCTCCTG | NO: |
| | | | | | | | | | | TTCCAGGGGGTTGTTGGTGG | 11 |
| | | | | | | | | | | GTAAC | |
| chr | 194511 | A | T | UBR4 | p.A314 | 0.011 | 0.008 | 4.27 | 1.38 | TTTCTGTTAGAAGCTGAGTA | SEQ |
| 1 | 76 | | | | 9A | 27 | 21 | E−02 | [1.02- | TAGGCCTCAAACACATCAGC | ID |
| | | | | | | | | | 1.86] | [A/T]GCATGACCCTGGGAG | NO: |
| | | | | | | | | | | AAGAAAATTTGCATGAGAAC | 12 |
| | | | | | | | | | | CTGTG | |
| chr | 195040 | T | C | UBR4 | p.M84 | 0.011 | 0.008 | 4.24 | 1.38 | CGAGCCAAGATAAGCGGCAC | SEQ |
| 1 | 62 | | | | 4V | 27 | 19 | E−02 | [1.03- | GAAGCGCATCTGAGCATCCA | ID |
| | | | | | | | | | 1.86] | [T/C]GTTGACGCTCAACTC | NO: |
| | | | | | | | | | | CTGGATGATCTGGACAAAAA | 13 |
| | | | | | | | | | | GCGAC | |
| chr | 195458 | G | A | EMC1 | p.Y961 | 0.011 | 0.008 | 2.81 | 1.4 | TGGCAAAAACCAGGCCAAAG | SEQ |
| 1 | 93 | | | | Y | 52 | 23 | E−02 | [1.05- | AGGACGCTGCTGATTAACAC | ID |
| | | | | | | | | | 1.89] | [G/A]TAGTCATAGTCATCC | NO: |
| | | | | | | | | | | TTCAGAACGTCAAACTGCTT | 14 |
| | | | | | | | | | | GGATG | |
| chr | 204428 | C | T | PLA2G | p.G45S | 0.009 | 0.006 | 3.60 | 1.41 | TCTTTGGGTTGGCCTCTGCC | SEQ |
| 1 | 78 | | | 20 | | 80 | 95 | E−02 | [1.03- | ACCTAGTCCGCAGTGACAGC | ID |
| | | | | | | | | | 1.95] | [C/T]GTAGGGCCAGTAGGA | NO: |
| | | | | | | | | | | GAGGATGGGCATTTTCCCAG | 15 |
| | | | | | | | | | | TCACT | |
| chr | 238455 | A | T | E2F-2 | p.A257 | 0.011 | 0.008 | 4.78 | 135 | CCTTGACGGCAATCACTGTC | SEQ |
| 1 | 89 | | | | A | 52 | 56 | E−02 | [1.01- | TGCTCCTTAAAGTTGCCAAC | ID |
| | | | | | | | | | 1.81] | [A/T]GCACGGATATCCTGG | NO: |
| | | | | | | | | | | TAAGTCACATAGGCCAGCGT | 16 |
| | | | | | | | | | | AGGGC | |
| chr | 244881 | C | T | IFNLR1 | p.E137 | 0.009 | 0.006 | 3.42 | 1.46 | TGCAGGGGGGCAGCTGGTAC | SEQ |
| 1 | 31 | | | | E | 31 | 39 | E−02 | [1.04- | GTGGCATTGGCACTCAGGAT | ID |
| | | | | | | | | | 2.05] | [C/T]TCCTCCGTCTGGGTG | NO: |
| | | | | | | | | | | AGCACCAGGACAGGTGGGGC | 17 |
| | | | | | | | | | | CGGCT | |
| chr | 266088 | A | G | UBXN1 | p.G490 | 0.008 | 0.000 | 5.67 | 44.62 | CGGGACTGGGGCCGGGACCG | SEQ |
| 1 | 83 | | | 1 | G | 82 | 20 | E−34 | [23.65- | GGACCGGGACTGGGGCCGGG | ID |
| | | | | | | | | | 84.2] | [A/G]CCGGGACCGGGACAG | NO: |
| | | | | | | | | | | GGACCAGGACTGAATTTCAG | 18 |
| | | | | | | | | | | GCTGG | |
| chr | 266714 | G | C | AIM1L | p.P579 | 0.018 | 0.000 | 5.40 | Inf | TGAGGCAGCAGGAGCACCAG | SEQ |
| 1 | 13 | | | | R | 63 | 00 | E−89 | | GGCCCTTCACAACCTCTTTT | ID |
| | | | | | | | | | | [G/C]GGGTGGTGGACAAGG | NO: |
| | | | | | | | | | | CAGCAGGAGCACCAGACCCC | 19 |
| | | | | | | | | | | TGCAC | |
| chr | 266716 | A | G | AIM1L | p.S508 | 0.025 | 0.000 | 4.40 | 127.41 | CAGGAGCACTGGACCCCTGC | SEQ |
| 1 | 25 | | | | S | 98 | 21 | E−88 | [55.94- | ACCACCTCCTTCTGGGTGGG | ID |
| | | | | | | | | | 290.2] | [A/G]GATGAGGCAGCAGGA | NO: |
| | | | | | | | | | | GCACCAGGGCCCTTCACGAC | 20 |
| | | | | | | | | | | CTCTT | |
| chr | 276743 | G | A | SYTL1 | p.A126 | 0.005 | 0.000 | 1.34 | Inf | CCCAGGAGACCAGGCTCCAG | SEQ |
| 1 | 34 | | | | T | 88 | 00 | E−35 | | GCCACGACAGGGAGGCTGAG | ID |
| | | | | | | | | | | [G/A]CTGCTGTGAAAGAGA | NO: |
| | | | | | | | | | | AGGAAGAGGGGCCAGAGCCC | 21 |
| | | | | | | | | | | AGGTG | |
| chr | 289319 | T | A | TAF12 | p.T145 | 0.006 | 0.004 | 4.77 | 1.48 | CCAGGGCTCTGGCATTTCCT | SEQ |
| 1 | 01 | | | | S | 86 | 65 | E−02 | [1.01- | CACCTGTTTGTGAGCTTCTG | ID |
| | | | | | | | | | 2.17] | [T/A]GGTGCAAGCTTTTTT | NO: |
| | | | | | | | | | | GTAGGGTCGGATTTCTTCAG | 22 |
| | | | | | | | | | | AGCCA | |
| chr | 294477 | G | A | TMEM | p.C183 | 0.005 | 0.002 | 4.90 | 1.94 | TACCCCGACGCGGGGACGGG | SEQ |
| 1 | 92 | | | 200B | C | 64 | 91 | E−03 | [1.27- | TCCCAGATTTCTGGCTCTGC | ID |
| | | | | | | | | | 2.97] | [G/A]CAGCCTACGGCTCGG | NO: |
| | | | | | | | | | | GGACTCCTAGGGCCGGGGCT | 23 |
| | | | | | | | | | | GGGAA | |
| chr | 314096 | A | 3 | PUM1 | p.A109 | 0.005 | 0.003 | 3.69 | 1.64 | GGGGACCGTCGTTCATGGTG | SEQ |
| 1 | 34 | | | | 7A | 39 | 29 | E−02 | [1.07- | CACACCTCATCGATGAGCAC | ID |
| | | | | | | | | | 2.53] | [A/G]GCGCGCTCCGTACGT | NO: |
| | | | | | | | | | | GAGGCGTGAGTAACACACTT | 24 |
| | | | | | | | | | | CTCCA | |
| chr | 353707 | C | A | DLGAP | p.G83 | 0.013 | 0.000 | 1.13 | 301.95 | TGGCCAGGGTACATCCTGGG | SEQ |
| 1 | 38 | | | 3 | W | 24 | 04 | E−63 | [94.37- | GAAGGTGCTGCTACCCCCCC | ID |
| | | | | | | | | | 966.13] | [C/A]AACCCCGGCCCCCGC | NO: |
| | | | | | | | | | | TGGCCCTCCCTCAGGGCCTA | 25 |
| | | | | | | | | | | CCGAC | |
| chr | 405332 | C | G | CAP1 | p.C236 | 0.029 | 0.000 | 5.89 | Inf | GACCCTCTGCCGGATCATGT | SEQ |
| 1 | 89 | | | | W | 90 | 00 | E− | | CCTCCTCCCCCTCCACCATG | ID |
| | | | | | | | | 176 | | [C/G]CCCCCTCGTCCCCCA | NO: |
| | | | | | | | | | | GTCTCTACCATTTCATGCTC | 26 |
| | | | | | | | | | | ATATG | |
| chr | 407023 | A | G | RLF | p.T656 | 0.011 | 0.007 | 3.11 | 1.41 | TGAATGACCAAGCCAAAGGA | SEQ |
| 1 | 42 | | | | T | 03 | 87 | E−02 | [1.04- | GAGTCTCATGAATATGTCAC | ID |
| | | | | | | | | | 1.9] | [A/G]TTCAGCAAATTAGAA | NO: |
| | | | | | | | | | | GATTGCCACCTGCAAGACAG | 27 |
| | | | | | | | | | | AGATT | |
| chr | 409289 | A | G | ZFP698 | p.Q43 | 0.006 | 0.004 | 1.30 | 1.67 | AGTAAAACCTTCAGCCATAG | SEQ |
| 1 | 69 | | | | 8R | 86 | 12 | E−02 | [1.14- | TACATACCTAACTCAACACC | ID |
| | | | | | | | | | 2.45] | [A/G]GAGAACTCATACTGG | NO: |
| | | | | | | | | | | AGAAAGACCATATAAATGTA | 28 |
| | | | | | | | | | | AGGAA | |
| chr | 476914 | G | C | TAL1 | p.A27 | 0.005 | 0.000 | 6.06 | 460.35 | CTCCTTGGCGACGCGGTTCA | SEQ |
| 1 | 81 | | | | G | 15 | 01 | E−28 | [61.91- | GCAGGACCAGGTGCGGGGGG | ID |
| | | | | | | | | | 3423.16] | [G/C]CCATGCTGGCCTCGG | NO: |
| | | | | | | | | | | CCGCGTCCCGTCCCTCTAGC | 29 |
| | | | | | | | | | | TGGGG | |
| chr | 477168 | C | T | STIL | p.T126 | 0.009 | 0.006 | 4.22 | 1.41 | AGAAGGTGCCTACTGAATTC | SEQ |
| 1 | 89 | | | | 2T | 56 | 79 | E−02 | [1.02- | ATGCTATTCATCTGCTTTAG | ID |
| | | | | | | | | | 1.95] | [C/T]GTTTCAGAAGGTTGC | NO: |
| | | | | | | | | | | AAACTTTCAGGAAAAATTGT | 30 |
| | | | | | | | | | | AATGT | |
| chr | 556436 | T | C | USP24 | p.T158 | 0.007 | 0.004 | 4.65 | 1.51 | GTTCTAAGGTCTGAAAACTT | SEQ |
| 1 | 58 | | | | A | 11 | 71 | E−02 | [1.03- | ACCAAGTCTTGCTAGGTAGG | ID |
| | | | | | | | | | 2.22] | [T/C]AGATGCCAACAGGCA | NO: |
| | | | | | | | | | | TTTGCCTAGTGATTCTTCTC | 31 |
| | | | | | | | | | | GCTTG | |
| chr | 953304 | C | T | SLC44A | p.N42 | 0.006 | 0.004 | 2.78 | 1.57 | TGGTGAGGATTCCGAGAATC | SEQ |
| 1 | 40 | | | 3 | 4N | 62 | 23 | E−02 | [1.06- | ATTGTCATGTACATGCAAAA | ID |
| | | | | | | | | | 2.32] | [C/T]GCACTGAAAGAACAG | NO: |
| | | | | | | | | | | GTAAGGCTACCTCCTGATAC | 32 |
| | | | | | | | | | | ACAGC | |
| chr | 109792 | T | C | CELSR2 | p.L17P | 0.009 | 0.000 | 2.93 | 21.06 | CCGGCCACCGGCGTCCCCCT | SEQ |
| 1 | 751 | | | | | 80 | 47 | E−32 | [13.61- | CCCAACGCCGCCGCCGCCGC | ID |
| | | | | | | | | | 32.59] | [T/C]GCTGCTGCTGTTGCT | NO: |
| | | | | | | | | | | GCTGCTGCTGCCGCCGCCAC | 33 |
| | | | | | | | | | | TATTG | |
| chr | 110302 | A | T | EPS8L3 | p.F55I | 0.006 | 0.003 | 3.20 | 1.92 | AAGTCTTGGCTCCACACCCG | SEQ |
| 1 | 392 | | | | | 13 | 20 | E−03 | [1.28- | GCCCTGTGCATCCATCTCGA | ID |
| | | | | | | | | | 2.88] | [A/T]CAGCTTCTGCAAGGC | NO: |
| | | | | | | | | | | ATCCTCGGGCCCCTGGACTC | 34 |
| | | | | | | | | | | TCTGA | |
| chr | 117122 | T | C | IGSF3 | p.K102 | 0.025 | 0.000 | 1.05 | Inf | CTTTCCTCTTCCTGTTCTTC | SEQ |
| 1 | 350 | | | | 0E | 25 | 00 | E− | | CAGGCCAGGGCTGCTCCTTT | ID |
| | | | | | | | | 150 | | [T/C]CCCCCCAGCTTTAGT | NO: |
| | | | | | | | | | | CCTCAGGGAATACCAGGCCA | 35 |
| | | | | | | | | | | CAGCG | |
| chr | 120054 | G | T | HSD3B | p.R71I | 0.010 | 0.007 | 1.85 | 1.48 | GTGCTGGAAGGAGACATTCT | SEQ |
| 1 | 192 | | | 1 | | 54 | 17 | E−02 | [1.08- | GGATGAGCCATTCCTGAAGA | ID |
| | | | | | | | | | 2.01] | [G/T]AGCCTGCCAGGACGT | NO: |
| | | | | | | | | | | CTCGGTCATCATCCACACCG | 36 |
| | | | | | | | | | | CCTGT | |
| chr | 144856 | C | T | PDE4DI | p.A210 | 0.009 | 0.005 | 1.71 | 1.54 | TTACCTCTGTGCCTTGGGCT | SEQ |
| 1 | 852 | | | P | 5A | 07 | 92 | E−02 | [1.1- | TCAAGGCCAGGGAAGCTGCA | ID |
| | | | | | | | | | 2.14] | [C/T]GCTGATCTCACAAGA | NO: |
| | | | | | | | | | | GACACTATCTTTTTGACCAG | 37 |
| | | | | | | | | | | CAGCT | |
| chr | 144912 | G | T | PDE4DI | p.P695 | 0.005 | 0.002 | 5.35 | 2.39 | ACAGGCAGTGGGGGTAACTT | SEQ |
| 1 | 191 | | | P | H | 15 | 16 | E−04 | [1.53- | CAGCTTGTTGGTTAGAGATG | ID |
| | | | | | | | | | 3.74] | [G/T]GTGCTTGGGACATCA | NO: |
| | | | | | | | | | | GGGAGTCTCTCCCTCCTAAA | 38 |
| | | | | | | | | | | TATTG | |
| chr | 144930 | A | C | PDE4DI | p.S244 | 0.007 | 0.004 | 7.25 | 1.73 | CTTTCTGTTGTGGAGGGCTA | SEQ |
| 1 | 977 | | | P | S | 35 | 27 | E−03 | [1.19- | GCCTGGACGCTTGCATCCAA | ID |
| | | | | | | | | | 2.5] | [A/C]GATTCCACAGAGGAA | NO: |
| | | | | | | | | | | CCAGGCGTCTCTTCCTCCAT | 39 |
| | | | | | | | | | | GCTTT | |
| chr | 145537 | C | A | ITGA10 | p.S841 | 0.009 | 0.006 | 2.01 | 1.5 | CAACTCTGGAGAACAGAAAG | SEQ |
| 1 | 513 | | | | R | 31 | 22 | E−02 | [1.08- | GAAAATGCTTACAATACGAG | ID |
| | | | | | | | | | 2.09] | [C/A]CTGAGTCTCATCTTC | NO: |
| | | | | | | | | | | TCTAGAAACCTCCACCTGGC | 40 |
| | | | | | | | | | | CAGTC | |
| chr | 149897 | G | A | SF3B4 | p.P245 | 0.007 | 0.005 | 2.66 | 1.52 | GGGGTATCCCAGGTGGGAGG | SEQ |
| 1 | 906 | | | | P | 84 | 17 | E−02 | [1.06- | GCTCCAGGAGGTGGCACTGG | ID |
| | | | | | | | | | 2.18] | [G/A]GGTGGGAAGGAGCCA | NO: |
| | | | | | | | | | | GGAGGAGGCATGCCTATAGA | 41 |
| | | | | | | | | | | GGAAA | |
| chr | 152080 | C | T | TCHH | p.E180 | 0.010 | 0.000 | 2.67 | Inf | TTCCGTCACGCTGTTGGGGG | SEQ |
| 1 | 275 | | | | 6E | 54 | 00 | E−63 | | CGCAGCTGCTGTTCTTCCCT | ID |
| | | | | | | | | | | [C/T]TCCTGGCGTAGCTGT | NO: |
| | | | | | | | | | | TCCTCCTCGCGGAATTTTCT | 42 |
| | | | | | | | | | | GTCAG | |
| chr | 152082 | T | C | TCHH | p.K108 | 0.013 | 0.000 | 1.95 | 28.95 | CTCAGCAGCTGCTCTTCCTC | SEQ |
| 1 | 449 | | | | 2E | 24 | 46 | E−48 | [19.46- | CTGCTGCAGCTCCTCTTCCT | ID |
| | | | | | | | | | 43.05] | [T/C]CCGATATTGCCTCTC | NO: |
| | | | | | | | | | | CAGCTCCTGGCGCCTTCTCG | 43 |
| | | | | | | | | | | TCTCC | |
| chr | 152083 | G | T | TCHH | p.P789 | 0.010 | 0.000 | 1.16 | Inf | CTCCTCGGCCCTCAGCTGCC | SEQ |
| 1 | 327 | | | | Q | 29 | 00 | E−61 | | TCTCCCGCTGCTCCCGCAAT | ID |
| | | | | | | | | | | [G/T]GGGGCCTGGCCGACA | NO: |
| | | | | | | | | | | GCCTCTGACGGCCCCTCTCG | 44 |
| | | | | | | | | | | CTCTT | |
| chr | 152083 | G | T | TCHH | p.R622 | 0.019 | 0.000 | 1.65 | Inf | TTCAGCAGCTGCTGGCGCCT | SEQ |
| 1 | 829 | | | | S | 36 | 00 | E− | | CTCTTCCTCCGGCTCCTCGC | ID |
| | | | | | | | | 115 | | [G/T]CTTCAGCCGCTGCTC | NO: |
| | | | | | | | | | | GCGCCTCTCCTCCTGCTCGA | 45 |
| | | | | | | | | | | GTCTC | |
| chr | 152084 | C | G | TCHH | p.E494 | 0.014 | 0.000 | 4.56 | 164.52 | AGTTGCTGCTCGCGCCTCTC | SEQ |
| 1 | 213 | | | | Q | 71 | 09 | E−70 | [75.16- | CTGCTGCTCGCGCCTCTCCT | ID |
| | | | | | | | | | 360.14] | [C/G]CTCCTCGAGCTTCAG | NO: |
| | | | | | | | | | | CCAACGTTCGCGCCTCTCCT | 46 |
| | | | | | | | | | | CCTCC | |
| chr | 152325 | G | C | FLG2 | p.T169 | 0.007 | 0.000 | 1.95 | 799.16 | TAATCCATGATGATAGTGGG | SEQ |
| 1 | 166 | | | | 9R | 11 | 01 | E−41 | [108.84- | CATGTCTAGTGGTATCTCCT | ID |
| | | | | | | | | | 5868.08] | [G/C]TCTGTCCATGAGTAG | NO: |
| | | | | | | | | | | TTCCATGTCTCTCAGGAACT | 47 |
| | | | | | | | | | | ATGGA | |
| chr | 156011 | G | A | UBQLN | p.P514 | 0.005 | 0.003 | 3.43 | 1.63 | CTGTTGGAGAAGATGTGGCT | SEQ |
| 1 | 387 | | | 4 | P | 15 | 16 | E−02 | [1.05- | GGCGTGGCTGGTGAGGAAGT | ID |
| | | | | | | | | | 2.54] | [G/A]GGGGCCTCGGGCGTA | NO: |
| | | | | | | | | | | GACCCTGCGTTGCTGCCTGC | 48 |
| | | | | | | | | | | TGAGG | |
| chr | 156046 | T | C | MEX3A | p.G485 | 0.005 | 0.002 | 1.14 | 182 | CGCAGATGCGTACTGCACAC | SEQ |
| 1 | 473 | | | | G | 15 | 83 | E−02 | [1.17- | TCCATGCAGAACAGGTTGTG | ID |
| | | | | | | | | | 2.84] | [T/C]CCGCAGGGCACAAGG | NO: |
| | | | | | | | | | | GCGGCAGTCACTTCGCTCTC | 49 |
| | | | | | | | | | | AAAGC | |
| chr | 156438 | C | T | MEF2D | p.Q38 | 0.010 | 0.000 | 2.23 | 1107.97 | GTTGCGGCTGCTGAGGCTGC | SEQ |
| 1 | 664 | | | | 5Q | 05 | 01 | E−58 | [152.37- | TGTGGCTGTGGCTGCTGTGG | ID |
| | | | | | | | | | 8056.7] | [C/T]TGCGGTGGCTGCTGC | NO: |
| | | | | | | | | | | TGTGGAGGCTGTGGCTGCTG | 50 |
| | | | | | | | | | | CGGCT | |
| chr | 156521 | C | T | IQGAP | p.A562 | 0.005 | 0.003 | 3.53 | 1.6 | TGCCTTTTGGCTGCCACAAG | SEQ |
| 1 | 547 | | | 3 | T | 88 | 68 | E−02 | [1.06- | GAGGAGATGGTACCGAGGGG | ID |
| | | | | | | | | | 2.42] | [C/T]GACAGGGAGGCTGAC | NO: |
| | | | | | | | | | | ATCATCTAGGCCAGCTGCAG | 51 |
| | | | | | | | | | | GAAGC | |
| chr | 156779 | G | A | SH2D2 | p.G293 | 0.006 | 0.003 | 1.36 | 1.7 | CCACATAGATGTTGCTGGGG | SEQ |
| 1 | 118 | | | A | G | 37 | 76 | E−02 | [1.14- | GCTTCCCCAGGGCTGCCCCG | ID |
| | | | | | | | | | 2.53] | [G/A]CCCATGGCATAGAAA | NO: |
| | | | | | | | | | | GCTATGGGTTCATCAGGCTC | 52 |
| | | | | | | | | | | ATTGT | |
| chr | 157069 | G | A | ETV3L | p.S32L | 0.012 | 0.008 | 3.56 | 137 | GATGAAGTGCCACAGCTGGA | SEQ |
| 1 | 134 | | | | | 25 | 99 | E−02 | [1.03- | TCTGCCGGGAGCCTGGGGAC | ID |
| | | | | | | | | | 1.82] | [G/A]ACTCGGCTTTGTAGG | NO: |
| | | | | | | | | | | CCCAATCAGGGAAGGCCAAC | 53 |
| | | | | | | | | | | CCTGG | |
| chr | 157738 | G | T | FCRL2 | p.L260 | 0.005 | 0.001 | 6.20 | 2.69 | TATTTGCCGGCATCACTCTC | SEQ |
| 1 | 309 | | | | M | 21 | 94 | E−04 | [1.51- | TTTCACAGCTGGGATCTCCA | ID |
| | | | | | | | | | 4.48] | [G/T]CTCTGCTGACAGGGA | NO: |
| | | | | | | | | | | ACGCTGGGTTTTCTTTCCCA | 54 |
| | | | | | | | | | | TACTG | |
| chr | 158669 | G | C | OR6K2 | p.A224 | 0.005 | 0.000 | 3.16 | 596.28 | TGTGCGGCGGCCTCCAGCTG | SEQ |
| 1 | 772 | | | | G | 39 | 01 | E−31 | [80.36- | AATGAATACGTAGAATTACA | ID |
| | | | | | | | | | 4424.77] | [G/C]CCACAATACCATCGT | NO: |
| | | | | | | | | | | AGGACATGAAGATGAGCATC | 55 |
| | | | | | | | | | | ACAGC | |
| chr | 161336 | A | G | C1orf1 | p.Y10Y | 0.005 | 0.003 | 2.89 | 1.66 | GAGACCAGTTCTGCAGATAC | SEQ |
| 1 | 289 | | | 92 | | 64 | 41 | E−02 | [1.09- | TTGGATGAGAAAGCCTTTTC | ID |
| | | | | | | | | | 2.53] | [A/G]TACTGTGGAGAGAAA | NO: |
| | | | | | | | | | | GATAAGTAGCCCTATGAGAC | 56 |
| | | | | | | | | | | TTCAA | |
| chr | 161476 | C | T | FCGR2 | p.S69S | 0.005 | 0.003 | 4.84 | 1.61 | CTGTGACTCTGACATGCCAG | SEQ |
| 1 | 227 | | | A | | 15 | 20 | E−02 | [1.04- | GGGGCTCGCAGCCCTGAGAG | ID |
| | | | | | | | | | 2.5] | [C/T]GACTCCATTCAGTGG | NO: |
| | | | | | | | | | | TTCCACAATGGGAATCTCAT | 57 |
| | | | | | | | | | | TCCCA | |
| chr | 161641 | G | A | ECGR2 | p.Q63 | 0.010 | 0.003 | 2.83 | 3.19 | CTGTGCTGAAACTCGAGCCC | SEQ |
| 1 | 237 | | | B | Q | 78 | 40 | E−10 | [2.33- | CAGTGGATCAACGTGCTCCA | ID |
| | | | | | | | | | 4.37] | [G/A]GAGGACTCTGTGACT | NO: |
| | | | | | | | | | | CTGACATGCCGGGGGACTCA | 58 |
| | | | | | | | | | | CAGCC | |
| chr | 169697 | A | G | SELE | p.L404 | 0.005 | 0.003 | 3.16 | 1.67 | TCCCCTGTGGGGCCACATTG | SEQ |
| 1 | 268 | | | | L | 15 | 10 | E−02 | [1.07- | GAGCCTTTTGGATCCCTTCA | ID |
| | | | | | | | | | 2.59] | [A/G]CACAAAACCCTGCTC | NO: |
| | | | | | | | | | | ACAGGAGAACTCACAGCTGG | 59 |
| | | | | | | | | | | ACCCA | |
| chr | 170115 | G | C | METTL | p.D18 | 0.000 | 0.000 | 1.00 | 1 | GGGAGCCCATTTTGCCTTTA | SEQ |
| 1 | 300 | | | 11B | H | 74 | 74 | E+00 | [0.31- | GATCCCGCTGGCAGAAGACC | ID |
| | | | | | | | | | 3.22] | [G/C]ACGATGAACTCTGTA | NO: |
| | | | | | | | | | | GACATAGCATGTCTTTTATC | 60 |
| | | | | | | | | | | CTTCA | |
| chr | 170129 | T | C | METTL | p.M66 | 0.008 | 0.006 | 1.44 | 1.29 | AAATTGTACGCTTTAACAAG | SEQ |
| 1 | 701 | | | 11B | T | 82 | 84 | E−01 | [0.92- | CCAAGTCATCAATGGTGAGA | ID |
| | | | | | | | | | 1.82] | [T/C]GCAGTTCTATGCCAG | NO: |
| | | | | | | | | | | AGCTAAACTTTTCTACCAAG | 61 |
| | | | | | | | | | | AAGTA | |
| chr | 170136 | T | C | METTL | p.L277 | 0.010 | 0.010 | 1.00 | 0.99 | GGCTTCCCAGAGCAGTGCAT | SEQ |
| 1 | 876 | | | 11B | P | 78 | 87 | E+00 | [0.73- | CCCCGTGTGGATGTTCGCAC | ID |
| | | | | | | | | | 1.35] | [T/C]GCACAGCGACAGACA | NO: |
| | | | | | | | | | | CTCCTGAAAAAGCAGTGGGA | 62 |
| | | | | | | | | | | ATGAA | |
| chr | 176563 | G | A | PAPPA | p.V347 | 0.008 | 0.005 | 2.96 | 1.51 | GCGGGATGCTCGCTTCTTCT | SEQ |
| 1 | 779 | | | 2 | M | 09 | 37 | E−02 | [1.06- | TCTCCCTCTGCACCGACCGC | ID |
| | | | | | | | | | 2.15] | [G/A]TGAAGAAAGCCACCA | NO: |
| | | | | | | | | | | TCTTGATTAGCCACAGTCGC | 63 |
| | | | | | | | | | | TACCA | |
| chr | 176833 | T | C | ASTN1 | p.E129 | 0.006 | 0.003 | 1.03 | 1.72 | TCATTCTGGCAGCAGCTCCC | SEQ |
| 1 | 427 | | | | 3G | 62 | 85 | E−02 | [1.17- | TGGCCTTATGGTGCTAGATC | ID |
| | | | | | | | | | 2.54] | [T/C]CTTTGCTGTCCCCAT | NO: |
| | | | | | | | | | | AGTCGTTGTAGGGGATACTC | 64 |
| | | | | | | | | | | AGGGT | |
| chr | 176833 | C | T | ASTN1 | p.T127 | 0.006 | 0.004 | 4.58 | 1.52 | CATAGTCGTTGTAGGGGATA | SEQ |
| 1 | 480 | | | | 5T | 13 | 04 | E−02 | [1.02- | CTCAGGGTCTGCTCCTCACA | ID |
| | | | | | | | | | 2.28] | [C/T]GTCTTCCTGAGGTCC | NO: |
| | | | | | | | | | | CGGCTGAGCTCCGCCCAGTC | 65 |
| | | | | | | | | | | AAGTC | |
| chr | 176852 | T | G | ASTN1 | p.M10 | 0.006 | 0.003 | 4.39 | 1.54 | GAGATGGTGGTGAGCTGCTT | SEQ |
| 1 | D74 | | | | 95L | 13 | 99 | E−02 | [1.03- | GTCCGGCACCTGAGATGGCA | ID |
| | | | | | | | | | 2.3] | [T/G]TGCACAAGGAGACTT | NO: |
| | | | | | | | | | | TGCTCCAGAGATGATGTCGT | 66 |
| | | | | | | | | | | CCACA | |
| chr | 186276 | G | A | PRG4 | p.E473 | 0.006 | 0.000 | 3.12 | Inf | TACACCCACCACTCCCAAGG | SEQ |
| 1 | 268 | | | | K | 62 | 00 | E−39 | | AGCCTGCACCCACCACCAAG | ID |
| | | | | | | | | | | [G/A]AGCCTGCACCCACCA | NO: |
| | | | | | | | | | | CTCCCAAAGAGCCTGCACCC | 67 |
| | | | | | | | | | | ACTGC | |
| chr | 198222 | C | G | NEK7 | p.R35 | 0.012 | 0.008 | 2.08 | 1.42 | CTTACGACCGGATATGGGCT | SEQ |
| 1 | 215 | | | | G | 25 | 67 | E−02 | [1.07- | ATAATACATTAGCCAACTTT | ID |
| | | | | | | | | | 1.89] | [C/G]GAATAGAAAAGAAAA | NO: |
| | | | | | | | | | | TTGGTCGCGGACAATTTAGT | 68 |
| | | | | | | | | | | GAAGT | |
| chr | 201178 | A | G | IGFN1 | p.E155 | 0.009 | 0.000 | 6.26 | Inf | GGGAGTAAGGCAGGTTTTAC | SEQ |
| 1 | 688 | | | | 6G | 80 | 00 | E−47 | | GGATGGTTTAGGAGGTTCTG | ID |
| | | | | | | | | | | [A/G]AGAAATGGGGTCAGT | NO: |
| | | | | | | | | | | GAATAAGGCAGGTTATAGGA | 69 |
| | | | | | | | | | | AGGAT | |
| chr | 201180 | A | G | IGFN1 | p.N20 | 0.008 | 0.000 | 6.77 | 476.2 | TAGGGATGGTTTAGGGAGTT | SEQ |
| 1 | 217 | | | | 66D | 58 | 02 | E−40 | [65.22- | CTGTAGAAATGGGGTCAGTG | ID |
| | | | | | | | | | 3476.77] | [A/G]ATGAGGCAGGTTATA | NO: |
| | | | | | | | | | | GGAAGGATTTAGGGGCTCCT | 70 |
| | | | | | | | | | | AAGGG | |
| chr | 203194 | C | T | CHIT1 | p. E74K | 0.006 | 0.003 | 9.72 | 1.74 | CACATCTTCTTCAGGCCATT | SEQ |
| 1 | 834 | | | | | 62 | 80 | E−03 | [1.18- | GAACTCCTGGTAGAGAGTCT | ID |
| | | | | | | | | | 2.58] | [C/T]GTCATTCCACTCAGT | NO: |
| | | | | | | | | | | GGTGCTCAGCTGGTGGTTGG | 71 |
| | | | | | | | | | | TCATG | |
| chr | 203691 | A | G | ATP2B | p.K940 | 0.005 | 0.002 | 4.39 | 2.02 | ACTTAACCTCCAGTGCTTCT | SEQ |
| 1 | 612 | | | 4 | R | 15 | 55 | E−03 | [1.3- | CCTCTCCCCACTAGGTGAGA | ID |
| | | | | | | | | | 3.15] | [A/G]ATTCTTTGATATTGA | NO: |
| | | | | | | | | | | TAGTGGGAGGAAGGCACCTC | 72 |
| | | | | | | | | | | TACAT | |
| chr | 204923 | G | A | NFASC | p.D81 | 0.005 | 0.000 | 3.59 | Inf | CCACTGGACACGAAACAGCA | SEQ |
| 1 | 359 | | | | N | 64 | 00 | E−34 | | GATTCTTCAACATCGCCAAG | ID |
| | | | | | | | | | | [G/A]ACCCCCGGGTGTCCA | NO: |
| | | | | | | | | | | TGAGGAGGAGGTCTGGGACC | 73 |
| | | | | | | | | | | CTGGT | |
| chr | 204923 | C | T | NFASC | p.R115 | 0.005 | 0.000 | 1.05 | Inf | GCGGCCGGAGGAATATGAGG | SEQ |
| 1 | 461 | | | | C | 39 | 00 | E−32 | | GGGAATATCAGTGCTTCGCC | ID |
| | | | | | | | | | | [C/T]GCAACAAATTTGGCA | NO: |
| | | | | | | | | | | CGGCCCTGTCCAATAGGATC | 74 |
| | | | | | | | | | | CGCCT | |
| chr | 206658 | G | A | IKBKE | p.T514 | 0.010 | 0.006 | 2.08 | 1.47 | AGCTAGCGGAGGTCCTCTCC | SEQ |
| 1 | 569 | | | | T | 05 | 84 | E−02 | [1.07- | AGATGCTCCCAAAATATCAC | ID |
| | | | | | | | | | 2.02] | [G/A]GAGACCCAGGAGAGC | NO: |
| | | | | | | | | | | CTGAGCAGCCTGAACCGGGA | 75 |
| | | | | | | | | | | GCTGG | |
| chr | 222712 | G | T | HHIPL2 | p.L487 | 0.010 | 0.006 | 1.35 | 1.71 | ACTGACTTCCCCACTGCATG | SEQ |
| 1 | 108 | | | | M | 78 | 33 | E−03 | [1.26- | GCCATAAGCATAGATTGGCA | ID |
| | | | | | | | | | 2.32] | [G/T]AACATCATCTGTCCA | NO: |
| | | | | | | | | | | GGAGAGAGGAAAGAGAGTGA | 76 |
| | | | | | | | | | | GTGTC | |
| chr | 227843 | T | A | ZNF67 | p.F413 | 0.009 | 0.000 | 1.18 | 1063 | GGAGAGAAACCCTACAAATG | SEQ |
| 1 | 024 | | | 8 | Y | 56 | 01 | E−55 | [146.01- | TGAAGAATGTGGCAGAACCT | ID |
| | | | | | | | | | 7739.02] | [T/A]TACTCAATTCTCAAA | NO: |
| | | | | | | | | | | CCTCACTCAGCATAAAAGAA | 77 |
| | | | | | | | | | | TTCAT | |
| chr | 231057 | C | T | TTC13 | p.G553 | 0.012 | 0.000 | 5.31 | Inf | TTTCTCAAAATATTCTAGGT | SEQ |
| 1 | 248 | | | | D | 99 | 00 | E−75 | | ATCTCATGTTGATCACCTGA | ID |
| | | | | | | | | | | [C/T]CCCTATAAGGCAAAA | NO: |
| | | | | | | | | | | ATAATAAAATTAAGAATATT | 78 |
| | | | | | | | | | | TTTAT | |
| chr | 236144 | G | T | NID1 | p.S107 | 0.005 | 0.002 | 8.73 | 1.93 | AGAGATGCACACACATATTT | SEQ |
| 1 | 919 | | | | 3S | 15 | 68 | E−03 | [1.24- | ACACAAAGATACCCTCTCAC | ID |
| | | | | | | | | | 3] | [G/T]GAATCCGTTACAATG | NO: |
| | | | | | | | | | | CCTCTGGGATTCACCAAGTC | 79 |
| | | | | | | | | | | AGTCT | |
| chr | 236433 | T | G | FRO1L | p.K63 | 0.005 | 0.003 | 3.47 | 1.62 | ACCTTACCTTGTAATAACGA | SEQ |
| 1 | 208 | | | B | N | 88 | 65 | E−02 | [1.07- | AAATAGTCTCTCTCTTGCAA | ID |
| | | | | | | | | | 2.44] | [T/G]TTTTTTATTTTGGGG | NO: |
| | | | | | | | | | | AAGATTTTGTAGGTATTGAA | 80 |
| | | | | | | | | | | GTTAT | |
| chr | 246907 | A | G | SCCPD | p.I183 | 0.005 | 0.002 | 2.17 | 1.91 | TCTTTTAGGTACTTTGACTG | SEQ |
| 1 | 410 | | | H | V | 21 | 73 | E−02 | [1.08- | CTGTGGAAAGTTTCCTGACT | ID |
| | | | | | | | | | 3.17] | [A/G]TACATTCAGGACCTG | NO: |
| | | | | | | | | | | AGGTTGGTTTTTTGGTTTGT | 81 |
| | | | | | | | | | | CTTGT | |
| chr | 248436 | G | A | OR2T3 | p.N28 | 0.008 | 0.002 | 5.54 | 3.43 | CTCCCTTCACCTCACTGTTC | SEQ |
| 1 | 265 | | | 3 | 4N | 33 | 45 | E−09 | [2.4- | TTCACACTGTAGATGAGGGG | ID |
| | | | | | | | | | 4.9] | [G/A]TTTAGTAAAGGGGTG | NO: |
| | | | | | | | | | | AACATAGTATAGAAGGCTGA | 82 |
| | | | | | | | | | | CACAA | |
| chr | 592504 | G | A | ANKRD | p.F257 | 0.005 | 0.003 | 2.59 | 1.66 | TGGCTCTCACATCTACATCG | SEQ |
| 10 | 7 | | | 16 | F | 39 | 25 | E−02 | [1.08- | ACGCCAAGTTCAGAGACCAA | ID |
| | | | | | | | | | 2.56] | [G/A]AATCGGATGGCTTCG | NO: |
| | | | | | | | | | | TCCTGCCCTGTGACAGCTGC | 83 |
| | | | | | | | | | | CCTGT | |
| chr | 597922 | C | 3 | FBXO1 | p.A963 | 0.005 | 0.003 | 1.08 | 1.79 | AGCGCACTGTGGAGAACATC | SEQ |
| 10 | 2 | | | 8 | A | 64 | 16 | E−02 | [1.17- | GTACTGCCCCGGCATGAGGC | ID |
| | | | | | | | | | 2.73] | [C/G]CTGCTCTTCCTCGTC | NO: |
| | | | | | | | | | | TTCTGAGGACAAGGCGCACG | 84 |
| | | | | | | | | | | TTCTC | |
| chr | 777195 | C | T | ITIH2 | p.N44 | 0.006 | 0.003 | 2.30 | 1.61 | AACTAAAACTGTCAAAAATT | SEQ |
| 10 | 8 | | | | 1N | 37 | 96 | E−02 | [1.09- | CAGAAAAACGTTAAGGAGAA | ID |
| | | | | | | | | | 2.4] | [C/T]ATCCAAGACAATATC | NO: |
| | | | | | | | | | | TCCTTGTTCAGTTTGGGCAT | 85 |
| | | | | | | | | | | GGGAT | |
| chr | 210975 | G | A | NEBL | p.S885 | 0.006 | 0.004 | 3.59 | 1.55 | TGACCTGTCGTCTCCGAGAC | SEQ |
| 10 | 46 | | | | F | 37 | 13 | E−02 | [1.04- | CTGTACCGAAAGTACTGCTG | ID |
| | | | | | | | | | 2.3] | [G/A]AATGGGATCGAGACC | NO: |
| | | | | | | | | | | AGTGTCGCCTATAGTGACTC | 86 |
| | | | | | | | | | | GCCTT | |
| chr | 345587 | C | T | PARD3 | p.G101 | 0.005 | 0.002 | 1.96 | 1.74 | CTAGCGTTGAGAGCCATGGA | SEQ |
| 10 | 15 | | | | 7R | 15 | 97 | E−02 | [1.12- | ACCTTCATAAGAAGAAACTC | ID |
| | | | | | | | | | 2.7] | [C/T]CCCATACATTAACTC | NO: |
| | | | | | | | | | | ATCATCACAGCCAAATGTCC | 87 |
| | | | | | | | | | | GATGA | |
| chr | 353221 | C | T | CUL2 | p.M34 | 0.009 | 0.004 | 5.26 | 2.06 | TACCATGCACTTCCAAAACT | SEQ |
| 10 | 99 | | | | 8I | 778 | 78 | E−04 | [1.37- | GACTCCACAAATAGTGTTGG | ID |
| | | | | | | | | | 2.98] | [C/T]ATCTAAAAATGAAAT | NO: |
| | | | | | | | | | | ATAAGTACAAAACCACATTT | 88 |
| | | | | | | | | | | TAAGA | |
| chr | 454730 | C | G | C10orf | p.M14 | 0.019 | 0.000 | 7.99 | 2197.65 | CAGGCATCCTGGCTTCACAG | SEQ |
| 10 | 44 | | | 10 | 5I | 36 | 01 | E− | [305.69- | AGCCTCCCTCTGGGGGCCCC | ID |
| | | | | | | | | 114 | 15799.34] | [C/G]ATGGGCTTGCTGCTG | NO: |
| | | | | | | | | | | TCCATCTGTCTATGTGGACC | 89 |
| | | | | | | | | | | CCAGA | |
| chr | 469992 | G | A | GPRIN | p.8110 | 0.007 | 0.005 | 3.98 | 1.46 | AATGTGTCCACCATGGGCGG | SEQ |
| 10 | 09 | | | 2 | Q | 84 | 38 | E−02 | [1.02- | CAGTGACCTGTGTCGCCTGC | ID |
| | | | | | | | | | 2.09] | [G/A]GGCCCCTAGTGCTGC | NO: |
| | | | | | | | | | | TGCTATGCAGAGGAGCCATT | 90 |
| | | | | | | | | | | CAGAC | |
| chr | 469993 | A | G | GPRIN | p.A170 | 0.010 | 0.003 | 4.09 | 2.99 | AGCCAGGTGGTACTTCTGGC | SEQ |
| 10 | 90 | | | 2 | A | 29 | 47 | E−09 | [2.17- | CAGGGTGGCCAGGCCCCTGC | ID |
| | | | | | | | | | 4.12] | [A/G]GGCCTGGAAAGGGAC | NO: |
| | | | | | | | | | | CTGGCTCCTGAGGATGAGAC | 91 |
| | | | | | | | | | | TTCTA | |
| chr | 470872 | G | C | LOC10 | p.L172 | 0.006 | 0.003 | 3.19 | 1.88 | GGATTGTGCTCATCTGGGTC | SEQ |
| 10 | 99 | | | 09967 | L | 62 | 53 | E−03 | [1.27- | ATTGCCTGTGTCCTCTCCCT | ID |
| | | | | 58 | | | | | 2.78] | [G/C]CCCTTCCTGGCCAAC | NO: |
| | | | | | | | | | | AGCATCCTGGAGAATGTCTT | 92 |
| | | | | | | | | | | CCACA | |
| chr | 518279 | A | G | FAM21 | p.P13P | 0.022 | 0.002 | 5.96 | 9.18 | TGCAGATGAACCGGACGACC | SEQ |
| 10 | 00 | | | A | | 30 | 48 | E−49 | [7.2- | CCCGACCAGGAGCTGGCGCC | ID |
| | | | | | | | | | 11.7] | [A/G]GCGTCGGAGCCCGTG | NO: |
| | | | | | | | | | | TGGGAGCGGCCGTGGTCGGT | 93 |
| | | | | | | | | | | GGAGG | |
| chr | 734648 | G | A | CDH23 | p.E960 | 0.008 | 0.004 | 2.90 | 1.94 | GGTGGTCACCACCACCGAGC | SEQ |
| 10 | 12 | | | | K | 133 | 201 | E−03 | [1.24- | TGGACCGCGAGCGCATCGCG | ID |
| | | | | | | | | | 2.91] | [G/A]AGTACCAGCTGCGGG | NO: |
| | | | | | | | | | | TGGTGGCCAGTGATGCAGGC | 94 |
| | | | | | | | | | | ACGCC | |
| chr | 750106 | G | C | MRPS1 | p.T130 | 0.008 | 0.004 | 8.85 | 1.74 | CTAAAGTCAGCTCATTTATG | SEQ |
| 10 | 35 | | | 6 | 8 | 458 | 873 | E−03 | [1.13- | TTTCTGTAGCCTCTGTATCT | ID |
| | | | | | | | | | 2.59] | [G/C]TAGCTTCTGCATCTG | NO: |
| | | | | | | | | | | TTTTCTGAGAAGCTAACAGG | 95 |
| | | | | | | | | | | ACTTC | |
| chr | 795887 | G | A | DLG5 | p.A741 | 0.007 | 0.004 | 8.01 | 1.69 | GGGACCCTTCTTTAGCGGCA | SEQ |
| 10 | 06 | | | | A | 35 | 35 | E−03 | [1.17- | GGGCTTCCAGGCAGCACAGC | ID |
| | | | | | | | | | 2.45] | [G/A]GCAGCATACACTCCA | NO: |
| | | | | | | | | | | TTCTCCAGACTGATGCCACT | 96 |
| | | | | | | | | | | GTCTG | |
| chr | 995312 | C | T | SFRP5 | p.D103 | 0.010 | 0.006 | 2.15 | 1.46 | CAGACGGGCGCAAAGAGCGA | SEQ |
| 10 | 84 | | | | N | 05 | 89 | E−02 | [1.07- | GCACAGGAAGACCTGCGTAT | ID |
| | | | | | | | | | 2.01] | [C/T]CGAGTGGCAGCGCTT | NO: |
| | | | | | | | | | | GGCCAGCAGCGGCAGCCAGC | 97 |
| | | | | | | | | | | TGCTC | |
| chr | 999696 | A | G | R3HCC | p.L593 | 0.006 | 0.003 | 2.22 | 1.9 | TGTTTAACGATGATGGTGAC | SEQ |
| 10 | 50 | | | 1L | L | 86 | 60 | E−03 | 1[1.3- | TGCCTGGATCCACGTCTTCT | ID |
| | | | | | | | | | 2.81] | [A/G]CAAGAGGTATGTTTA | NO: |
| | | | | | | | | | | ATTGAAATTGCTTGATGCTT | 98 |
| | | | | | | | | | | AGTTA | |
| chr | 102770 | A | G | PDZD7 | p.R777 | 0.011 | 0.000 | 2.35 | 126.17 | ACTTGCCTTGACCCCGGCTG | SEQ |
| 10 | 315 | | | | R | 03 | 09 | E−44 | [45.36- | CTGCGGCTGCGGCTGCGGCT | ID |
| | | | | | | | | | 350.99] | [A/G]CGGCTGCGGCTACGG | NO: |
| | | | | | | | | | | CTCTGAGCCCGGCCCCGGAT | 99 |
| | | | | | | | | | | CTGGC | |
| chr | 104230 | G | A | TMEM | p.T139 | 0.010 | 0.007 | 4.44 | 1.39 | AGTTCTTGCTGTGCCTGTGC | SEQ |
| 10 | 587 | | | 180 | T | 54 | 62 | E−02 | [1.02- | CTCTATGATGGCTTCCTGAC | ID |
| | | | | | | | | | 1.89] | [G/A]CTCGTGGACCTGCAC | NO: |
| | | | | | | | | | | CACCATGCCTTGCTGGCCGA | 100 |
| | | | | | | | | | | CCTGG | |
| chr | 125780 | G | C | CHST1 | p.P453 | 0.008 | 0.000 | 3.19 | 793.53 | GCTCCTTCTGCCAGGGGCCA | SEQ |
| 10 | 760 | | | 5 | P | 58 | 01 | E−47 | [108.69- | GCTCGGGGGGTACGGGGGGG | ID |
| | | | | | | | | | 5793.56] | [G/C]GGGGTACACACAGGC | NO: |
| | | | | | | | | | | ATGGCGTTGTTGAGGGTGTT | 101 |
| | | | | | | | | | | GTTGT | |
| chr | 135106 | G | A | TUBGC | p.H360 | 0.005 | 0.003 | 2.61 | 1.66 | CCTGCGCCTGGCTGTCCCCT | SEQ |
| 10 | 137 | | | P2 | H | 39 | 26 | E−02 | [1.08- | GTGTAGCTGAAGCTCCTGTC | ID |
| | | | | | | | | | 2.55] | [G/A]TGGAGCAGGCTCAGC | NO: |
| | | | | | | | | | | GTGGACCCCCCAAGACATTC | 102 |
| | | | | | | | | | | GCCTT | |
| chr | 135368 | G | C | SYCE1 | p.V289 | 0.008 | 0.005 | 2.96 | 1.51 | GGCCAGCCTCTTCCTCTTGT | SEQ |
| 10 | 906 | | | | V | 09 | 37 | E−02 | [1.06- | GTGCTCTGGGCTTGGGCAGG | ID |
| | | | | | | | | | 2.15] | [G/C]ACTTGCATTCCATGC | NO: |
| | | | | | | | | | | TTTTCCAGCTCTTCCTTCAG | 103 |
| | | | | | | | | | | CCTGG | |
| chr | 394511 | C | T | PKP3 | p.A73 | 0.006 | 0.000 | 6.27 | Inf | AGCCGCGGCACAACGGGGCC | SEQ |
| 11 | | | | | A | 86 | 00 | E−11 | | GCTGAGCCCGAGCCTGAGGC | ID |
| | | | | | | | | | | [C/T]GAGACTGCCAGAGGT | NO: |
| | | | | | | | | | | AGGCGGTGGGGACAGCGGCG | 104 |
| | | | | | | | | | | GGGAT | |
| chr | 610300 | A | G | PHRF1 | p.S145 | 0.006 | 0.003 | 2.18 | 1.93 | CACAGGGGTCAGGCAGGTGT | SEQ |
| 11 | | | | | 5G | 86 | 57 | E−03 | [1.3- | TCTCCGAGCTGCCCTTTCCC | ID |
| | | | | | | | | | 2.85] | [A/G]GTCACGTGCTTCCGG | NO: |
| | | | | | | | | | | AACCCGGGTTCCCAGACACA | 105 |
| | | | | | | | | | | GACCC | |
| chr | 614967 | C | G | IRF7 | p.R88T | 0.005 | 0.000 | 4.94 | Inf | GCGCTCCGCAGTCTCAGCCT | SEQ |
| 11 | | | | | | 88 | 00 | E−32 | | CGGGGGGCGGGCCACCTCCC | ID |
| | | | | | | | | | | [C/G]TGCTGCTAGGCGGCC | NO: |
| | | | | | | | | | | ACCTGCCGCGGGCCACAGCC | 106 |
| | | | | | | | | | | CAGGC | |
| chr | 764414 | A | G | TALDO | p.K321 | 0.006 | 0.003 | 1.66 | 1.71 | CTCTCTGACGGGATCCGCAA | SEQ |
| 11 | | | | 1 | R | 13 | 59 | E−02 | [1.14- | GTTTGCCGCTGATGCAGTGA | ID |
| | | | | | | | | | 2.56] | [A/G]GCTGGAGCGGATGCT | NO: |
| | | | | | | | | | | GACAGTGAGTCTTGTGTGTG | 107 |
| | | | | | | | | | | GGTAC | |
| chr | 101685 | G | A | MUC6 | p.P198 | 0.011 | 0.000 | 1.29 | Inf | GGATAGGTAGTGGTGGTCTG | SEQ |
| 11 | 4 | | | | 3S | 27 | 00 | E−67 | | GAAGGATGTTGCAGTCATAG | ID |
| | | | | | | | | | | [G/A]ACCTGTGGAAGAGAA | NO: |
| | | | | | | | | | | GGGACTGCTCCCTGTAGGTG | 108 |
| | | | | | | | | | | GGGAG | |
| chr | 101708 | G | A | MUC6 | p.P190 | 0.007 | 0.001 | 3.28 | 4.53 | GGTAGGGATGTAGAAGTTTT | SEQ |
| 11 | 5 | | | | 6S | 84 | 74 | E−11 | [3.11- | GGCCGTGCTAAATGAGCTTG | ID |
| | | | | | | | | | 6.59] | [G/A]GGATTGGCTGGTCCC | NO: |
| | | | | | | | | | | ACTGGTGGTCGGTGTCATTG | 109 |
| | | | | | | | | | | GTGGG | |
| chr | 101754 | G | A | MUC6 | p.T175 | 0.025 | 0.000 | 8.09 | Inf | GGTAGAAGTTGAGGTGACTT | SEQ |
| 11 | 3 | | | | 3I | 25 | 00 | E− | | CAGGATGGTGTGTGGAGGAA | ID |
| | | | | | | | | 151 | | [G/A]TGTGTGAATGTAGGG | NO: |
| | | | | | | | | | | ATGTAGAGGTTTTGGCCGTG | 110 |
| | | | | | | | | | | CTAAA | |
| chr | 101776 | T | C | MUC6 | p.Q16 | 0.009 | 0.000 | 1.12 | 180.29 | GGGATGTAGAGGTTTTGGCT | SEQ |
| 11 | 1 | | | | 80Q | 80 | 05 | E−51 | [76.39- | GTGTTTAATGAGCTCAGGGC | ID |
| | | | | | | | | | 425.47] | [T/C]TGGCTGGTCCCGCTG | NO: |
| | | | | | | | | | | GTGGTCAGCGTCATTGTTGG | 111 |
| | | | | | | | | | | CGCTG | |
| chr | 101778 | C | T | MUC6 | p.T167 | 0.009 | 0.000 | 1.86 | 27.78 | TTAATGAGCTCAGGGCTTGG | SEQ |
| 11 | 5 | | | | 2T | 80 | 36 | E−36 | [17.86- | CTGGTCCCGCTGGTGGTCAG | ID |
| | | | | | | | | | 43.24] | [C/T]GTCATTGTTGGCGCT | NO: |
| | | | | | | | | | | GTGTGGGTGGACCCTGTGGC | 112 |
| | | | | | | | | | | CTTGA | |
| chr | 101791 | G | A | MUC6 | p.T163 | 0.014 | 0.000 | 6.50 | 51.65 | GGCAGAAGTGGCCATCTGTG | SEQ |
| 11 | 2 | | | | 0I | 95 | 29 | E−49 | [26.44- | CATGGGTAGGGGTGATGACT | ID |
| | | | | | | | | | 100.88] | [G/A]TGTGAGTACTTGGAG | NO: |
| | | | | | | | | | | TCACCAAAGAGGTGGAGAAA | 113 |
| | | | | | | | | | | GGTGG | |
| chr | 101797 | C | G | MUC6 | p.Q16 | 0.007 | 0.000 | 2.56 | 15.72 | AAGAGGTGGAGAAAGGTGGA | SEQ |
| 11 | 4 | | | | 09H | 60 | 49 | E−23 | [10.08- | ACGTGAGTGGGAAGTGTGGT | ID |
| | | | | | | | | | 24.51] | [C/G]TGAGGGTGTGATGGG | NO: |
| | | | | | | | | | | GTTGGATAGGTAGTGGTGGT | 114 |
| | | | | | | | | | | CTTGA | |
| chr | 102362 | G | A | MUC6 | p.T113 | 0.009 | 0.007 | 4.58 | 1.4 | GGCCTCCTGTGTGTACTGGT | SEQ |
| 11 | 2 | | | | 8M | 80 | 03 | E−02 | [1.02- | ACTCGCCATGGCCGTCCTGC | ID |
| | | | | | | | | | 1.92] | [G/A]TGTGCGTGTTGTAGA | NO: |
| | | | | | | | | | | AGCCGCAGTAGATGGCTGGG | 115 |
| | | | | | | | | | | AGGAA | |
| chr | 109353 | A | C | MUC2 | p.K178 | 0.007 | 0.000 | 4.33 | 94.81 | CACCACTACGATGACCCCAA | SEQ |
| 11 | 7 | | | | 6Q | 11 | 08 | E−27 | [28.87- | CCCCAACACCCACCAGCACA | ID |
| | | | | | | | | | 311.37] | [A/C]AGAGTACAACCGTGA | NO: |
| | | | | | | | | | | CACCCATCACCACCACAACT | 116 |
| | | | | | | | | | | ACGGT | |
| chr | 126418 | C | T | MUC5 | p.T202 | 0.006 | 0.003 | 1.15 | 1.69 | ACTCCAGAGACTGCCCACAC | SEQ |
| 11 | 7 | | | B | 6M | 62 | 93 | E−02 | [1.14- | CTCCACAGTGCTTACCGCCA | ID |
| | | | | | | | | | 2.49] | [C/T]GGCCACCACAACTGG | NO: |
| | | | | | | | | | | GGCCACCGGCTCTGTGGCCA | 117 |
| | | | | | | | | | | CCCCC | |
| chr | 126996 | G | A | MUC5 | p.T395 | 0.006 | 0.004 | 4.28 | 1.52 | CCAGTGGTACTCCCCCATCA | SEQ |
| 11 | 9 | | | B | 3T | 86 | 53 | E−02 | [1.03- | CTGATCACCACGGCCACTAC | ID |
| | | | | | | | | | 2.24] | [G/A]ATCACGGCCACCGGC | NO: |
| | | | | | | | | | | TCCACCACCAACCCCTCCTC | 118 |
| | | | | | | | | | | AACTC | |
| chr | 127131 | A | G | MUC5 | p.T440 | 0.014 | 0.000 | 2.18 | Inf | CGACCTGGATCCTCACAGAG | SEQ |
| 11 | 3 | | | B | 1T | 95 | 00 | E−89 | | CTGACCACAGCAGCCACTAC | ID |
| | | | | | | | | | | [A/G]ACTGCAGCCACTGGC | NO: |
| | | | | | | | | | | CCCACGGCCACCCCGTCCTC | 119 |
| | | | | | | | | | | CACCC | |
| chr | 160615 | G | A | KRTAP | p.G110 | 0.005 | 0.000 | 6.34 | Inf | CACAGCCGGAACCACAGCCA | SEQ |
| 11 | 0 | | | 5-1 | G | 39 | 00 | E−31 | | CCCTTGGATCCCCCACAAGA | ID |
| | | | | | | | | | | [G/A]CCACAGCCCCCCTTG | NO: |
| | | | | | | | | | | GAGCCCCCACAGGAGCCACA | 120 |
| | | | | | | | | | | ACCCC | |
| chr | 160640 | G | A | KRTAP | p.S26S | 0.004 | 0.000 | 2.01 | 42.77 | AGCCAGAACCTCCACAGCCA | SEQ |
| 11 | 2 | | | 5-1 | | 64 | 10 | E−11 | [16.27- | GAGCCACAGCCCCCACAGCC | ID |
| | | | | | | | | | 112.48] | [G/A]GAGCCACAGCCCCCA | NO: |
| | | | | | | | | | | CAGCCGGAGCCACAGCCCCC | 121 |
| | | | | | | | | | | ACAGC | |
| chr | 161943 | A | G | KRTAP | p.C17C | 0.012 | 0.000 | 1.27 | 137366 | AGCCCCCACAGCCAGAGCCA | SEQ |
| 11 | 0 | | | 5-2 | | 25 | 01 | E−71 | [189.71- | CAACCCCCACAGCTGGAGCC | ID |
| | | | | | | | | | 9946.24] | [A/G]CAGCCCCCACAGCCG | NO: |
| | | | | | | | | | | GAGCCACAGCCTCTGGAGCA | 122 |
| | | | | | | | | | | GCCAC | |
| chr | 162916 | G | A | KRTAP | p.C151 | 0.010 | 0.000 | 5.33 | 1023.61 | AGCAGGGCTTACAGCAGCTG | SEQ |
| 11 | 3 | | | 5-3 | C | 29 | 01 | E−58 | [140.85- | GACTGGGAGCAGCTGGGCTT | ID |
| | | | | | | | | | 7439.08] | [G/A]CAGCAGCTGGACTGG | NO: |
| | | | | | | | | | | CAGCAGGATGACCCACAGCC | 123 |
| | | | | | | | | | | TGAGG | |
| chr | 162936 | C | A | KRTAP | p.K84 | 0.013 | 0.000 | 1.22 | Inf | AGCAGCAGACGGGCACACAG | SEQ |
| 11 | 4 | | | 5-3 | N | 48 | 00 | E−80 | | CAGCTGGAGCCACAGCCCCC | ID |
| | | | | | | | | | | [C/A]TTGGAGCCTCCACAG | NO: |
| | | | | | | | | | | GAGCCACAGCCCCCCTTGCA | 124 |
| | | | | | | | | | | GCCCC | |
| chr | 164288 | A | G | KRTAP | p.S148 | 0.011 | 0.000 | 1.28 | Inf | TACAGCAGCTGGACTGGCAG | SEQ |
| 11 | 0 | | | 5-4 | S | 27 | 00 | E−67 | | CAGGATGACCCACAGCCTGA | ID |
| | | | | | | | | | | [A/G]GAGAAGCAGCAGGGC | NO: |
| | | | | | | | | | | TTACAGCAGCTGCACTGGGA | 125 |
| | | | | | | | | | | GCAGC | |
| chr | 165135 | A | G | KRTAP | p.R97 | 0.027 | 0.000 | 1.04 | Inf | CTGTGGCAAAGGGGGCTGTG | SEQ |
| 11 | 9 | | | 5-5 | G | 94 | 00 | E− | | GCTCTTGCGGGGGCTCCAAG | ID |
| | | | | | | | | 166 | | [A/G]GAGGCTGTGTCTCCT | NO: |
| | | | | | | | | | | GTGGGGTGTCCAAGGGGGCC | 126 |
| | | | | | | | | | | TGTGG | |
| chr | 216143 | G | A | IGF2 | p.Q33 | 0.016 | 0.000 | 9.89 | 19.8 | CGTCTAAGTAGCTCGCCTTT | SEQ |
| 11 | 0 | | | | X | 68 | 90 | E−16 | [11.5- | GCGGCCCACCCAAAATATCT | ID |
| | | | | | | | | | 34.2] | [G/A]GATAATGGTTACCCC | NO: |
| | | | | | | | | | | GTCCTCAGTGCGTTGGACTT | 127 |
| | | | | | | | | | | GCATA | |
| chr | 438911 | G | A | OR52B | p.T139 | 0.005 | 0.002 | 2.82 | 1.79 | CAGAGAGACAGTCACACAAA | SEQ |
| 11 | 0 | | | 4 | I | 21 | 91 | E−02 | [1.01- | TTTTCTTGATCAGAGCATTT | ID |
| | | | | | | | | | 2.96] | [G/A]TAAGAATGGTGGTGT | NO: |
| | | | | | | | | | | ACCTCAGTGGGTAGCATATG | 128 |
| | | | | | | | | | | GCAAT | |
| chr | 544404 | C | T | ORS1Q | p.L204 | 0.008 | 0.005 | 1.36 | 1.57 | CTGTGCTGACATCAGGCTCA | SEQ |
| 11 | 0 | | | 1 | F | 58 | 50 | E−02 | [1.11- | ACAGCTGGTATGGATTTGCT | ID |
| | | | | | | | | | 2.2] | [C/T]TTGCCTTGCTCATTA | NO: |
| | | | | | | | | | | TTATCGTGGATCCTCTGCTC | 129 |
| | | | | | | | | | | ATTGT | |
| chr | 691328 | T | C | OR2D2 | p.S151 | 0.008 | 0.004 | 1.80 | 1.67 | AGTATGAAGGTGGTGTCTAC | SEQ |
| 11 | 1 | | | | G | 133 | 873 | E−02 | [1.07- | CACAGACACCAGAATGCCAC | ID |
| | | | | | | | | | 2.5] | [T/C]GGTCCATGATCCTGT | NO: |
| | | | | | | | | | | TGCCAGCTGGACACACACTT | 130 |
| | | | | | | | | | | TCCAG | |
| chr | 694291 | C | T | OR2D3 | p.S228 | 0.014 | 0.010 | 5.32 | 1.47 | ATCTTTTCAATGGGCGTGGT | SEQ |
| 11 | 5 | | | | F | 71 | 03 | E−03 | [1.13- | AATCCTCCTGGCCCCTGTCT | ID |
| | | | | | | | | | 1.92] | [C/T]CCTGATTCTTGGTTC | NO: |
| | | | | | | | | | | TTATTGGAATATTATCTCCA | 131 |
| | | | | | | | | | | CTGTT | |
| chr | 122463 | G | A | MICAL | p. R559 | 0.008 | 0.005 | 2.51 | 1.5 | CGCAGTGGGTTGGCCCTGTG | SEQ |
| 11 | 55 | | | 2 | Q | 33 | 56 | E−02 | [1.06- | TGCCATCATCCACCGCTTCC | ID |
| | | | | | | | | | 2.13] | [G/A]GCCTGAGCTCATGTG | NO: |
| | | | | | | | | | | AGTCTGGGGCCCAGGCTGGC | 132 |
| | | | | | | | | | | CCCTG | |
| chr | 341650 | G | A | NAT10 | p.A983 | 0.008 | 0.003 | 5.80 | 2.17 | TGAAGAGTGGAATGAAGTTT | SEQ |
| 11 | 53 | | | | T | 133 | 762 | E−04 | [1.39- | TGAACAAAGCTGGGCCGAAC | ID |
| | | | | | | | | | 3.26] | [G/A]CCTCGATCATCAGCC | NO: |
| | | | | | | | | | | TGAAAAGGTGAGGGCCCAGG | 133 |
| | | | | | | | | | | GTCTG | |
| chr | 354560 | T | A | PAMR1 | p.0534 | 0.007 | 0.005 | 3.47 | 1.49 | CAAGCCCTCTCTTACCTGTA | SEQ |
| 11 | 85 | | | | V | 60 | 11 | E−02 | [1.04- | GGCTCTGGATGGTCTTCTCA | ID |
| | | | | | | | | | 2.14] | [T/A]CCCGGTCATCATCCC | NO: |
| | | | | | | | | | | GGTAGAATTTCCCCAAAACA | 134 |
| | | | | | | | | | | ACTTT | |
| chr | 474696 | G | T | RAPSN | p.N88 | 0.005 | 0.002 | 5.29 | 1.96 | TCTTGTGAAACTCGCACAGC | SEQ |
| 11 | 31 | | | | K | 15 | 63 | E−03 | [1.26- | TTCTCGTTGCTGCGTGCCAG | ID |
| | | | | | | | | | 3.06] | [G/T]TTCAGGTAGCTCTCC | NO: |
| | | | | | | | | | | AGGAGGAAGTCGGCATCCTC | 135 |
| | | | | | | | | | | CAGCT | |
| chr | 619595 | A | C | SCGB1 | p.N20T | 0.005 | 0.002 | 1.60 | 2.21 | TCCTTACACAAATTATATTT | SEQ |
| 11 | 31 | | | D1 | | 15 | 33 | E−03 | [1.42- | TTATTCTTTTGCTCCAGCAA | ID |
| | | | | | | | | | 3.46] | [A/C]TGCAGTGGTCTGCCA | NO: |
| | | | | | | | | | | ACCTCTTGGTTCTGAAATCA | 136 |
| | | | | | | | | | | CAGGC | |
| chr | 622880 | G | A | AHNAK | p.P462 | 0.007 | 0.004 | 2.38 | 1.54 | GGACATCAATGTCCACTTTG | SEQ |
| 11 | 14 | | | | 5P | 60 | 94 | E−02 | [1.07- | GGGTCCCTGATGTCAACTTC | ID |
| | | | | | | | | | 2.22] | [G/A]GGGCCCTTGAGGTCG | NO: |
| | | | | | | | | | | CCTTCCACTTTGGGCAGAGA | 137 |
| | | | | | | | | | | AATGT | |
| chr | 624339 | C | T | METTL | p.R38 | 0.005 | 0.002 | 2.15 | 9.2 | ACTGGCTGATAGTTGCCTGG | SEQ |
| 11 | 12 | | | 12 | W | 21 | 72 | E−02 | [1.08- | CGGACCGCTGTCTCTGGGAT | ID |
| | | | | | | | | | 3.18] | [C/T]GGCTGCATGCCCAGC | NO: |
| | | | | | | | | | | CTCGTTTGGGCACTGTCCCC | 138 |
| | | | | | | | | | | ACCTT | |
| chr | 624443 | C | G | UBXN1 | p.E249 | 0.012 | 0.008 | 3.42 | 1.38 | CTGAGCAATTGCACAGGGTC | SEQ |
| 11 | 84 | | | | Q | 25 | 88 | E−02 | [1.04- | CTGGCCCCCACCTAGTTCCT | ID |
| | | | | | | | | | 1.84] | [C/G]CCCACGGTGGAGCTC | NO: |
| | | | | | | | | | | CACATAGAGCCTCACAGCTG | 139 |
| | | | | | | | | | | CCAGC | |
| chr | 627608 | C | T | SLC22A | p. R422 | 0.005 | 0.003 | 1.35 | 1.75 | GGCCTTTTCCACCTCTGGCT | SEQ |
| 11 | 00 | | | 8 | Q | 88 | 38 | E−02 | [1.15- | CCTGCTTTGGCTTCTTTGCC | ID |
| | | | | | | | | | 2.64] | [C/T]GCAGGGACCTAGGGA | NO: |
| | | | | | | | | | | CAGAGAGCTAAGGAAAAGCC | 140 |
| | | | | | | | | | | CTGGG | |
| chr | 634874 | G | C | RTN3 | p.0501 | 0.010 | 0.007 | 4.56 | 1.38 | ATTGGGAGAAATCACAGAAG | SEQ |
| 11 | 75 | | | | H | 54 | 68 | E−02 | [1.01- | CTGATAGTTCTGGTGAGTCT | ID |
| | | | | | | | | | 1.87] | [G/C]ATGACACAGTAATAG | NO: |
| | | | | | | | | | | AGGACATCACAGCAGATACA | 141 |
| | | | | | | | | | | TCATT | |
| chr | 636815 | C | T | RCOR2 | p.T271 | 0.009 | 0.004 | 1.13 | 2.3 | GGAGCGTGAGGTTGGCAAGG | SEQ |
| 11 | 04 | | | | T | 31 | 07 | E−05 | [1.65- | TCCGGGCTTCCTGACACTGC | ID |
| | | | | | | | | | 3.2] | [C/T]GTGAGGCCTTCAGGG | NO: |
| | | | | | | | | | | CTCAGGTACATGCCCTTGGG | 142 |
| | | | | | | | | | | TGGGC | |
| chr | 640518 | G | A | GPR13 | p.G17 | 0.006 | 0.000 | 6.66 | 48.83 | CTGTGAGGACAAGATGTTAC | SEQ |
| 11 | 89 | | | 7 | G | 04 | 10 | E−15 | [20.22- | GTAGTCAAGGCACAGCTGGG | ID |
| | | | | | | | | | 117.93] | [G/A]CCAACGGTGGCCCTG | NO: |
| | | | | | | | | | | GAAGGCAGAGGCAGGTACCC | 143 |
| | | | | | | | | | | CTGGC | |
| chr | 640832 | G | T | ESRRA | p.R376 | 0.018 | 0.000 | 4.17 | 28.9 | GAAGCCGGCCGGGCTGGCCC | SEQ |
| 11 | 93 | | | | L | 87 | 67 | E−69 | [20.87- | CGGAGGGGGTGCTGAGCGGC | ID |
| | | | | | | | | | 40] | [G/T]GCGGGCGGGCAGGCT | NO: |
| | | | | | | | | | | GCTGCTCACGCTACCGCTCC | 144 |
| | | | | | | | | | | TCCGC | |
| chr | 640833 | G | A | ESRRA | p.A378 | 0.016 | 0.000 | 5.66 | 27.17 | GCCGGGCTGGCCCCGGAGGG | SEQ |
| 11 | 00 | | | | A | 91 | 63 | E−61 | [19.38- | GGTGCTGAGCGGCGGCGGGC | ID |
| | | | | | | | | | 38.08] | [G/A]GGCAGGCTGCTGCTC | NO: |
| | | | | | | | | | | ACGCTACCGCTCCTCCGCCA | 145 |
| | | | | | | | | | | GACAG | |
| chr | 649850 | G | A | SLC22A | p.A184 | 0.005 | 0.003 | 4.82 | 1.61 | GGTCCTACCTGCAGCTGGCA | SEQ |
| 11 | 72 | | | 20 | A | 15 | 20 | E−02 | [1.04- | GCTTCGGGGGCCGCCACAGC | ID |
| | | | | | | | | | 2.51] | [G/A]TATTTCAGCTCCTTC | NO: |
| | | | | | | | | | | AGTGCCTATTGCGTCTTCCG | 146 |
| | | | | | | | | | | GTTCC | |
| chr | 724060 | C | T | ARAP1 | p.V122 | 0.005 | 0.002 | 2.50 | 2.09 | CAAGCCCAGCGTCACCCACC | SEQ |
| 11 | 46 | | | | 51 | 15 | 47 | E−03 | [1.34- | TGCCTCCTCCCTCTCGTTGA | ID |
| | | | | | | | | | 3.25] | [C/T]CTCAAAGCAGGTCCA | NO: |
| | | | | | | | | | | ATAGTCCTTCTCCCTGATGC | 147 |
| | | | | | | | | | | CCACG | |
| chr | 738439 | C | T | C2CD3 | p.R371 | 0.009 | 0.006 | 4.99 | 1.41 | CAGTTGAAGGGAGGAGGTGA | SEQ |
| 11 | 93 | | | | R | 31 | 62 | E−02 | [1.02- | TCTTCAATGTGGTCTTTAAA | ID |
| | | | | | | | | | 1.96] | [C/T]CGATTCCTAGAAAAG | NO: |
| | | | | | | | | | | GCTCTGATCCTAAGGTGTGG | 148 |
| | | | | | | | | | | AAAAA | |
| chr | 740535 | G | A | PGM2L | p.T522 | 0.005 | 0.002 | 4.03 | 2.06 | ATATCCAGTGGTAACGTCCC | SEQ |
| 11 | 73 | | | 1 | I | 15 | 51 | E−03 | [1.32- | GTACATGCAATATAGCAAAT | ID |
| | | | | | | | | | 3.21] | [G/A]TTCCACAAAATTTTG | NO: |
| | | | | | | | | | | GATATTCTTTTGGAGAATCA | 149 |
| | | | | | | | | | | AAATT | |
| chr | 747175 | A | T | NEU3 | p.X462 | 0.006 | 0.004 | 2.55 | 1.61 | CCAGCCCTGGTAGGAACCCA | SEQ |
| 11 | 37 | | | | Y | 62 | 13 | E−02 | [1.09- | AGCCAATTCAAAAGCAATTA | ID |
| | | | | | | | | | 2.37] | [A/T]TTGGCTTAGGACCCA | NO: |
| | | | | | | | | | | ATTTCCATAGATGCAAATGG | 150 |
| | | | | | | | | | | CAGTT | |
| chr | 755093 | C | T | DGAT2 | p.F247 | 0.012 | 0.000 | 1.70 | Inf | ACTCCTTTGGAGAGAATGAA | SEQ |
| 11 | 32 | | | | F | 25 | 00 | E−73 | | GTGTACAAGCAGGTGATCTT | ID |
| | | | | | | | | | | [C/T]GAGGAGGGCTCCTGG | NO: |
| | | | | | | | | | | GGCCGATGGGTCCAGAAGAA | 151 |
| | | | | | | | | | | GTTCC | |
| chr | 755093 | C | T | DGAT2 | p.G250 | 0.018 | 0.000 | 1.61 | Inf | GAGAGAATGAAGTGTACAAG | SEQ |
| 11 | 41 | | | | G | 14 | 00 | E− | | CAGGTGATCTTCGAGGAGGG | ID |
| | | | | | | | | 108 | | [C/T]TCCTGGGGCCGATGG | NO: |
| | | | | | | | | | | GTCCAGAAGAAGTTCCAGAA | 152 |
| | | | | | | | | | | ATACA | |
| chr | 768348 | C | A | CAPN5 | p.L632 | 0.007 | 0.004 | 9.41 | 1.67 | GCAGCCCAGCAACCTGCCAG | SEQ |
| 11 | 87 | | | | I | 60 | 56 | E−03 | [1.16- | GCACTGTGGCCGTGCACATT | ID |
| | | | | | | | | | 2.41] | [C/A]TCAGCAGCACCTCCC | NO: |
| | | | | | | | | | | TCATGGCTGTCTGACACCTG | 153 |
| | | | | | | | | | | CCCAC | |
| chr | 828797 | C | T | PCF11 | p.P795 | 0.007 | 0.005 | 3.85 | 1.47 | GGACCTCCCACACCAGCTTC | SEQ |
| 11 | 61 | | | | L | 84 | 34 | E−02 | [1.03- | TCTTCGGTTTGATGGGTCAC | ID |
| | | | | | | | | | 2.11] | [C/T]AGGACAAATGGGGGG | NO: |
| | | | | | | | | | | AGGAGGCCCTTTGAGATTTG | 154 |
| | | | | | | | | | | AGGGG | |
| chr | 896073 | C | T | TRIM6 | p.E205 | 0.008 | 0.003 | 3.95 | 2.81 | ATTCTCACTTGACTGTCTTG | SEQ |
| 11 | 39 | | | 4B | K | 82 | 16 | E−07 | [1.96- | TAGTTGTTGGAAAAGCTCTT | ID |
| | | | | | | | | | 4.02] | [C/T]TGCTTCTCTTTCCAG | NO: |
| | | | | | | | | | | TGCCTGCAGATGCCGTTGCT | 155 |
| | | | | | | | | | | CCTCC | |
| chr | 947598 | G | A | KDM4E | p.C381 | 0.008 | 0.003 | 1.56 | 2.17 | GCTCTGGGCCTGAGGCTTCT | SEQ |
| 11 | 63 | | | | Y | 09 | 74 | E−04 | [1.5- | CCCAAACCTCACAGCCCAGT | ID |
| | | | | | | | | | 3.14] | [G/A]TCCCACACAGCCTGT | NO: |
| | | | | | | | | | | GTCCTCAGGGCACTGTTACA | 156 |
| | | | | | | | | | | ACCCA | |
| chr | 961175 | A | C | CCDC8 | p.D125 | 0.006 | 0.002 | 2.43 | 2.96 | TGTTGAGATCATTATCCTCT | SEQ |
| 11 | 37 | | | 2 | E | 62 | 24 | E−06 | [1.99- | TGACTTAAATGTTTTTCCTG | ID |
| | | | | | | | | | 4.42] | [A/C]TCTTGTAAGTCAATA | NO: |
| | | | | | | | | | | TTCCTATGTTTGATTTTGTT | 157 |
| | | | | | | | | | | CGTTT | |
| chr | 107381 | G | T | ALKBH | p.H474 | 0.006 | 0.004 | 2.37 | 1.63 | AGAGAAAGAAAGACCATACT | SEQ |
| 11 | 630 | | | 8 | N | 62 | 08 | E−02 | [1.09- | TACTGCTGTTGCAAAATGAT | ID |
| | | | | | | | | | 2.43] | [G/T]AATAACAGCAATGGA | NO: |
| | | | | | | | | | | GATGCAGGCATCACAAGACC | 158 |
| | | | | | | | | | | CACTG | |
| chr | 114451 | T | C | NXPE4 | p.I31V | 0.005 | 0.003 | 3.81 | 1.62 | ACAGGATTCCATGTGTTTCT | SEQ |
| 11 | 010 | | | | | 39 | 33 | E−02 | [1.05- | CCAGACATGCCCACTGGGGA | ID |
| | | | | | | | | | 2.5] | [T/C]TGTGGATGTCATTCC | NO: |
| | | | | | | | | | | AAACTTGCATTTCTCTTTCA | 159 |
| | | | | | | | | | | TTGCA | |
| chr | 116744 | A | G | SIK3 | p.L518 | 0.005 | 0.003 | 3.98 | 1.6 | TGTCTAGGTACCTTGTACTC | SEQ |
| 11 | 648 | | | | L | 39 | 38 | E−02 | [1.04- | AAGTTGCCCGGTTGGTTGCA | ID |
| | | | | | | | | | 2.46] | [A/G]GTTTTGCATAGGCAA | NO: |
| | | | | | | | | | | CAGGTTGTGCATGAAGTTCA | 160 |
| | | | | | | | | | | CATTA | |
| chr | 117054 | G | A | SIDT2 | p.8235 | 0.005 | 0.003 | 2.25 | 1.73 | ATGATGATGAAGAAGATATT | SEQ |
| 11 | 496 | | | | H | 39 | 13 | E−02 | [1.12- | TATCATCATCATCCTGCAGC | ID |
| | | | | | | | | | 2.66] | [G/A]CAAAGACTTCCCCAG | NO: |
| | | | | | | | | | | CAACAGCTTTTATGTGGTGG | 161 |
| | | | | | | | | | | TGGTG | |
| chr | 117057 | C | T | SIDT2 | p.R333 | 0.005 | 0.000 | 3.63 | 533.81 | ATGCAGGCAGAAGAAGAAGA | SEQ |
| 11 | 334 | | | | X | 64 | 01 | E−31 | [72.07- | CCCTGCTGGTGGCCATTGAC | ID |
| | | | | | | | | | 3953.67] | [C/T]GAGCCTGCCCAGAAA | NO: |
| | | | | | | | | | | GCGGTACCTCCAGGGGGCCT | 162 |
| | | | | | | | | | | GGGTG | |
| chr | 118516 | G | A | pHLDB | p.A110 | 0.005 | 0.002 | 2.98 | 2.47 | CCTGCCTGCGGGGCGGGAGC | SEQ |
| 11 | 274 | | | 1 | 8T | 39 | 19 | E−04 | [1.59- | GTGGGGAGGAGGGTGAGCAC | ID |
| | | | | | | | | | 3.82] | [G/A]CCTATGATACGCTGA | NO: |
| | | | | | | | | | | GTCTGGAGAGCTCTGACAGC | 163 |
| | | | | | | | | | | ATGGA | |
| chr | 118850 | C | G | FOXR1 | p.A153 | 0.005 | 0.000 | 7.54 | 287.7 | GACAGCTCCTCTATGGCTCT | SEQ |
| 11 | 225 | | | | G | 15 | 02 | E−29 | [67.44- | CCCATCCCCTCACAAAAGGG | ID |
| | | | | | | | | | 1227.4] | [C/G]CCCCCTCCAGAGTCG | NO: |
| | | | | | | | | | | GAGGCTTCGGCAAGCCAGCA | 164 |
| | | | | | | | | | | GCCAG | |
| chr | 120188 | T | A | POU2F | p.F422 | 0.018 | 0.000 | 5.03 | 2148.21 | TCAAAATAACTCCAAAGCAG | SEQ |
| 11 | 060 | | | 3 | I | 87 | 01 | E− | [298.71- | CAGTGAACTCCGCCTCCAGT | ID |
| | | | | | | | | 111 | 15448.88] | [T/A]TTAACTCTTCAGGGT | NO: |
| | | | | | | | | | | AAGGTGAAGGGGACGGTGCA | 165 |
| | | | | | | | | | | GAGAC | |
| chr | 123476 | C | T | GRAM | p.A295 | 0.006 | 0.003 | 8.40 | 1.76 | TCACCAACAGCACACTAACA | SEQ |
| 11 | 177 | | | D1B | A | 37 | 64 | E−03 | [1.18- | TCCACAGGGAGCAGTGAGGC | ID |
| | | | | | | | | | 2.62] | [C/T]CCCGTCTCGGTATGG | NO: |
| | | | | | | | | | | GCAGTCAGCCTTTGACTTCT | 166 |
| | | | | | | | | | | ACCCC | |
| chr | 124266 | A | G | OR8B3 | p.P286 | 0.009 | 0.003 | 1.44 | 3.04 | GTGCAACTTTGACATCCTTG | SEQ |
| 11 | 390 | | | | P | 31 | 09 | E−08 | [2.17- | TTCCTCAAACTGTAGATGAG | ID |
| | | | | | | | | | 4.25] | [A/G]GGATTGAGCATGGGC | NO: |
| | | | | | | | | | | ACCACATTAGTGTAGAAAAC | 167 |
| | | | | | | | | | | AGAAG | |
| chr | 124620 | G | T | VSIG2 | p.N97 | 0.005 | 0.000 | 6.91 | 288.96 | CGTCAGTCAGTTTCAGTGTG | SEQ |
| 11 | 746 | | | | K | 15 | 02 | E−29 | [67.73- | GCCACCCCCACTGTGGGGGG | ID |
| | | | | | | | | | 1232.76] | [G/T]TTCTGAAGCAGGCTG | NO: |
| | | | | | | | | | | ACCCGCTTTGACTTAGAACC | 168 |
| | | | | | | | | | | AGTTG | |
| chr | 368928 | T | C | SLC6A1 | p.P97P | 0.008 | 0.005 | 2.84 | 1.72 | CCTCTAAGCGTCCTCCTACC | SEQ |
| 12 | | | | 3 | | 82 | 15 | E−03 | [1.23- | TCCAGAATTCTATACATCTA | ID |
| | | | | | | | | | 2.41] | [T/C]GGGACTCCCCAGAGG | NO: |
| | | | | | | | | | | GGCCGTAAGTGCAGGAGATG | 169 |
| | | | | | | | | | | GAAGT | |
| chr | 704483 | C | T | ATN1 | p.Y136 | 0.011 | 0.007 | 1.73 | 1.46 | ATATCGACCAGGACAACCGA | SEQ |
| 12 | 8 | | | | Y | 03 | 57 | E−02 | [1.08- | AGCACGTCCCCCAGTATCTA | ID |
| | | | | | | | | | 1.98] | [C/T]AGCCCTGGAAGTGTG | NO: |
| | | | | | | | | | | GAGAATGACTCTGACTCATC | 170 |
| | | | | | | | | | | TTCTG | |
| chr | 109594 | C | A | TAS2R | p.R55I | 0.007 | 0.004 | 1.11 | 1.65 | TACAATGCCATTTACAACCA | SEQ |
| 12 | 16 | | | 8 | | 84 | 77 | E−02 | [1.15- | TTACACTGATCAAACAAATT | ID |
| | | | | | | | | | 2.36] | [C/A]TGGCGATAACTAAAT | NO: |
| | | | | | | | | | | TGGTAAGGATGTAGTCAACT | 171 |
| | | | | | | | | | | GTGGA | |
| chr | 114617 | G | T | PRB4 | p.P50T | 0.026 | 0.006 | 4.29 | 3.98 | TGTGGGGGTGGTCCTTGTGG | SEQ |
| 12 | 69 | | | | | 72 | 86 | E−29 | [3.23- | CTTTCCTGGAGGAGGTGGGG | ID |
| | | | | | | | | | 4.9] | [G/T]ACGTTGGGGCTGGTT | NO: |
| | | | | | | | | | | TCCTCCTTGTGGGCGTCGTC | 172 |
| | | | | | | | | | | CTTCT | |
| chr | 130615 | A | G | GPRC5 | p.I134 | 0.013 | 0.009 | 1.04 | 1.45 | CAAGCTCGTCCGGGGGAGGA | SEQ |
| 12 | 83 | | | A | V | 48 | 32 | E−02 | [1.11- | AGCCCCTTTCCCTGTTGGTG | ID |
| | | | | | | | | | 1.91] | [A/G]TTCTGGGTCTGGCCG | NO: |
| | | | | | | | | | | TGGGCTTCAGCCTAGTCCAG | 173 |
| | | | | | | | | | | GATGT | |
| chr | 152623 | C | T | RERG | p.V95V | 0.009 | 0.006 | 1.84 | 1.49 | TGGGCTTTTTGATCTCATCT | SEQ |
| 12 | 59 | | | | | 80 | 58 | E−02 | [1.09- | AGGATGTTCTTAAGTGGCAG | ID |
| | | | | | | | | | 2.06] | [C/T]ACTTCCTCAAAACTT | NO: |
| | | | | | | | | | | CCTCGGTCAGTAATGTCGTA | 174 |
| | | | | | | | | | | GACCA | |
| chr | 482402 | G | A | VDR | p.A353 | 0.005 | 0.003 | 1.83 | 1.71 | GGAACTTGATGAGGGGCTCA | SEQ |
| 12 | 33 | | | | A | 64 | 30 | E−02 | [1.11- | ATCAGCTCCAGGCTGTGTCC | ID |
| | | | | | | | | | 2.64] | [G/A]GCTGTGAGAGACAAT | NO: |
| | | | | | | | | | | GGCCAGGTACTGCGGGCAGA | 175 |
| | | | | | | | | | | GCTGA | |
| chr | 494255 | C | T | KMT2D | p.V430 | 0.005 | 0.002 | 1.83 | 2.1 | TTTGGCTCTTGAGGGCTGGA | SEQ |
| 12 | 75 | | | | 5I | 39 | 57 | E−03 | [1.36- | TGGTGGAGGTGTGGGATGGA | ID |
| | | | | | | | | | 3.25] | [C/T]AGGGCCAAGGACTGG | NO: |
| | | | | | | | | | | TCCTGTAGATAAGGCTCCTG | 176 |
| | | | | | | | | | | GTGGG | |
| chr | 504801 | G | T | SMARC | p.Q11 | 0.007 | 0.004 | 8.42 | 1.8 | CCCGCAAGAGACCTGCCCCT | SEQ |
| 12 | 02 | | | D1 | 2H | 807 | 359 | E−03 | [1.14- | CAGCAGATCCAGCAGGTCCA | ID |
| | | | | | | | | | 2.71] | [G/T]CAGCAGGCGGTCCAA | NO: |
| | | | | | | | | | | AATCGAAACCACAAGTAAGA | 177 |
| | | | | | | | | | | TGATC | |
| chr | 507457 | G | A | FAM18 | p.A160 | 0.005 | 0.000 | 3.41 | 10.56 | CTGGGCCTGCTGAGGGGTGA | SEQ |
| 12 | 92 | | | 6A | 8V | 88 | 56 | E−14 | [6.19- | GAGGGATCCCCTGAGCCTGC | ID |
| | | | | | | | | | 18.02] | [G/A]CCTGCTGAGGGGTGA | NO: |
| | | | | | | | | | | GAGGGATCCCCAGTTCCTGC | 178 |
| | | | | | | | | | | GCCTG | |
| chr | 507468 | A | G | FAM18 | p.V126 | 0.025 | 0.000 | 7.89 | 399.44 | CTGGGCCTGCTGAGGAGTAA | SEQ |
| 12 | 36 | | | 6A | 0A | 74 | 07 | E− | [126.72- | GAGGGATCCCCAGTTCCTGA | ID |
| | | | | | | | | 110 | 1259.11] | [A/G]CCTGCTTAGGGGTGA | NO: |
| | | | | | | | | | | GAGTGATTCCGAGAGCCTGC | 179 |
| | | | | | | | | | | GCCTG | |
| chr | 507481 | T | G | FAM18 | p.K816 | 0.005 | 0.002 | 1.29 | 1.97 | TCTTGCAAATATTGCTCCTG | SEQ |
| 12 | 69 | | | 6A | Q | 21 | 65 | E−02 | [1.11- | CCTTTGTTTTTCCTTCTCCT | ID |
| | | | | | | | | | 3.26] | [T/G]GTGGTCTTTCTGTAC | NO: |
| | | | | | | | | | | TGTTGAGACTGTTGGAATAT | 180 |
| | | | | | | | | | | CTCTT | |
| chr | 529608 | C | A | KRT74 | p.G507 | 0.005 | 0.002 | 9.47 | 2.09 | GGCTGGGGTGCTCTTGCCCT | SEQ |
| 12 | 23 | | | | V | 21 | 49 | E−03 | [1.18- | GGGTGTCCTTGAGGTCTCCC | ID |
| | | | | | | | | | 3.47] | [C/A]CTCGCGCCTCTGTGG | NO: |
| | | | | | | | | | | TCTTGGTCTGCCCGCTCTGG | 181 |
| | | | | | | | | | | GTGCT | |
| chr | 529620 | G | A | KRT74 | p.R420 | 0.008 | 0.005 | 2.50 | 1.51 | AGTTTCAGGCTCATGAGCTC | SEQ |
| 12 | 50 | | | | W | 33 | 55 | E−02 | [1.06- | CTGGTACTCGCGCAGCATCC | ID |
| | | | | | | | | | 2.13] | [G/A]CGCCAGCTCCTCCTT | NO: |
| | | | | | | | | | | GGCCTGGTGCAGGGCGCCCT | 182 |
| | | | | | | | | | | CCAGC | |
| chr | 534481 | G | A | TENC1 | p.T13T | 0.005 | 0.003 | 1.35 | 1.79 | TCATGGAGCGGCGCTGGGAC | SEQ |
| 12 | 14 | | | | | 39 | 01 | E−02 | [1.16- | TTAGACCTCACCTACGTGAC | ID |
| | | | | | | | | | 2.77] | [G/A]GAGCGCATCTTGGCC | NO: |
| | | | | | | | | | | GCCGCCTTCCCCGCGCGGCC | 183 |
| | | | | | | | | | | CGATG | |
| chr | 535169 | C | T | SOAT2 | p.V455 | 0.010 | 0.007 | 2.52 | 1.45 | TGGGGTTCTTCTATCCCGTC | SEQ |
| 12 | 93 | | | | V | 54 | 32 | E−02 | [1.06 | ATGCTGATACTCTTCCTTGT | ID |
| | | | | | | | | | 1.97] | [C/T]ATTGGAGGTGAGCTG | NO: |
| | | | | | | | | | | GTCTCTGTGCCACTGGAAGG | 184 |
| | | | | | | | | | | GAGCC | |
| chr | 537144 | G | T | AAAS | p.T57N | 0.009 | 0.000 | 5.15 | Inf | GATGAAGGCAGTTCTTGTGC | SEQ |
| 12 | 30 | | | | | 31 | 00 | E−56 | | CATGGTCCAGCCTTCCAGGG | ID |
| | | | | | | | | | | [G/T]TCTTTAGGGGATCCT | NO: |
| | | | | | | | | | | TTGTCAGTTGTAGGACAGGA | 185 |
| | | | | | | | | | | AGATT | |
| chr | 558464 | C | A | OR6C2 | p.L164 | 0.005 | 0.002 | 1.70 | 1.8 | TGATGATCATTGTTCCACCA | SEQ |
| 12 | 89 | | | | L | 15 | 87 | E−02 | [1.16- | CTTAGCTTAGGCCTCCAGCT | ID |
| | | | | | | | | | 2.8] | [C/A]GAATTCTGTGACTCC | NO: |
| | | | | | | | | | | AATGCCATTGATCATTTTAG | 186 |
| | | | | | | | | | | CTGTG | |
| chr | 563509 | C | C | PMEL | p.E370 | 0.005 | 0.002 | 5.00 | 2.3 | CCTCTGAAACTGGCACCTTC | SEQ |
| 12 | 77 | | | | D | 88 | 57 | E−04 | [1.51- | TCAGGTGTCATACCTGTGCT | ID |
| | | | | | | | | | 3.49] | [C/G]TCTGCAGTTGGCATC | NO: |
| | | | | | | | | | | TGCACAGGTGCAGTGCTTAT | 187 |
| | | | | | | | | | | GACTT | |
| chr | 570092 | G | A | BAZ2A | p.N10 | 0.007 | 0.004 | 4.23 | 1.48 | GTCCCCCCGAGAACTGGGAG | SEQ |
| 12 | 16 | | | | 6N | 35 | 97 | E−02 | [1.02- | AGAAGGGGTGGGTCCTTGAG | ID |
| | | | | | | | | | 2.14] | [G/A]TTGCTGCCAGGATTG | NO: |
| | | | | | | | | | | GCAGATGGGTACTGTGAGTA | 188 |
| | | | | | | | | | | GTTCC | |
| chr | 575693 | G | A | LRP1 | p.G121 | 0.008 | 0.005 | 8.35 | 1.64 | GAAGGCATTGTGTGTTCCTG | SEQ |
| 12 | 39 | | | | SE | 58 | 26 | E−03 | [1.16- | CCCTCTGGGCATGGAGCTGG | ID |
| | | | | | | | | | 2.3] | [G/A]GCCCGACAACCACAC | NO: |
| | | | | | | | | | | CTGCCAGATCCAGAGCTACT | 189 |
| | | | | | | | | | | GTGCC | |
| chr | 667251 | G | A | HELB | p.G959 | 0.005 | 0.003 | 2.31 | 1.66 | TCGTTTGAAACATTTCTTGC | SEQ |
| 12 | 38 | | | | S | 88 | 56 | E−02 | [1.1- | AAAGTAAGCTCTCCTCTAGC | ID |
| | | | | | | | | | 2.51] | [G/A]GCGCACCTCCAGCAG | NO: |
| | | | | | | | | | | ATTTTCCGTCCCCACGGAAG | 190 |
| | | | | | | | | | | AGCTC | |
| chr | 856951 | C | T | ALX1 | p.N27 | 0.009 | 0.000 | 1.86 | Inf | TTTCAAACCACCAGAACCAG | SEQ |
| 12 | 06 | | | | 8N | 56 | 00 | E−57 | | TTCAGCCACGTGCCCCTCAA | ID |
| | | | | | | | | | | [C/T]AATTTTTTCACTGAC | NO: |
| | | | | | | | | | | TCTCTTCTTACTGGGGCAAC | 191 |
| | | | | | | | | | | CAATG | |
| chr | 899169 | G | A | POC1B- | p.I450I | 0.006 | 0.004 | 3.83 | 1.55 | GGTTGTTGTCAGGAGAATTA | SEQ |
| 12 | 68 | | | GALNT | | 62 | 28 | E−02 | [1.05- | TAATCTAAACATTCAGACGA | ID |
| | | | | 4 | | | | | 2.29] | [G/A]ATCCCTCTACTGCGA | NO: |
| | | | | | | | | | | ATAGCCCCATGCCAGCCTGG | 192 |
| | | | | | | | | | | TCTAT | |
| chr | 956942 | C | T | VEZT | p.P712 | 0.001 | 0.000 | 1.23 | 41.5 | TGAACCACAAGCAGATGGAA | SEQ |
| 12 | 43 | | | | S | 96 | 05 | E−05 | [11.7- | GTGGTCTGACCACTGCCCCT | ID |
| | | | | | | | | | 147] | [C/T]CAACTCCCAGGGACT | NO: |
| | | | | | | | | | | CATTACAGCCCTCCATTAAG | 193 |
| | | | | | | | | | | CAGAG | |
| chr | 104144 | C | T | STAB2 | p.P217 | 0.006 | 0.003 | 1.77 | 1.68 | CTATGTCGGAGATGGGCTGA | SEQ |
| 12 | 426 | | | | 0S | 13 | 66 | E−02 | [1.12- | ACTGTGAGCCGGAGCAGCTG | ID |
| | | | | | | | | | 2.52] | [C/T]CCATTGACCGCTGCT | NO: |
| | | | | | | | | | | TACAGGACAATGGGCAGTGC | 194 |
| | | | | | | | | | | CATGC | |
| chr | 108920 | G | A | SART3 | p.F691 | 0.005 | 0.003 | 3.10 | 1.62 | TGATGCTGTCCTTGCTGCTG | SEQ |
| 12 | 173 | | | | D | 64 | 48 | E−02 | [1.06- | TCGTGCAGCACCTTGGGCAT | ID |
| | | | | | | | | | 2.47] | [G/A]TCCCTCTTCAGGGAG | NO: |
| | | | | | | | | | | GCTGCCTTCTCCTTCTGCTT | 195 |
| | | | | | | | | | | CGAAG | |
| chr | 111317 | T | C | CCDC6 | p.L172 | 0.007 | 0.004 | 4.98 | 1.49 | CTCCAGCACTGCCTGTTGAT | SEQ |
| 12 | 855 | | | 3 | S | 11 | 78 | E−02 | [1.03- | GGAGAAGAAAACCATGAACT | ID |
| | | | | | | | | | 2.17] | [T/C]GGCCATTGAGCAATC | NO: |
| | | | | | | | | | | TTCTCAGGCCTATGAGCAGA | 196 |
| | | | | | | | | | | GGTGG | |
| chr | 119594 | C | T | SRRM4 | p.S529 | 0.013 | 0.000 | 4.82 | Inf | CCATCCCCTACTATCGGCCC | SEQ |
| 12 | 354 | | | | S | 48 | 00 | E−80 | | AGCCCCTCCTCATCCGGCAG | ID |
| | | | | | | | | | | [C/T]CTCAGCAGCACCTCC | NO: |
| | | | | | | | | | | TCCTGGTACAGCAGCAGCAG | 197 |
| | | | | | | | | | | TAGCC | |
| chr | 122361 | C | T | WDR6 | p.R188 | 0.012 | 0.008 | 5.94 | 1.53 | TGAAAGGCAGCCCTCAGGAG | SEQ |
| 12 | 711 | | | 6 | W | 25 | 07 | E−03 | [1.15- | AGCTTGAGGAGAAAACCGAC | ID |
| | | | | | | | | | 2.03] | [C/T]GGATGCCCCAAGATG | NO: |
| | | | | | | | | | | AACTGGGACAAGAAAGAAGG | 198 |
| | | | | | | | | | | GACTT | |
| chr | 122404 | C | T | WDR6 | P.R860 | 0.012 | 0.008 | 1.00 | 1.49 | ACAAGTCCTCCCAGTGAGAA | SEQ |
| 12 | 946 | | | 6 | C | 01 | 07 | E−02 | [1.12- | GCATGGCGGAGCTACAGAAA | ID |
| | | | | | | | | | 1.99] | [C/T]GCTACTTGGTGTTTA | NO: |
| | | | | | | | | | | TTAACAGAGACAAGGTAACA | 199 |
| | | | | | | | | | | GCGCT | |
| chr | 122676 | A | G | L33C4 | p.Y159 | 0.005 | 0.002 | 3.52 | 2.01 | CCCGAAGGCCCTTTCATCAC | SEQ |
| 12 | 056 | | | 3 | C | 39 | 69 | E−03 | [1.3- | TTACAACTATTACGTGACCT | ID |
| | | | | | | | | | 3.1] | [A/G]TGATTTTGTGAAAGA | NO: |
| | | | | | | | | | | TGAAGAAGGCGAAATGAATG | 200 |
| | | | | | | | | | | AGTCC | |
| chr | 123706 | T | G | MPHO | p.S160 | 0.006 | 0.000 | 8.30 | 14.36 | GTGGATTCAGGATAATGGAT | SEQ |
| 12 | 313 | | | SPH9 | R | 51 | 46 | E−15 | [7.8- | AACAGATTCATTTCTCTCAC | ID |
| | | | | | | | | | 25.78] | [T/G]GCTTAGAGAAAAAAA | NO: |
| | | | | | | | | | | ACCCATTTGAcTTTCCGAAG | 201 |
| | | | | | | | | | | ATACT | |
| chr | 124364 | C | T | DNAH1 | p.H273 | 0.007 | 0.004 | 1.93 | 1.59 | GGGATCCCATATTGTTTGGA | SEQ |
| 12 | 285 | | | 0 | 9H | 35 | 64 | E−02 | [1.1- | GACTTCCAGATGGCTCTGCA | ID |
| | | | | | | | | | 2.3] | [C/T]GAAGGAGAACCACGC | NO: |
| | | | | | | | | | | ATTTATGAAGACATCCAGGA | 202 |
| | | | | | | | | | | CTACG | |
| chr | 125396 | G | A | UBC | p.D495 | 0.028 | 0.012 | 4.07 | 2.27 | CATCTTCCAGCTGTTTCCCA | SEQ |
| 12 | 833 | | | | D | 92 | 95 | E−08 | [1.69- | GCAAAGATCAACCTCTGCTG | ID |
| | | | | | | | | | 3.06] | [G/A]TCAGGAGGGATGCCT | NO: |
| | | | | | | | | | | TCCTTGTCTTGGATCTTTGC | 203 |
| | | | | | | | | | | CTTGA | |
| chr | 125397 | T | C | UBC | p.025 | 0.005 | 0.000 | 1.03 | 71.98 | AGATCAACCTCTGCTGGTCA | SEQ |
| 12 | 541 | | | | 9Q | 15 | 07 | E−24 | [31.86- | GGAGGAATGCCTTCCTTGTC | ID |
| | | | | | | | | | 162.59] | [T/C]TGGATCTTTGCTTTG | NO: |
| | | | | | | | | | | ACGTTCTCGATAGTGTCACT | 204 |
| | | | | | | | | | | GGGCT | |
| chr | 125398 | A | G | UBC | p.T7T | 0.012 | 0.000 | 1.46 | 94.03 | CACTGGGCTCAACCTCGAGG | SEQ |
| 12 | 297 | | | | | 53 | 10 | E−33 | [44.17- | GTGATGGTCTTACCAGTCAG | ID |
| | | | | | | | | | 200.19] | [A/G]GTCTTCACGAAGATC | NO: |
| | | | | | | | | | | TGCATTGTCTAACAAAAAAG | 205 |
| | | | | | | | | | | CCAAA | |
| chr | 132625 | G | A | DDX51 | p.S487 | 0.022 | 0.000 | 2.59 | 2540.86 | CCAGGACCAGGTGCAGGACG | SEQ |
| 12 | 260 | | | | S | 30 | 01 | E− | [354- | ACCAGCGGCTTAGAGCTGAG | ID |
| | | | | | | | | 131 | 18237.12] | [G/A]CTGCAGGGCACGTAG | NO: |
| | | | | | | | | | | TGGTGCTACAGGGACGGCAG | 206 |
| | | | | | | | | | | GGGGT | |
| chr | 368717 | G | T | CCDC1 | p.V25V | 0.006 | 0.003 | 1.29 | 1.72 | GGGACCCCACACCGCGCCGC | SEQ |
| 13 | 82 | | | 69 | | 37 | 72 | E−02 | [1.14- | CCGCCGACTCACTTCTTGCG | ID |
| | | | | | | | | | 2.59] | [G/T]ACTTCTTCCAGCAAC | NO: |
| | | | | | | | | | | TGCTGTTTCAGGCGGTTGGT | 207 |
| | | | | | | | | | | GCTCA | |
| chr | 423521 | T | C | VWA8 | p.M76 | 0.005 | 0.003 | 2.56 | 1.62 | ACCAATAATAAGTGTTCTCC | SEQ |
| 13 | 71 | | | | 7V | 88 | 63 | E−02 | [1.07- | AAGGAGAAAGTCTTTCAGCA | ID |
| | | | | | | | | | 2.45] | [T/C]ATCTTCCATCACTAT | NO: |
| | | | | | | | | | | CACATGCTAGAGAAAAAGGA | 208 |
| | | | | | | | | | | ACTAG | |
| chr | 492817 | T | A | CYSLTR | p. L278 | 0.016 | 0.001 | 1.09 | 10.37 | CACACTGAGGACCGTCCACT | SEQ |
| 13 | 85 | | | 2 | I | 93 | 66 | E−30 | [7.32- | TGACGACATGGAAAGTGGGT | ID |
| | | | | | | | | | 14.5] | [T/A]TATGCAAAGACAGAC | NO: |
| | | | | | | | | | | TGCATAAAGCTTTGGTTATC | 209 |
| | | | | | | | | | | ACACT | |
| chr | 763816 | T | C | LMO7 | p.H187 | 0.008 | 0.004 | 7.04 | 1.9 | TCCAAACATACTCTGATGAC | SEQ |
| 13 | 79 | | | | H | 82 | 66 | E−04 | [1.36- | ATCTTGTCTTCTGAAACACA | ID |
| | | | | | | | | | 2.67] | [T/C]ACCAAAATTGATCCC | NO: |
| | | | | | | | | | | ACTTCTGGCCCAAGGCTCAT | 210 |
| | | | | | | | | | | AACCC | |
| chr | 995404 | G | T | DOCK9 | p.P679 | 0.008 | 0.000 | 3.06 | Inf | CGTAGGTGAACATATATTAA | SEQ |
| 13 | 20 | | | | T | 33 | 00 | E−49 | | AAAAAAACAAACCTTAAGGG | ID |
| | | | | | | | | | | [G/T]CTGAGAGTCTTCCTC | NO: |
| | | | | | | | | | | ATCTGAATCTTTGAATTCAA | 211 |
| | | | | | | | | | | TGCAA | |
| chr | 103382 | T | C | CCDC1 | p.K699 | 0.000 | 0.000 | 1.26 | 14.31 | TTTTCTTTCAGAATAGAAGT | SEQ |
| 13 | 057 | | | 68 | 7R | 25 | 02 | E−01 | [0.89- | TGATATCGTCATGATGAGGT | ID |
| | | | | | | | | | 228.77] | [T/C]TTGATGCTGATTTAT | NO: |
| | | | | | | | | | | GTTTGCTTTGGAAACAATCC | 212 |
| | | | | | | | | | | AATCT | |
| chr | 103382 | G | A | CCDC1 | p.T685 | 0.000 | 0.000 | 2.60 | 2.18 | TCTATATTTCCTGCTTTTGT | SEQ |
| 13 | 483 | | | 68 | 5I | 49 | 22 | E−01 | [0.49- | GGGACTTACAGGAAGGTGGT | ID |
| | | | | | | | | | 9.67] | [G/A]TAATAATTAAGGTTT | NO: |
| | | | | | | | | | | CCTTTCTGCACTCTCTAGTA | 213 |
| | | | | | | | | | | CAATG | |
| chr | 103382 | A | G | CCDC1 | p.V679 | 0.009 | 0.008 | 4.27 | 1.13 | TTCTGATTCCTGACTTAAAT | SEQ |
| 13 | 660 | | | 68 | 6A | 56 | 43 | E−01 | [0.82- | AAGAGTTGGCTTCCAGAAAC | ID |
| | | | | | | | | | 4.57] | [A/G]CACATTCCTCACTCT | NO: |
| | | | | | | | | | | CACTTACTTCAAGACATGAA | 214 |
| | | | | | | | | | | CACTC | |
| chr | 103382 | C | T | CCDC1 | p.E678 | 0.000 | 0.000 | 2.43 | 4.63 | ACACATTCCTCACTCTCACT | SEQ |
| 13 | 700 | | | 68 | 3K | 25 | 05 | E−01 | [0.48- | TACTTCAAGACATGAACACT | ID |
| | | | | | | | | | 44.52] | [C/T]GTCCAAGTCAGCTGG | NO: |
| | | | | | | | | | | ACTCTCAATATCTGTCTGAA | 215 |
| | | | | | | | | | | TATCA | |
| chr | 103383 | C | T | CCDC1 | p.E660 | 0.000 | 0.000 | 1.87 | 7 | TATTGTAAATCAAGATCTAT | SEQ |
| 13 | 228 | | | 68 | 7K | 25 | 04 | E−01 | [0.63- | TTGATGGAGAGATTTCTCCT | ID |
| | | | | | | | | | 77.21] | [C/T]AGAAAGTAACAAAAT | NO: |
| | | | | | | | | | | TCTGTTTTGTCGTTTTGGTC | 216 |
| | | | | | | | | | | CTGTG | |
| chr | 103383 | T | C | CCDC1 | p.R657 | 0.000 | 0.000 | 2.19 | 2.5 | TTCTTTCTCTCATGAGCACT | SEQ |
| 13 | 339 | | | 68 | 0G | 49 | 20 | E−01 | [0.55- | GGTCATTGCATAAGATTCTC | ID |
| | | | | | | | | | 11.27] | [T/C]TACAATTCTGGGAAA | NO: |
| | | | | | | | | | | GGCTTTCATTTGTATCTCCA | 217 |
| | | | | | | | | | | ATGTT | |
| chr | 103383 | T | G | CCDC1 | p.E650 | 0.002 | 0.002 | 1.00 | 0.96 | ATTTTCTAGCTTATTAATAC | SEQ |
| 13 | 524 | | | 68 | 8A | 70 | 81 | E+00 | [0.52- | TCTGTAGCTTTGTGATTGTC | ID |
| | | | | | | | | | 1.77] | [T/G]CCTCACTGTCACTTG | NO: |
| | | | | | | | | | | AAACATCAACAATCAGTGTC | 218 |
| | | | | | | | | | | TTCAT | |
| chr | 103383 | A | C | CCDC1 | p.S646 | 0.000 | 0.000 | 1.89 | 6.91 | GTCCCTTCTAGAGACATAAA | SEQ |
| 13 | 666 | | | 68 | 1A | 25 | 04 | E−01 | [0.63- | GTTCATTGTTTTATGTCTAG | ID |
| | | | | | | | | | 76.19] | [A/C]ATAGAACCTCCAACT | NO: |
| | | | | | | | | | | GTTATCTTTTGAAATAGTCC | 219 |
| | | | | | | | | | | CTTTT | |
| chr | 103383 | G | A | CCDC1 | p.H641 | 0.002 | 0.000 | 1.49 | 9.81 | ATCAGATTCAGTTGTATTTC | SEQ |
| 13 | 792 | | | 68 | 9Y | 94 | 30 | E−07 | [4.68- | AAGTGCTTTTGACTCTAAAT | ID |
| | | | | | | | | | 20.56] | [G/A]ACTAGTAAGCTTATT | NO: |
| | | | | | | | | | | TTTTTCTTTGGGAGTAAACT | 220 |
| | | | | | | | | | | GTTCT | |
| chr | 103383 | T | G | CCDC1 | p.E641 | 0.000 | 0.000 | 6.73 | Inf | AAGTGCTTTTGACTCTAAAT | SEQ |
| 13 | 812 | | | 68 | 2A | 25 | 00 | E−02 | [NaN- | GACTAGTAAGCTTATTTTTT | ID |
| | | | | | | | | | Inf] | [T/G]CTTTGGGAGTAAACT | NO: |
| | | | | | | | | | | GTTCTAAAAGGGATTTGTGC | 221 |
| | | | | | | | | | | TGCGT | |
| chr | 103383 | C | T | CCDC1 | p.D636 | 0.001 | 0.002 | 2.75 | 0.61 | AAGTCGTCAGGCTTATAGGC | SEQ |
| 13 | 951 | | | 68 | 6N | 72 | 82 | E−01 | [0.28- | TTGTATGTTATCTAGTTTAT | ID |
| | | | | | | | | | 1.3] | [C/T]AGAAGAAACTTTGTC | NO: |
| | | | | | | | | | | TTGGATCATATTTTTAACCT | 222 |
| | | | | | | | | | | GGGAC | |
| chr | 103384 | C | T | CCDC1 | p.S632 | 0.000 | 0.000 | 4.28 | 1.98 | ATGTTCTGCNTTTGTACTGT | SEQ |
| 13 | 070 | | | 68 | 6N | 25 | 12 | E−01 | [0.24- | CTGCAACTATTTTGACTTCG | ID |
| | | | | | | | | | 16.06] | [C/T]TACTTTTAACTTGAG | NO: |
| | | | | | | | | | | GCGGTATGGGCACAGTTCCT | 223 |
| | | | | | | | | | | GGGAA | |
| chr | 103384 | G | A | CCDC1 | p.T611 | 0.021 | 0.024 | 1.58 | 0.85 | ATACTCTAATTTCTTTCTAT | SEQ |
| 13 | 712 | | | 68 | 2M | 32 | 94 | E−01 | [0.68- | TGCTTGGTGTACCACGCCCC | ID |
| | | | | | | | | | 1.06] | [G/A]TGATATTAAGCATCT | NO: |
| | | | | | | | | | | GTGGAATTGGGTGATTCTGG | 224 |
| | | | | | | | | | | ATTTT | |
| chr | 103385 | T | C | CCDC1 | p.K599 | 0.003 | 0.004 | 5.30 | 0.81 | GGGTGTGCACTACTGCTTGT | SEQ |
| 13 | 064 | | | 68 | SE | 43 | 24 | E−01 | [0.47- | GTCCATTCTTCCTCTCTCCT | ID |
| | | | | | | | | | 1.39] | [T/C]CTCCAGATTGGCAGT | NO: |
| | | | | | | | | | | CCTGGCCTTGTGCATCTCTG | 225 |
| | | | | | | | | | | TTTTC | |
| chr | 103385 | G | A | CCDC1 | p.P591 | 0.000 | 0.000 | 6.72 | Inf | TGATTGAAATTGAAAAGTCC | SEQ |
| 13 | 294 | | | 68 | 8L | 25 | 00 | E−02 | [NaN- | AGGGAGGGAATAGGGACTTC | ID |
| | | | | | | | | | Inf] | [G/A]GAAGAAATTCCAGAA | NO: |
| | | | | | | | | | | CACCTTCCTCTTGTTCTGAA | 226 |
| | | | | | | | | | | ATGAG | |
| chr | 103385 | C | A | CCDC1 | p.A590 | 0.000 | 0.000 | 4.26 | 1.99 | AATTCCAGAACACCTTCCTC | SEQ |
| 13 | 340 | | | 68 | 3S | 25 | 12 | E−01 | [0.24- | TTGTTCTGAAATGAGCAATG | ID |
| | | | | | | | | | 16.16] | [C/A]CTGCTTCCTTCCCCC | NO: |
| | | | | | | | | | | TTTTGCAGGGTCAATCTCTG | 227 |
| | | | | | | | | | | TCATA | |
| chr | 103385 | C | T | CCDC1 | p.G584 | 0.000 | 0.000 | 1.31 | 13.75 | GGAAACTTAGAAAGGATAGT | SEQ |
| 13 | 520 | | | 68 | 3R | 25 | 02 | E−01 | [0.86- | GTTCGTCCTGGTCTTGTGCC | ID |
| | | | | | | | | | 219.86] | [C/T]ATGTTCACACCGTCG | NO: |
| | | | | | | | | | | GATCACTTGCTTTTTCATGA | 228 |
| | | | | | | | | | | CAATA | |
| chr | 103385 | G | T | CCDC1 | p.S579 | 0.000 | 0.000 | 1.00 | 0.86 | TTTGAGTGATCCCTTTGTCT | SEQ |
| 13 | 654 | | | 68 | 8Y | 25 | 28 | E+00 | [0.11- | GTGGTGCTAACACTTTGGGA | ID |
| | | | | | | | | | 6.5] | [G/T]AAAACATTTTGCTGA | NO: |
| | | | | | | | | | | TTCTATCATTACTTTGTCCA | 229 |
| | | | | | | | | | | TCTTC | |
| chr | 103386 | C | T | CCDC1 | p.V560 | 0.000 | 0.000 | 6.74 | Inf | GCCTCTGGGCGGGGCACATA | SEQ |
| 13 | 222 | | | 68 | 9I | 25 | 00 | E−02 | [NaN- | CTGTTCTGCTTGCTTAACAA | ID |
| | | | | | | | | | Inf] | [C/T]GTTTTTATCAACGCC | NO: |
| | | | | | | | | | | TTCAACTGAGTCTCTATTTG | 230 |
| | | | | | | | | | | TTATT | |
| chr | 103387 | C | T | CCDC1 | p.V534 | 0.000 | 0.000 | 2.98 | 3.42 | TGCTTTTCATTTTTAACATC | SEQ |
| 13 | 002 | | | 68 | 9I | 25 | 07 | E−01 | [0.38- | TTTTGGGATATCACCAACGA | ID |
| | | | | | | | | | 30.56] | [C/T]GGACTCTCTATGTAC | NO: |
| | | | | | | | | | | AGTCTCCCCTATGTGTGATA | 231 |
| | | | | | | | | | | TTCTC | |
| chr | 103387 | C | T | CCDC1 | p.R533 | 0.002 | 0.004 | 1.64 | 0.63 | GGACTCTCTATGTACAGTCT | SEQ |
| 13 | 043 | | | 68 | 5Q | 70 | 28 | E−01 | [0.34- | CCCCTATGTGTGATATTCTC | ID |
| | | | | | | | | | 1.15] | [C/T]GCAAAATAGGTCTTT | NO: |
| | | | | | | | | | | TAAGTCTTAGCATTTCATTA | 232 |
| | | | | | | | | | | CCTAA | |
| chr | 103387 | G | A | CCDC1 | p.P528 | 0.020 | 0.017 | 2.99 | 1.13 | TTCACCTTCACATTCCTGCA | SEQ |
| 13 | 196 | | | 68 | 4L | 10 | 80 | E−01 | [0.9- | CCTTCTCTTCCTGATGTTTG | ID |
| | | | | | | | | | 1.42] | [G/A]GGAATATTAAGATGC | NO: |
| | | | | | | | | | | TTACTATTTGCACGTCATCC | 233 |
| | | | | | | | | | | TCTTC | |
| chr | 103387 | C | A | CCDC1 | p.G524 | 0.000 | 0.000 | 4.64 | Inf | GATTAAAATATCACCAGCAA | SEQ |
| 13 | 313 | | | 68 | 5V | 49 | 00 | E−03 | [NaN- | TTGGCCTTATACATGTGCCT | ID |
| | | | | | | | | | Inf] | [C/A]CCTCAGTATCTGGTG | NO: |
| | | | | | | | | | | ATACCTGGAGTTTTACTAGG | 234 |
| | | | | | | | | | | GGAAA | |
| chr | 103387 | C | T | CCDC1 | p.V509 | 0.000 | 0.000 | 5.68 | 6.92 | GACCGTGACTGTGGGAGAGA | SEQ |
| 13 | 767 | | | 68 | 4M | 49 | 07 | E−02 | [1.27- | CACTTTTGCAATTCTTATCA | ID |
| | | | | | | | | | 37.81] | [C/T]GTTCTCCTGTCCTTC | NO: |
| | | | | | | | | | | TGTTGTATCAAACTTAAGAT | 235 |
| | | | | | | | | | | ATGGT | |
| chr | 103388 | C | G | CCDC1 | p.G501 | 0.035 | 0.034 | 7.24 | 1.03 | TTTGTCTTCCATATCTATTC | SEQ |
| 13 | 015 | | | 68 | 1A | 78 | 78 | E−01 | [0.87- | TGAGTCCACCTTTCTCTTCT | ID |
| | | | | | | | | | 1.22] | [C/G]CCTGTGCTGTGGGTT | NO: |
| | | | | | | | | | | GCACTGGTCCTTTTGAGTTG | 236 |
| | | | | | | | | | | CTTAA | |
| chr | 103388 | A | T | CCDC1 | p.L490 | 0.000 | 0.000 | 1.30 | 13.83 | CCATTGCATAGAAGTGCAAG | SEQ |
| 13 | 343 | | | 68 | 2M | 25 | 02 | E−01 | [0.87- | TGGGAGTGCCTCTGCCCTCA | ID |
| | | | | | | | | | 221.2] | [A/T]ATGTATCCTTTTGGG | NO: |
| | | | | | | | | | | GAGTATTCTACCTTCCCTGC | 237 |
| | | | | | | | | | | CTTCT | |
| chr | 103388 | C | T | CCDC1 | p.G489 | 0.002 | 0.003 | 3.31 | 0.7 | CCTCAAATGTATCCTTTTGG | SEQ |
| 13 | 378 | | | 68 | 00 | 45 | 50 | E−01 | [0.37- | GGAGTATTCTACCTTCCCTG | ID |
| | | | | | | | | | 1.32] | [C/T]CTTCTATTTTTACTC | NO: |
| | | | | | | | | | | TGTCCTTTGCCTCTTTATAT | 238 |
| | | | | | | | | | | GGCAT | |
| chr | 103388 | G | A | CCDC1 | p.P472 | 0.002 | 0.003 | 6.78 | 0.85 | GTTTGCCTTGAAGGCAATGA | SEQ |
| 13 | 877 | | | 68 | 4S | 94 | 48 | E−01 | [0.47- | TTCCTGGATCTCAAGATGTG | ID |
| | | | | | | | | | 1.52] | [G/A]CATAAAGCTTCTTGT | NO: |
| | | | | | | | | | | TATTCGTGGTTCACCTTCCT | 239 |
| | | | | | | | | | | CTTCT | |
| chr | 103388 | T | C | CCDC1 | p.M47 | 0.043 | 0.041 | 5.14 | 1.05 | TGCCTTGAAGGCAATGATTC | SEQ |
| 13 | 880 | | | 68 | 23V | 14 | 03 | E−01 | [0.9- | CTGGATCTCAAGATGTGGCA | ID |
| | | | | | | | | | 1.23] | [T/C]AAAGCTTCTTGTTAT | NO: |
| | | | | | | | | | | TCGTGGTTCACCTTCCTCTT | 240 |
| | | | | | | | | | | CTTTT | |
| chr | 103389 | G | A | CCDC1 | p.P465 | 0.001 | 0.000 | 6.03 | 7.86 | TTCACCTGCAGTTCCTTTGT | SEQ |
| 13 | 072 | | | 68 | 9S | 96 | 25 | E−05 | [3.3- | TTTTAGTATATGGGAAAGGG | ID |
| | | | | | | | | | 18.75] | [G/A]TGATTTCTCTGCCTT | NO: |
| | | | | | | | | | | TACAGCTATGTACTCGGGAT | 241 |
| | | | | | | | | | | GCATT | |
| chr | 103389 | T | G | CCDC1 | p.K462 | 0.004 | 0.002 | 6.72 | 1.6 | TGAAATATTTGCTTTATCCT | SEQ |
| 13 | 164 | | | 68 | 8T | 41 | 76 | E−02 | [0.98- | TTTGGATCTGGGCCATGTAT | ID |
| | | | | | | | | | 2.61] | [T/G]TTGTTCTGTTTGAAT | NO: |
| | | | | | | | | | | CACCTGTGATATCATTCAAA | 242 |
| | | | | | | | | | | TATGA | |
| chr | 103389 | G | A | CCDC1 | p.R458 | 0.000 | 0.000 | 2.68 | 2.13 | GATCTTGTTACTCCTTGTTC | SEQ |
| 13 | 306 | | | 68 | 1X | 49 | 23 | E−01 | [0.48- | CTCTTTTTTGCCTGCTGTTC | ID |
| | | | | | | | | | 9.44] | [G/A]TTTGTCTAATTTACA | NO: |
| | | | | | | | | | | GTGAGATAGAGAAGGTATTG | 243 |
| | | | | | | | | | | TCAGA | |
| chr | 103389 | A | G | CCDC1 | p.C457 | 0.000 | 0.000 | 3.09 | 9.25 | TGTTCCTCTTTTTTGCCTGC | SEQ |
| 13 | 321 | | | 68 | 6R | 98 | 11 | E−03 | [2.61- | TGTTCGTTTGTCTAATTTAC | ID |
| | | | | | | | | | 32.79] | [A/G]GTGAGATAGAGAAGG | NO: |
| | | | | | | | | | | TATTGTCAGAAACACATCCA | 244 |
| | | | | | | | | | | GTTCA | |
| chr | 103389 | C | A | CCDC1 | p.V448 | 0.000 | 0.000 | 1.89 | 6.93 | TTGTATTCTTGTACTGTTTT | SEQ |
| 13 | 594 | | | 68 | 5L | 25 | 04 | E−01 | [0.63- | TACATCATTTGAGCTATCCA | ID |
| | | | | | | | | | 76.4] | [C/A]CCCAAAAGACTTTGT | NO: |
| | | | | | | | | | | ATGTGCTATTTTCCCTGCAT | 245 |
| | | | | | | | | | | CAAAT | |
| chr | 103389 | A | G | CCDC1 | p.L446 | 0.002 | 0.001 | 8.43 | 1.8 | TATTTTCCCTGCATCAAATG | SEQ |
| 13 | 656 | | | 68 | 45 | 45 | 36 | E−02 | [0.93- | ATTTCTGCTGCCTTAGTTGC | ID |
| | | | | | | | | | 3.48] | [A/G[AAGTAGCAGATTTTA | NO: |
| | | | | | | | | | | TTATTCCTTGTAAGTCTTCC | 246 |
| | | | | | | | | | | TCTCC | |
| chr | 103389 | C | T | CCDC1 | p.E439 | 0.000 | 0.000 | 1.30 | 13.87 | TGTTGCTCTTCAGTTTCTCC | SEQ |
| 13 | 867 | | | 68 | 4K | 25 | 02 | E−01 | [0.87- | ATCCCTGTTCCCTTGCTCCT | ID |
| | | | | | | | | | 221.8] | [C/T]ACCTTCTCCGTCCTC | NO: |
| | | | | | | | | | | TTTCCCTTTCTCCTGGCCTT | 247 |
| | | | | | | | | | | CTCCA | |
| chr | 103389 | T | G | CCDC1 | p.K438 | 0.011 | 0.014 | 7.81 | 0.76 | CCATCCCTGTTCCCTTGCTC | SEQ |
| 13 | 885 | | | 68 | 8Q | 27 | 80 | E−02 | [0.56- | CTCACCTTCTCCGTCCTCTT | ID |
| | | | | | | | | | 1.02] | [T/G]CCCTTGCTCCTGGCC | NO: |
| | | | | | | | | | | TTCTCCATCCCTTTTCCCTG | 248 |
| | | | | | | | | | | GCTCT | |
| chr | 103390 | C | T | CCDC1 | p.G432 | 0.004 | 0.003 | 2.99 | 1.27 | ATGTAATCTTTTGCTTTTTG | SEQ |
| 13 | 083 | | | 68 | 2S | 90 | 86 | E−01 | [0.8- | TACTTCACTTGCGCTATCAC | ID |
| | | | | | | | | | 2.01] | [C/T]CTCACTGGGCACCCC | NO: |
| | | | | | | | | | | ATTTGCTTTTTTCCCTGTCT | 249 |
| | | | | | | | | | | CTGAT | |
| chr | 103390 | C | T | CCDC1 | p.E432 | 0.012 | 0.010 | 2.45 | 1.19 | TAATCTTTTGCTTTTTGTAC | SEQ |
| 13 | 086 | | | 68 | 1K | 99 | 98 | E−01 | [0.89- | TTCACTTGCGCTATCACCCT | ID |
| | | | | | | | | | 1.57] | [C/T]ACTGGGCACCCCATT | NO: |
| | | | | | | | | | | TGCTTTTTTCCCTGTCTCTG | 250 |
| | | | | | | | | | | ATGAT | |
| chr | 103390 | G | C | CCDC1 | p.Q42 | 0.000 | 0.000 | 7.59 | 1.07 | TGCCTTGGTTGTAAAATACC | SEQ |
| 13 | 173 | | | 68 | 92E | 74 | 69 | E−01 | [0.33- | AGGTCTGATTATTCCTTGTT | ID |
| | | | | | | | | | 3.46] | [G/C]GTCTTCCTCTCCTTC | NO: |
| | | | | | | | | | | TATTCTTGTGTCCAATATAT | 251 |
| | | | | | | | | | | AATGG | |
| chr | 103390 | C | A | CCDC1 | p.E426 | 0.000 | 0.000 | 2.94 | 3.47 | AGAGAAGAATTGGAAGGCAA | SEQ |
| 13 | 257 | | | 68 | 4X | 25 | 07 | E−01 | [0.39- | ATATAGGAACAGAACTCTTT | ID |
| | | | | | | | | | 31.08] | [C/A]CTGTTCATTCTTGTC | NO: |
| | | | | | | | | | | TCCATCCATTTTCCCTTGCT | 252 |
| | | | | | | | | | | CTATG | |
| chr | 103390 | T | C | CCDC1 | p.E424 | 0.006 | 0.009 | 1.25 | 0.74 | TTTCCCTTGCTCTATGCCTA | SEQ |
| 13 | 322 | | | 68 | 26 | 86 | 27 | E−01 | [0.5- | CTCCATCTGCTTTCTGTTGC | ID |
| | | | | | | | | | 1.08] | [T/C]CTTCAACTTCGTGAT | NO: |
| | | | | | | | | | | CCATTTTCCCTTGCTCTTTG | 253 |
| | | | | | | | | | | TCTTC | |
| chr | 103390 | C | T | CCDC1 | p.E423 | 0.000 | 0.000 | 6.59 | 1.37 | TCTATGCCTACTCCATCTGC | SEQ |
| 13 | 332 | | | 68 | 9K | 49 | 36 | E−01 | [0.32- | TTTCTGTTGCTCTTCAACTT | ID |
| | | | | | | | | | 5.87] | [C/T]GTGATCCATTTTCCC | NO: |
| | | | | | | | | | | TTGCTCTTTGTCTTCTCTAT | 254 |
| | | | | | | | | | | CAACC | |
| chr | 103390 | T | C | CCDC1 | p.I414 | 0.000 | 0.000 | 7.53 | 2.76 | TGTTGCATGTAATCTTTTGC | SEQ |
| 13 | 626 | | | 68 | 1V | 98 | 36 | E−02 | [0.94- | TTTTTGTACTTTGATTGTGA | ID |
| | | | | | | | | | 8.07] | [T/C]ATCACCCTTACTGGC | NO: |
| | | | | | | | | | | CACTCCATCTGCTTTTTCCC | 255 |
| | | | | | | | | | | CTGCC | |
| chr | 103390 | A | T | CCDC1 | p.Y411 | 0.004 | 0.004 | 5.32 | 1.17 | CCTGCCTCTGATGATTTTTG | SEQ |
| 13 | 701 | | | 68 | 6N | 90 | 21 | E−01 | [0.74- | GTGTGATAGTTCTGGAAGAT | ID |
| | | | | | | | | | 1.84] | [A/T]GTATCTTGTTATTTC | NO: |
| | | | | | | | | | | AGTGACATACTCTGCTTTTT | 256 |
| | | | | | | | | | | CTCTC | |
| chr | 103390 | A | T | CCDC1 | p.L403 | 0.000 | 0.000 | 1.00 | 0.6 | GCCCTAATTTTTTCCATTTT | SEQ |
| 13 | 938 | | | 68 | 7M | 25 | 41 | E+00 | [0.08- | TTGCCTCTGTTCTTTTTGCA | ID |
| | | | | | | | | | 4.42] | [A/T]TATAGATTCTAGGGC | NO: |
| | | | | | | | | | | CTTTTTTACACTGTTTGAGA | 257 |
| | | | | | | | | | | TATTA | |
| chr | 103391 | G | A | CCDC1 | p.P391 | 0.000 | 0.000 | 6.02 | 1.14 | TTTTTCCAAAGCCTTTTCCA | SEQ |
| 13 | 300 | | | 68 | 6L | 25 | 22 | E−01 | [0.15- | CTCTGTCTTTCTCTTTCTGC | ID |
| | | | | | | | | | 8.74] | [G/A]GCATATGTTTTGCTT | NO: |
| | | | | | | | | | | TTTCAATACTGCTTAAACTA | 258 |
| | | | | | | | | | | TCATC | |
| chr | 103391 | T | A | CCDC1 | p.K389 | 0.000 | 0.000 | 1.45 | 3.42 | TTCAATACTGCTTAAACTAT | SEQ |
| 13 | 357 | | | 68 | 71 | 49 | 14 | E−01 | [0.73- | CATCAATTGGCTGCTCACAT | ID |
| | | | | | | | | | 16.13] | [T/A]TTTCCATTGTATCTG | NO: |
| | | | | | | | | | | ATAATTCCTGCTGTGTTGAT | 259 |
| | | | | | | | | | | GATGA | |
| chr | 103392 | C | G | CCDC1 | p.G364 | 0.000 | 0.000 | 1.00 | 0.86 | TATGTGTTGTTTTGTACTTT | SEQ |
| 13 | 113 | | | 68 | 5A | 25 | 29 | E+00 | [0.11- | TAACATTACTTGAGATCACC | ID |
| | | | | | | | | | 6.47] | [C/G]CATCAATTGTTTCTT | NO: |
| | | | | | | | | | | TATTCAATTTGAAGTGAGGT | 260 |
| | | | | | | | | | | AAAGA | |
| chr | 103392 | C | A | CCDC1 | p.M34 | 0.021 | 0.026 | 5.76 | 0.81 | TTGATATTAAATCAAAGACC | SEQ |
| 13 | 562 | | | 68 | 95I | 08 | 02 | E−02 | [0.65- | TGTACCCCATCTGATGATTT | ID |
| | | | | | | | | | 1] | [C/A]ATTCCTTTTGGAAAT | NO: |
| | | | | | | | | | | AAGAGACTTGCATATTTTAT | 261 |
| | | | | | | | | | | AGTTT | |
| chr | 103392 | G | C | CCDC1 | p.P343 | 0.000 | 0.000 | 1.90 | 6.88 | ATAGTGCTTAGCTGATCTGC | SEQ |
| 13 | 735 | | | 68 | 8A | 25 | 04 | E−01 | [0.62- | AGAAAACAAGTCTAGTCCTG | ID |
| | | | | | | | | | 75.88] | [G/C]TGTCCGGCTTGATAA | NO: |
| | | | | | | | | | | ATTACCTCCTTCTGATAATG | 262 |
| | | | | | | | | | | CTTCC | |
| chr | 103392 | G | A | CCDC1 | p.R343 | 0.008 | 0.008 | 9.31 | 1.01 | CTTAGCTGATCTGCAGAAAA | SEQ |
| 13 | 741 | | | 68 | 6W | 82 | 75 | E−01 | [0.72- | CAAGTCTAGTCCTGGTGTCC | ID |
| | | | | | | | | | 1.42] | [G/A]GCTTGATAAATTACC | NO: |
| | | | | | | | | | | TCCTTCTGATAATGCTTCCT | 263 |
| | | | | | | | | | | TTTCC | |
| chr | 103393 | A | T | CCDC1 | p.D323 | 0.001 | 0.000 | 4.33 | 5.77 | CTTTAATATTCAAATGTATT | SEQ |
| 13 | 330 | | | 68 | 9E | 23 | 21 | E−03 | [2.03- | CCTTCTGAACATGGAGGTTG | ID |
| | | | | | | | | | 16.38] | [A/T]TCCACCGGAATACCT | NO: |
| | | | | | | | | | | ACTTCATGTGATGCTTTCTC | 264 |
| | | | | | | | | | | TACCA | |
| chr | 103393 | G | A | CCDC1 | p.P323 | 0.000 | 0.000 | 5.03 | 1.54 | ATTCAAATGTATTCCTTCTG | SEQ |
| 13 | 337 | | | 68 | 7L | 25 | 16 | E−01 | [0.19- | AACATGGAGGTTGATCCACC | ID |
| | | | | | | | | | 12.13] | [G/A]GAATACCTACTTCAT | NO: |
| | | | | | | | | | | GTGATGCTTTCTCTACCATT | 265 |
| | | | | | | | | | | GGGCT | |
| chr | 103393 | C | G | CCDC1 | p.V322 | 0.000 | 0.000 | 1.31 | 13.79 | CCTACTTCATGTGATGCTTT | SEQ |
| 13 | 383 | | | 68 | 2L | 25 | 02 | E−01 | [0.86- | CTCTACCATTGGGCTTAGAA | ID |
| | | | | | | | | | 220.58] | [C/G]TTTTGAACTCATGAT | NO: |
| | | | | | | | | | | TTCTTCTGCTGAGCCTTCTT | 266 |
| | | | | | | | | | | TCTTG | |
| chr | 103393 | T | C | CCDC1 | p.Q31 | 0.000 | 0.000 | 2.20 | 2.49 | TTTCTGTCTATTTGATTTTA | SEQ |
| 13 | 580 | | | 68 | 56R | 49 | 20 | E−01 | [0.55- | ATGTAATATCCAACTTTGAT | ID |
| | | | | | | | | | 11.26] | [T/C]GCTCTTTTCCCCAAA | NO: |
| | | | | | | | | | | GATTTTCATTGAAACTTTCA | 267 |
| | | | | | | | | | | GAGAT | |
| chr | 103393 | C | T | CCDC1 | p.V310 | 0.000 | 0.000 | 7.28 | 0.41 | TCAGAATCCAGAATACTTTC | SEQ |
| 13 | 731 | | | 68 | 6M | 25 | 59 | E−01 | [0.06- | GGGAACATGATCTGGATTCA | ID |
| | | | | | | | | | 3.03] | [C/T]CTGTTCTTTCTGCTC | NO: |
| | | | | | | | | | | TGCAGGCACTTTGTGCTGTA | 268 |
| | | | | | | | | | | CCTCT | |
| chr | 103394 | A | G | CCDC1 | p.M29 | 0.000 | 0.000 | 2.44 | 4.6 | TTCTCTAATATCTTGTTCCT | SEQ |
| 13 | 336 | | | 68 | 04T | 25 | 05 | E−01 | [0.48- | GTTTTCTAAGAATGCTGGAC | ID |
| | | | | | | | | | 44.19] | [A/G]TATCAGTACAACCTG | NO: |
| | | | | | | | | | | ACAATGACCTTTGCATTTCT | 269 |
| | | | | | | | | | | TTTAG | |
| chr | 103394 | T | C | CCDC1 | p.K287 | 0.003 | 0.004 | 6.99 | 0.85 | TTCTCCAGCTTTGGCTGTGG | SEQ |
| 13 | 421 | | | 68 | 6E | 43 | 04 | E−01 | [0.49- | AAGAATGCATGTCCTGTCTT | ID |
| | | | | | | | | | 1.46] | [T/C]TGGCTTGTCTTTCTC | NO: |
| | | | | | | | | | | CATTTTTACTTCTGTAACCT | 270 |
| | | | | | | | | | | TTTTA | |
| chr | 103394 | G | A | CCDC1 | p.Q28 | 0.001 | 0.001 | 2.15 | 1.63 | ACTCGATGTACTGCATTTTT | SEQ |
| 13 | 544 | | | 68 | 35X | 72 | 05 | E−01 | [0.74- | ACTCAGCTGGAATGACTTCT | ID |
| | | | | | | | | | 3.57] | [G/A]CTGCTGGATGTTACC | NO: |
| | | | | | | | | | | TCTCAGTTCTTTTTTATTGC | 271 |
| | | | | | | | | | | TTGCA | |
| chr | 103395 | T | G | CCDC1 | p.K256 | 0.002 | 0.003 | 5.88 | 0.8 | TTTGTTTTTTTCTATTTTTA | SEQ |
| 13 | 359 | | | 68 | 3T | 94 | 69 | E−01 | [0.44- | CATTTTTTTCTGAATTCCCT | ID |
| | | | | | | | | | 1.43] | [T/G]TGTAAATCTGACTTT | NO: |
| | | | | | | | | | | TTGAGAAAAAAGTTTCTCCC | 272 |
| | | | | | | | | | | AAAAG | |
| chr | 103395 | C | T | CCDC1 | p.R254 | 0.001 | 0.001 | 5.07 | 1.28 | AGTTTCTCCCAAAAGCACAT | SEQ |
| 13 | 425 | | | 68 | 1H | 72 | 34 | E−01 | [0.59- | CCTCTGATTTACCAAGATGA | ID |
| | | | | | | | | | 2.77] | [C/T]GATCCTTTCTAAGAT | NO: |
| | | | | | | | | | | ATGTGTTTGCCATGAAGTTT | 273 |
| | | | | | | | | | | TCTGC | |
| chr | 103395 | G | C | CCDC1 | p.L242 | 0.001 | 0.001 | 1.00 | 0.96 | TGCCACATTGCTTTCAGTTT | SEQ |
| 13 | 789 | | | 68 | 0V | 23 | 28 | E+00 | [0.39- | GGTTTTTAAATTGGATTCAA | ID |
| | | | | | | | | | 2.38] | [G/C]TTTCTTCCTATGTTT | NO: |
| | | | | | | | | | | TGTAGTAAACTGCCCACTGA | 274 |
| | | | | | | | | | | TTTTA | |
| chr | 103396 | T | C | CCDC1 | p. K229 | 0.000 | 0.000 | 3.90 | 2.28 | CTGTGAAATTGACGACTTCT | SEQ |
| 13 | 163 | | | 68 | 5R | 25 | 11 | E−01 | [0.27- | TTTCCTTCATAGTTAAACAT | ID |
| | | | | | | | | | 18.94] | [T/C]TGGCATTGAATATAA | NO: |
| | | | | | | | | | | TTTCTTTTTCTGATAACTGT | 275 |
| | | | | | | | | | | GCTGT | |
| chr | 103396 | C | T | CCDC1 | p.R214 | 0.003 | 0.005 | 1.77 | 0.68 | ACTCATACTTTTCTTGCCTA | SEQ |
| 13 | 628 | | | 68 | 0Q | 68 | 37 | E−01 | [0.41- | TAAACTCTAATGTATAGCTC | ID |
| | | | | | | | | | 1.15] | [C/T]GGCTTTCATATTCAG | NO: |
| | | | | | | | | | | ATGACATGAGGCTGGAGAAA | 276 |
| | | | | | | | | | | TCTAA | |
| chr | 103397 | C | T | CCDC1 | p.R200 | 0.000 | 0.000 | 1.43 | 3.46 | TTTGCAAGGGTCAGGATCTT | SEQ |
| 13 | 030 | | | 68 | 6H | 49 | 14 | E−01 | [0.73- | TCATTTGATGTGTACTGAAA | ID |
| | | | | | | | | | 16.3] | [C/T]GGAGGTGTTGACTAT | NO: |
| | | | | | | | | | | AGCATGGAACTGATTCTGTT | 277 |
| | | | | | | | | | | AACAT | |
| chr | 103397 | C | T | CCDC1 | p.D192 | 0.000 | 0.000 | 4.85 | 1.39 | CCTTTACCTGAATTGTGCTG | SEQ |
| 13 | 280 | | | 68 | 3N | 74 | 53 | E−01 | [0.42- | TTCCCCCATACATTTCCTAT | ID |
| | | | | | | | | | 4.55] | [C/T]AGTTGGTACACCACG | NO: |
| | | | | | | | | | | TTTTATTGCACCAGTTAAAA | 278 |
| | | | | | | | | | | CTTCA | |
| chr | 103397 | T | G | CCDC1 | p.Q18 | 0.021 | 0.026 | 5.77 | 0.81 | AGGAAGAAGTTTTGAATTTA | SEQ |
| 13 | 387 | | | 68 | 87P | 08 | 03 | E−02 | [0.65- | CTGTACATATTGTGCCATTT | ID |
| | | | | | | | | | 1] | [T/G]GGGTCTGGAGGCATT | NO: |
| | | | | | | | | | | TCTTTGTCTCCTCTCTTTGT | 279 |
| | | | | | | | | | | ATTGG | |
| chr | 103398 | G | A | CCDC1 | p.A167 | 0.000 | 0.000 | 6.79 | Inf | TTTAGGTGTAGATAAAGCAG | SEQ |
| 13 | 023 | | | 68 | 5V | 25 | 00 | E−02 | [NaN- | GCATGCAGGAACCAAAAATC | ID |
| | | | | | | | | | Inf] | [G/A]CTGTCTCTTTCTTTT | NO: |
| | | | | | | | | | | CAGTACCACCAGCCTCTTCC | 280 |
| | | | | | | | | | | TTTTG | |
| chr | 103398 | T | C | CCDC1 | p.T159 | 0.001 | 0.001 | 1.74 | 1.62 | GTTTGTGTAAAATGTGTTTG | SEQ |
| 13 | 261 | | | 68 | 6A | 96 | 21 | E−01 | [0.78- | TGGTTGTACCTGAATATTTG | ID |
| | | | | | | | | | 3.37] | [T/C]ACTTCCTGGTTGGTT | NO: |
| | | | | | | | | | | CAGTTCCTCATCTGATTTGA | 281 |
| | | | | | | | | | | CAAGC | |
| chr | 103398 | C | T | CCDC1 | p.D157 | 0.000 | 0.000 | 1.00 | 0.66 | AGCTCATTATCCTTCTGATA | SEQ |
| 13 | 339 | | | 68 | 0N | 25 | 37 | E+00 | [0.09- | TGCATTGAGTATTAAGCCAT | ID |
| | | | | | | | | | 4.91] | [C/T]GCTGTTCTCCAGAGC | NO: |
| | | | | | | | | | | CTGTAAAGCTTTGGGAGGTG | 282 |
| | | | | | | | | | | GAATC | |
| chr | 103398 | C | A | CCDC1 | p.G153 | 0.000 | 0.000 | 3.93 | 9.28 | GTTTCGTTGGCTTTTTGTAG | SEQ |
| 13 | 453 | | | 68 | 2C | 49 | 05 | E−02 | [1.55- | TTCTTCAGCTTCTAAAGGAC | ID |
| | | | | | | | | | 55.53] | [C/A]CATTTGGAGACTAGT | NO: |
| | | | | | | | | | | CTCTAAAGTAGTTTGTTCAA | 283 |
| | | | | | | | | | | AACCT | |
| chr | 103399 | G | A | CCDC1 | p.T124 | 0.010 | 0.011 | 4.45 | 0.88 | AGATAGTTCCATTATGGGAG | SEQ |
| 13 | 313 | | | 68 | 5I | 05 | 47 | E−01 | [0.64- | AAACAACAGACTCAATAATA | ID |
| | | | | | | | | | 1.2] | [G/A]TTTCTGTGAATGGGA | NO: |
| | | | | | | | | | | TTGGTTGATGCATTTCTTTC | 284 |
| | | | | | | | | | | TCTGT | |
| chr | 103399 | A | G | CCDC1 | p.I116 | 0.000 | 0.000 | 4.36 | 0.5 | TTCTTCCCTTTCAATTTGCG | SEQ |
| 13 | 553 | | | 68 | 51 | 49 | 99 | E−01 | [0.12- | ATTCCTCTTGGACTAGCTTG | ID |
| | | | | | | | | | 2.04] | [A/G]TATGACTGTGATTCT | NO: |
| | | | | | | | | | | CTGCATTTAATCTGCTATAC | 285 |
| | | | | | | | | | | ATTCT | |
| chr | 103399 | A | T | CCDC1 | p.N11 | 0.000 | 0.000 | 6.72 | 2.89 | ATTCCTCTTGGACTAGCTTG | SEQ |
| 13 | 573 | | | 68 | 58K | 98 | 34 | E−02 | [0.98- | ATATGACTGTGATTCTCTGC | ID |
| | | | | | | | | | 8.48] | [A/T]TTTAATCTGCTATAC | NO: |
| | | | | | | | | | | ATTCTAGTATTAGGCAAAAT | 286 |
| | | | | | | | | | | AGACA | |
| chr | 103399 | G | T | CCDC1 | p.P109 | 0.006 | 0.007 | 5.66 | 0.87 | GTACCACATATATTAATATA | SEQ |
| 13 | 761 | | | 68 | 61 | 37 | 35 | E−01 | [0.58- | AGGCATCAGTGAGATTGCTG | ID |
| | | | | | | | | | 1.29] | [G/T]CTTCTTTACTTTCAT | NO: |
| | | | | | | | | | | AATTACATATTTGACACTGA | 287 |
| | | | | | | | | | | GTACA | |
| chr | 103399 | A | G | CCDC1 | p.Y106 | 0.000 | 0.000 | 1.89 | 6.91 | GTTTCTGATAATTTTTTTTT | SEQ |
| 13 | 848 | | | 68 | 7H | 25 | 04 | E−01 | [0.63- | AATTTCCTGCCTTTTAAAAT | ID |
| | | | | | | | | | 76.19] | [A/G]TGGTAAAGTAAGCAA | NO: |
| | | | | | | | | | | GTGGTTATTGAAAGACCCCA | 288 |
| | | | | | | | | | | GGGCA | |
| chr | 103399 | G | A | CCDC1 | p.T103 | 0.000 | 0.000 | 2.94 | 3.47 | TCTTTTTACATCTTCCTTTT | SEQ |
| 13 | 943 | | | 68 | 5M | 25 | 07 | E−01 | [0.39- | CTTCTGCAATATGACTATCC | ID |
| | | | | | | | | | 31.06] | [G/A]TTGTCTTTTGGAGGT | NO: |
| | | | | | | | | | | TTCCACCAAATGGGACACTA | 289 |
| | | | | | | | | | | TACTC | |
| chr | 103400 | T | A | CCDC1 | p.D100 | 0.000 | 0.000 | 9.71 | 4.61 | AACTGGCAAGTTCTCTGGCA | SEQ |
| 13 | 048 | | | 68 | 0V | 49 | 11 | E−02 | [0.93- | TTGTAAGTGGATTCTTTGGA | ID |
| | | | | | | | | | 22.84] | [T/A]CTCCGGCACTCTCTC | NO: |
| | | | | | | | | | | TGTCTGTAGGTCTATCTGTG | 290 |
| | | | | | | | | | | CTTTG | |
| chr | 103400 | T | G | CCDC1 | p.K950 | 0.001 | 0.000 | 8.40 | 6.95 | AAGAGTTTGTGGTTGGACTT | SEQ |
| 13 | 198 | | | 68 | T | 47 | 21 | E−04 | [2.61- | CTTGCTCTTTATTTGGGGCT | ID |
| | | | | | | | | | 18.53] | [T/G]TACTACTTCCTGAAC | NO: |
| | | | | | | | | | | TGATCTGTTCCATTTGGAAT | 291 |
| | | | | | | | | | | TTGAC | |
| chr | 103400 | C | G | CCDC1 | p.0839 | 0.000 | 0.000 | 2.95 | 1.78 | AGTTGAGAAATGGTAGTGTA | SEQ |
| 13 | 532 | | | 68 | H | 98 | 55 | E−01 | [0.63- | AGTGGCACTGTGAAATGCAT | ID |
| | | | | | | | | | 5.05] | [C/G]AGACGTTTCTTTATC | NO: |
| | | | | | | | | | | TTGATGCATATTTGTTATGT | 292 |
| | | | | | | | | | | TACTT | |
| chr | 103400 | C | A | CCDC1 | p.D756 | 0.000 | 0.000 | 6.77 | Inf | AAACCGACATTTGACAACTC | SEQ |
| 13 | 781 | | | 68 | Y | 25 | 00 | E−02 | [NaN- | CAGAACAAGTTCCAAAAAAT | ID |
| | | | | | | | | | Inf] | [C/A]TTTTTGTTTCTGTGT | NO: |
| | | | | | | | | | | ATTTTCCCTTGGAAAGCACC | 293 |
| | | | | | | | | | | TTTGC | |
| chr | 103400 | T | C | CCDC1 | p.Q75 | 0.000 | 0.000 | 2.95 | 3.45 | TGACAACTCCAGAACAAGTT | SEQ |
| 13 | 792 | | | 68 | 2R | 25 | 07 | E−01 | [0.39- | CCAAAAAATCTTTTTGTTTC | ID |
| | | | | | | | | | 30.84] | [T/C]GTGTATTTTCCCTTG | NO: |
| | | | | | | | | | | GAAAGCACCTTTGCGTTTTT | 294 |
| | | | | | | | | | | GGTGT | |
| chr | 103400 | T | A | CCDC1 | p.K741 | 0.000 | 0.000 | 2.95 | 3.45 | TTGTTTCTGTGTATTTTCCC | SEQ |
| 13 | 825 | | | 68 | I | 25 | 07 | E−01 | [0.39- | TTGGAAAGCACCTTTGCGTT | ID |
| | | | | | | | | | 30.91] | [T/A]TTGGTGTACTGGTTG | NO: |
| | | | | | | | | | | GTAACTCCTCTCCATTTGAA | 295 |
| | | | | | | | | | | AGTTG | |
| chr | 103400 | C | A | CCDC1 | p.E734 | 0.000 | 0.000 | 1.82 | 2.18 | GGAAAGCACCTTTGCGTTTT | SEQ |
| 13 | 847 | | | 68 | X | 74 | 34 | E−01 | [0.65- | TGGTGTACTGGTTGGTAACT | ID |
| | | | | | | | | | 7.38] | [C/A]CTCTCCATTTCAAAG | NO: |
| | | | | | | | | | | TTGAAGATGGGAATTTTCTG | 296 |
| | | | | | | | | | | AACTT | |
| chr | 103401 | C | G | CCDC1 | p.E586 | 0.000 | 0.000 | 2.96 | 3.43 | ATTCCTGTCTCCTCAAGAGG | SEQ |
| 13 | 291 | | | 68 | Q | 25 | 07 | E−01 | [0.38- | ACCTGCATAATTGATTTTCT | ID |
| | | | | | | | | | 30.71] | [C/G]TGTATCTGGTGACTT | NO: |
| | | | | | | | | | | ATTTTGCTTCTGCAGAAAAT | 297 |
| | | | | | | | | | | GTCCA | |
| chr | 103401 | T | C | CCDC1 | p.N52 | 0.000 | 0.001 | 5.28 | 0.64 | ATATCTTTCCTTTCATGTAA | SEQ |
| 13 | 480 | | | 68 | 3D | 98 | 54 | E−01 | [0.23- | TTCTTTCTTCTCAGTGTTAT | ID |
| | | | | | | | | | 1.74] | [T/C]CTTGCATCCTAACTC | NO: |
| | | | | | | | | | | ATTCCTATTTTTTAAAGTGT | 298 |
| | | | | | | | | | | GACAT | |
| chr | 103401 | A | G | CCDC1 | p.V373 | 0.001 | 0.001 | 8.33 | 1.01 | CAGGCCCTTTACTGAATATT | SEQ |
| 13 | 929 | | | 68 | A | 47 | 45 | E−01 | [0.44- | TTGCCTCAACAATTGATGGA | ID |
| | | | | | | | | | 2.33] | [A/G]CTTCAACAAAATGTT | NO: |
| | | | | | | | | | | GGTTCCTATCCAGATCTTGG | 299 |
| | | | | | | | | | | GACTG | |
| chr | 103402 | A | G | CCDC1 | p.Y169 | 0.000 | 0.000 | 5.95 | 1.16 | TGCTCTGTATGGCTTAGACA | SEQ |
| 13 | 542 | | | 68 | H | 25 | 21 | E−01 | [0.15- | CGTTTCCTCTACTTCTGAAT | ID |
| | | | | | | | | | 8.89] | [A/G]AAACAATGGCAAAGA | NO: |
| | | | | | | | | | | TGAGCTGATTCCATTTGAAG | 300 |
| | | | | | | | | | | ATGGC | |
| chr | 103402 | A | G | CCDC1 | p.L167 | 0.000 | 0.000 | 1.00 | 0.82 | TGTATGGCTTAGACACGTTT | SEQ |
| 13 | 547 | | | 68 | S | 25 | 30 | E+00 | [0.11- | CCTCTACTTCTGAATAAAAC | ID |
| | | | | | | | | | 6.13] | [A/G]ATGGCAAAGATGAGC | NO: |
| | | | | | | | | | | TGATTCCATTTGAAGATGGC | 301 |
| | | | | | | | | | | ACATG | |
| chr | 103402 | A | G | CCDC1 | p.W13 | 0.000 | 0.000 | 3.71 | 0.31 | GAGGGACTTACTTGATCTTC | SEQ |
| 13 | 638 | | | 68 | 7R | 25 | 80 | E−01 | [0.04- | ACTTTCACTAGTACCTGACC | ID |
| | | | | | | | | | 2.22] | [A/G]TAGTATTTCACGTGA | NO: |
| | | | | | | | | | | GAATAAAATTCTATCTTCAA | 302 |
| | | | | | | | | | | AGTTA | |
| chr | 103411 | G | A | CCDC1 | p.A39 | 0.000 | 0.000 | 2.46 | 13.91 | TATCTCAAAAATAATTCCTA | SEQ |
| 13 | 167 | | | 68 | V | 49 | 04 | E−02 | [1.96- | GTAAAATTATAAAGAAAATT | ID |
| | | | | | | | | | 98.81] | [G/A]CCACCCAATCATTTT | NO: |
| | | | | | | | | | | GAATAATCCAGGACTCTAGA | 303 |
| | | | | | | | | | | AAGTC | |
| chr | 103514 | C | T | BIVM- | p.H769 | 0.007 | 0.005 | 3.78 | 1.48 | AAGTGGATTCAGAGTCTCTT | SEQ |
| 13 | 444 | | | ERCC5 | H | 84 | 31 | E−02 | [1.04- | CCTTCTTCCAGCAAAATGCA | ID |
| | | | | | | | | | 2.12] | [C/T]GGCATGTCTTTTGAC | NO: |
| | | | | | | | | | | GTGAAGTCATCTCCATGTGA | 304 |
| | | | | | | | | | | AAAAC | |
| chr | 103701 | A | G | SLC10A | p.F304 | 0.005 | 0.003 | 3.18 | 1.61 | ATCATGAAATGGGATTGGCA | SEQ |
| 13 | 648 | | | 2 | L | 64 | 50 | E−02 | [1.06- | TGATTCCTTACATCCTAAGA | ID |
| | | | | | | | | | 2.46] | [A/G]TATTGCGGCAAAGGC | NO: |
| | | | | | | | | | | GAGCTGGAAAATGCTGTAGA | 305 |
| | | | | | | | | | | TGAGC | |
| chr | 110864 | C | T | COL4A | p.E131 | 0.010 | 0.006 | 3.86 | 1.62 | CAGCGAAACCAGGCAAGCCA | SEQ |
| 13 | 264 | | | 1 | E | 29 | 37 | E−03 | [1.19- | GGAGGCCCGAGCGGCCCTCT | ID |
| | | | | | | | | | 2.22] | [C/T]TCCCCCTGGGGAGAC | NO: |
| | | | | | | | | | | AGGAGAGCATCATTCATACG | 306 |
| | | | | | | | | | | CACTG | |
| chr | 113201 | C | T | TUBGC | p.R413 | 0.011 | 0.000 | 1.08 | 14.5 | GGGAAAGACGCGCGTGGGAA | SEQ |
| 13 | 864 | | | P3 | H | 52 | 80 | E−30 | [9.66- | AGACGTGCATGGGAAAGTCG | ID |
| | | | | | | | | | 21.75] | [C/T]GCGTGGGAAAGTCGC | NO: |
| | | | | | | | | | | GCGTGGGAAAGTCGCGCGTG | 307 |
| | | | | | | | | | | GGAAA | |
| chr | 114175 | G | A | TMCO | p.P436 | 0.012 | 0.008 | 3.24 | 1.39 | CGCAGGACGTGCAGCTCGGG | SEQ |
| 13 | 013 | | | 3 | P | 01 | 69 | E−02 | [1.04- | CTCTTCATGGCCGTCATGCC | ID |
| | | | | | | | | | 1.85] | [G/A]ACTCTCATACAGGCG | NO: |
| | | | | | | | | | | GGCGCCAGTGCATCTTCTAG | 308 |
| | | | | | | | | | | GTAAA | |
| chr | 212161 | G | A | EDDM | p.V133 | 0.007 | 0.004 | 1.38 | 1.62 | CTTCAGCTACATTGAATTCC | SEQ |
| 14 | 36 | | | 3A | I | 35 | 56 | E−02 | [1.12- | ATTGTGGCGTAGATGGATAT | ID |
| | | | | | | | | | 2.34] | [G/A]TTGATAACATAGAAG | NO: |
| | | | | | | | | | | ACCTGAGGATTATAGAACCT | 309 |
| | | | | | | | | | | ATCAG | |
| chr | 233538 | G | A | REM2 | p.T39T | 0.009 | 0.004 | 1.55 | 2.02 | TTTCTTTGCCCTCCCATTTT | SEQ |
| 14 | 96 | | | | | 07 | 52 | E−04 | [1.44- | ATTTTAGAAGCAGATGCCAC | ID |
| | | | | | | | | | 2.82] | [G/A]CTACTAAAGAAGTCA | NO: |
| | | | | | | | | | | GAGAAACTGTTGGCAGAGTT | 310 |
| | | | | | | | | | | GGACC | |
| chr | 244643 | C | T | DHRS4 | p.T29T | 0.008 | 0.001 | 3.09 | 7.44 | CTGCTGTCAACCCTTTCTTT | SEQ |
| 14 | 24 | | | L2 | | 33 | 13 | E−17 | [5.09- | GGAAGCCTAATGGATGTCAC | ID |
| | | | | | | | | | 10.89] | [C/T]GAGGAGGTGTGGGAC | NO: |
| | | | | | | | | | | AAGGTGAGAGGGGATTAAAG | 311 |
| | | | | | | | | | | AAGCG | |
| chr | 247723 | C | T | NOP9 | p.R413 | 0.007 | 0.004 | 3.19 | 1.61 | GGGCCACCCAGGGGTAGTCA | SEQ |
| 14 | 73 | | | | C | 482 | 658 | E−02 | [1.01- | TTGCCCTGGTGGGGGCCTGT | ID |
| | | | | | | | | | 2.45] | [C/T]GCAGAGTTGGGGCCT | NO: |
| | | | | | | | | | | ACCAAGCCAAGGTCCTACAG | 312 |
| | | | | | | | | | | CTCTT | |
| chr | 449751 | G | A | FSCB | p.P363 | 0.010 | 0.000 | 7.71 | Inf | AGGAGACTTTTCAGCTGGTG | SEQ |
| 14 | 03 | | | | L | 29 | 00 | E−62 | | GAGGCAGAATTTCAGCAGGA | ID |
| | | | | | | | | | | [G/A]GCTCTTCTGAAGGGG | NO: |
| | | | | | | | | | | ACTCTTCAGCTGATGGAGGC | 313 |
| | | | | | | | | | | AGAAT | |
| chr | 449751 | G | A | FSCB | p. P359 | 0.024 | 0.000 | 1.52 | 2806.41 | AGCTGGTGGAGGCAGAATTT | SEQ |
| 14 | 15 | | | | L | 51 | 01 | E− | [391.38- | CAGCAGGAGGCTCTTCTGAA | ID |
| | | | | | | | | 144 | 20123.7] | [G/A]GGGACTCTTCAGCTG | NO: |
| | | | | | | | | | | ATGGAGGCAGAATTTCAGCC | 314 |
| | | | | | | | | | | AGAAG | |
| chr | 505810 | A | C | VCPKM | p.Y188 | 0.010 | 0.006 | 2.01 | 1.48 | ACTACAAAGATAATAGAGTA | SEQ |
| 14 | 11 | | | T | D | 05 | 79 | E−02 | [1.08- | CTTAATACTTACCTCAAAAT | ID |
| | | | | | | | | | 2.04] | [A/C]TTTTTTCTCAATTTC | NO: |
| | | | | | | | | | | TGGATTTTTCCCCATTGTTC | 315 |
| | | | | | | | | | | GTTGT | |
| chr | 524954 | C | T | NID2 | p.8830 | 0.005 | 0.003 | 4.83 | 1.61 | GATGCAAGTATGCCGGTCAT | SEQ |
| 14 | 81 | | | | Q | 15 | 20 | E−02 | [1.04- | CTGCAAACTCATAACCACTC | ID |
| | | | | | | | | | 2.51] | [C/T]GGCACTCACACCTGT | NO: |
| | | | | | | | | | | AGCTTCCAGGCAAGTTGATA | 316 |
| | | | | | | | | | | CATAC | |
| chr | 524963 | T | C | NID2 | p.D756 | 0.011 | 0.007 | 2.33 | 1.44 | CATGTGGCTCCCATCATAGC | SEQ |
| 14 | 99 | | | | G | 03 | 71 | E−02 | [1.06- | AAGGATTCCCCGGAGTGGGG | ID |
| | | | | | | | | | 1.94] | [T/C]CTGAATCCTCTGCAT | NO: |
| | | | | | | | | | | GAGTAGAGGGGAAATAAAAG | 317 |
| | | | | | | | | | | CACAA | |
| chr | 525096 | C | T | NID2 | p.R493 | 0.011 | 0.008 | 4.93 | 1.35 | AGTGGCATAGTCCGTGCAGA | SEQ |
| 14 | 01 | | | | K | 76 | 72 | E−02 | [1.01- | AGGCATGCCGGGAGCATTGT | ID |
| | | | | | | | | | 1.81] | [C/T]TGTGGTTGTGTTCAC | NO: |
| | | | | | | | | | | AGGTTTCCTTGTTGGCAGCA | 318 |
| | | | | | | | | | | TTATA | |
| chr | 609218 | T | G | Cl4orf | p.E462 | 0.006 | 0.004 | 4.35 | 1.52 | TAAGAAAAGAAAGTCCAGGG | SEQ |
| 14 | 36 | | | 39 | D | 86 | 52 | E−02 | [1.04- | GATTCCTTTTCTGTTTGAAC | ID |
| | | | | | | | | | 2.23] | [T/G[TCAGGTACTGCATTT | NO: |
| | | | | | | | | | | CTATTTCTGTTACTGAGAAA | 319 |
| | | | | | | | | | | TAAGA | |
| chr | 622448 | C | T | SNAPC | p.T253 | 0.005 | 0.003 | 4.52 | 1,72 | AATGATGGAGAAGAAAAAAT | SEQ |
| 14 | 54 | | | 1 | M | 21 | 03 | E−02 | [0.97- | GGAAGGAAATTCACAAGAAA | ID |
| | | | | | | | | | 2.84] | [C/T]GGAGGTCAGAAAACT | NO: |
| | | | | | | | | | | TTGCAATTCATATTATGTGT | 320 |
| | | | | | | | | | | GGCTG | |
| chr | 695216 | C | T | DCAF5 | p.R589 | 0.006 | 0.003 | 7.18 | 1.78 | TGGGGCACTGGGCTTGTCTT | SEQ |
| 14 | 37 | | | | H | 86 | 88 | E−03 | [1.21- | CTCGGGTTGTCTTCTGTCGG | ID |
| | | | | | | | | | 2.6] | [C/T]GCCGCATGGCATTCC | NO: |
| | | | | | | | | | | GCTGCCAGGTAGAGGCTCGG | 321 |
| | | | | | | | | | | CGTTC | |
| chr | 704189 | C | T | SMOC1 | p.P77L | 0.005 | 0.003 | 3.93 | 1.61 | GAGTCCATGTGTGAGTACCA | SEQ |
| 14 | 85 | | | | | 39 | 36 | E−02 | [1.04- | GCGAGCCAAGTGCCGAGACC | ID |
| | | | | | | | | | 2.47] | [C/T]GACCCTGGGCGTGGT | NO: |
| | | | | | | | | | | GCATCGAGGTAGATGCAAAG | 322 |
| | | | | | | | | | | GTGAG | |
| chr | 751512 | C | T | AREL1 | p.V50 | 0.007 | 0.004 | 1.58 | 1.74 | GAGACTTTGCAAGACCGGGG | SEQ |
| 14 | 52 | | | | M | 157 | 135 | E−02 | [1.08- | ATCCAGGTAATTTCCCCGCA | ID |
| | | | | | | | | | 2.67] | [C/T]GTAGTCATAAATAGT | NO: |
| | | | | | | | | | | CCGGTCCCCTCGGCGCTCGC | 323 |
| | | | | | | | | | | GGTCC | |
| chr | 860881 | C | A | FLRT2 | p.L107 | 0.006 | 0.003 | 2.85 | 1.61 | CTACCTGTATGGCAACCAAC | SEQ |
| 14 | 77 | | | | I | 13 | 82 | E−02 | [1.07- | TGGACGAATTCCCCATGAAC | ID |
| | | | | | | | | | 2.41] | [C/A]TTCCCAAGAATGTCA | NO: |
| | | | | | | | | | | GAGTTCTCCATTTGCAGGAA | 324 |
| | | | | | | | | | | AACAA | |
| chr | 888929 | C | T | SPATA | p.8211 | 0.005 | 0.003 | 4.13 | 1.59 | CTGAACTCTTTTCTAACAAA | SEQ |
| 14 | 32 | | | 7 | R | 39 | 41 | E−02 | [1.03- | CAATTGCCATTCACTCCTCG | ID |
| | | | | | | | | | 2.44] | [C/T]ACTTTAAAAACAGAA | NO: |
| | | | | | | | | | | GCAAAATCTTTCCTGTCACA | 325 |
| | | | | | | | | | | GTATC | |
| chr | 891108 | T | C | EML5 | p.V136 | 0.009 | 0.006 | 3.12 | 1.45 | AGTGAGTTTTCCTTACCTCT | SEQ |
| 14 | 01 | | | | 1V | 56 | 63 | E−02 | [1.05- | ATAGGTCTCTTTTTCTTGCC | ID |
| | | | | | | | | | 2] | [T/C]ACATTGTTTGTCTGG | NO: |
| | | | | | | | | | | AGTTTCTCTGGCTGTGGTGG | 326 |
| | | | | | | | | | | GGCCC | |
| chr | 101004 | A | G | BEGAI | p.F568 | 0.005 | 0.000 | 2.13 | 607.53 | CTGTCCTTGCGGCTCAGCCC | SEQ |
| 14 | 386 | | | N | L | 88 | 01 | E−33 | [82.17- | CGAGCCACCAGTCCGCGGAA | ID |
| | | | | | | | | | 4491.9] | [A/G]GGCCTGCTGGGGGCT | NO: |
| | | | | | | | | | | GAGGCGGGCGGCAGGATGCA | 327 |
| | | | | | | | | | | TTTCC | |
| chr | 103593 | T | A | TNFAIP | p.V79E | 0.009 | 0.000 | 1.74 | Inf | GTGGGCTGGGGCCGGGGCTG | SEQ |
| 14 | 342 | | | 2 | | 80 | 00 | E−07 | | ACGCGGCTTTCCCGGCGCAG | ID |
| | | | | | | | | | | [T/A]GGAGGAGCTGAAGGC | NO: |
| | | | | | | | | | | GGCGCTGGAGCGCGGGCAGC | 328 |
| | | | | | | | | | | TGGAG | |
| chr | 105415 | C | T | AHNAK | p.K218 | 0.011 | 0.000 | 4.90 | 43.07 | GGTCCCCCTGCATGGAGGGG | SEQ |
| 14 | 242 | | | 2 | 2K | 27 | 26 | E−47 | [27.03- | AGACTCATGTCGGCCTCCAC | ID |
| | | | | | | | | | 68.62] | [C/T]TTGGGTGGAGACACA | NO: |
| | | | | | | | | | | TCCACCGAGGCCTCGATGGA | 329 |
| | | | | | | | | | | CTTGC | |
| chr | 105415 | T | C | AHNAK | p.K215 | 0.019 | 0.000 | 7.30 | 21.36 | CACCCCAAACGACGGCATCT | SEQ |
| 14 | 333 | | | 2 | 2R | 61 | 94 | E−63 | [15.62- | TGAACTTGGGCATTTTGAAC | ID |
| | | | | | | | | | 29.19] | [T/C]TGCTGTCTTTGGTAG | NO: |
| | | | | | | | | | | TCAGGTCCTTGTTGGCCAGG | 330 |
| | | | | | | | | | | GTCAG | |
| chr | 105415 | A | T | AHNAK | p.D201 | 0.005 | 0.003 | 1.74 | 1.74 | AGGGGAGACTCACGTCGGCC | SEQ |
| 14 | 752 | | | 2 | 2E | 64 | 25 | E−02 | [1.14- | TCCACCTTGGGTGCAGGCAC | ID |
| | | | | | | | | | 2.65] | [A/T]TCCACCGAGGCCTCG | NO: |
| | | | | | | | | | | ATGGACCTCCCTGGGGCCGA | 331 |
| | | | | | | | | | | TACCC | |
| chr | 105418 | G | C | AHNAK | p.L120 | 0.008 | 0.001 | 3.11 | 4.88 | GGTCAGCGGAAGGGGGCTGA | SEQ |
| 14 | 170 | | | 2 | 6L | 82 | 82 | E−13 | [3.41- | ATGCTGAGGTCAGTGGTCTT | ID |
| | | | | | | | | | 6.97] | [G/C]AGGTCCCCCTGCATG | NO: |
| | | | | | | | | | | GAGGGGAGACTCACGTCGGC | 332 |
| | | | | | | | | | | CTCCA | |
| chr | 315155 | G | A | LOC28 | p.L124 | 0.011 | 0.000 | 2.03 | Inf | TGGGATCAGTGCGGCCTGTC | SEQ |
| 15 | 19 | | | 3710 | F | 52 | 00 | E−51 | | GTCTGCTGTTGTCATGTGGA | ID |
| | | | | | | | | | | [G/A]CTCAGCAAACGGTGG | NO: |
| | | | | | | | | | | GAGTCCTAGGGGACAACATA | 333 |
| | | | | | | | | | | CACAG | |
| chr | 387768 | T | A | FAM98 | p.G425 | 0.007 | 0.000 | 7.32 | 61.29 | ATCCATATGGAGGAGGTGGT | SEQ |
| 15 | 33 | | | B | G | 35 | 12 | E−27 | [23.77- | GGTGGTGGTGGTGGTGGTGG | ID |
| | | | | | | | | | 158.06] | [T/A]GGAGGAGGTGGATAT | NO: |
| | | | | | | | | | | AGAAGATACTAAAAACTATA | 334 |
| | | | | | | | | | | AAAAT | |
| chr | 418623 | G | A | TYRO3 | p.T458 | 0.008 | 0.005 | 1.15 | 1.6 | CCCTGGCCCTCATCCTGCTT | SEQ |
| 15 | 46 | | | | T | 33 | 24 | E−02 | [1.13- | CGAAAGAGACGGAAAGAGAC | ID |
| | | | | | | | | | 2.26] | [G/A]CGGTTTGGGTAAGGG | NO: |
| | | | | | | | | | | GATGGGGATETGGAGGGAGA | 335 |
| | | | | | | | | | | GGCAG | |
| chr | 436533 | C | T | ZSCAN | p.R842 | 0.005 | 0.003 | 4.15 | 1.58 | AGGGGCTTACTTGGGAGCTG | SEQ |
| 15 | 05 | | | 29 | Q | 39 | 41 | E−02 | [1.03- | ACTGTGTCAGAAGCTTTTCC | ID |
| | | | | | | | | | 2.44] | [C/T]GTGCATGGATTTCTC | NO: |
| | | | | | | | | | | CGTGCTTATTAAGGGCAGAG | 336 |
| | | | | | | | | | | CTTTT | |
| chr | 484704 | G | T | MYEF2 | p.A2E | 0.026 | 0.000 | 2.09 | Inf | GCCACCAGTGGCCCCGGGCA | SEQ |
| 15 | 30 | | | | | 23 | 00 | E−39 | | CCTCGGCCTTGTTGGCGTCC | ID |
| | | | | | | | | | | [G/T]CCATCCCGCCGCCGC | NO: |
| | | | | | | | | | | TGCCTCCGCCTCGGCCGCCT | 337 |
| | | | | | | | | | | GAGCT | |
| chr | 525101 | A | G | MYO5 | p.L129 | 0.005 | 0.003 | 3.12 | 1.67 | TTACACTTGACTTCACTTTC | SEQ |
| 15 | 96 | | | C | 2L | 15 | 08 | E−02 | [1.08- | AGTTTCAAATTGTTTCTTCA | ID |
| | | | | | | | | | 2.6] | [A/G]GTGGTCACTGGCCTC | NO: |
| | | | | | | | | | | CTGCATTTCTTGAATCTTAT | 338 |
| | | | | | | | | | | CAATC | |
| chr | 651578 | G | A | PLEKH | p.S420 | 0.010 | 0.007 | 1.60 | 1.47 | AACGGCTATATCGGGCCCAG | SEQ |
| 15 | 74 | | | O2 | S | 78 | 36 | E−02 | [1.08- | CTGGAGGTGAAGGTGGCCTC | ID |
| | | | | | | | | | 1.99] | [G/A]GAACAGACGGAGAAA | NO: |
| | | | | | | | | | | CTGTTGAACAAGGTGCTGGG | 339 |
| | | | | | | | | | | CAGTG | |
| chr | 720235 | G | A | THSD4 | p.V526 | 0.005 | 0.003 | 2.01 | 1.83 | GATACACCAGCAGCCAAACC | SEQ |
| 15 | 02 | | | | M | 53 | 02 | E−02 | [1.05- | CAGGCGTGCACTACGAGTAC | ID |
| | | | | | | | | | 2.99] | [G/A]TGATCATGGGGACCA | NO: |
| | | | | | | | | | | ACGCCATCAGCCCCCAGGTG | 340 |
| | | | | | | | | | | CCACC | |
| chr | 721922 | C | G | MYO9 | p.R109 | 0.005 | 0.002 | 2.23 | 1.89 | GTAATCTCTCCATTTCTGCT | SEQ |
| 15 | 05 | | | A | 8P | 21 | 75 | E−02 | [1.07- | GGATAACGATGGCTGCAGCC | ID |
| | | | | | | | | | 3.13] | [C/G]GTAACTCCAAGTACC | NO: |
| | | | | | | | | | | GCTGCCTCTCTAAGTGAGCA | 341 |
| | | | | | | | | | | CGCCA | |
| chr | 725021 | T | C | PKM | p.N15 | 0.005 | 0.003 | 3.24 | 1.61 | CACCACCTTGCAGATGTTCT | SEQ |
| 15 | 15 | | | | 5S | 64 | 52 | E−02 | [1.05- | TGTAGTCCAGCCACAGGATG | ID |
| | | | | | | | | | 2.45] | [T/C]TCTCGTCACACTTTT | NO: |
| | | | | | | | | | | CCATGTAGGCGTTATCCAGC | 342 |
| | | | | | | | | | | GTGAT | |
| chr | 725136 | T | A | PKM | p.T36S | 0.017 | 0.011 | 2.57 | 1.5 | CTTGGCCTCACTAGCAAAGA | SEQ |
| 15 | 12 | | | | | 16 | 53 | E−03 | [1.16- | CCGCTCAGAGCTGAATACGG | ID |
| | | | | | | | | | 1.93] | [T/A]GTGCCCTGGAGAGCT | NO: |
| | | | | | | | | | | GCACAAGGATTAAGGAAAAA | 343 |
| | | | | | | | | | | GCTGA | |
| chr | 759815 | C | A | CSPG4 | p.G632 | 0.005 | 0.000 | 7.77 | Inf | TCCATCGCTGACCCGGAACG | SEQ |
| 15 | 11 | | | | V | 15 | 00 | E−31 | | TCAAGTCCTGTGCAGGACCA | ID |
| | | | | | | | | | | [C/A]CGCGGTGGACATAGA | NO: |
| | | | | | | | | | | CTAGGCTGCCGGCCTCCAAC | 344 |
| | | | | | | | | | | TCCCG | |
| chr | 759820 | A | G | CSPG4 | p.H451 | 0.006 | 0.004 | 4.39 | 1.52 | TGCGCAGCTCAGCCTCCATC | SEQ |
| 15 | 53 | | | | H | 86 | 53 | E−02 | [1.04- | AGGTCCAGCGTGGGCTGCAC | ID |
| | | | | | | | | | 2.23] | [A/G]TGCCTCCACTCAAGC | NO: |
| | | | | | | | | | | CAGGCTGTGCCCCCCTCGGC | 345 |
| | | | | | | | | | | CACCA | |
| chr | 784613 | C | T | IDH3A | p.R360 | 0.006 | 0.003 | 7.88 | 1.74 | AGGCAATGCAAAATGCTCAG | SEQ |
| 15 | 24 | | | | C | 86 | 96 | E−03 | [1.18- | ACTTCACAGAGGAAATCTGT | ID |
| | | | | | | | | | 2.55] | [C/T]GCCGAGTAAAAGATT | NO: |
| | | | | | | | | | | TAGATTAACACTTCTACAAC | 346 |
| | | | | | | | | | | TGGCA | |
| chr | 790589 | A | T | ADAM | p.A110 | 0.007 | 0.000 | 2.49 | 10.56 | GAGGCTCTGTGGCAGGCACG | SEQ |
| 15 | 44 | | | TS7 | 3A | 89 | 80 | E−11 | [6.04- | GGGCTACCCGTGGAGGGCGC | ID |
| | | | | | | | | | 18.49] | [A/T]GCAGGATGGCTGTGT | NO: |
| | | | | | | | | | | GGTGGGGGTGTCCGGTCCCC | 347 |
| | | | | | | | | | | TGTCC | |
| chr | 796037 | G | A | TMED3 | TMED | 0.006 | 0.004 | 3.23 | 1.54 | GGAGGTGGAGCAGGGCGTGA | SEQ |
| 15 | 60 | | | | 3(NM_ | 86 | 47 | E−02 | [1.05- | AGTTCTCCCTGGATTACCAG | ID |
| | | | | | 00736 | | | | 2.26] | [G/A]TGAGGCCGGGCGCCC | NO: |
| | | | | | 4:exon | | | | | GGCAGCGCTCCCTTCTCCCT | 348 |
| | | | | | 1:c.16 | | | | | CCACT | |
| | | | | | 8 + | | | | | | |
| | | | | | 1G > A) | | | | | | |
| chr | 891697 | G | A | AEN | p.G100 | 0.006 | 0.004 | 2.72 | 1.58 | TGGATCTGGCAGTGCCCCAT | SEQ |
| 15 | 38 | | | | R | 62 | 20 | E−02 | [1.07- | GCAGCAGAAGGCCTGCTCCC | ID |
| | | | | | | | | | 2.33] | [G/A]GGAAAGCCTCAGGGC | NO: |
| | | | | | | | | | | CCTTGCCCAGCAAGTGTGTG | 349 |
| | | | | | | | | | | GCTAT | |
| chr | 102346 | C | T | OR4F6 | p.R54C | 0.005 | 0.003 | 2.56 | 1.62 | GGGAAATCTCCTCATTGTGC | SEQ |
| 15 | 082 | | | | | 88 | 63 | E−02 | [1.07- | TAACTGTGACCTCTGACCCT | ID |
| | | | | | | | | | 2.45] | [C/T]GTTTACAGTCCCCCA | NO: |
| | | | | | | | | | | TGTACTTCCTGCTGGCCAAC | 350 |
| | | | | | | | | | | CTTTC | |
| chr | 315001 | C | T | ITFG3 | p.R547 | 0.005 | 0.003 | 3.86 | 1.62 | AGACAGTGACCAAGCCATCA | SEQ |
| 16 | | | | | W | 39 | 35 | E−02 | [1.05- | GGGACCGGTTCTCCCGGCTG | ID |
| | | | | | | | | | 2.49] | [C/T]GGTACCAGAGTGAGG | NO: |
| | | | | | | | | | | CGTAGAGGCACGCCAGCCAG | 351 |
| | | | | | | | | | | AGCCT | |
| chr | 863362 | C | G | PRR25 | p.P237 | 0.020 | 0.000 | 1.86 | Inf | GACATCCCCTCTGCTATTGC | SEQ |
| 16 | | | | | R | 34 | 00 | E− | | TGCGGGACCGGCAAGGACGC | ID |
| | | | | | | | | 108 | | [C/G]GGACCGACACGGCCT | NO: |
| | | | | | | | | | | CCCCATCCCTGGGTCCACCC | 352 |
| | | | | | | | | | | CGACT | |
| chr | 225857 | G | A | MLST8 | p.G275 | 0.005 | 0.002 | 7.86 | 1.86 | GAGCGGCAACCCCGGGGAGT | SEQ |
| 16 | 5 | | | | S | 39 | 90 | E−03 | [1.21- | CCTCCCGCGGCTGGATGTGG | ID |
| | | | | | | | | | 2.88] | [G/A]GCTGCGCCTTCTCGG | NO: |
| | | | | | | | | | | GGGACTCCCAGTACATCGTC | 353 |
| | | | | | | | | | | ACTGG | |
| chr | 228764 | A | C | DNASE | p.D197 | 0.005 | 0.003 | 3.19 | 1.61 | TACGACGTGTACCTGGACGT | SEQ |
| 16 | 9 | | | 1L2 | A | 64 | 51 | E−02 | [1.06- | GATCGACAAGTGGGGCACCG | ID |
| | | | | | | | | | 2.46] | [A/C]CGTAAGCCCACCCCT | NO: |
| | | | | | | | | | | CGGTCCCGGGGTCCCTGCAG | 354 |
| | | | | | | | | | | GCGCG | |
| chr | 236959 | C | T | ABCA3 | p.R288 | 0.014 | 0.009 | 1.71 | 1.56 | GAGTGTTGGGGAGCCAAAGC | SEQ |
| 16 | 2 | | | | K | 46 | 32 | E−03 | [1.2- | GGGCAGTCACCTTCAGCCTC | ID |
| | | | | | | | | | 2.03] | [C/T]TTTCCTTCTCCTGCA | NO: |
| | | | | | | | | | | CGACAGCACGGGCAATGGTG | 355 |
| | | | | | | | | | | AGCGC | |
| chr | 284851 | G | T | PRSS41 | p.A10 | 0.016 | 0.000 | 6.97 | Inf | GAGAGGAGGCCATGGGCGCG | SEQ |
| 16 | 5 | | | | A | 67 | 00 | E−73 | | CGCGGGGCGCTGCTGCTGGC | ID |
| | | | | | | | | | | [G/T]CTGCTGCTGGCTCGG | NO: |
| | | | | | | | | | | GCTGGACTCGGGAAGCCGGG | 356 |
| | | | | | | | | | | TGAGC | |
| chr | 363905 | C | T | SLX4 | p.P152 | 0.005 | 0.002 | 4.17 | 2.04 | CTTCGGGCTTCTGAGCTCCA | SEQ |
| 16 | 8 | | | | 7P | 15 | 53 | E−03 | [1.31- | CCAGCGCTTGGCATCTGGGC | ID |
| | | | | | | | | | 3.18] | [C/T]GGAGGAGGGGTCTCT | NO: |
| | | | | | | | | | | GGAGGCCTCTGCTCTTCCCC | 357 |
| | | | | | | | | | | GTCCC | |
| chr | 363937 | T | A | SLX4 | p.I142 | 0.011 | 0.001 | 8.31 | 11.09 | GAGAGGGGCTCCATGTGCCA | SEQ |
| 16 | 8 | | | | 1F | 76 | 07 | E−30 | [7.9- | GCAGCAGTCGTCAATTGGAA | ID |
| | | | | | | | | | 15.56] | [T/A]TGGGGGGTCACTGTC | NO: |
| | | | | | | | | | | CAGTGGGGGGCTTCTGTTGG | 358 |
| | | | | | | | | | | CCTGA | |
| chr | 364081 | C | G | SLX4 | p.E942 | 0.005 | 0.002 | 1.53 | 2.14 | TGGCCAAGCGCCTCCTCTGG | SEQ |
| 16 | 5 | | | | Q | 39 | 53 | E−03 | [1.39- | CGCCTCCTGCTCAGGGGCCT | ID |
| | | | | | | | | | 3.31] | [C/G]TGCTCCCCGTGCCCC | NO: |
| | | | | | | | | | | TGAGTGCTGGCCCTGGGGTG | 359 |
| | | | | | | | | | | GCGGG | |
| chr | 370719 | G | A | DNASE | p.V185 | 0.008 | 0.004 | 4.80 | 1.69 | CGCATGTCCCAGGGCCACAG | SEQ |
| 16 | 1 | | | 1 | I | 33 | 95 | E−03 | [1.19- | GCAGCGTTTCCTGGTAGGAC | ID |
| | | | | | | | | | 2.39] | [G/A]TCATGTTGATGGGCG | NO: |
| | | | | | | | | | | ACTTCAATGCGGGCTGCAGC | 360 |
| | | | | | | | | | | TATGT | |
| chr | 373608 | C | T | TRAP1 | p.R128 | 0.005 | 0.002 | 9.00 | 1.91 | CATTTCTGGCAGTGCTTGGC | SEQ |
| 16 | 5 | | | | H | 15 | 70 | E−03 | [1.22- | CGTCAGACACCAGTTTGTGA | ID |
| | | | | | | | | | 2.97] | [C/T]GCAGTTTTTCCAAGG | NO: |
| | | | | | | | | | | CATCGCTGGCATTGGAGATC | 361 |
| | | | | | | | | | | AGCTC | |
| chr | 491077 | A | G | UBN1 | p.R262 | 0.024 | 0.000 | 1.26 | 2748.75 | GCTAAAGAAATTTCAGAAAG | SEQ |
| 16 | 7 | | | | G | 02 | 01 | E− | [383.26- | AGAAAGAGGCTCAGAAAAAA | ID |
| | | | | | | | | 141 | 19714.18] | [A/G]GGGAGGAGGAGCATA | NO: |
| | | | | | | | | | | AGCCTGTTGCGGTCCCATCA | 362 |
| | | | | | | | | | | GCGGA | |
| chr | 209965 | G | A | DNAH3 | p.D251 | 0.006 | 0.004 | 4.15 | 1.51 | CGATGTCAGCCTTCTCGTCA | SEQ |
| 16 | 25 | | | | 3D | 62 | 39 | E−02 | [1.03- | GCAGGGAAGATGTTAGGCAC | ID |
| | | | | | | | | | 2.23] | [G/A]TCACCTGTGTTCAGA | NO: |
| | | | | | | | | | | AGCATGTTGATGTCCTCCAC | 363 |
| | | | | | | | | | | GAATG | |
| chr | 209965 | G | A | DNAH3 | p.A249 | 0.007 | 0.004 | 1.02 | 1.68 | TGATGTCCTCCACGAATGAT | SEQ |
| 16 | 88 | | | | 2A | 11 | 23 | E−02 | [1.16- | TCATCCTTGATCTGGTTGTC | ID |
| | | | | | | | | | 2.45] | [G/A]GCGAAGAGGAACACG | NO: |
| | | | | | | | | | | GTGCTCTTGGTGGCCACACC | 364 |
| | | | | | | | | | | GACCT | |
| chr | 217476 | A | C | OTOA | p.T706 | 0.007 | 0.000 | 5.19 | 75.13 | CCTTCTGCAAGCAGCTTCCA | SEQ |
| 16 | 33 | | | | P | 35 | 10 | E−35 | [37.62- | AGATGGCCAGGACCCTGCCC | ID |
| | | | | | | | | | 150.01] | [A/C]CTAAAGAATTCCTCT | NO: |
| | | | | | | | | | | GGGCTGTCTTTCAGTCTGTT | 365 |
| | | | | | | | | | | CGGAA | |
| chr | 217476 | G | T | OTOA | p.E708 | 0.007 | 0.000 | 1.12 | 413.18 | GCAAGCAGCTTCCAAGATGG | SEQ |
| 16 | 39 | | | | X | 35 | 02 | E−41 | [98.71- | CCAGGACCCTGCCCACTAAA | ID |
| | | | | | | | | | 1729.48] | [G/T]AATTCCTCTGGGCTG | NO: |
| | | | | | | | | | | TCTTTCAGTCTGTTCGGAAC | 366 |
| | | | | | | | | | | AGCAG | |
| chr | 217476 | G | A | OTOA | p.Q71 | 0.007 | 0.000 | 5.17 | 136.27 | GGACCCTGCCCACTAAAGAA | SEQ |
| 16 | 62 | | | | 5Q | 35 | 05 | E−38 | [56.69- | TTCCTCTGGGCTGTCTTTCA | ID |
| | | | | | | | | | 327.58] | [G/A]TCTGTTCGGAACAGC | NO: |
| | | | | | | | | | | AGTGATAAGATCCCCAGCTA | 367 |
| | | | | | | | | | | TGACC | |
| chr | 289438 | C | G | CD19 | p.P102 | 0.019 | 0.000 | 1.99 | Inf | CAACAGATGGGGGGCTTCTA | SEQ |
| 16 | 83 | | | | R | 36 | 00 | E− | | CCTGTGCCAGCCGGGGCCCC | ID |
| | | | | | | | | 114 | | [C/G]CTCTGAGAAGGCCTG | NO: |
| | | | | | | | | | | GCAGCCTGGCTGGACAGTCA | 368 |
| | | | | | | | | | | ATGTG | |
| chr | 289962 | G | C | LAT | p.L15F | 0.017 | 0.000 | 7.89 | Inf | AGGCCACGGCTGCCAGCTGG | SEQ |
| 16 | 27 | | | | | 89 | 00 | E−80 | | CAGGTGGCTGTCCCCGTCTT | ID |
| | | | | | | | | | | [G/C]GGGGGGGCCAGCAGA | NO: |
| | | | | | | | | | | CCCTTGGTGAGTGCCTGGGG | 369 |
| | | | | | | | | | | TGGCT | |
| chr | 307932 | C | G | ZNF62 | p.Q79 | 0.014 | 0.000 | 4.59 | 1291.24 | CTGCCTCTGGAGGGGGGTCC | SEQ |
| 16 | 73 | | | 9 | 2H | 22 | 01 | E−78 | [178.81- | TCGGGATTGGGGGGTTTTTC | ID |
| | | | | | | | | | 9324.5] | [C/G]TGGGTGTGGGTTTCT | NO: |
| | | | | | | | | | | TGGTGCCGGGTGAGGGCCAC | 370 |
| | | | | | | | | | | GCGGT | |
| chr | 307942 | G | T | ZNF62 | p.T481 | 0.022 | 0.000 | 7.33 | Inf | AGCTCTTGCCGCACTCGGGG | SEQ |
| 16 | 06 | | | 9 | T | 55 | 00 | E− | | CACTTGTAGGGCTTCTCGCC | ID |
| | | | | | | | | 134 | | [G/T]GTGTGCGTGCGGCGG | NO: |
| | | | | | | | | | | TGCTGGATAAGGTGGGAGCT | 371 |
| | | | | | | | | | | GCGGA | |
| chr | 620552 | G | A | CDH8 | p.P24S | 0.015 | 0.000 | 1.88 | 451.14 | ACTTGAGACTGATTCATCGG | SEQ |
| 16 | 38 | | | | | 93 | 04 | E−89 | [164.28- | AGCCATGTAAATGCAAGGGG | ID |
| | | | | | | | | | 1238.87] | [G/A]AAGAGTAATCCATAA | NO: |
| | | | | | | | | | | TATTATTAATGGAGTCCAGA | 372 |
| | | | | | | | | | | GATCC | |
| chr | 672368 | C | T | ELMO3 | p.T600 | 0.006 | 0.004 | 4.80 | 1.48 | CTGATCCGCCAGCAGCGCTT | SEQ |
| 16 | 72 | | | | M | 86 | 65 | E−02 | [1.01- | GCTCCGCCTCTGTGAGGGGA | ID |
| | | | | | | | | | 2.16] | [C/T]GCTCTTCCGCAAGAT | NO: |
| | | | | | | | | | | CAGCAGCCGGCGGCGCCAGG | 373 |
| | | | | | | | | | | GTCTC | |
| chr | 689615 | C | T | TANGO | p.R745 | 0.008 | 0.006 | 3.26 | 1.45 | ATACCCTGATCCGGTCATCC | SEQ |
| 16 | 76 | | | 6 | C | 82 | 09 | E−02 | [1.04- | AAGAACTCGCTGTTGATCTC | ID |
| | | | | | | | | | 2.03] | [C/T]GCATCACCATCTCTA | NO: |
| | | | | | | | | | | CCCATGGAGCCTTTGCCACT | 374 |
| | | | | | | | | | | GAGGC | |
| chr | 705088 | A | G | FUK | p.T772 | 0.009 | 0.006 | 1.39 | 1.54 | TGAGCTGTGGCTGGCGGTGG | SEQ |
| 16 | 51 | | | | A | 31 | 05 | E−02 | [1.11- | GGCCTCGGCAGGATGAGATG | ID |
| | | | | | | | | | 2.15] | [A/G]CTGTGAAGATAGTGT | NO: |
| | | | | | | | | | | GCCGGTGCCTGGCTGACCTG | 375 |
| | | | | | | | | | | CGGGA | |
| chr | 708947 | C | T | HYDIN | p.P393 | 0.025 | 0.000 | 4.43 | 656.67 | GGCAGATGGGCAAGGTGCTC | SEQ |
| 16 | 71 | | | | 7P | 98 | 04 | E−89 | [91.63- | CGCCCTTTTGCTACCAGGAC | ID |
| | | | | | | | | | 4706.3] | [C/T]GGACCTTGCTCTCCA | NO: |
| | | | | | | | | | | GGTGGCAGGTTGGGAATCCT | 376 |
| | | | | | | | | | | GAGAG | |
| chr | 708970 | C | T | HYDIN | p.8383 | 0.005 | 0.000 | 1.11 | Inf | TGAGGTATCTTCTGAGACCC | SEQ |
| 16 | 62 | | | | 2H | 39 | 00 | E−32 | | AGCTGAATTCCAGCTGGACA | ID |
| | | | | | | | | | | [C/T]GTCCTGAATTAATCA | NO: |
| | | | | | | | | | | CATCGAACCTGCAAATCGAT | 377 |
| | | | | | | | | | | CAGGG | |
| chr | 709350 | C | T | HYDIN | p.8295 | 0.005 | 0.002 | 9.20 | 2.95 | AGGCCACAGGCAGGAGCGTG | SEQ |
| 16 | 93 | | | | 48 | 88 | 00 | E−06 | [1.93- | ACATTGCGGAGAAGAACTAC | ID |
| | | | | | | | | | 4.51] | [C/T]CTGGATTCCTGTCTG | NO: |
| | | | | | | | | | | CAGAGACAAAAGGAAAGTTG | 378 |
| | | | | | | | | | | CAATT | |
| chr | 709550 | G | A | HYDIN | p.I240 | 0.017 | 0.000 | 2.60 | Inf | TCTCAGACATTGTTTGTTCC | SEQ |
| 16 | 79 | | | | 0I | 40 | 00 | E−94 | | CTAACAGATATTTTCCTTTC | ID |
| | | | | | | | | | | [G/A]ATTGTCTCCATCTTG | NO: |
| | | | | | | | | | | ACATCCACTTTGGTGAGCGG | 379 |
| | | | | | | | | | | AGGAA | |
| chr | 709960 | G | A | HYDIN | p.S193 | 0.006 | 0.001 | 1.77 | 5.05 | CGATGTCCTCTTTGTGCTAT | SEQ |
| 16 | 23 | | | | 6L | 37 | 27 | E−09 | [3.17- | TGGAGGTTCCCTGATCTGAT | ID |
| | | | | | | | | | 8.05] | [G/A]AGGTTATATCTTCCT | NO: |
| | | | | | | | | | | CTTCTGCCAGGTAGCAAAGG | 380 |
| | | | | | | | | | | ATGAA | |
| chr | 711012 | G | A | HYDIN | p.A713 | 0.005 | 0.000 | 2.94 | 93.92 | AGAGCAAGCTGGGGAGCAAT | SEQ |
| 16 | 11 | | | | V | 88 | 06 | E−29 | [40.44- | ACCTTGCTGTAATTAAGAGC | ID |
| | | | | | | | | | 218.1] | [G/A]CCAGCACCTCTTCTC | NO: |
| | | | | | | | | | | CGATGCCCTCCACGTCCACC | 381 |
| | | | | | | | | | | ACGAG | |
| chr | 851007 | C | T | KIAA05 | p.D40 | 0.005 | 0.001 | 1.06 | 3.2 | CACCCCCTGTGCTGCAGGAC | SEQ |
| 16 | 97 | | | 13 | D | 15 | 61 | E−05 | [2.03- | GGCGATGGCTCCCTGGGGGA | ID |
| | | | | | | | | | 5.03] | [C/T]GGTGCATCAGAGAGT | NO: |
| | | | | | | | | | | GAGACCACTGAGTCTGCGGA | 382 |
| | | | | | | | | | | CAGTG | |
| chr | 887197 | T | c | MVD | p.K368 | 0.011 | 0.007 | 1.19 | 1.51 | CTGGGTGAGCCCCAGGCCTC | SEQ |
| 16 | 26 | | | | K | 03 | 34 | E−02 | [1.11- | ACCTGAGTGACAATGATGTA | ID |
| | | | | | | | | | 2.04] | [T/C]TTGACCCCACCGGGG | NO: |
| | | | | | | | | | | GTCGGCTCCATGGCCAGCGC | 383 |
| | | | | | | | | | | AGCCT | |
| chr | 168710 | C | T | SMYD4 | p.V645 | 0.006 | 0.003 | 2.00 | 1.95 | TGTAGGTCCTGTAACCGAGA | SEQ |
| 17 | 7 | | | | I | 86 | 53 | E−03 | [1.33- | GACCAGGTGGTCCCTGCTGA | ID |
| | | | | | | | | | 2.87] | [C/T]GGCGGATTCTGCACA | NO: |
| | | | | | | | | | | AGATCTGCTGCCACAGCGCA | 384 |
| | | | | | | | | | | GCACG | |
| chr | 227571 | G | C | SGSM2 | p.R530 | 0.005 | 0.000 | 8.71 | 571.47 | TGTCGGCGCTGGTGCACCAT | SEQ |
| 17 | 9 | | | | R | 88 | 01 | E−33 | [77.29- | AGCGTTATCCCACCTGACCG | ID |
| | | | | | | | | | 4225.3] | [G/C]CCCCCGGGGGCCTCC | NO: |
| | | | | | | | | | | GCGGGCCTCACCAAGGACGT | 385 |
| | | | | | | | | | | GTGGA | |
| chr | 319577 | A | T | OR3A1 | p.F34I | 0.011 | 0.006 | 5.13 | 1.74 | CTGAGGTTGCCCCTGACCGT | SEQ |
| 17 | 7 | | | | | 76 | 79 | E−04 | [1.3- | GACCAGGTAGGCAAAGAGGA | ID |
| | | | | | | | | | 2.33] | [A/T]GACCACAAAGACAAC | NO: |
| | | | | | | | | | | TGGCTGCAGCCCTGGCGCCT | 386 |
| | | | | | | | | | | CCAGC | |
| chr | 722237 | G | A | NEURL | p.L122 | 0.006 | 0.003 | 1.18 | 1.72 | CCTGGTCCTGTTCCTTCTCT | SEQ |
| 17 | 4 | | | 4 | 5F | 13 | 57 | E−02 | [1.15- | CTGGCTCCTACTCACCTTGA | ID |
| | | | | | | | | | 2.58] | [G/A]ACCGTTGTGGAAGAC | NO: |
| | | | | | | | | | | CCCACGGCCCCGCAGCAGCC | 387 |
| | | | | | | | | | | AGGCT | |
| chr | 819320 | G | A | RANGR | p.Q17 | 0.005 | 0.003 | 9.03 | 1.78 | ATCTGTCACCTGCACCCTGG | SEQ |
| 17 | 3 | | | F | 0Q | 88 | 31 | E−03 | [1.18- | AGCCTGGGTGACTTTGAACA | ID |
| | | | | | | | | | 2.69] | [G/A]CTGGTGACCAGTCTG | NO: |
| | | | | | | | | | | ACCCTTCACGATCCTAACAT | 388 |
| | | | | | | | | | | CTTTG | |
| chr | 117846 | C | T | DNAH9 | p.A358 | 0.008 | 0.005 | 4.28 | 1.45 | TCACCGTGACCAGGGATGGC | SEQ |
| 17 | 88 | | | | 8A | 09 | 58 | E−02 | [1.02- | CTGGAGGACCAGTTGCTGGC | ID |
| | | | | | | | | | 2.07] | [C/T]GCTGTGGTCAGCATG | NO: |
| | | | | | | | | | | GAGAGGCCAGACTTGGAGCA | 389 |
| | | | | | | | | | | GCTGA | |
| chr | 142048 | C | T | HS3ST | p.C11C | 0.005 | 0.002 | 3.72 | 2 | GGCAGCGCATGGGGCAGCGC | SEQ |
| 17 | 68 | | | 381 | | 39 | 71 | E−03 | [1.29- | CTGAGTGGCGGCAGATCTTG | ID |
| | | | | | | | | | 3.1] | [C/T]CTCGATGTCCCCGGC | NO: |
| | | | | | | | | | | CGGCTCCTACCGCAGCCGCC | 390 |
| | | | | | | | | | | GCCGC | |
| chr | 171844 | C | T | COPS3 | p.A2A | 0.008 | 0.005 | 4.22 | 1.46 | AGAGCTGTCGGACACTGTTC | SEQ |
| 17 | 95 | | | | | 09 | 56 | E−02 | [1.03- | ACGAACTGCTCCAGGGCAGA | ID |
| | | | | | | | | | 2.07] | [C/T]GCCATGTTTTCCCCC | NO: |
| | | | | | | | | | | GGGCGGCCCGAGCGGCGAAG | 391 |
| | | | | | | | | | | GCAGC | |
| chr | 188746 | C | T | FAM83 | p.D819 | 0.011 | 0.000 | 1.70 | 1177.01 | TGGCTCCAGGCTGGGACATG | SEQ |
| 17 | 89 | | | G | N | 03 | 01 | E−63 | [162.21- | CTGCTAGGGGTCTTTGCGGT | ID |
| | | | | | | | | | 8540.78] | [C/T]CCGGGGGGCTTGAGC | NO: |
| | | | | | | | | | | CCTCCGTTTAGAATCCGATG | 392 |
| | | | | | | | | | | AGGCC | |
| chr | 212039 | G | A | MAP2K | p.M90I | 0.009 | 0.004 | 9.33 | 2.28 | TGGTAGAGAAGGTGCGGCAC | SEQ |
| 17 | 61 | | | 3 | | 56 | 22 | E−06 | [1.64- | GCCCAGAGCGGCACCATCAT | ID |
| | | | | | | | | | 3.16] | [G/A]GCCGTGAAGGTGAGC | NO: |
| | | | | | | | | | | AGGGCCTGGAGGCAGCTGGG | 393 |
| | | | | | | | | | | AGGGC | |
| chr | 212154 | C | G | MAP2K | p.T273 | 0.005 | 0.002 | 1.78 | 2.05 | AGATGGCCATCCTGCGGTTC | SEQ |
| 17 | 98 | | | 3 | T | 88 | 88 | E−03 | [1.35- | CCTTACGAGTCCTGGGGGAC | ID |
| | | | | | | | | | 3.11] | [C/G]CCGTTCCAGCAGCTG | NO: |
| | | | | | | | | | | AAGCAGGTGGTGGAGGAGCC | 394 |
| | | | | | | | | | | GTCCC | |
| chr | 213186 | G | A | KCNJ1 | p.R61 | 0.012 | 0.002 | 2.45 | 4.42 | AGCCAGGGTCCCCCAACCCC | SEQ |
| 17 | 71 | | | 8 | | 04 | 75 | E−12 | [3.02- | CGGGATGACCGCGGCCAGCC | ID |
| | | | | | | | | | 6.32] | [G/A]GGCCAACCCCTACAG | NO: |
| | | | | | | | | | | CATCGTGTCATCGGAGGAGG | 395 |
| | | | | | | | | | | ACGGG | |
| chr | 213188 | G | A | KCNJ1 | p.A58T | 0.017 | 0.000 | 4.39 | 49.8 | CCGCTTCGTCAAGAAGAATG | SEQ |
| 17 | 26 | | | 2 | | 16 | 35 | E−73 | [33.51- | GCCAGTGCAACATTGAGTTC | ID |
| | | | | | | | | | 74.01] | [G/A]CCAACATGGACGAGA | NO: |
| | | | | | | | | | | AGTCACAGCGCTACCTGGCT | 396 |
| | | | | | | | | | | GACAT | |
| chr | 213197 | G | A | KCNJ1 | p.E380 | 0.010 | 0.000 | 9.65 | 31.56 | GTTCCTGCTGCCCAGCGCCA | SEQ |
| 17 | 92 | | | 2 | K | 05 | 32 | E−39 | [20.08- | ACTCCTTCTGCTACGAGAAC | ID |
| | | | | | | | | | 49.6] | [G/A]AGCTGGCCTTCCTGA | NO: |
| | | | | | | | | | | GCCGTGACGAGGAGGATGAG | 397 |
| | | | | | | | | | | GCGGA | |
| chr | 275807 | G | A | CRYBA | p.G159 | 0.006 | 0.004 | 4.80 | 1.53 | CCCCTCCTTGCAAGCCATGG | SEQ |
| 17 | 75 | | | 1 | S | 37 | 16 | E−02 | [1.03- | GCTGGTTCAACAACGAAGTC | ID |
| | | | | | | | | | 2.28] | [G/A]GCTCCATGAAGATAC | NO: |
| | | | | | | | | | | AAAGTGGGGCGTAAGTACAA | 398 |
| | | | | | | | | | | AAACA | |
| chr | 276138 | T | C | NUFIP | p.T392 | 0.006 | 0.004 | 3.46 | 1.56 | GCTGACATAGGGACCTGGGA | SEQ |
| 17 | 38 | | | 2 | A | 37 | 10 | E−02 | [1.05- | TAAGCGACTTGATGATTGGG | ID |
| | | | | | | | | | 2.32] | [T/C]CTGAGTTTCCCCGGT | NO: |
| | | | | | | | | | | AGATGATGAAGATGATGAAG | 399 |
| | | | | | | | | | | ATGAA | |
| chr | 368296 | A | C | C17orf | p.M35 | 0.020 | 0.000 | 2.25 | Inf | GAATTTGAGGCCAGGGGGCT | SEQ |
| 17 | 76 | | | 96 | 88 | 34 | 00 | E−80 | | CAGGGACAGCGGGACCCCCC | ID |
| | | | | | | | | | | [A/C]TCTGCCACCTCCACA | NO: |
| | | | | | | | | | | GCGGGTGGGCGGGCGGGGGC | 400 |
| | | | | | | | | | | TTAGA | |
| chr | 389534 | G | C | KRT28 | p.P251 | 0.019 | 0.000 | 7.38 | 2199.94 | CGCTCGCATGTTGTTCAACA | SEQ |
| 17 | 72 | | | | R | 36 | 01 | E− | [306.01- | AAACCGCGAGGTCTACGCCC | ID |
| | | | | | | | | 114 | 15815.81] | [G/C]GGGCGGCGTTCATCT | NO: |
| | | | | | | | | | | CCACGTTCACGTTGCCCCCA | 401 |
| | | | | | | | | | | GCCGC | |
| chr | 391908 | A | G | KRTAP | p.S59S | 0.006 | 0.000 | 1.98 | Inf | GCTGGCAGCAGCTGGTCTCA | SEQ |
| 17 | 97 | | | 1-3 | | 86 | 00 | E−41 | | CAGCAGCTTGGCTGGCAGCA | ID |
| | | | | | | | | | | [A/G]CTGGAGCTGCAGGTC | NO: |
| | | | | | | | | | | CCACTAGTTGAGAAGCTAGG | 402 |
| | | | | | | | | | | AAATC | |
| chr | 392743 | C | T | KRTAP | p.R66 | 0.005 | 0.000 | 3.87 | 143.18 | GCAGCTGGGGCGACAGCAGC | SEQ |
| 17 | 71 | | | 4-11 | H | 15 | 04 | E−27 | [49.13- | TGGAGATGCAGCATCTGGGG | ID |
| | | | | | | | | | 417.29] | [C/T]GGCAGCAGGTGGGCT | NO: |
| | | | | | | | | | | GGCAGCACACAGACTGGCAG | 403 |
| | | | | | | | | | | CACTG | |
| chr | 392744 | T | A | KRTAP | p.S48C | 0.015 | 0.000 | 1.04 | Inf | TGGCAGCACACAGACTGGCA | SEQ |
| 17 | 26 | | | 4-11 | | 20 | 00 | E−90 | | GCACTGGGGCCTGCAGCAGC | ID |
| | | | | | | | | | | [T/A]GGACACACAGCAGCT | NO: |
| | | | | | | | | | | GGGGCGACAGTAGGTGGTCC | 404 |
| | | | | | | | | | | TGCAG | |
| chr | 392744 | A | T | KRTAP | p.C45S | 0.005 | 0.000 | 3.82 | Inf | ACAGACTGGCAGCACTGGGG | SEQ |
| 17 | 35 | | | 441 | | 64 | 00 | E−34 | | CCTGCAGCAGCTGGACACAC | ID |
| | | | | | | | | | | [A/T]GCAGCTGGGGCGACA | NO: |
| | | | | | | | | | | GTAGGTGGTCCTGCAGCAGG | 405 |
| | | | | | | | | | | TGGTC | |
| chr | 392744 | C | T | KRTAP | p.C44Y | 0.005 | 0.000 | 3.08 | Inf | AGACTGGCAGCACTGGGGCC | SEQ |
| 17 | 37 | | | 4-11 | | 15 | 00 | E−31 | | TGCAGCAGCTGGACACACAG | ID |
| | | | | | | | | | | [C/T]AGCTGGGGCGACAGT | NO: |
| | | | | | | | | | | AGGTGGTCCTGCAGCAGGTG | 406 |
| | | | | | | | | | | GTCTC | |
| chr | 393166 | C | T | KRTAP | p.R107 | 0.011 | 0.000 | 4.62 | Inf | AGCAGGTGGGCTGGCAGCAC | SEQ |
| 17 | 23 | | | 4-4 | R | 52 | 00 | E−69 | | ACAGACTGGCAGCACTGGGG | ID |
| | | | | | | | | | | [C/T]CTGCAGCAGCTGGGG | NO: |
| | | | | | | | | | | CGGCAGCAGGTGGTCCTACA | 407 |
| | | | | | | | | | | GCAGG | |
| chr | 393462 | A | C | KRTAP | p.T21T | 0.005 | 0.000 | 6.33 | 577.37 | GCTGTCAGCCTACATGCTGC | SEQ |
| 17 | 01 | | | 9-1 | | 15 | 01 | E−30 | [77.65- | AGGACCACCTGCTGCAGGAC | ID |
| | | | | | | | | | 4293.3] | [A/C]ACCTGCTGGAAGCCC | NO: |
| | | | | | | | | | | ACCACTGTGACCACCTGCAG | 408 |
| | | | | | | | | | | CAGCA | |
| chr | 393465 | A | C | KRTAP | p.N14 | 0.024 | 0.000 | 2.06 | Inf | TGCTGCCAGCCTACCTGCTG | SEQ |
| 17 | 75 | | | 9-1 | 6T | 51 | OD | E− | | CCAGCCCACCTGCTGCAGGA | ID |
| | | | | | | | | 133 | | [A/C]CACCTCTTGCCAGCC | NO: |
| | | | | | | | | | | CACCTGCTGTGGGTCCAGCT | 409 |
| | | | | | | | | | | GCTGC | |
| chr | 422392 | A | G | C17orf | p.S645 | 0.007 | 0.004 | 2.63 | 1.55 | ACTCCTGAGTGAGCTTCCTG | SEQ |
| 17 | 92 | | | 53 | G | 11 | 60 | E−02 | [1.06- | AAGACTTCTTCTGTGGGACC | ID |
| | | | | | | | | | 2.25] | [A/G]GTAGTTGAGACTGCC | NO: |
| | | | | | | | | | | CCAACGCAGGACAACCCACC | 410 |
| | | | | | | | | | | ATGAG | |
| chr | 428829 | G | A | GJC1 | p.L71L | 0.008 | 0.004 | 3.63 | 1.74 | CCACCAGGATGATCTGGAAC | SEQ |
| 17 | 73 | | | | | 09 | 66 | E−03 | [1.22- | ACCCAGAAGCGTACATGGGA | ID |
| | | | | | | | | | 2.48] | [G/A]AGAGGTGCAAACGCA | NO: |
| | | | | | | | | | | TCATAACAGACATTCTCACA | 411 |
| | | | | | | | | | | GCCCG | |
| chr | 439234 | C | T | SPPL2C | p.L380 | 0.011 | 0.007 | 4.61 | 1.59 | TGTGCGGCTGCCCACTCTCA | SEQ |
| 17 | 10 | | | | L | 27 | 13 | E−03 | [1.18- | AGAACTGCTCCTCCTTCCTG | ID |
| | | | | | | | | | 2.14] | [C/T]TGGCCCTGCTGGCCT | NO: |
| | | | | | | | | | | TTGATGTCTTCTTTGTCTTC | 412 |
| | | | | | | | | | | GTCAC | |
| chr | 452145 | A | C | CDC27 | p.N57 | 0.022 | 0.000 | 2.16 | Inf | ATACGACTTTGTCTTTGTAC | SEQ |
| 17 | 23 | | | | 5K | 55 | 00 | E− | | TTCATTACCACTTACCATGC | ID |
| | | | | | | | | 134 | | [A/C]TTATAATGTCTAGGA | NO: |
| | | | | | | | | | | TTGACTCTGATAGCATTTCG | 413 |
| | | | | | | | | | | AAAAC | |
| chr | 452146 | C | T | CDC27 | p.A532 | 0.005 | 0.000 | 1.38 | Inf | AGAGTATAGGCATAAGCGTA | SEQ |
| 17 | 54 | | | | T | 88 | 00 | E−35 | | ATTTGGATCAACTTGGATAG | ID |
| | | | | | | | | | | [C/T]TCTCTGGAAGAATTT | NO: |
| | | | | | | | | | | AATTGCAATATCATGTTCCC | 414 |
| | | | | | | | | | | GTTGC | |
| chr | 452146 | T | C | CDC27 | p.S517 | 0.015 | 0.000 | 3.52 | Inf | TGGAAGAATTTAATTGCAAT | SEQ |
| 17 | 99 | | | | G | 93 | 00 | E−95 | | ATCATGTTCCCGTTGCAGAC | ID |
| | | | | | | | | | | [T/C]GAAACAGTTCCCTGC | NO: |
| | | | | | | | | | | AGCACACCAGGCCTTAAAAA | 415 |
| | | | | | | | | | | AATGG | |
| chr | 452162 | A | G | CDC27 | p.Y470 | 0.008 | 0.000 | 1.83 | Inf | GCCAAAGTGTTGTAGAGTAG | SEQ |
| 17 | 16 | | | | Y | 58 | 00 | E−51 | | ATCTCCATGCCTTCAACTCT | ID |
| | | | | | | | | | | [A/G]TAATTCTCAATCCTT | NO: |
| | | | | | | | | | | CTAACCTCTGAGAATATTCT | 416 |
| | | | | | | | | | | TTCAG | |
| chr | 452192 | T | C | CDC27 | p.Y435 | 0.015 | 0.000 | 2.76 | Inf | ATAGGCCCTTCCAATTTGGC | SEQ |
| 17 | 83 | | | | C | 93 | 00 | E−95 | | ACAGTACCCAACCAGTATTG | ID |
| | | | | | | | | | | [T/C]AGTGGTGAGAAGGTA | NO: |
| | | | | | | | | | | GATGGCTCAAAATATTTATA | 417 |
| | | | | | | | | | | GCTTC | |
| chr | 452292 | A | G | CDC27 | p.T266 | 0.028 | 0.000 | 3.43 | 1102.44 | CTCGGCTATTTCCACTCTGT | SEQ |
| 17 | 61 | | | | T | 92 | 03 | E− | [350.36- | GAGAAGACAGACTTTGTTCC | ID |
| | | | | | | | | 167 | 3468.91] | [A/G]GTTTGGCCGATTCTG | NO: |
| | | | | | | | | | | GCAACAGACTGTAAAACACG | 418 |
| | | | | | | | | | | AAAAG | |
| chr | 452342 | G | C | CDC27 | p.L214 | 0.017 | 0.000 | 6.35 | 2015.93 | AAAGTATCTTGTTTGACTTA | SEQ |
| 17 | 98 | | | | V | 89 | 01 | E− | [280.13- | CCTTGGGGTTAATGGACTAA | ID |
| | | | | | | | | 105 | 14507.61] | [G/C]AGCTGCTGGTCCTCC | NO: |
| | | | | | | | | | | TAATAAACTTCGACCAGTTT | 419 |
| | | | | | | | | | | TTGGT | |
| chr | 452493 | T | G | CDC27 | p.A54 | 0.005 | 0.000 | 1.90 | 32,69 | TAGTACAACTGTGTCCTTTC | SEQ |
| 17 | 72 | | | | A | 64 | 17 | E−22 | [17.63- | AAGAGTCTATATGCTTTATA | ID |
| | | | | | | | | | 60.62] | [T/G]GCCTTTCCTGAGCGG | NO: |
| | | | | | | | | | | TAATAACAGGTTGCCAGTAA | 420 |
| | | | | | | | | | | AAACA | |
| chr | 486534 | G | C | CACNA | p.G548 | 0.010 | 0.000 | 1.36 | Inf | CCACCACCCTCGACGCCTGC | SEQ |
| 17 | 06 | | | 1G | A | 05 | 00 | E−53 | | CCTCTCCGGGGCCCCCCCTG | ID |
| | | | | | | | | | | [G/C]TGGCGCAGAGTCTGT | NO: |
| | | | | | | | | | | GCACAGCTTCTACCATGCCG | 421 |
| | | | | | | | | | | ACTGC | |
| chr | 559172 | G | A | MRPS2 | p.H142 | 0.012 | 0.007 | 1.02 | 1.65[ | CACTCAAGTGTTCGGATTTC | SEQ |
| 17 | 91 | | | 3 | H | 50 | 59 | E−03 | 1.25- | CGGGAAACGTGACTACCTCC | ID |
| | | | | | | | | | 2.2] | [G/A]TGTTGCTTAAAAGAC | NO: |
| | | | | | | | | | | CAGATTTAAGTATCACAGAG | 422 |
| | | | | | | | | | | ATGTT | |
| chr | 560566 | C | T | VEZF1 | p.Q34 | 0.010 | 0.000 | 2.60 | 18.2 | TCCCTGGCCAGCTTGTCACA | SEQ |
| 17 | 07 | | | | 8Q | 29 | 57 | E−32 | [12.13- | TGTTGTTGTTGTTGTTGTTG | ID |
| | | | | | | | | | 27.32] | [C/T]TGCTGCTGCTGCTGC | NO: |
| | | | | | | | | | | TGCTGCTGCTGCTGCTGCTG | 423 |
| | | | | | | | | | | CTTTT | |
| chr | 615685 | C | T | ACE | p.T342 | 0.009 | 0.006 | 1.34 | 1.52 | CCCCAGTTTGGGCAGAACTC | SEQ |
| 17 | 77 | | | | M | 80 | 47 | E−02 | [1.1- | CCTCTGCTTGCAGGGCTGGA | ID |
| | | | | | | | | | 2.09] | [C/T]GCCCAGGAGGATGTT | NO: |
| | | | | | | | | | | TAAGGAGGCTGATGATTTCT | 424 |
| | | | | | | | | | | TCACC | |
| chr | 616837 | T | C | TACO1 | p.H166 | 0.006 | 0.003 | 4.17 | 1.84 | TATCTAACAGTAGCCACAAG | SEQ |
| 17 | 83 | | | | H | 86 | 75 | E−03 | [1.25- | TGCCAAGCAGACATTAGACA | ID |
| | | | | | | | | | 2.69] | [T/C]ATCCTGAATAAGAAT | NO: |
| | | | | | | | | | | GGGTAAGTGTGCGTCTGGGA | 425 |
| | | | | | | | | | | GGAGT | |
| chr | 620386 | T | C | SCN4A | p.H599 | 0.007 | 0.004 | 3.76 | 1.5 | CACAGTGAGCACGTTGTCAA | SEQ |
| 17 | 02 | | | | R | 11 | 73 | E−02 | [1.03- | AGTGCTCCGTCATGGGGTAA | ID |
| | | | | | | | | | 2.19] | [T/C]GTTCCATGGCCATGA | NO: |
| | | | | | | | | | | AGAGGGTGTTGAGCACGATG | 426 |
| | | | | | | | | | | CAGAT | |
| chr | 742881 | G | A | QRICH | p.D721 | 0.009 | 0.000 | 2.53 | 1105.65 | AACCAGGCTGATCTGCACCA | SEQ |
| 17 | 47 | | | 2 | D | 80 | 01 | E−57 | [151.96- | GGTTGGATCAAACCACGCTG | ID |
| | | | | | | | | | 8044.57] | [G/A]TCCATTCCAGGTTGG | NO: |
| | | | | | | | | | | ACCAAACCACGCTGATCCAC | 427 |
| | | | | | | | | | | TCCAG | |
| chr | 742881 | C | I | QRICH | p.R713 | 0.006 | 0.000 | 5.40 | Inf | GATCAAACCACGCTGGTCCA | SEQ |
| 17 | 72 | | | 2 | H | 62 | 00 | E−40 | | TTCCAGGTTGGACCAAACCA | ID |
| | | | | | | | | | | [C/T]GCTGATCCACTCCAG | NO: |
| | | | | | | | | | | GTTGCACCAAACCACGCTGA | 428 |
| | | | | | | | | | | TCCAC | |
| chr | 742882 | C | T | QRICH | p.R703 | 0.017 | 0.000 | 8.11 | 407 | GACCAAACCACGCTGATCCA | SEQ |
| 17 | 02 | | | 2 | H | 89 | 04 | E− | [164.39- | CTCCAGGTTGCACCAAACCA | ID |
| | | | | | | | | 100 | 1007.67] | [C/T]GCTGATCCACTCCAG | NO: |
| | | | | | | | | | | GTTGGACCAAACCACGCTGA | 429 |
| | | | | | | | | | | TCTGC | |
| chr | 742884 | A | T | QRICH | p.V631 | 0.009 | 0.000 | 5.96 | Inf | ACCACGCTGAACTGCACCAG | SEQ |
| 17 | 18 | | | 2 | D | 31 | 00 | E−56 | | GTTGCACCAAACCACGCTGA | ID |
| | | | | | | | | | | [A/T]CTATACCAGGTTGCA | NO: |
| | | | | | | | | | | CCAAACTACGCTGAACTTCA | 430 |
| | | | | | | | | | | CCAGG | |
| chr | 742885 | C | T | QRICH | p.R572 | 0.007 | 0.000 | 1.92 | 799.67 | CAAACCACGCTGATGATCTG | SEQ |
| 17 | 95 | | | 2 | H | 11 | 01 | E−41 | [108.91- | CACGAGGTTGTGCCAAACCA | ID |
| | | | | | | | | | 5871.76] | [C/T]GCTGATCTACTCCAG | NO: |
| | | | | | | | | | | GTTGGACCAAACCATGCTGA | 431 |
| | | | | | | | | | | ACTGC | |
| chr | 743831 | T | C | SPHK1 | p.R285 | 0.005 | 0.002 | 1.15 | 1.82 | GTCTGGGGGAGATGCGCTTC | SEQ |
| 17 | 09 | | | | R | 15 | 84 | E−02 | [1.17- | ACTCTGGGCACCTTCCTGCG | ID |
| | | | | | | | | | 2.83] | [T/C]CTGGCAGCCCTGCGC | NO: |
| | | | | | | | | | | ACCTACCGCGGCCGACTGGC | 432 |
| | | | | | | | | | | CTACC | |
| chr | 768883 | G | A | LOC10 | p.G89 | 0.010 | 0.007 | 4.44 | 1.39 | TCCACAGCTTGGCATCCGCT | SEQ |
| 17 | 19 | | | 06535 | G | 78 | 79 | E−02 | [1.02- | CTTCTCTGCAGAGCGAGATC | ID |
| | | | | 15 | | | | | 1.9] | [G/A]CCTTTGCCCCGGGCT | NO: |
| | | | | | | | | | | TGTAGCAATTTGTGCTTTTT | 433 |
| | | | | | | | | | | CCTCC | |
| chr | 792545 | C | T | SLC38A | p.V169 | 0.006 | 0.003 | 1.29 | 1.72 | CACTGCCCACTGAAGAGGCC | SEQ |
| 17 | 30 | | | 10 | M | 37 | 72 | E−02 | [1.15- | GTGCTTGAGAGAGGAGAGCA | ID |
| | | | | | | | | | 2.56] | [C/T]GATCTGCAGAGGGAG | NO: |
| | | | | | | | | | | AGGGGAGAGAGCACGGGGCA | 434 |
| | | | | | | | | | | GGTCA | |
| chr | 796820 | T | C | SLC25A | p.I57T | 0.005 | 0.002 | 1.51 | 2.24 | ATGACGGGCATGGCGCTGCG | SEQ |
| 17 | 59 | | | 10 | | 15 | 31 | E−03 | [1.43- | GGTGGTGCGTACCGACGGCA | ID |
| | | | | | | | | | 3.5] | [T/C]CCTGGCACTCTACAG | NO: |
| | | | | | | | | | | CGGCCTGAGCGCCTCGCTGT | 435 |
| | | | | | | | | | | GCAGA | |
| chr | 798471 | G | A | ALYREF | p.R148 | 0.005 | 0.003 | 1.10 | 1.78 | GCTCAAAGTGCACGTCTGCT | SEQ |
| 17 | 52 | | | | R | 64 | 17 | E−02 | [1.17- | GTTCCTAAGCTGCGACCAGA | ID |
| | | | | | | | | | 2.72] | [G/A]CGATCATAGTGCACA | NO: |
| | | | | | | | | | | GCCGCCTTCTTCAGCGTTCC | 436 |
| | | | | | | | | | | AAATT | |
| chr | 799545 | G | A | ASPSC | p.L252 | 0.018 | 0.000 | 1.09 | 2063.13 | CTGCCCCCTTTGTTCCTTTC | SEQ |
| 17 | 45 | | | R1 | L | 38 | 01 | E− | [286.79- | TCGGGTGGGGGACAGAGACT | ID |
| | | | | | | | | 107 | 14842.01] | [G/A]GGGGGCCCTCCTGGG | NO: |
| | | | | | | | | | | CCCACGAGGCCTCTGACATC | 437 |
| | | | | | | | | | | ATCTT | |
| chr | 805296 | G | T | FOXK2 | p.P259 | 0.009 | 0.006 | 1.23 | 1.55 | GTTTTGTGTTTGTTTTTTAA | SEQ |
| 17 | 14 | | | | P | 80 | 35 | E−02 | [1.12- | ATACAGGATGATTCAAAGCC | ID |
| | | | | | | | | | 2.14] | [G/T]CCTTACTCCTACGCG | NO: |
| | | | | | | | | | | CAGCTGATAGTTCAGGCGAT | 438 |
| | | | | | | | | | | TACGA | |
| chr | 808993 | T | C | TBCD | p.L118 | 0.011 | 0.006 | 3.07 | 1.62 | AACCGTCTGTGTGACCTTCT | SEQ |
| 17 | 49 | | | | 5P | 27 | 98 | E−03 | [1.2- | GGGCGTACCCAGGCCCCAGC | ID |
| | | | | | | | | | 2.19] | [T/C]GGTGCCCCAGGTAAC | NO: |
| | | | | | | | | | | CCTGTCACCTTCACAGCATG | 439 |
| | | | | | | | | | | AGGTG | |
| chr | 345222 | T | C | TGIF1 | p.P82P | 0.006 | 0.000 | 1.05 | 9.21 | CGACCCCCTCTGCGCTCCTG | SEQ |
| 18 | 3 | | | | | 13 | 67 | E−14 | [5.81- | GGGTCCTCCTGCGCCCCCCC | ID |
| | | | | | | | | | 14.59] | [T/C]CCTCCACCGGCGCGC | NO: |
| | | | | | | | | | | TGCCCACAGCCGCGTGCCCT | 440 |
| | | | | | | | | | | CTCCC | |
| chr | 939652 | C | T | TWSG1 | p.A157 | 0.006 | 0.003 | 4.36 | 1.54 | CACCACCAGAATGTGTCTGT | SEQ |
| 18 | 4 | | | | V | 13 | 99 | E−02 | [1.03- | CCCCAGCAATAATGTTCACG | ID |
| | | | | | | | | | 2.31] | [C/T]GCCTTATTCCAGTGA | NO: |
| | | | | | | | | | | CAAAGGTAACTGCCAACAGT | 441 |
| | | | | | | | | | | TGACT | |
| chr | 988737 | C | A | TXNDC | p.L232 | 0.010 | 0.000 | 4.36 | 99.44 | CAAGTCCCCAGAAGAAGCCA | SEQ |
| 18 | 1 | | | 2 | I | 29 | 10 | E−49 | [49.86- | TCCAGCCCAAGGAGGGTGAC | ID |
| | | | | | | | | | 198.33] | [C/A]TCCCCAAGTCCCTAG | NO: |
| | | | | | | | | | | AGGAAGCCATCCAGCCCAAG | 442 |
| | | | | | | | | | | GAGGG | |
| chr | 125467 | A | G | SPIRE1 | p.A46 | 0.005 | 0.002 | 1.16 | 1.82 | CTTCTTCTGCAGCCTCATAG | SEQ |
| 18 | 78 | | | | A | 15 | 84 | E−02 | [1.17- | CCCTCATCATTGCTACCGTC | ID |
| | | | | | | | | | 2.83] | [A/G]GCTTCCACCGTGTTG | NO: |
| | | | | | | | | | | GCCATGTGATCGATAAGCTG | 443 |
| | | | | | | | | | | CTCTA | |
| chr | 189642 | G | A | GREB1 | p.E93K | 0.007 | 0.004 | 1.32 | 1.65 | CAATCTAACAGTTAATGAAA | SEQ |
| 18 | 86 | | | L | | 60 | 61 | E−02 | [1.14- | TGGAAGATGATGAAGACGAT | ID |
| | | | | | | | | | 2.4] | [G/A]AAGAAATGTCTGATT | NO: |
| | | | | | | | | | | CAAACAGCCCACCAATTCCC | 444 |
| | | | | | | | | | | TATTC | |
| chr | 289343 | A | G | DSG1 | p.I739 | 0.011 | 0.007 | 8.43 | 1.54 | TGTAGGTTCCCCTGCTGGCT | SEQ |
| 18 | 74 | | | | V | 03 | 18 | E−03 | [1.14- | CTGTGGGTTGTTGTAGCTTC | ID |
| | | | | | | | | | 2.09] | [A/G]TTGGAGAAGACCTGG | NO: |
| | | | | | | | | | | ATGACAGCTTCTTGGATACC | 445 |
| | | | | | | | | | | CTGGG | |
| chr | 337850 | G | A | MOCO | p.Q35 | 0.012 | 0.009 | 1.86 | 1.42 | GAATGGAGAATATAAAGCAG | SEQ |
| 18 | 83 | | | S | 4Q | 75 | 01 | E−02 | [1.07- | CACACCTTCACCTTGGCTCA | ID |
| | | | | | | | | | 1.88] | [G/A]TATACCTACGTGGCC | NO: |
| | | | | | | | | | | CTGTCCTCTCTCCAGTACCC | 446 |
| | | | | | | | | | | CAATG | |
| chr | 641789 | C | A | CDH19 | p.V487 | 0.005 | 0.000 | 1.39 | 618.95 | ATGGATTCATCTCTATCCAC | SEQ |
| 18 | 22 | | | | L | 88 | 01 | E−33 | [83.71- | TGCACTGATAGTCTGAATTA | ID |
| | | | | | | | | | 4576.34] | [C/A]CTAAAAAAAAAGGGG | NO: |
| | | | | | | | | | | GATAGATTTTTGTTGTTGTT | 447 |
| | | | | | | | | | | TGGAT | |
| chr | 721140 | G | A | FAM69 | p.A221 | 0.006 | 0.003 | 1.29 | 1.75 | TGCCCTGTGGTGGGGGCTGC | SEQ |
| 18 | 55 | | | C | V | 62 | 79 | E−02 | [1.16- | CCGCGGCCAGGAACTCCACC | ID |
| | | | | | | | | | 2.65] | [G/A]CGTAGAAGTGGCCGC | NO: |
| | | | | | | | | | | AGGAACCCAGCACGGGCAGC | 448 |
| | | | | | | | | | | ACGTG | |
| chr | 723467 | T | C | ZNF40 | p.G124 | 0.008 | 0.005 | 1.66 | 1.56 | ATTGTGAGGGTGAAGGAGGA | SEQ |
| 18 | 01 | | | 7 | 2G | 33 | 35 | E−02 | [1.1- | AACGCAGGAGACGGTGGAGG | ID |
| | | | | | | | | | 2.21] | [T/C]GTTGTCCCCCACAGA | NO: |
| | | | | | | | | | | CACCTGTGCCCTGTGACGCT | 449 |
| | | | | | | | | | | CGATG | |
| chr | 287703 | G | A | PPAP2 | p.R85C | 0.010 | 0.006 | 9.91 | 1.53 | ACCTTGTATACAGCAGCCAC | SEQ |
| 19 | | | | C | | 54 | 93 | E−03 | [1.12- | GTAGTTGTTGAAGTCCGAGC | ID |
| | | | | | | | | | 2.08] | [G/A]AGAATAGAGCCGGTC | NO: |
| | | | | | | | | | | TGTGTACACCAGGTAGGCTT | 450 |
| | | | | | | | | | | CCCCG | |
| chr | 474688 | T | G | ODF3L | p.R20R | 0.012 | 0.006 | 1.39 | 1.85 | ACTTCCTCAGGCCGGTCTCC | SEQ |
| 19 | | | | 2 | | 25 | 67 | E−04 | [1.38- | GGAATCTGGCCCTCCGTCAC | ID |
| | | | | | | | | | 2.47] | [T/G]CGCCGGCCAAGGGGG | NO: |
| | | | | | | | | | | GCTGTGGCCAGCCGTGGGGT | 451 |
| | | | | | | | | | | GGAGT | |
| chr | 104374 | C | T | ABCA7 | p.L318 | 0.005 | 0.003 | 1.35 | 1.79 | GGGGGTGCTGTCCACAGGTG | SEQ |
| 19 | 7 | | | | L | 39 | 01 | E−02 | [1.16- | AACCGGACCTTCGAGGAGCT | ID |
| | | | | | | | | | 2.76] | [C/T]ACCCTGCTGAGGGAT | NO: |
| | | | | | | | | | | GTCCGGGAGGTGTGGGAGAT | 452 |
| | | | | | | | | | | GCTGG | |
| chr | 143033 | C | T | DAZAP | p.F280 | 0.007 | 0.000 | 5.26 | Inf | TGTCCACCCCTCCTGGAGGC | SEQ |
| 19 | 0 | | | 1 | F | 11 | 00 | E−40 | | TTTCCCCCTCCCCAGGGCTT | ID |
| | | | | | | | | | | [C/T]CCTCAGGGCTACGGT | NO: |
| | | | | | | | | | | GCCCCGCCACAGTTCAGTAA | 453 |
| | | | | | | | | | | GTCTA | |
| chr | 145711 | C | A | APC2 | p.P359 | 0.029 | 0.000 | 8.67 | 2568.75 | CGCGCCAACGCGGCGCTGCA | SEQ |
| 19 | 1 | | | | Q | 90 | 01 | E− | [358.86- | CAACATCGTCTTCTCGCAGC | ID |
| | | | | | | | | 162 | 18387.26] | [C/A]GGACCAGGGCCTGGC | NO: |
| | | | | | | | | | | GCGCAAGGAGATGCGCGTCC | 454 |
| | | | | | | | | | | TGCAC | |
| chr | 162098 | G | T | TCF3 | p.P360 | 0.015 | 0.000 | 1.11 | 103.81 | TCCCCTCCCCCCAAAACCCT | SEQ |
| 19 | 0 | | | | P | 20 | 15 | E−64 | [51.56- | CACAGACCTGCCAGGCCCTG | ID |
| | | | | | | | | | 209.02] | [G/T]GGGGAGCCCACGGGG | NO: |
| | | | | | | | | | | GTAGAAGGGCTGGACGAGAA | 455 |
| | | | | | | | | | | GTTAT | |
| chr | 177540 | C | G | ONECU | p.G483 | 0.006 | 0.000 | 1.74 | Inf | TGAACCGCTGGGCTGAGGAG | SEQ |
| 19 | 8 | | | T3 | G | 13 | 00 | E−27 | | CCCAGCACGGCCCCCGGGGG | ID |
| | | | | | | | | | | [C/G]CCCGCCGGCGCCACG | NO: |
| | | | | | | | | | | GCCACTTTCTCCAAGGCCTG | 456 |
| | | | | | | | | | | AGGCG | |
| chr | 224844 | A | G | SF3A2 | p.N43 | 0.012 | 0.000 | 3.76 | Inf | CCTGGGGTCCACCCTCAGCC | SEQ |
| 19 | 5 | | | | 2S | 25 | 00 | E−47 | | TCCGGGAGTTCACCCCTCAA | ID |
| | | | | | | | | | | [A/G]TCCTGGGGTGCACCC | NO: |
| | | | | | | | | | | CCCAACTCCCATGCCCCCAA | 457 |
| | | | | | | | | | | TGCTG | |
| chr | 225042 | A | G | AMH | p.Y167 | 0.007 | 0.000 | 2.29 | 171.42 | GGAGGAGCTGGCCCCCCAGA | SEQ |
| 19 | 3 | | | | C | 84 | 05 | E−36 | [52.47- | GCTGGCGCTGCTGGTGCTGT | ID |
| | | | | | | | | | 560.02] | [A/G]CCCTGGGCCTGGCCC | NO: |
| | | | | | | | | | | TGAGGTCACTGTGACGAGGG | 458 |
| | | | | | | | | | | CTGGG | |
| chr | 287732 | C | T | ZNF55 | p.R122 | 0.008 | 0.005 | 4.26 | 1.46 | AAGGGTGGAGAGACCATGTA | SEQ |
| 19 | 0 | | | 6 | C | 09 | 57 | E−02 | [1.02- | AAAGCAGTAAAGGTAATAAA | ID |
| | | | | | | | | | 2.07] | [C/T]GTGGAAGAACCTTCA | NO: |
| | | | | | | | | | | GAAAGACTCGAAATTGTAAT | 459 |
| | | | | | | | | | | CGTCA | |
| chr | 395944 | G | A | DAPK3 | p.R340 | 0.007 | 0.005 | 2.43 | 1.55 | CCACGTCCTCGTGGCAGAGC | SEQ |
| 19 | 4 | | | | 8 | 84 | 07 | E−02 | [1.08- | CGCCGGCTGCGCTGCAGCTC | ID |
| | | | | | | | | | 2.24] | [G/A]CGCAGGCCCTCCTCG | NO: |
| | | | | | | | | | | GCGGCCGCCGCCTCCTCCAG | 460 |
| | | | | | | | | | | CACCT | |
| chr | 451121 | C | G | PLIN4 | p.S906 | 0.007 | 0.000 | 3.99 | Inf | ACTGCAGACGGTGTCCTTGG | SEQ |
| 19 | 3 | | | | T | 84 | 00 | E−45 | | TACCGGTCAGGACAGTCTTG | ID |
| | | | | | | | | | | [C/G]TGGTGTCCACGCCGG | NO: |
| | | | | | | | | | | TCTGGACAGTCCCTTTGGCC | 461 |
| | | | | | | | | | | AAGTT | |
| chr | 451351 | C | T | PLIN4 | p.K137 | 0.006 | 0.000 | 1.43 | 681.44 | GGACAGCCTTCGAGGTGTCC | SEQ |
| 19 | 9 | | | | K | 13 | 01 | E−35 | [92.31- | AGACCCCCTTGGACGGCCCC | ID |
| | | | | | | | | | 5030.26] | [C/T]TTAGCCATGTCCATG | NO: |
| | | | | | | | | | | GCCCCTGTGACCCCGCTGGA | 462 |
| | | | | | | | | | | CACCA | |
| chr | 572022 | C | T | LONP1 | NM_0 | 0.012 | not | 2.62 | Inf | CGCCGCGAAACGCACGTGAC | SEQ |
| 19 | 9 | | | | 01276 | 76 | found | E−24 | | GCCCGGCGCGTGCCTCGGTA | ID |
| | | | | | 480:c.- | | | | | [C/T]CCGATGGGCGCGTGG | NO: |
| | | | | | 160 + | | | | | CTCGAAACAGCCGCTTCAGG | 463 |
| | | | | | 1G > A | | | | | GAGCT | |
| chr | 583160 | G | A | FUT6 | p.T324 | 0.009 | 0.006 | 1.69 | 1.5[ | GCAGAAAGCGAGTGCCCAGC | SEQ |
| 19 | 8 | | | | M | 56 | 38 | E−02 | 1.09- | TGAAGGAGCGAGGCCGCAGC | ID |
| | | | | | | | | | 2.08] | [G/A]TCTCCCGCCAGCGAA | NO: |
| | | | | | | | | | | AGTAGCTCAGGTAGCGGGCG | 464 |
| | | | | | | | | | | TGGTC | |
| chr | 813810 | C | T | FBN3 | p.11259 | 0.007 | 0.004 | 9.73 | 1.66 | CCCCCGAGGGCCTGATCAAA | SEQ |
| 19 | 4 | | | | 4I | 60 | 59 | E−03 | [1.15- | GTCAAAGCCAGAGGGGCAGA | ID |
| | | | | | | | | | 2.39] | [C/T]GCAGCGGAAGCCACC | NO: |
| | | | | | | | | | | AAGAGTGTTGCGACAGGAGG | 465 |
| | | | | | | | | | | CGCTC | |
| chr | 815480 | G | A | FBN3 | p.P207 | 0.006 | 0.000 | 1.44 | Inf | TGGGTGAGGGGCTCACCTTC | SEQ |
| 19 | 2 | | | | 6S | 86 | 00 | E−40 | | TCGGGAGTCATCCGGGCCTG | ID |
| | | | | | | | | | | [G/A]GACTGCCCCGTGGCC | NO: |
| | | | | | | | | | | AAAGGGGCAGAGCTCCTGAA | 466 |
| | | | | | | | | | | AGGCA | |
| chr | 837316 | C | T | CD320 | p.G4D | 0.006 | 0.003 | 2.31 | 1.77 | CAGAGCCCCTGTTCGCCACG | SEQ |
| 19 | 4 | | | | | 51 | 69 | E−02 | [1.07- | CTCCAACCTGCGCCATCCAA | ID |
| | | | | | | | | | 2.78] | [C/T]CGCCGCTCATGCTGT | NO: |
| | | | | | | | | | | CCCCACAGCGGCGCCGGCCA | 467 |
| | | | | | | | | | | CGCGC | |
| chr | 839896 | A | G | KANK3 | p.D489 | 0.022 | 0.000 | 1.53 | Inf | AGCTACCCGGGGGCTCGGCG | SEQ |
| 19 | 1 | | | | D | 30 | 00 | E− | | CCACCGTTCTCGCTGTCGCC | ID |
| | | | | | | | | 101 | | [A/G]TCGCTGTCGCTGGCG | NO: |
| | | | | | | | | | | TCCTCGCTGGAGGAGCTCTC | 468 |
| | | | | | | | | | | GTACC | |
| chr | 856436 | G | T | PRAM1 | p.P109 | 0.006 | 0.000 | 1.85 | 373.54 | CAGTTTGGACGGCTTCTTGG | SEQ |
| 19 | 6 | | | | Q | 86 | 02 | E−38 | [88.96- | GGAGGTCAGTGACCTCAGGC | ID |
| | | | | | | | | | 1568.57] | [G/T]GCGGGGGCTTCTTGG | NO: |
| | | | | | | | | | | GGAGGTCAGTGACCTCAGGC | 469 |
| | | | | | | | | | | GGCGG | |
| chr | 905982 | A | G | MUC1 | p.S920 | 0.013 | 0.009 | 9.32 | 1.47 | GTATCTGTAGTGACTTCAGT | SEQ |
| 19 | 7 | | | 6 | 7P | 24 | 05 | E−03 | [1.11- | GATGGCCAGTATTTCAGCTG | ID |
| | | | | | | | | | 1.93] | [A/G]GGTGCTGCTCAAATT | NO: |
| | | | | | | | | | | TGGGGGTGAACTGGTTTCAG | 470 |
| | | | | | | | | | | GTTCT | |
| chr | 907288 | G | C | MUC1 | p.P485 | 0.005 | 0.003 | 4.88 | 1.54 | ATGGTGGAGGTGGTAACATT | SEQ |
| 19 | 6 | | | 6 | 4A | 64 | 66 | E−02 | [1.01- | TGGAGATGTGACTTTAGATG | ID |
| | | | | | | | | | 2.35] | [G/C]CTCTGGGTAAGCTGA | NO: |
| | | | | | | | | | | GACAGTAGAATGTGATTCAA | 471 |
| | | | | | | | | | | ATGCT | |
| chr | 923755 | G | A | OR7G3 | p.P25S | 0.008 | 0.004 | 1.80 | 1.67 | GTGGCCAGGTACATGGACAG | SEQ |
| 19 | 4 | | | | | 133 | 873 | E−02 | [1.07- | GAACAGCATGAAGAGGATGG | ID |
| | | | | | | | | | 2.5] | [G/A]CTGCAGCTCCGGATC | NO: |
| | | | | | | | | | | CCCTGACAATCCCAAGAGAA | 472 |
| | | | | | | | | | | AGAAT | |
| chr | 114887 | G | A | EPOR | p.P488 | 0.005 | 0.003 | 2.60 | 1.66 | GGCAGAGGCTCAGCGGCTGG | SEQ |
| 19 | 25 | | | | S | 39 | 25 | E−02 | [1.08- | GATAAGGCTGTTCTCATAAG | ID |
| | | | | | | | | | 2.56] | [G/A]GTTGGAGTAGGGGCC | NO: |
| | | | | | | | | | | ATCGGATAAGCCCCCTTGGG | 473 |
| | | | | | | | | | | CTCCC | |
| chr | 120606 | A | G | ZNF70 | p.Q59 | 0.005 | 0.000 | 4.11 | 6.32 | AAAGGACTCACACTGGAGAG | SEQ |
| 19 | 27 | | | 0 | 6Q | 15 | 82 | E−10 | [3.93- | AAACCCTATGAGTGTAAGCA | ID |
| | | | | | | | | | 10.16] | [A/G]TGTGGGAAAGCCTTC | NO: |
| | | | | | | | | | | AGTTGTGCCTCAAACCTTCG | 474 |
| | | | | | | | | | | AAAGC | |
| chr | 121556 | A | G | ZNF87 | p.C173 | 0.007 | 0.000 | 2.32 | Inf | GAACAGAACTGGGAAAACTG | SEQ |
| 19 | 97 | | | 8 | C | 35 | 00 | E−44 | | AATGCTTTCCCACACTGCTT | ID |
| | | | | | | | | | | [A/G]CATTCATAGGGTTTT | NO: |
| | | | | | | | | | | TTTGCAGAGTGGATTCTTTC | 475 |
| | | | | | | | | | | ATGTC | |
| chr | 125014 | C | T | ZNF79 | p.P587 | 0.005 | 0.000 | 1.69 | 115.34 | TGAGAGAAGCAAATGCTTTC | SEQ |
| 19 | 51 | | | 9 | P | 15 | 04 | E−26 | [43.47- | CCACATTCCTTACATTCATA | ID |
| | | | | | | | | | 306.02] | [C/T]GGGTTCTCTCCAGTA | NO: |
| | | | | | | | | | | TGAGTTTTTTCATGTCCTTG | 476 |
| | | | | | | | | | | AAGAA | |
| chr | 125411 | C | T | ZNF44 | p.P615 | 0.013 | 0.000 | 1.75 | 1489.83 | TGAGAGAAGCAAATGCTTTC | SEQ |
| 19 | 41 | | | 3 | P | 24 | 01 | E−77 | [206.05- | CCACATTCCTTACATTCATA | ID |
| | | | | | | | | | 10771.89] | [C/T]GGGTTCTCTCCAGTA | NO: |
| | | | | | | | | | | TGAGTTTTTTCATGTCCTTG | 477 |
| | | | | | | | | | | AAGAA | |
| chr | 141045 | G | C | RFX1 | p.P34A | 0.006 | 0.000 | 9.57 | Inf | GCAGCGGTGGGTGGCTGCGG | SEQ |
| 19 | 56 | | | | | 37 | 00 | E−37 | | GGGCTGGGGTGCCGCTGGGG | ID |
| | | | | | | | | | | [G/C]TGGTGGCGGTGGCGG | NO: |
| | | | | | | | | | | CTGGGGCTGGGCTTGTGGCG | 478 |
| | | | | | | | | | | GGGCC | |
| chr | 153539 | G | A | BRD4 | p.P982 | 0.017 | 0.000 | 8.48 | Inf | CGTGGAGGGGGCTGATGCTG | SEQ |
| 19 | 36 | | | | S | 65 | 00 | E−60 | | CTGCTGGGGTGGAGGCTGGG | ID |
| | | | | | | | | | | [G/A]CTGGGGTGGTGGGGG | NO: |
| | | | | | | | | | | TGGTGGCGGCTGCTGCTGCA | 479 |
| | | | | | | | | | | GCTGC | |
| chr | 162756 | C | T | CIB3 | p.G139 | 0.007 | 0.000 | 3.10 | 88.16 | ACCTTCTCACATACCAGGCT | SEQ |
| 19 | 56 | | | | R | 84 | 09 | E−38 | [43.31- | CACCTCCTCGGCACTCAGCC | ID |
| | | | | | | | | | 179.45] | [C/T]CCCCCGCGTCAGTTT | NO: |
| | | | | | | | | | | GGTCACCGTCTGCTCCAGGT | 480 |
| | | | | | | | | | | CCCAC | |
| chr | 170390 | A | C | CPAM | p.S110 | 0.005 | 0.003 | 2.24 | 1.67 | GGCCTCGGGAGGGTCCAGGC | SEQ |
| 19 | 23 | | | D8 | 3A | 88 | 53 | E−02 | [1.11- | CACAATGACAGACTCATTGG | ID |
| | | | | | | | | | 2.53] | [A/C]TGGCTCTGGACCATG | NO: |
| | | | | | | | | | | GCCAACCTGGAAAAAGAAAC | 481 |
| | | | | | | | | | | CAAGG | |
| chr | 178816 | G | A | FCHO1 | p.R186 | 0.009 | 0.006 | 4.21 | 1.41 | GAGAGCCTGCGGCGCTCAGT | SEQ |
| 19 | 68 | | | | Q | 56 | 78 | E−02 | [1.02- | GGAAAAATACAACTCAGCCC | ID |
| | | | | | | | | | 1.95] | [G/A]AGCTGACTTTGAGCA | NO: |
| | | | | | | | | | | GAAGATGCTGGACTCAGCCC | 482 |
| | | | | | | | | | | TGGTA | |
| chr | 178889 | A | G | FCHO1 | p.E423 | 0.006 | 0.004 | 4.13 | 1.51 | AGAAGCAGCCCTCTTGGCCT | SEQ |
| 19 | 54 | | | | G | 62 | 38 | E−02 | [1.03- | CACCCTCTCTAGCTGTGCAG | ID |
| | | | | | | | | | 2.23] | [A/G]GAGATTGCAGTCAGA | NO: |
| | | | | | | | | | | GGAGCAGGTGTCCAAGAACC | 483 |
| | | | | | | | | | | TCTTT | |
| chr | 197446 | C | T | GMIP | p.E795 | 0.009 | 0.005 | 8.03 | 1.61 | AGGCCCTCTCCATAGCTGTG | SEQ |
| 19 | 14 | | | | K | 07 | 65 | E−03 | [1.15- | GGCCCAGTGGGTTCTTACCT | ID |
| | | | | | | | | | 2.26] | [C/T]GGTAGGTGTGGCCGT | NO: |
| | | | | | | | | | | GGGATGCTGCTCCAGGGTAC | 484 |
| | | | | | | | | | | TGTGG | |
| chr | 202294 | C | A | ZNF90 | p.G347 | 0.018 | 0.000 | 3.39 | Inf | TCCATACTGGAGAGAAACCC | SEQ |
| 19 | 04 | | | | G | 14 | 00 | E− | | TACAAATGTGAAGAATGTGG | ID |
| | | | | | | | | 108 | | [C/A]AAAGCCTTCAGGCGC | NO: |
| | | | | | | | | | | TCCTTAGTCCTTCGTACACA | 485 |
| | | | | | | | | | | TAAGA | |
| chr | 202295 | C | A | ZNF90 | p.G403 | 0.008 | 0.000 | 2.64 | Inf | GTCATAGTGAAAAGAAACCC | SEQ |
| 19 | 72 | | | | G | 82 | 00 | E−52 | | TACAAATGTGAAGAATGTGG | ID |
| | | | | | | | | | | [C/A]AAAGCCTTCAAGCGC | NO: |
| | | | | | | | | | | TCCTCAACACTTACTATACA | 486 |
| | | | | | | | | | | TAAGA | |
| chr | 212400 | T | C | ZNF43 | p.F298 | 0.011 | 0.000 | 4.36 | Inf | TGGAGAGAAACCCTACAGAT | SEQ |
| 19 | 06 | | | 0 | L | 03 | 00 | E−66 | | GTGAAGAATGTGGCAAAACC | ID |
| | | | | | | | | | | [T/C]TTAACCGGTCCTCAC | NO: |
| | | | | | | | | | | ACCTTACTACACATAAAAGA | 487 |
| | | | | | | | | | | ATTCA | |
| chr | 217194 | T | A | ZNF42 | p.H195 | 0.010 | 0.007 | 3.83 | 1.41 | TTTGCATGCTTTCACAACTA | SEQ |
| 19 | 40 | | | 9 | Q | 05 | 15 | E−02 | [1.03- | ACTCAACATAAGAAAATTCA | ID |
| | | | | | | | | | 1.93] | [T/A]ATTAGAGAGAATACC | NO: |
| | | | | | | | | | | TACAGATGTAAAGAATTTGG | 488 |
| | | | | | | | | | | CAATG | |
| chr | 221543 | A | C | ZNF20 | p.V116 | 0.024 | 0.000 | 1.90 | Inf | AAAGCCTTTGCCACATTCTT | SEQ |
| 19 | 42 | | | 8 | 5G | 02 | 00 | E− | | CACATTTGTAGGGTTTCTCT | ID |
| | | | | | | | | 142 | | [A/C]CAGTATGAATTTTCT | NO: |
| | | | | | | | | | | TATGATAACTAAGGGTTGAG | 489 |
| | | | | | | | | | | GACCA | |
| chr | 221548 | A | T | ZNF20 | p.C100 | 0.005 | 0.003 | 4.87 | 1.54 | AGGTTTGATGACCAGTTGAA | SEQ |
| 19 | 29 | | | 8 | 3S | 64 | 66 | E−02 | [1.01- | AGCTTTGCCACATTCTTCAC | ID |
| | | | | | | | | | 2.35] | [A/T]TTTGTAGGGTTTCTC | NO: |
| | | | | | | | | | | TCCAGTATGAATTACCTTAT | 490 |
| | | | | | | | | | | GTTTA | |
| chr | 221556 | A | G | ZNF20 | p.H715 | 0.017 | 0.000 | 2.21 | 1917.14 | TTTTGCCACATTCTTCACAT | SEQ |
| 19 | 91 | | | 8 | H | 65 | 01 | E− | [266.35- | TTGTAGGGTTTCTCTCCAGT | ID |
| | | | | | | | | 102 | 13799.28] | [A/G]TGAATTCTCTTATGT | NO: |
| | | | | | | | | | | TCCATAAGGTTTGAGGACCA | 491 |
| | | | | | | | | | | GTTGA | |
| chr | 222719 | G | A | ZNF25 | p.E456 | 0.014 | 0.000 | 6.24 | Inf | GTCTTCATACCTTATTCGAC | SEQ |
| 19 | 18 | | | 7 | K | 71 | 00 | E−88 | | ATAAGATAATTCATACTGGA | ID |
| | | | | | | | | | | [G/A]AGAAACCCTACAAAT | NO: |
| | | | | | | | | | | GTGAAGAGTGTGGCAAAGCC | 492 |
| | | | | | | | | | | TTTAA | |
| chr | 222720 | A | G | LNF25 | p.I507 | 0.016 | 0.000 | 8.46 | 926.98 | CAAAGCCTTTAACCGGTCTT | SEQ |
| 19 | 71 | | | 7 | V | 42 | 02 | E−95 | [227.04- | CACACCTTTCTCAACATAAG | ID |
| | | | | | | | | | 3784.7] | [A/G]TAATTCATACTGGAG | NO: |
| | | | | | | | | | | AGAAACCCTACAAATGTGAA | 493 |
| | | | | | | | | | | GAATG | |
| chr | 228476 | G | A | ZNF49 | p.K391 | 0.008 | 0.000 | 2.33 | Inf | CACACCTTACTACACATAAG | SEQ |
| 19 | 44 | | | 2 | K | 09 | 00 | E−48 | | AGAATTCATACTGGAGAGAA | ID |
| | | | | | | | | | | [G/A]CCCTACAAATGTGAA | NO: |
| | | | | | | | | | | GAATGTGGCAAAGCTTTTAA | 494 |
| | | | | | | | | | | CCTAT | |
| chr | 351753 | G | A | ZNF30 | p.D122 | 0.008 | 0.004 | 4.44 | 1.71 | ATTTTCAAATTCTAATAAGA | SEQ |
| 19 | 06 | | | 2 | N | 33 | 89 | E−03 | [1.21- | ATTTGGAATATACAGAATGC | ID |
| | | | | | | | | | 2.42] | [G/A]ACACATTTAGAAGCA | NO: |
| | | | | | | | | | | CCTTTCATTCAAAGTCTACT | 495 |
| | | | | | | | | | | CTTTC | |
| chr | 360024 | T | C | DMKN | p.S276 | 0.006 | 0.001 | 1.79 | 6.01 | CTGCCACCACTGCTGCCGCC | SEQ |
| 19 | 05 | | | | G | 37 | 07 | E−11 | [3.87- | ACTGCTGCCGCCACTGCTGC | ID |
| | | | | | | | | | 9.32] | [T/C]GCCACTGCTGCTGCC | NO: |
| | | | | | | | | | | ACCACTGCTGCTGCCATTGT | 496 |
| | | | | | | | | | | TGTTG | |
| chr | 383774 | C | T | WDR8 | p.E229 | 0.009 | 0.000 | 5.77 | Inf | CCTCCTCCTTCCTTTCCTCC | SEQ |
| 19 | 17 | | | 7 | 8E | 07 | 00 | E−42 | | TCCTCCTCCCTTACCTCCTC | ID |
| | | | | | | | | | | [C/T]TCCTCCCTTTCCTCT | NO: |
| | | | | | | | | | | TCTTCCTCCCTTTCCTCCTC | 497 |
| | | | | | | | | | | CTCCT | |
| chr | 383792 | C | T | WDR8 | p.A169 | 0.005 | 0.003 | 8.94 | 1.86 | ATTTCTTGGCCAGTTTCTTC | SEQ |
| 19 | 29 | | | 7 | 4A | 64 | 03 | E−03 | [1.21- | CTTTTCTGGGCCAATTTCTC | ID |
| | | | | | | | | | 2.88] | [C/T]GCCTCCTGGCTTAGC | NO: |
| | | | | | | | | | | TTCTCCCCTCTTTGGGCCAG | 498 |
| | | | | | | | | | | TGTTT | |
| chr | 388172 | G | A | KCNK6 | KCNK6 | 0.006 | 0.000 | 1.69 | 108.13 | AAAAGAAAAAGATTTACCCT | SEQ |
| 19 | 32 | | | | (NM_0 | 86 | 06 | E−34 | [47.2- | TTACTCTCTTTACTCCCCTA | ID |
| | | | | | 04823: | | | | 247.68] | [G/A]GCTATGGGTACACAA | NO: |
| | | | | | exon2: | | | | | CGCCACTGACTGATGCGGGC | 499 |
| | | | | | c.323- | | | | | AAGGC | |
| | | | | | 1G > A) | | | | | | |
| chr | 404084 | G | A | FCGBP | p.S147 | 0.006 | 0.000 | 6.61 | 702.38 | AATCTTTCAAGGGACCCTGG | SEQ |
| 19 | 20 | | | | 3S | 37 | 01 | E−37 | [95.29- | GGATCCACCAGCTTGTGGCA | ID |
| | | | | | | | | | 5177.19] | [G/A]GAGGACAGTGGCCCT | NO: |
| | | | | | | | | | | GTGGGGCTGGAGAGGAGCCC | 500 |
| | | | | | | | | | | ACAGA | |
| chr | 404086 | T | A | FCGBP | p.Q13 | 0.006 | 0.003 | 8.36 | 2.11 | CTTGGGGTCGCCGTTGTAGT | SEQ |
| 19 | 85 | | | | 85L | 37 | 03 | E−04 | [1.41- | TCCCACACAGGCCACACATC | ID |
| | | | | | | | | | 3.15] | [T/A]GCTGGTAGTAGTTTC | NO: |
| | | | | | | | | | | CGGGGACGGTGACCCGCACA | 501 |
| | | | | | | | | | | TAGTA | |
| chr | 405805 | A | T | ZN78 | p.C615 | 0.006 | 0.004 | 2.82 | 1.57 | AGCTGGGTGGGAAGACTAAA | SEQ |
| 19 | 06 | | | 0A | S | 62 | 24 | E−02 | [1.06- | AACCTTTCCACATTCCTTAC | ID |
| | | | | | | | | | 2.31] | [A/T]TTCAAAGGGTTTCTC | NO: |
| | | | | | | | | | | ACCAGTATGCAATTTCTGAT | 502 |
| | | | | | | | | | | GTCGA | |
| chr | 413558 | A | G | CYP2A | p.L73L | 0.005 | 0.002 | 1.18 | 2.55 | GCATCATGTCCACACAGCAC | SEQ |
| 19 | 49 | | | 6 | | 88 | 32 | E−04 | [1.67- | CACGACCCGCCGGGGCCCCA | ID |
| | | | | | | | | | 3.88] | [A/G]GTGAATGGTGAACAC | NO: |
| | | | | | | | | | | GGGGCCATAGCGCTCACTGA | 503 |
| | | | | | | | | | | TCTGA | |
| chr | 416339 | A | G | CYP2F1 | p.P472 | 0.008 | 0.004 | 1.79 | 1.84 | TGCAGCCGCTGGGTGCGCCC | SEQ |
| 19 | 27 | | | | P | 09 | 41 | E−03 | [1.29- | GAGGACATCGACGTGACCCC | ID |
| | | | | | | | | | 2.62] | [A/G]CTCAGCTCAGGTCTT | NO: |
| | | | | | | | | | | GGCAATTTGCCGCGGCCTTT | 504 |
| | | | | | | | | | | CCAGC | |
| chr | 428553 | C | T | MEGF8 | p.P847 | 0.009 | 0.000 | 3.25 | Inf | TGGGGTTCTGACTCCTCTGC | SEQ |
| 19 | 73 | | | | P | 31 | 00 | E−47 | | CCAACTGACCCCCAGGACCC | ID |
| | | | | | | | | | | [C/T]TTCTGTGAGTGGCAT | NO: |
| | | | | | | | | | | CAGAGCACCAGCCGCAAAGG | 505 |
| | | | | | | | | | | GGACG | |
| chr | 434117 | C | T | PSG6 | p.L325 | 0.005 | 0.001 | 8.18 | 3.69 | CTGGCCCACAGAGGAACAAA | SEQ |
| 19 | 38 | | | | L | 39 | 47 | E−07 | [2.36- | GGATACTCACAGAGGACATT | ID |
| | | | | | | | | | 5.76] | [C/T]AGGGTGACTGGGTTA | NO: |
| | | | | | | | | | | CTGCGGATGCCACCATATCG | 506 |
| | | | | | | | | | | GTCCC | |
| chr | 434117 | G | A | PSG6 | p.T324 | 0.005 | 0.001 | 2.40 | 4.64 | CCCACAGAGGAACAAAGGAT | SEQ |
| 19 | 42 | | | | I | 39 | 17 | E−08 | [2.95- | ACTCACAGAGGACATTCAGG | ID |
| | | | | | | | | | 7.29] | [G/A]TGACTGGGTTACTGC | NO: |
| | | | | | | | | | | GGATGCCACCATATCGGTCC | 507 |
| | | | | | | | | | | CGTAT | |
| chr | 440651 | C | T | XRCC21 | p.E50E | 0.006 | 0.004 | 2.90 | 1.56 | CATCATTCCCAATGTCCACA | SEQ |
| 19 | 67 | | | | | 62 | 26 | E−02 | [1.06- | CTGTGTATCTGCTCCTCCTT | ID |
| | | | | | | | | | 2.3] | [C/T]TCCAACTGTGGGCAG | NO: |
| | | | | | | | | | | AGAGAGAGGCCACTGTCAGT | 508 |
| | | | | | | | | | | GCCTG | |
| chr | 445006 | A | T | ZNF15 | p.Q22 | 0.005 | 0.002 | 1.84 | 1.76 | GGCAAGGAATTTAGTCAAAG | SEQ |
| 19 | 77 | | | 5 | 3L | 15 | 93 | E−02 | [1.13- | CTCACATCTGCAAACTCATC | ID |
| | | | | | | | | | 2.74] | [A/T]GAGAGTCCACACTGG | NO: |
| | | | | | | | | | | AGAGAAACCATTCAAATGTG | 509 |
| | | | | | | | | | | AGCAA | |
| chr | 448906 | A | G | ZNF28 | p.L578 | 0.008 | 0.005 | 1.46 | 1.57 | TTATAATGTTTCTCTCTGCT | SEQ |
| 19 | 74 | | | 5 | P | 82 | 64 | E−02 | [1.12- | CATGTAGTCTTTGATGAGTC | ID |
| | | | | | | | | | 2.2] | [A/G]GAAGGTCCTTTCCAC | NO: |
| | | | | | | | | | | GCTCACAATGTGTGTACTGT | 510 |
| | | | | | | | | | | GTCTC | |
| chr | 458987 | A | G | PPP1R | p.P435 | 0.008 | 0.000 | 1.83 | 26.8 | CAGGGGGCCATGTCTGTTGG | SEQ |
| 19 | 43 | | | 13L | P | 33 | 31 | E−22 | [12.4- | GGATGCTGGGGGGCTGGGGT | ID |
| | | | | | | | | | 57.93] | [A/G]GGGGTTTGGGGTTGG | NO: |
| | | | | | | | | | | GTCTGGGGCTGTGGGGGCAG | 511 |
| | | | | | | | | | | CTGGG | |
| chr | 461377 | G | A | EML2 | p.R213 | 0.006 | 0.000 | 1.59 | Inf | TCCCCGGTGGGCAGCAAATA | SEQ |
| 19 | 13 | | | | X | 62 | 00 | E−39 | | AAGGTTGGCCCGGCAGTCTC | ID |
| | | | | | | | | | | [G/A]GCCACGGTAGCCATA | NO: |
| | | | | | | | | | | GCTGGAGCCACCCAGGGGCT | 512 |
| | | | | | | | | | | GGTTA | |
| chr | 462154 | G | C | FBXO4 | p.P420 | 0.005 | 0.000 | 3.35 | 595.7 | GCCGGGCGCAGTGGCCGGGG | SEQ |
| 19 | 95 | | | 6 | R | 88 | 01 | E−33 | [80.57- | AGTCGGCCGGGGGTGGCTCC | ID |
| | | | | | | | | | 4404.4] | [G/C]GGGGCCCGTCCGGCC | NO: |
| | | | | | | | | | | CGCGGTTCTGGAGAAAGAAG | 513 |
| | | | | | | | | | | AGCTG | |
| chr | 463139 | C | G | RSPH16 | p.A277 | 0.006 | 0.003 | 3.10 | 1.58 | CCTGTTCGCCTTCAGTGCCG | SEQ |
| 19 | 18 | | | A | A | 13 | 90 | E−02 | [1.05- | CCTCCACTCCGGGTGAACAG | ID |
| | | | | | | | | | 2.36] | [C/G]GCCTTCTGTTTCTCC | NO: |
| | | | | | | | | | | GCCATCTTGTAGGTGGGCTG | 514 |
| | | | | | | | | | | CATCT | |
| chr | 472042 | C | T | PRKD2 | p.V324 | 0.011 | 0.008 | 4.45 | 1.36 | TTGTCAGCCTCGCTGAAATC | SEQ |
| 19 | 07 | | | | M | 27 | 30 | E−02 | [1.01- | GGTGGCCTCCTCCATCGGCA | ID |
| | | | | | | | | | 1.83] | [C/T]ATCTGTGGGGACGGA | NO: |
| | | | | | | | | | | GGCATCAGAGGGGTCTCCAC | 515 |
| | | | | | | | | | | CCAGT | |
| chr | 475752 | A | G | ZC3H4 | p.H629 | 0.005 | 0.002 | 4.03 | 2.49 | CAGGGTGCATGTCCGGGTGC | SEQ |
| 19 | 94 | | | | H | 15 | 07 | E−04 | [1.58- | ATGTCGGGGTGCATGTCAGG | ID |
| | | | | | | | | | 3.93] | [A/G]TGCATTGGACCGCCC | NO: |
| | | | | | | | | | | ATTGGCCCTGGGGGTCCCAT | 516 |
| | | | | | | | | | | GTTGG | |
| chr | 486245 | C | T | LIG1 | p.V685 | 0.013 | 0.009 | 1.28 | 1.44 | AGGTAGGCGCCGATCACCAC | SEQ |
| 19 | 55 | | | | M | 24 | 24 | E−02 | [1.09- | CAGGTCCAGGGTGTCACCCA | ID |
| | | | | | | | | | 1.89] | [C/T]GCCATCAAGGTAGTC | NO: |
| | | | | | | | | | | CTTCTTCAGCTGGGAGAAGG | 517 |
| | | | | | | | | | | GGAGG | |
| chr | 486433 | G | A | LIg1 | p.L304 | 0.005 | 0.002 | 1.28 | 1.97 | CCAAGCTCCAGGCCCTGCTG | SEQ |
| 19 | 12 | | | | F | 21 | 65 | E−02 | [1.11- | GGGTGGCCCAAGGTGGTTGA | ID |
| | | | | | | | | | 3.26] | [G/A]GCTGAGGTAGAGGAC | NO: |
| | | | | | | | | | | AGGGAGGAGGTCTGGAGGCG | 518 |
| | | | | | | | | | | ACAGG | |
| chr | 499318 | T | G | GFY | p.L456 | 0.006 | 0.001 | 1.97 | 3.86 | CCAGAGATGACCACGCCCCT | SEQ |
| 19 | 84 | | | | V | 37 | 66 | E−07 | [2.44- | TTGCACCCACAGTTCTGCAT | ID |
| | | | | | | | | | 6.11] | [T/G]TGGACGCCCCGAAAG | NO: |
| | | | | | | | | | | ACCCCTACGACCTCTACTTT | 519 |
| | | | | | | | | | | TATGC | |
| chr | 515180 | T | C | KLK10 | p.N27 | 0.013 | 0.000 | 4.10 | 525.15 | CATAACATCTGGATCAGCTG | SEQ |
| 19 | 60 | | | | 6S | 97 | 03 | E−79 | [164.39- | GAGCGTAGCATCTGGATCAG | ID |
| | | | | | | | | | 1677.55] | [T/C]TGGAGCGTATGACTT | NO: |
| | | | | | | | | | | TATTGATCCAGGACATGTAT | 520 |
| | | | | | | | | | | TTGCA | |
| chr | 516283 | G | T | SIGLEC | p.G54 | 0.009 | 0.000 | 5.23 | Inf | TGCTCCTTCTCCTACCCCTC | SEQ |
| 19 | 92 | | | 9 | V | 31 | 00 | E−56 | | GCATGGCTGGATTTACCCTG | ID |
| | | | | | | | | | | [G/T]CCCAGTAGTTCATGG | NO: |
| | | | | | | | | | | CTACTGGTTCCGGGAAGGGG | 521 |
| | | | | | | | | | | CCAAT | |
| chr | 519197 | C | A | LOC10 | p.C38X | 0.005 | 0.002 | 3.44 | 1.98 | GTGTGGACCAGACGCCATTC | SEQ |
| 19 | 82 | | | 01290 | | 88 | 98 | E−03 | [1.3- | CCATCCCCCTCCCAGGGCTG | ID |
| | | | | 83 | | | | | 3.02] | [C/A]GGCGGCATCCTGGGA | NO: |
| | | | | | | | | | | CCCCACAGCTTCCTCTCCCT | 522 |
| | | | | | | | | | | GGATG | |
| chr | 519197 | G | C | LOC10 | p.G39 | 0.005 | 0.002 | 3.33 | 1.99 | GTGGACCAGACGCCATTCCC | SEQ |
| 19 | 84 | | | 01290 | A | 88 | 97 | E−03 | [1.3- | ATCCCCCTCCCAGGGCTGCG | ID |
| | | | | 83 | | | | | 3.04] | [G/C]CGGCATCCTGGGACC | NO: |
| | | | | | | | | | | CCACAGCTTCCTCTCCCTGG | 523 |
| | | | | | | | | | | ATGCT | |
| chr | 519198 | G | A | LOC1 | p.A58T | 0.008 | 0.005 | 3.85 | 1.72 | CCACAGCTTCCTCTCCCTGG | SEQ |
| 19 | 40 | | | 01290 | | 82 | 15 | E−03 | [1.23- | ATGCTCCTGAGCTGGGAGCC | ID |
| | | | | 83 | | | | | 2.42] | [G/A]CTCACTGTCCCACTG | NO: |
| | | | | | | | | | | GGCTCCTCCACCTCCCCACC | 524 |
| | | | | | | | | | | CACCG | |
| chr | 528880 | T | A | ZNF88 | p.F1399 | 0.018 | 0.000 | 1.62 | 106.63 | GCAAGGTCTTCAGGCACAAG | SEQ |
| 19 | 30 | | | 0 | Q | 63 | 18 | E−81 | [57.96- | TTTTGTCTAACCAATCATCA | ID |
| | | | | | | | | | 196.18] | [T/A]AGAATCCACACGGGA | NO: |
| | | | | | | | | | | GAGCAACCTTACAAATGTAA | 525 |
| | | | | | | | | | | TGAAT | |
| chr | 528880 | A | G | ZNF88 | p.M40 | 0.018 | 0.000 | 5.01 | 102.4 | GGTCTTCAGGCACAAGTTTT | SEQ |
| 19 | 34 | | | 0 | 1V | 87 | 19 | E−81 | [55.69- | GTCTAACCAATCATCATAGA | ID |
| | | | | | | | | | 188.29] | [A/G]TGCACACGGGAGAGC | NO: |
| | | | | | | | | | | AACCTTACAAATGTAATGAA | 526 |
| | | | | | | | | | | TGTGG | |
| chr | 528880 | G | T | ZNF88 | p.M40 | 0.019 | 0.000 | 1.04 | 99.05 | TCTTCAGGCACAAGTTTTGT | SEQ |
| 19 | 36 | | | 0 | 1I | 85 | 20 | E−84 | [55.1- | CTAACCAATCATCATAGAAT | ID |
| | | | | | | | | | 178.05] | [G/T]CACACGGGAGAGCAA | NO: |
| | | | | | | | | | | CCTTACAAATGTAATGAATG | 527 |
| | | | | | | | | | | TGGCA | |
| chr | 531165 | C | T | ZNF83 | p.G435 | 0.007 | 0.004 | 2.91 | 1.65 | CCGATGATGTGCTAGGGATG | SEQ |
| 19 | 14 | | | | E | 482 | 537 | E−02 | [1.04- | AGTTTAGACCGAAGACCTTC | ID |
| | | | | | | | | | 2.52] | [C/T]CACATTCATTACATT | NO: |
| | | | | | | | | | | TATAAGCTTTTTCTCCAGTA | 528 |
| | | | | | | | | | | TGAAT | |
| chr | 532689 | G | A | ZNF60 | p.P693 | 0.012 | 0.000 | 4.13 | 1466.88 | CTGCTTGCTAAAGGCTTTGC | SEQ |
| 19 | 31 | | | 0 | L | 99 | 01 | E−76 | [202.81- | CACACTCATTACACTTGTAA | ID |
| | | | | | | | | | 10609.54] | [G/A]GTTTCTCTCCAGTGT | NO: |
| | | | | | | | | | | GAAGTCCAGTATGTTGTTTC | 529 |
| | | | | | | | | | | AGGTG | |
| chr | 536445 | C | T | ZNF34 | p.K512 | 0.007 | 0.000 | 3.82 | 264.49 | TTTGAGTGAAGACCTTGCCA | SEQ |
| 19 | 48 | | | 7 | K | 35 | 03 | E−40 | [80.69- | CATTCATTACATTTGTAAGG | ID |
| | | | | | | | | | 866.98] | [C/T]TTTTCTCCAGTATGG | NO: |
| | | | | | | | | | | ATGACCTGATGGGTAGTTAG | 530 |
| | | | | | | | | | | GTTTG | |
| chr | 537931 | C | T | BIRC8 | p.A156 | 0.000 | 0.000 | 3.71 | Inf | GAAGTCTGATTCAATTCATT | SEQ |
| 19 | 62 | | | | T | 25 | 00 | E−02 | [NaN- | TTCTGTAGTGTCTTTCTGAG | ID |
| | | | | | | | | | Inf] | [C/T]GCTCACTAGATCTGC | NO: |
| | | | | | | | | | | AACAAGAACCTCAAGCGTTT | 531 |
| | | | | | | | | | | TATAG | |
| chr | 552392 | C | T | KIR3DL | p.H172 | 0.009 | 0.000 | 1.52 | 829.79 | GGATCACTGAGGACCCCTTG | SEQ |
| 19 | 37 | | | 3 | H | 80 | 01 | E−52 | [114.05- | CGCCTCGTTGGACAGCTCCA | ID |
| | | | | | | | | | 6037.46] | [C/T]GATGCGGGTTCCCAG | NO: |
| | | | | | | | | | | GTCAACTATTCCATGGGTCC | 532 |
| | | | | | | | | | | CATGA | |
| chr | 552509 | C | A | KIR2DL | p.P21T | 0.010 | 0.000 | 8.87 | Inf | ATCTTTCTTTCCAGGGTTCT | SEQ |
| 19 | 79 | | | 3 | | 29 | 00 | E−55 | | TCTTGCTGCAGGGGGCCTGG | ID |
| | | | | | | | | | | [C/A]CACATGAGGGTGAGT | NO: |
| | | | | | | | | | | CCTTCTCCAAACCTTCGGGT | 533 |
| | | | | | | | | | | GTCAT | |
| chr | 552848 | G | A | KIR2DL | p.G36 | 0.005 | 0.002 | 7.72 | 2.26 | CTAGGAGTCCACAGAAAACC | SEQ |
| 19 | 21 | | | 1 | D | 64 | 50 | E−04 | [1.47- | TTCCCTCCTGGCCCACCCAG | ID |
| | | | | | | | | | 3.49] | [G/A]TCGCCTGGTGAAATC | NO: |
| | | | | | | | | | | AGAAGAGACAGTCATCCTGC | 534 |
| | | | | | | | | | | AGTGT | |
| chr | 552867 | G | T | KIR2DL | p.G174 | 0.007 | 0.002 | 4.86 | 3.64 | TCCAGGGAAGGGGAGGCCCA | SEQ |
| 19 | 67 | | | 1 | V | 84 | 17 | E−09 | [2.5- | TGAACGTAGGCTCCCTGCAG | ID |
| | | | | | | | | | 5.28] | [G/T]GCCCAAGGTCAACGG | NO: |
| | | | | | | | | | | AACATTCCAGGCTGACTTTC | 535 |
| | | | | | | | | | | CTCTG | |
| chr | 552951 | A | G | KIR2DL | p.T301 | 0.006 | 0.003 | 1.28 | 2.04 | CTCTCCAGGACTCTGATGAA | SEQ |
| 19 | 21 | | | 1 | T | 62 | 25 | E−03 | [1.37- | CAAGACCCTCAGGAGGTGAC | ID |
| | | | | | | | | | 3.04] | [A/G]TACACACAGTTGAAT | NO: |
| | | | | | | | | | | CACTGCGTTTTCACACAGAG | 536 |
| | | | | | | | | | | AAAAA | |
| chr | 553300 | G | A | KIR3DL | p.V113 | 0.026 | 0.000 | 5.79 | 69.95 | CCCACACTCCCCCACTGGGT | SEQ |
| 19 | 36 | | | 1 | M | 23 | 38 | E− | [48.58- | GGTCGGCACCCAGCAACCCC | ID |
| | | | | | | | | 118 | 100.73] | [G/A]TGGTGATCATGGTCA | NO: |
| | | | | | | | | | | CAGGTCAGAGGCTTTCCGTC | 537 |
| | | | | | | | | | | TGGGC | |
| chr | 553330 | C | T | KIR3DL | p.P220 | 0.028 | 0.000 | 9.70 | 1523.42 | AGAACCTCCCTGAGGAAACT | SEQ |
| 19 | 23 | | | 1 | L | 68 | 02 | E− | [376.4- | GCCTCTTCTCCTTCCAGGTC | ID |
| | | | | | | | | 164 | 6165.8] | [C/T]ATATGAGAAACCTTC | NO: |
| | | | | | | | | | | TCTCTCAGCCCAGCCGGGCC | 538 |
| | | | | | | | | | | CCAAG | |
| chr | 554941 | T | G | NLRP2 | p.I330 | 0.007 | 0.001 | 8.85 | 4.3 | AGGGCCCTGAGGGACCTCCG | SEQ |
| 19 | 21 | | | | S | 85 | 80 | E−04 | [2.1- | GATCCTGGCGGAGGAGCCGA | ID |
| | | | | | | | | | 8.8] | [T/G]CTACATAAGGGTGGA | NO: |
| | | | | | | | | | | GGGCTTCCTGGAGGAGGACA | 539 |
| | | | | | | | | | | GGAGG | |
| chr | 560296 | A | C | SSC5D | p.T132 | 0.016 | 0.000 | 1.11 | Inf | CCACCACTACTCCTGATCCC | SEQ |
| 19 | 21 | | | | 6T | 67 | 00 | E−80 | | ACCACGACCCCTCACCCCAC | ID |
| | | | | | | | | | | [A/C]ACTCCTGACCCTTCC | NO: |
| | | | | | | | | | | TCAACCCCTGTCATCACTAC | 540 |
| | | | | | | | | | | TGTGT | |
| chr | 564163 | G | A | NLRP1 | p.A860 | 0.006 | 0.003 | 4.86 | 1.79 | CTCCAGTCTCTCTAAGGCAC | SEQ |
| 19 | 47 | | | 3 | V | 86 | 84 | E−03 | [1.22- | ACTTGGGGTGAGTCAGGGCC | ID |
| | | | | | | | | | 2.63] | [G/A]CACACAATAGCTTTA | NO: |
| | | | | | | | | | | TGCCATCATCTTGGAGCCGA | 541 |
| | | | | | | | | | | TTAAA | |
| chr | 579108 | T | G | ZNF54 | p.F402 | 0.007 | 0.000 | 8.20 | Inf | TGGAGAAAGGCCTTATAAAT | SEQ |
| 19 | 59 | | | 8 | V | 60 | 00 | E−46 | | GCAGTGAATGTGGGAAATCA | ID |
| | | | | | | | | | | [T/G]TTAGGTACCACTGCA | NO: |
| | | | | | | | | | | GGCTCATTAGACACCAGAGA | 542 |
| | | | | | | | | | | GTCCA | |
| chr | 581183 | T | C | ZNF53 | p.S499 | 0.005 | 0.000 | 3.59 | Inf | CTGGAGAAAGGCCTTATGAG | SEQ |
| 19 | 90 | | | 0 | S | 64 | 00 | E−34 | | TGCAGTGTATGTGGGAAATC | ID |
| | | | | | | | | | | [T/C]TTTATCCGAAAAACC | NO: |
| | | | | | | | | | | CACCTCATTCGACACCAGAC | 543 |
| | | | | | | | | | | TGTTC | |
| chr | 583862 | T | C | ZNF81 | p.A158 | 0.017 | 0.009 | 3.97 | 1.86 | AGACAGATGACTCCCCTGAC | SEQ |
| 19 | 84 | | | 4 | A | 16 | 32 | E−06 | [1.45- | ACATGCAACTTACACCTCTT | ID |
| | | | | | | | | | 2.37] | [T/C]GCAAACAACGCCTCC | NO: |
| | | | | | | | | | | TCAACACTCCCTCTGTAGGG | 544 |
| | | | | | | | | | | TTTCT | |
| chr | 584385 | C | T | ZNF41 | p.G348 | 0.007 | 0.000 | 6.65 | Inf | GTTGATGTTGAATGAGATTG | SEQ |
| 19 | 05 | | | 8 | G | 11 | 00 | E−43 | | CCCTTCTGAGTAAAACATTT | ID |
| | | | | | | | | | | [C/T]CCACATTCTTCACAC | NO: |
| | | | | | | | | | | TCATAAGGTCTTTCTCCAGT | 545 |
| | | | | | | | | | | GTGAA | |
| chr | 587723 | C | A | ZNF54 | p.P117 | 0.005 | 0.002 | 1.59 | 1.93 | ATCCCACCACGTGGAAGTGT | SEQ |
| 19 | 21 | | | 4 | T | 53 | 866 | E−02 | [.1.11- | ACAGGAGTGGACCGGAGGAG | ID |
| | | | | | | | | | 3.15] | [C/A]CACCCTCTTTGGTAT | NO: |
| | | | | | | | | | | TAGGAAAAGTGCAAGATCAG | 546 |
| | | | | | | | | | | AGCAA | |
| chr | 141821 | G | A | TPO | p.T10T | 0.009 | 0.005 | 3.81 | 1.69 | TTAATTTTAGAATGAGAGCG | SEQ |
| 2 | 0 | | | | | 31 | 53 | E−03 | [1.22- | CTCGCTGTGCTGTCTGTCAC | ID |
| | | | | | | | | | 2.35] | [G/A]CTGGTTATGGCCTGC | NO: |
| | | | | | | | | | | ACAGAAGCCTTCTTCCCCTT | 547 |
| | | | | | | | | | | CATCT | |
| chr | 100450 | A | T | TAF1B | p.K279 | 0.005 | 0.000 | 2.18 | 201.64 | TCTTTTATTTCAGTCTTGGC | SEQ |
| 2 | 15 | | | | X | 39 | 03 | E−29 | [60.33- | CTGACTACGAGGACATCTAC | ID |
| | | | | | | | | | 673.95] | [A/T]AAAAAACAGTAGAAG | NO: |
| | | | | | | | | | | TTGGAACATTTTTAGATTTG | 548 |
| | | | | | | | | | | CCTCG | |
| chr | 117744 | C | T | GREB1 | p.S171 | 0.001 | 0.000 | 3.24 | 11.75 | TCCAGCAAGACCCGGGCCAG | SEQ |
| 2 | 03 | | | | 3F | 47 | 13 | E−03 | [3.37- | CGAGGTGCAAGAGCCCTTCT | ID |
| | | | | | | | | | 40.92] | [C/T]CCGCTGCCACGTGCA | NO: |
| | | | | | | | | | | CAACTTCATCATCCTGAACG | 549 |
| | | | | | | | | | | TGGAC | |
| chr | 179980 | C | T | MSGN | p.G72 | 0.005 | 0.002 | 8.49 | 1.84 | CTCCCTGTCCAGCTGTGGCT | SEQ |
| 2 | 01 | | | 1 | G | 39 | 93 | E−03 | [1.2- | GGGCTGCCCTGTGAGCACGG | ID |
| | | | | | | | | | 2.84] | [C/T]GGGGCCAGCAGTGGG | NO: |
| | | | | | | | | | | GGCAGCGAAGGCTGCAGTGT | 550 |
| | | | | | | | | | | CGGTG | |
| chr | 239295 | C | T | KLHL29 | p.C865 | 0.011 | 0.008 | 4.80 | 138 | TCCTCCCCCACATGCCCTGC | SEQ |
| 2 | 01 | | | | C | 03 | 04 | E−02 | [1.01- | CCTGTGTTCAGACACGGCTG | ID |
| | | | | | | | | | 1.87] | [C/T]GTCGTGATAAAGAAA | NO: |
| | | | | | | | | | | TATATTCAAAGCGGCTGACA | 551 |
| | | | | | | | | | | TCAGC | |
| chr | 243023 | G | A | TP53I3 | p.R258 | 0.003 | 0.000 | 2.03 | 16.4 | TTGTCCCTAGACCTCAGCAA | SEQ |
| 2 | 58 | | | | X | 93 | 20 | E−04 | [5.5- | ACTGGTGATCAGACTTCCTC | ID |
| | | | | | | | | | 49.2] | [G/A]CTTAAAAAGTAGCTT | NO: |
| | | | | | | | | | | TGAAAACAGGGGCCCATTGA | 552 |
| | | | | | | | | | | TGTCA | |
| chr | 249302 | C | T | NCOA1 | p.A641 | 0.009 | 0.006 | 3.26 | 1.43 | AAACCAGTCACAAACTAGTG | SEQ |
| 2 | 62 | | | | A | 56 | 69 | E−02 | [1.04- | CAGCTTTTGACAACAACTGC | ID |
| | | | | | | | | | 1.98] | [C/T]GAACAGCAGTTACGG | NO: |
| | | | | | | | | | | CATGCTGATATAGACACAAG | 553 |
| | | | | | | | | | | CTGCA | |
| chr | 264151 | G | A | HADHA | p.L661 | 0.010 | 0.007 | 3.77 | 1.41 | TAGCCACTCAAACGGACTTA | SEQ |
| 2 | 98 | | | | L | 05 | 13 | E−02 | [1.03- | CACTTCAGACTTAGGAGGCA | ID |
| | | | | | | | | | 1.94] | [G/A]CTTCAGACTCGCTAA | NO: |
| | | | | | | | | | | AATACTATCCATGTCAGAAT | 554 |
| | | | | | | | | | | TCAAA | |
| chr | 266633 | C | T | DRC1 | p.T331 | 0.005 | 0.003 | 1.29 | 1.91 | TACAACTTGCAGGTGCTGAA | SEQ |
| 2 | 49 | | | | I | 856 | 08 | E−02 | [1.11- | GAAGAGAGATGAAGAAAGCA | ID |
| | | | | | | | | | 3.07] | [C/T]AGTAATTAAATCCCA | NO: |
| | | | | | | | | | | GCAGAAGAGGAAGATCAATC | 555 |
| | | | | | | | | | | GGTAA | |
| chr | 268523 | C | G | CIB4 | p.G42 | 0.017 | 0.000 | 2.59 | Inf | ACCTGGTCCATGGTGAGCGT | SEQ |
| 2 | 40 | | | | R | 40 | 00 | E−95 | | TGCCTCCTTGTAGTACTTCC | ID |
| | | | | | | | | | | [C/G]AGGAGGGCAGAGCTT | NO: |
| | | | | | | | | | | CAGGAAGGTGTCATGGATGC | 556 |
| | | | | | | | | | | TGAAA | |
| chr | 292460 | G | A | FAM17 | p.V536 | 0.005 | 0.003 | 4.86 | 1.55 | AGGTCCTCACCGGGAACCTG | SEQ |
| 2 | 48 | | | 9A | V | 64 | 66 | E−02 | [1.01- | CACGACGTGTGCTTGGTGGT | ID |
| | | | | | | | | | 2.36] | [G/A]ACTGGGGAGGTGAGG | NO: |
| | | | | | | | | | | CCCCCCAGCCTGTGTGCTGT | 557 |
| | | | | | | | | | | GCATT | |
| chr | 315951 | C | T | XDH | p.R607 | 0.005 | 0.003 | 3.21 | 1.61 | TCACTTGATCTTGGCGTGGG | SEQ |
| 2 | 30 | | | | Q | 64 | 51 | E−02 | [1.06- | CCCGGGTGCTGGTGACCAGC | ID |
| | | | | | | | | | 2.45] | [C/T]GGAGAGACAGCTCAT | NO: |
| | | | | | | | | | | TCTCGTAGCGAGGAATGTCG | 558 |
| | | | | | | | | | | TCACA | |
| chr | 322890 | C | T | SPAST | p.P34P | 0.021 | 0.000 | 3.17 | 2161.13 | CTCCCAGGCCTCCGCCCCCT | SEQ |
| 2 | 02 | | | | | 81 | 01 | E− | [301.02- | TGCCTGGCCCCCGCCCCTCC | ID |
| | | | | | | | | 123 | 15515.36] | [C/T]GCCGCCGGGCCGGCC | NO: |
| | | | | | | | | | | CCTCCGCCCGAGTCGCCGCA | 559 |
| | | | | | | | | | | TAAGC | |
| chr | 489827 | A | T | LHCGR | p.L16Q | 0.008 | 0.000 | 3.94 | 29.63 | GAGCGCCTCGCGCAGCGCTC | SEQ |
| 2 | 64 | | | | | 58 | 29 | E−28 | [15.93- | GTGGCAGCGGCGGCTGCAGC | ID |
| | | | | | | | | | 55.12] | [A/T]GCAGCAGCAGCTTCA | NO: |
| | | | | | | | | | | GCAGCTGCAGCGCCGAGAAC | 560 |
| | | | | | | | | | | CGCTG | |
| chr | 624498 | C | T | B3GNT | p.N17 | 0.008 | 0.006 | 3.07 | 1.47 | GAAGGCAAGCAATCCGGGAA | SEQ |
| 2 | 65 | | | 2 | 0N | 82 | 00 | E−02 | [1.05- | TCCTGGGGCCAAGAAAGCAA | ID |
| | | | | | | | | | 2.06] | [C/T]GCAGGGAACCAAACG | NO: |
| | | | | | | | | | | GTGGTGCGAGTCTTCCTGCT | 561 |
| | | | | | | | | | | GGGCC | |
| chr | 743265 | C | T | TET3 | p.P115 | 0.019 | 0.000 | 5.92 | 2204.62 | AGGTGCTCACCGCCTTCCCC | SEQ |
| 2 | 94 | | | | 3P | 61 | 01 | E− | [306.7- | CGCGAGGTCCGACGCCTGCC | ID |
| | | | | | | | | 115 | 15847.05] | [C/T]GAGCCTGCCAAGTCC | NO: |
| | | | | | | | | | | TGCCGCCAGCGGCAGCTGGA | 562 |
| | | | | | | | | | | AGCCA | |
| chr | 744793 | G | A | SLC4A5 | p.S472 | 0.006 | 0.003 | 7.75 | 1.78 | CCCCGATTTCATGCATGGCT | SEQ |
| 2 | 68 | | | | S | 37 | 58 | E−03 | [1.2- | GGCATCTCTCCATCATCCCC | ID |
| | | | | | | | | | 2.66] | [G/A]CTGCTTGTTCCGCCG | NO: |
| | | | | | | | | | | GCCCCGCCACTGCCAGCCCC | 563 |
| | | | | | | | | | | GCCGC | |
| chr | 747513 | G | C | DQX1 | p.T158 | 0.005 | 0.003 | 2.25 | 1.67 | CCTCATCTAGTACCAGCACG | SEQ |
| 2 | 92 | | | | T | 88 | 53 | E−02 | [1.1- | CCCCAGGCTCCAGTGCCTCG | ID |
| | | | | | | | | | 2.52] | [G/C]GTCGAGGCCACCTCC | NO: |
| | | | | | | | | | | TGCAGAAGCAGCCTGTCCCA | 564 |
| | | | | | | | | | | GCAGA | |
| chr | 868317 | G | C | RNF10 | p.L421 | 0.006 | 0.003 | 5.86 | 1.8 | CTCTTCTTCTCAAAGTAATC | SEQ |
| 2 | 51 | | | 3 | V | 62 | 69 | E−03 | [1.22- | AATTAGTAAACCATGACCAA | ID |
| | | | | | | | | | 2.66] | [G/C]GTATGTACTGAGAAA | NO: |
| | | | | | | | | | | CAGGGCTGGGTGTGAAGAGT | 565 |
| | | | | | | | | | | AAAAC | |
| chr | 959456 | G | A | PROM | p.G450 | 0.005 | 0.000 | 8.10 | 571.38 | CTATTCGTGGTGCTCTGCAA | SEQ |
| 2 | 67 | | | 2 | D | 64 | 01 | E−32 | [77.15- | CCTGCTGGGCCTCAATCTGG | ID |
| | | | | | | | | | 4231.98] | [G/A]CATCTGGGGCCTGTC | NO: |
| | | | | | | | | | | TGCCAGGGACGACCCCAGCC | 566 |
| | | | | | | | | | | ACCCA | |
| chr | 981282 | G | C | ANKRD | p.L102 | 0.007 | 0.000 | 4.61 | Inf | GTCTTTGCCTGCTCTCTCTT | SEQ |
| 2 | 58 | | | 36B | 1L | 35 | 00 | E−27 | | TGCTTCTCCAGTTTGGAACG | ID |
| | | | | | | | | | | [G/C]AGCGTTGTGTTTTCA | NO: |
| | | | | | | | | | | TCTGTCAGAGCAGCAAGCTG | 567 |
| | | | | | | | | | | TCCAC | |
| chr | 981283 | G | A | ANKRD | p.T100 | 0.017 | 0.000 | 7.10 | 240.02 | ATCTGTCAGAGCAGCAAGCT | SEQ |
| 2 | 13 | | | 36B | 3M | 16 | 07 | E−60 | [58.84- | GTCCACTATAACAGGCTATC | ID |
| | | | | | | | | | 979.16] | [G/A]TTTTTGCTAATGTTT | NO: |
| | | | | | | | | | | CCCCATTCCGTTTTAGAGCC | 568 |
| | | | | | | | | | | TTTTG | |
| chr | 996517 | G | A | TSGA1 | p.S503 | 0.005 | 0.001 | 1.76 | 3.76 | TAATACAGAGTTCCCTAGTA | SEQ |
| 2 | 98 | | | 0 | S | 21 | 39 | E−05 | [2.09- | GAAGACAAATCTGCAAGAGC | ID |
| | | | | | | | | | 6.31] | [G/A]GACACTTTTTCAAAC | NO: |
| | | | | | | | | | | TGAACCTTCTGAAGCTCCTC | 569 |
| | | | | | | | | | | TTCCA | |
| chr | 108486 | G | T | RGPD4 | RGPD4 | 0.025 | 0.000 | 1.47 | 67.6 | ACTTTAACAGTGTTTTCTTT | SEQ |
| 2 | 338 | | | | (NM_1 | 25 | 38 | E−74 | [34.18- | CTTTTCTTTTTTTTTTTTTA | ID |
| | | | | | 82588: | | | | 133.72] | [G/T]TTGCAACTACTGGCC | NO: |
| | | | | | exon1 | | | | | CTTCAGTATATTATAGTCAG | 570 |
| | | | | | 9:c.26 | | | | | TCACC | |
| | | | | | 06- | | | | | | |
| | | | | | 1G > T) | | | | | | |
| chr | 109347 | T | G | RANBP | p.L96L | 0.014 | 0.000 | 1.94 | Inf | ATTAGCGTTCAGTGGAATTA | SEQ |
| 2 | 813 | | | 2 | | 95 | 00 | E−89 | | AACCCAACACAAAAAGATCT | ID |
| | | | | | | | | | | [T/G]GTGTTGAAGATTGCA | NO: |
| | | | | | | | | | | GAATTGCTTTGTAAAAATGA | 571 |
| | | | | | | | | | | TGTTA | |
| chr | 112922 | C | G | FBLN7 | p.P87A | 0.007 | 0.004 | 7.23 | 1.73 | TCCATCTCTCCTTACAGTTT | SEQ |
| 2 | 601 | | | | | 35 | 26 | E−03 | [1.19- | CCTGCCCGGCTCTGAACACC | ID |
| | | | | | | | | | 2.51] | [C/G]CCGCAGACGGCAGAA | NO: |
| | | | | | | | | | | AGTTTGGAAGCAAGTACTTA | 572 |
| | | | | | | | | | | GTGGA | |
| chr | 113940 | G | A | PSD4 | p.A52T | 0.022 | 0.000 | 6.94 | 2577.18 | CCATGAGGATCCACCGGAGC | SEQ |
| 2 | 187 | | | | | 55 | 01 | E− | [359.1- | CTTTCGAGGAGCAAACCTGG | ID |
| | | | | | | | | 133 | 18495.63] | [G/A]CCACTGACCCTCCTG | NO: |
| | | | | | | | | | | AACCTACCAGACAAAATGTT | 573 |
| | | | | | | | | | | CCTCC | |
| chr | 114500 | C | T | SLC35F | p.E224 | 0.009 | 0.006 | 4.53 | 1.43 | GCAGTAAGTTTCCCCACAGT | SEQ |
| 2 | 349 | | | 5 | K | 07 | 35 | E−02 | [1.03- | TTTCAGTATGGATTCTTGTT | ID |
| | | | | | | | | | 1.99] | [C/T]TTTCACAGGATATGA | NO: |
| | | | | | | | | | | CATGCGAGACAACTTTGCTT | 574 |
| | | | | | | | | | | CCAAT | |
| chr | 132238 | T | C | TUBA3 | p.A278 | 0.007 | 0.004 | 2.79 | 1.55 | TCCACTTCCCCCTGGCCACC | SEQ |
| 2 | 100 | | | D | A | 35 | 75 | E−02 | [1.07- | TATGCCCCAGTCATCTCAGC | ID |
| | | | | | | | | | 2.25] | [T/C]GAGAAGGCCTACCAC | NO: |
| | | | | | | | | | | GAGCAGCTGTCTGTGGCCGA | 575 |
| | | | | | | | | | | GATCA | |
| chr | 136418 | A | G | R3HD) | p.H596 | 0.005 | 0.002 | 1.00 | 2.18 | TTATGATCCTAGATGCCAGC | SEQ |
| 2 | 868 | | | M1 | R | 64 | 60 | E−03 | [1.42- | CTGTTATTGCGCTCCAGGCC | ID |
| | | | | | | | | | 3.33] | [A/G]CTATCACTCCAGCCA | NO: |
| | | | | | | | | | | ACCTCAGTATCGCCCAGTCC | 576 |
| | | | | | | | | | | CTTCT | |
| chr | 141232 | C | T | LRP1B | p.A317 | 0.007 | 0.011 | 2.07 | 0.67 | GCCCAGTAGAGTCTACGATT | SEQ |
| 2 | 800 | | | | 8T | 84 | 71 | E−02 | [0.47- | AACATAATCTATTGTTAGTG | ID |
| | | | | | | | | | 0.95] | [C/T]CATAGGTCTAGAAAT | NO: |
| | | | | | | | | | | CTTGGTTTCTATGACAACAC | 577 |
| | | | | | | | | | | TCTGA | |
| chr | 152982 | T | C | STAM2 | p.M39 | 0.006 | 0.003 | 9.98 | 1.73 | ATAATTTAGAAAATGTTCTC | SEQ |
| 2 | 745 | | | | 2V | 62 | 83 | E−03 | [1.17- | AAAAAACATGCTCACCTGCA | ID |
| | | | | | | | | | 2.56] | [T/C]TGGAACCCCAGATGA | NO: |
| | | | | | | | | | | TGCAGGTGGGTAATGTGCTG | 578 |
| | | | | | | | | | | GAGGG | |
| chr | 165984 | C | T | SCN3A | p.V108 | 0.012 | 0.007 | 7.31 | 1.71 | GGGTTGTTTATGAATGACAT | SEQ |
| 2 | 284 | | | | 41 | 25 | 22 | E−04 | [1.28- | ATAATCATTTTCATCGATTA | ID |
| | | | | | | | | | 2.27] | [C/T]GTATTTTTCAACACT | NO: |
| | | | | | | | | | | GCTTCCAGTACCTACACCAC | 579 |
| | | | | | | | | | | TGGTG | |
| chr | 171070 | G | A | MYO3 | p.G139 | 0.005 | 0.003 | 4.93 | 1.68 | CCAGCGGTTGGATGAAGCAA | SEQ |
| 2 | 982 | | | B | R | 205 | 108 | E−02 | [0.95- | TGATCTCATACATCTTGTAC | ID |
| | | | | | | | | | 2.77] | [G/A]GGGCCCTCTTGGTAA | NO: |
| | | | | | | | | | | GAACATCTATCAAATGGGGT | 580 |
| | | | | | | | | | | ATGAC | |
| chr | 178096 | G | A | NFE2L | p.L286 | 0.005 | 0.003 | 6.39 | 1.86 | AGATCAGAAACATCAATGGG | SEQ |
| 2 | 406 | | | 2 | F | 64 | 04 | E−03 | [1.22- | CCCATTTAGAAGTTCAGAGA | ID |
| | | | | | | | | | 2.84] | [G/A]TGAATGGCTTAAAGT | NO: |
| | | | | | | | | | | AGCAGGTGAGGGCATGCTGT | 581 |
| | | | | | | | | | | TGCTG | |
| chr | 186661 | A | G | FSIP2 | p.R333 | 0.006 | 0.003 | 1.12 | 1.72 | ATCGTGTTCTACTAGAAACA | SEQ |
| 2 | 602 | | | | 6G | 86 | 99 | E−02 | [1.16- | AAGTACAAGACCACAGACCA | ID |
| | | | | | | | | | 2.56] | [A/G]GGGAATCTAACTTTG | NO: |
| | | | | | | | | | | GTAGTTTTGATCAGACCATG | 582 |
| | | | | | | | | | | AAAGG | |
| chr | 186678 | A | T | FSIP2 | p.K680 | 0.025 | 0.000 | 3.65 | Inf | TTTCTCCTAAGTCAACACTA | SEQ |
| 2 | 577 | | | | 0N | 49 | 00 | E− | | AGCACGAGCAGCCTGAAAAA | ID |
| | | | | | | | | 151 | | [A/T]TTTTTGTCACTAAGT | NO: |
| | | | | | | | | | | AAATGTTGTCAGACCACAGC | 583 |
| | | | | | | | | | | CAGTG | |
| chr | 187605 | G | A | FAM17 | p.R95 | 0.007 | 0.004 | 2.40 | 1.58 | GTATTTATGTTGAAAGTCCA | SEQ |
| 2 | 000 | | | 1B | H | 11 | 51 | E−02 | [1.09- | GGTGAATGACATCATCAGTC | ID |
| | | | | | | | | | 2.3] | [G/A]TCAGTACCTGAGCCA | NO: |
| | | | | | | | | | | AGCAGTTGTAGAAGTGTTTG | 584 |
| | | | | | | | | | | TAAAC | |
| chr | 209302 | G | A | PTH2R | p.S82S | 0.006 | 0.000 | 1.50 | 743.52 | GACTCATTTGTTGGCCCAGA | SEQ |
| 2 | 329 | | | | | 62 | 01 | E−38 | [101.01- | GGAACAGTGGGGAAAATATC | ID |
| | | | | | | | | | 5472.96] | [G/A]GCTGTTCCATGCCCT | NO: |
| | | | | | | | | | | CCTTATATTTATGACTTCAA | 585 |
| | | | | | | | | | | CCATA | |
| chr | 211068 | C | A | ACADL | p.R311 | 0.007 | 0.002 | 4.63 | 3.5 | AACTGTTTTGCCAAAAGCTT | SEQ |
| 2 | 107 | | | | M | 11 | 04 | E−08 | [2.37- | TTCTTTGTTTAACATAGTTC | ID |
| | | | | | | | | | 5.16] | [C/A]TGGTTTCTTCAAACA | NO: |
| | | | | | | | | | | TGAATTCACTAGCTGAAATT | 586 |
| | | | | | | | | | | GCCAC | |
| chr | 216285 | C | T | FN1 | p.V527 | 0.001 | not | 4.03 | Inf | ATGTGCCCCTCTTCATGACG | SEQ |
| 2 | 492 | | | | M | 47 | found | E−06 | | CTTGTGGAATGTGTCGTTCA | ID |
| | | | | | | | | | | [C/T]ATTGTAAGTGATGTC | NO: |
| | | | | | | | | | | ATCAACAATGCACTGATCTG | 587 |
| | | | | | | | | | | TTTAG | |
| chr | 233246 | A | G | ALPP | p.E451 | 0.006 | 0.004 | 8.56 | 1.71 | AGCCCCGAGTATCGGCAGCA | SEQ |
| 2 | 249 | | | | G | 86 | 01 | E−03 | [1.17- | GTCAGCAGTGCCCCTGGACG | ID |
| | | | | | | | | | 2.52] | [A/G]AGAGACCCACGCAGG | NO: |
| | | | | | | | | | | CGAGGACGTGGCGGTGTTCG | 588 |
| | | | | | | | | | | CGCGC | |
| chr | 233498 | C | G | EFHD1 | p.P34R | 0.010 | 0.000 | 2.59 | Inf | GAGAGTGGCCCCCAGCTGGC | SEQ |
| 2 | 515 | | | | | 05 | 00 | E−36 | | TCCCCTCGGCGCCCCAGCCC | ID |
| | | | | | | | | | | [C/G]GGAGCCCAAGCCCGA | NO: |
| | | | | | | | | | | GCCCGAGCCTCCCGCCCGTG | 589 |
| | | | | | | | | | | CGCCC | |
| chr | 234229 | C | T | SAG | p.T125 | 0.005 | 0.003 | 1.25 | 1.78 | CTTAAAAAGCTGGGGAGCAA | SEQ |
| 2 | 468 | | | | M | 88 | 32 | E−02 | [1.17- | CACGTACCCCTTTCTCCTGA | ID |
| | | | | | | | | | 2.7] | [C/T]GGTGGGTGACTCCTC | NO: |
| | | | | | | | | | | CGGCCAGCCCTGCTTCCTTC | 590 |
| | | | | | | | | | | ACCCG | |
| chr | 237029 | C | T | AGAP1 | p.C711 | 0.025 | 0.000 | 9.57 | 943.45 | TGCTGGCACACGGCTCCCGG | SEQ |
| 2 | 013 | | | | C | 25 | 03 | E− | [299.22- | GACGAGGTGAACGAGACCTG | ID |
| | | | | | | | | 145 | 2974.8] | [C/T]GGGGAGGGAGACGGC | NO: |
| | | | | | | | | | | CGCACGGCGCTGCATCTGGC | 591 |
| | | | | | | | | | | CTGCC | |
| chr | 238973 | A | H | SCLY | p.K60E | 0.002 | 0.000 | 5.74 | 4.37 | AACGACTCCCCTGGAGCCAG | SEQ |
| 2 | 062 | | | | | 94 | 67 | E−05 | [2.37- | AAGTTATCCAGGCCATGACC | ID |
| | | | | | | | | | 8.05] | [A/G]AGGCCATGTGGGAAG | NO: |
| | | | | | | | | | | CCTGGGGAAATCCCAGCAGC | 592 |
| | | | | | | | | | | CCGTA | |
| chr | 240982 | G | A | PRR21 | p.R53 | 0.021 | 0.000 | 1.26 | 480.79 | GGGTGAAGAGCCGTGGATGA | SEQ |
| 2 | 243 | | | | W | 32 | 05 | E− | [176.38- | AGGGCCGTGGGTGAAGAGCC | ID |
| | | | | | | | | 112 | 1310.53] | [G/A]TGGATGAAGGGCCAT | NO: |
| | | | | | | | | | | GGGTGAAGAGCCGTGGATGA | 593 |
| | | | | | | | | | | AGGGC | |
| chr | 242154 | G | A | ANO7 | NM_0 | 0.005 | 0.000 | 3.42 | 7.1 | GCAAGCAGGTCATCAACAAC | SEQ |
| 2 | 318 | | | | 01001 | 89 | 80 | E−04 | [3- | ATGCAGGAGGTCCTCATCCC | ID |
| | | | | | 891:ex | | | | 16.5] | [G/A]TGAGTCCCCCACTCC | NO: |
| | | | | | on18:c | | | | | TCCCTGGGTGGCATCCAAGG | 594 |
| | | | | | .1988 + | | | | | ACCGA | |
| | | | | | 1G > A | | | | | | |
| chr | 242207 | T | A | HDLBP | p.T14S | 0.009 | 0.006 | 4.29 | 1.43 | ACCACACACCTCTTAATGCT | SEQ |
| 2 | 024 | | | | | 07 | 34 | E−02 | [1.02- | TACAAAATGCATCATGACAG | ID |
| | | | | | | | | | 2.02] | [T/A]TGCTACAAAAAGCCA | NO: |
| | | | | | | | | | | GCGGTCTCTCTCTGCAAGGT | 595 |
| | | | | | | | | | | GCATC | |
| chr | 242312 | C | T | FARP2 | p.H45Y | 0.008 | 0.006 | 4.12 | 1.45 | TGGGCAGACTCTCTTGCCCA | SEQ |
| 2 | 655 | | | | | 82 | 12 | E−02 | [1.03- | GAATGCAAGAGAAGCACCTG | ID |
| | | | | | | | | | 2.03] | [C/T]ACCTCAGAGTAAAGC | NO: |
| | | | | | | | | | | TGCTGGACAACACCATGGAA | 596 |
| | | | | | | | | | | ATATT | |
| chr | 314753 | G | A | LZTS3 | p.L93L | 0.009 | 0.006 | 1.14 | 1.55 | CACTGCCCCGCAGCTCACCA | SEQ |
| 20 | 1 | | | | | 56 | 19 | E−02 | [1.12- | TTGAGGTAGAGGGAGTTGGC | ID |
| | | | | | | | | | 2.14] | [G/A]AGACCCTTGTCCTCT | NO: |
| | | | | | | | | | | GAGGGGTAGCGGCCCGGCCT | 597 |
| | | | | | | | | | | CTCCC | |
| chr | 468011 | T | C | PRNP | p.S55P | 0.005 | 0.000 | 1.14 | 314.81 | GTGGCTGGGGGCAGCCCCAT | SEQ |
| 20 | 8 | | | | | 64 | 02 | E−31 | [74.2- | GGTGGTGGCTGGGGACAGCC | ID |
| | | | | | | | | | 1335.71] | [T/C]CATGGTGGTGGCTGG | NO: |
| | | | | | | | | | | GGTCAAGGAGGTGGCACCCA | 598 |
| | | | | | | | | | | CAGTC | |
| chr | 317569 | C | T | BPIFA2 | p.G12 | 0.005 | 0.002 | 9.96 | 1.86 | AAAAGATGCTTCAGCTTTGG | SEQ |
| 20 | 87 | | | | G | 15 | 77 | E−03 | [1.2- | AAACTTGTTCTCCTGTGCGG | ID |
| | | | | | | | | | 2.9] | [C/T]GTGCTCACTGGGACC | NO: |
| | | | | | | | | | | TCAGAGTCTCTTCTTGACAA | 599 |
| | | | | | | | | | | TCTTG | |
| chr | 340785 | G | A | CEP25 | p.E881 | 0.010 | 0.007 | 4.80 | 1.37 | CTGGCACCAGCAGGAGCTGG | SEQ |
| 20 | 17 | | | 0 | K | 78 | 88 | E−02 | [1.01- | CAAAGGCTCTGGAGAGCTTA | ID |
| | | | | | | | | | 1.86] | [G/A]AAAGGGAAAAAATGG | NO: |
| | | | | | | | | | | AGCTGGAAATGAGGCTAAAG | 600 |
| | | | | | | | | | | GAGCA | |
| chr | 341303 | T | C | ERGIC3 | p.F76F | 0.007 | 0.000 | 3.01 | 79.93 | CGCGGGGAGATAAACTGAAG | SEQ |
| 20 | 30 | | | | | 11 | 09 | E−34 | [38.93- | ATCAACATCGATGTACTTTT | ID |
| | | | | | | | | | 164.12] | [T/C]CCGCACATGCCTTGT | NO: |
| | | | | | | | | | | GCCTGTGAGTACCTCACCAT | 601 |
| | | | | | | | | | | GGGTG | |
| chr | 462798 | G | A | NCOA3 | p.Q12 | 0.011 | 0.000 | 5.51 | Inf | GGGTGGCTATGATGATGCAG | SEQ |
| 20 | 39 | | | | 55Q | 27 | 00 | E−65 | | CAGCAGCAGCAGCAGCAACA | ID |
| | | | | | | | | | | [G/A]CAGCAGCAGCAGCAG | NO: |
| | | | | | | | | | | CAGCAGCAGCAACAGCAACA | 602 |
| | | | | | | | | | | GCAAC | |
| chr | 485033 | G | A | SLC9A8 | p.S519 | 0.009 | 0.006 | 3.58 | 1.44 | GGCCGCCTTTCCTCCCTGCT | SEQ |
| 20 | 06 | | | | S | 07 | 33 | E−02 | [1.03- | CAGGGCAACACTGTGGAGTC | ID |
| | | | | | | | | | 2] | [G/A]GAGCACCTGTCGGAG | NO: |
| | | | | | | | | | | CTCACGGAGGAGGAGTACGA | 603 |
| | | | | | | | | | | GGCCC | |
| chr | 491978 | G | A | PTPN1 | p.G308 | 0.005 | 0.002 | 6.45 | 2.14 | CACTGAAGTTAGAAGTCGGG | SEQ |
| 20 | 54 | | | | S | 541 | 6 | E−03 | [1.23- | TCGTGGGGGGAAGTCTTCGA | ID |
| | | | | | | | | | 3.49] | [G/A]GTGCCCAGGCTGCCT | NO: |
| | | | | | | | | | | CCCCAGCCAAAGGGGAGCCG | 604 |
| | | | | | | | | | | TCACT | |
| chr | 609019 | C | T | LAMA5 | p.V173 | 0.011 | 0.007 | 2.52 | 1.43 | ACCCTGCCACATCATCTCAG | SEQ |
| 20 | 32 | | | | 5M | 27 | 93 | E−02 | [1.06- | CTCCCTCACCTGCAGCACCA | ID |
| | | | | | | | | | 1.92] | [C/T]ATCCGGCCTGCTCTC | NO: |
| | | | | | | | | | | CATGGGGACAAAGACATCTC | 605 |
| | | | | | | | | | | CCCGC | |
| chr | 612963 | C | A | SLCO4 | p.G401 | 0.011 | 0.008 | 4.76 | 1.35 | TCTGCCTGGCCGGGGCCACC | SEQ |
| 20 | 67 | | | A1 | G | 52 | 55 | E−02 | [1.01- | GAGGCCACTCTCATCACCGG | ID |
| | | | | | | | | | 1.81] | [C/A]ATGTCCACGTTCAGC | NO: |
| | | | | | | | | | | CCCAAGTTCTTGGAGTCCCA | 606 |
| | | | | | | | | | | GTTCA | |
| chr | 622005 | C | T | HELZ2 | p.S334 | 0.005 | 0.003 | 3.47 | 1.63 | GGTGCATCCTCTGCCGATAG | SEQ |
| 20 | 87 | | | | S | 15 | 16 | E−02 | [1.05- | TTGGTTGGTGAGATGGGGCC | ID |
| | | | | | | | | | 2.54] | [C/T]GAGGCCACGCTGCTG | NO: |
| | | | | | | | | | | CGGTTGAACTCCAGGGCCAG | 607 |
| | | | | | | | | | | GGCAG | |
| chr | 109429 | T | G | TPTE | p.Q17 | 0.005 | 0.000 | 9.09 | 14.43 | ACTTACCCGCCTTCTTATCA | SEQ |
| 21 | 55 | | | | 3P | 88 | 41 | E−18 | [8.78- | GCTTTTCAAGTTGTCTTTTT | ID |
| | | | | | | | | | 23.7] | [T/G]GATGAAACAGATGAA | NO: |
| | | | | | | | | | | AAATTCTTAACAGAATAATA | 608 |
| | | | | | | | | | | AGTCG | |
| chr | 109429 | C | A | TPTE | p.L164 | 0.012 | 0.000 | 1.16 | 16.39 | CAAGTTGTCTTTTTTGATGA | SEQ |
| 21 | 81 | | | | L | 75 | 79 | E−38 | [11.59- | AACAGATGAAAAATTCTTAA | ID |
| | | | | | | | | | 23.17] | [C/A]AGAATAATAAGTCGT | NO: |
| | | | | | | | | | | AGAAGTCGAAGTAAATGTGT | 609 |
| | | | | | | | | | | CCATC | |
| chr | 149827 | A | C | POTED | p.R58 | 0.022 | 0.000 | 8.43 | 216.23 | CACTTCTGGAGACCACGACG | SEQ |
| 21 | 21 | | | | G | 79 | 11 | E−67 | [53.26- | ACTCCTTTATGAAGATGCTC | ID |
| | | | | | | | | | 877.86] | [A/G]GGAGCAAGATGGGCA | NO: |
| | | | | | | | | | | AGTGTTGCCGCCACTGCTTC | 610 |
| | | | | | | | | | | CCCTG | |
| chr | 349274 | C | G | SON | p.R196 | 0.008 | 0.000 | 2.93 | Inf | GCATTTCCCCAAGCCGCCGC | SEQ |
| 21 | 26 | | | | 3R | 33 | 00 | E−36 | | AGCCGCACCCCCAGCCGCCG | ID |
| | | | | | | | | | | [C/G]AGCCGCACCCCCAGC | NO: |
| | | | | | | | | | | CGCCGCAGCCGCACCCCCAG | 611 |
| | | | | | | | | | | CCGCC | |
| chr | 427708 | G | A | MX2 | p.G408 | 0.010 | 0.006 | 1.46 | 1.51 | GGAGAGCCACCAGAAGGCGA | SEQ |
| 21 | 96 | | | | R | 05 | 66 | E−02 | [1.1- | CCGAGGAGCTGCGGCGTTGC | ID |
| | | | | | | | | | 2.08] | [G/A]GGGCTGACATCCCCA | NO: |
| | | | | | | | | | | GCCAGGAGGCCGACAAGATG | 612 |
| | | | | | | | | | | TTCTT | |
| chr | 434126 | G | C | ZBTB2 | p.A522 | 0.007 | 0.005 | 3.45 | 1.49 | ACCAAATTCGTCTTTATTCA | SEQ |
| 21 | 40 | | | 1 | G | 60 | 10 | E−02 | [1.04- | AATCAGAATCTGGAAAATCT | ID |
| | | | | | | | | | 2.15] | [G/C]CATCAAGGAGAGTAG | NO: |
| | | | | | | | | | | GGCTTGAGCCTTCCTCAAAA | 613 |
| | | | | | | | | | | TTATC | |
| chr | 456707 | G | A | DNMT | p.S276 | 0.024 | 0.000 | 1.25 | 2810.21 | GCACCAGATTGTCCACGAAC | SEQ |
| 21 | 74 | | | 3L | S | 75 | 01 | E− | [391.94- | ATCCAGAAGAAGGGCCTGGG | ID |
| | | | | | | | | 145 | 20149] | [G/A]CTGCCTGGCTTGGGC | NO: |
| | | | | | | | | | | CGTGCGTACTGCAGGAGCCG | 614 |
| | | | | | | | | | | GTGGA | |
| chr | 457866 | G | A | TRPM2 | p.V153 | 0.008 | 0.005 | 3.32 | 1.49[ | CCCGCAGTACGTCCGAGTCT | SEQ |
| 21 | 70 | | | | M | 33 | 61 | E−02 | 1.05- | CCCAGGACACGCCCTCCAGC | ID |
| | | | | | | | | | 2.11] | [G/A]TGATCTACCACCTCA | NO: |
| | | | | | | | | | | TGACCCAGCACTGGGGGCTG | 615 |
| | | | | | | | | | | GACGT | |
| chr | 459947 | T | C | KRTAP | p.P378 | 0.011 | 0.000 | 1.15 | 1313.63 | GCCGCCCCGTGTGCAGGCCC | SEQ |
| 21 | 69 | | | 10-4 | P | 76 | 01 | E−68 | [181.28- | GCCTGCTGCGTGCCCGTCCC | ID |
| | | | | | | | | | 9519.28] | [T/C]TCCTGCTGTGCTCCC | NO: |
| | | | | | | | | | | ACCTCCTCCTGCCAACCCAG | 616 |
| | | | | | | | | | | CTGCT | |
| chr | 459998 | T | A | KRTAP | p.T197 | 0.008 | 0.000 | 4.27 | Inf | CAGCAAGCCGGCTGACAGCT | SEQ |
| 21 | 67 | | | 10-5 | S | 82 | 00 | E−53 | | AGACTGCTGGCAGCATGAAG | ID |
| | | | | | | | | | | [T/A]GGAAGCCCCAGAGCA | NO: |
| | | | | | | | | | | GACGGGCACACAGCAGATGG | 617 |
| | | | | | | | | | | GTTTCG | |
| chr | 460000 | G | A | KRTAP | p.P138 | 0.026 | 0.000 | 3.02 | Inf | ATGAAGAGGAATCCTCAGAA | SEQ |
| 21 | 42 | | | 10-5 | P | 47 | 00 | E− | | CAGGTGGGCACACAGCACAC | ID |
| | | | | | | | | 158 | | [G/A]GGCTTGCAGCAGACA | NO: |
| | | | | | | | | | | GGCACACAGCAGGACTGCTG | 618 |
| | | | | | | | | | | GCAGG | |
| chr | 460206 | C | T | KRTAP | p.C42C | 0.012 | 0.001 | 7.61 | 10.24 | CCGACTCCTGGCAGGTGGAC | SEQ |
| 21 | 47 | | | 10-7 | | 75 | 26 | E−31 | [7.43- | GACTGCCCAGAGAGCTGCTG | ID |
| | | | | | | | | | 14.12] | [C/T]GAGCCCCCCTGCTGC | NO: |
| | | | | | | | | | | GCCCCCAGCTGCTGCGCCCC | 619 |
| | | | | | | | | | | GGCCC | |
| chr | 460324 | T | C | KRTAP | p.S153 | 0.014 | 0.000 | 3.77 | Inf | TGGAGCTTCCTCCCCATGCT | SEQ |
| 21 | 74 | | | 10-8 | P | 22 | 00 | E−85 | | GCCAGCAGTCTAGCTGCCAG | ID |
| | | | | | | | | | | [T/C]CAGCTTGCTGCACCT | NO: |
| | | | | | | | | | | TCTCCCCATGCCAACAGGCC | 620 |
| | | | | | | | | | | TGCTG | |
| chr | 461174 | T | C | KRTAP | p.S98P | 0.017 | 0.000 | 3.00 | 1974.74 | CTGCCAGCAGTCTAGCTGCC | SEQ |
| 21 | 08 | | | 10-12 | | 40 | 01 | E− | [274.3- | AGCCGGCTTGCTGCACCTCC | ID |
| | | | | | | | | 102 | 14216.51] | [T/C]CCCCCTGCCAGCAGG | NO: |
| | | | | | | | | | | CCTGCTGCGTGCCCGTCTGC | 621 |
| | | | | | | | | | | TGCAA | |
| chr | 461914 | G | A | UBE2G | p.P60P | 0.008 | 0.005 | 3.46 | 1.47 | ACATTTTGGACGCATCCACG | SEQ |
| 21 | 00 | | | 2 | | 33 | 68 | E−02 | [1.04- | TTAGCTCCACTTTCGTCATT | ID |
| | | | | | | | | | 2.08] | [G/A]GGCTCTGAAAGAAAA | NO: |
| | | | | | | | | | | GGGAACACCCTCCATGTAAA | 622 |
| | | | | | | | | | | AGGGA | |
| chr | 465964 | G | A | ADARB | p.K281 | 0.008 | 0.005 | 2.59 | 1.5 | TCGTGGATGGTCAGTTCTTT | SEQ |
| 21 | 59 | | | 1 | K | 33 | 59 | E−02 | [1.06- | GAAGGCTCGGGGAGAAACAA | ID |
| | | | | | | | | | 2.12] | [G/A]AAGCTTGCCAAGGCC | NO: |
| | | | | | | | | | | CGGGCTGCGCAGTCTGCCCT | 623 |
| | | | | | | | | | | GGCCG | |
| chr | 185627 | T | C | PEX26 | p.Y109 | 0.005 | 0.002 | 2.61 | 1.82 | AATGGATCGGTGGCAAGAAG | SEQ |
| 22 | 34 | | | | H | 21 | 87 | E−02 | [1.03- | TCCTCTCCTGGGTCCTTCAG | ID |
| | | | | | | | | | 3.01] | [T/C]ATTACCAGGTCCCTG | NO: |
| | | | | | | | | | | AAAAGCTACCCCCCAAAGTC | 624 |
| | | | | | | | | | | CTGGA | |
| chr | 240867 | G | A | ZNF70 | p.C198 | 0.013 | 0.000 | 4.80 | 1525.31 | TGAGGGCTGAGCTCTGGCGG | SEQ |
| 22 | 34 | | | | C | 48 | 01 | E−79 | [211.03- | AAGGCCTTCCCACACTCCCG | ID |
| | | | | | | | | | 11024.83] | [G/A]CACTCGTAGGGCTTC | NO: |
| | | | | | | | | | | TCCCCGGTGTGGATGATCTG | 625 |
| | | | | | | | | | | GTGCC | |
| chr | 250071 | G | A | GGT1 | p.A42T | 0.008 | 0.002 | 5.51 | 3.34 | AGCCTCCAAGGAACCTGACA | SEQ |
| 22 | 72 | | | | | 82 | 66 | E−09 | [2.34- | ACCATGTGTACACCAGGGCT | ID |
| | | | | | | | | | 4.76] | [G/A]CCGTGGCCGCGGATG | NO: |
| | | | | | | | | | | CCAAGCAGTGCTCGAAGATT | 626 |
| | | | | | | | | | | GGGAG | |
| chr | 250072 | A | G | GGT1 | p.K52E | 0.008 | 0.002 | 2.23 | 3.52 | CACCAGGGCTGCCGTGGCCG | SEQ |
| 22 | 02 | | | | | 82 | 52 | E−09 | [2.45- | CGGATGCCAAGCAGTGCTCG | ID |
| | | | | | | | | | 5.05] | [A/G]AGATTGGGAGGTGAG | NO: |
| | | | | | | | | | | CAGGGCAGGGCATGGGACAT | 627 |
| | | | | | | | | | | GGGCC | |
| chr | 268799 | A | G | SRRD | p.R37R | 0.007 | 0.000 | 1.96 | Inf | CTCGACGGCCGCGGCGGAGG | SEQ |
| 22 | 67 | | | | | 11 | 00 | E−08 | | GAGGCGGCGCCCCGGGGGAG | ID |
| | | | | | | | | | | [A/G]GAGGCGGCGCCCCGG | NO: |
| | | | | | | | | | | GGGAGAGAGGCGGCGCCCCG | 628 |
| | | | | | | | | | | GGGCC | |
| chr | 299132 | C | T | THOC5 | p.V523 | 0.010 | 0.007 | 4.97 | 1.38 | ACTCCTTCACCTACCATGTA | SEQ |
| 22 | 78 | | | | M | 05 | 25 | E−02 | [1.01- | ATCCTCATGGGCAACTGTCA | ID |
| | | | | | | | | | 1.9] | [C/T]CCATTTCACCAGGCG | NO: |
| | | | | | | | | | | AGAGACAACCTTGGCAGGGA | 629 |
| | | | | | | | | | | AGAGG | |
| chr | 325904 | C | T | RFPL2 | p.R50 | 0.005 | 0.003 | 3.92 | 1.56 | GGGCCTTTTATTGGTGAGAT | SEQ |
| 22 | 48 | | | | H | 88 | 78 | E−02 | [1.03- | TCCCACCTCCCACTGGGTCA | ID |
| | | | | | | | | | 2.35] | [C/T]GCCCTTCCACACCCT | NO: |
| | | | | | | | | | | CTAACCTGATGAGGCTTTGA | 630 |
| | | | | | | | | | | TTTAA | |
| chr | 325904 | G | A | RFPL2 | p.I42I | 0.005 | 0.003 | 3.59 | 1.96 | CACCTCCCACTGGGTCACGC | SEQ |
| 22 | 71 | | | | | 88 | 01 | E−03 | [1.29- | CCTTCCACACCCTCTAACCT | ID |
| | | | | | | | | | 2.97] | [G/A]ATGAGGCTTTGATTT | NO: |
| | | | | | | | | | | AATTATAACAGGGAATTAGG | 631 |
| | | | | | | | | | | TTTTT | |
| chr | 381203 | C | G | TRIOBP | p.T599 | 0.008 | 0.000 | 4.23 | 966.49 | AGAGCCTCCTCTCCCAATAG | SEQ |
| 22 | 59 | | | | R | 58 | 01 | E−50 | [132.38- | AGCTACACGAGACAACCCCA | ID |
| | | | | | | | | | 7056.38] | [C/G]AACATCCTGTGCCCA | NO: |
| | | | | | | | | | | GCGGGACAATCCCAGAGCCT | 632 |
| | | | | | | | | | | CCAGA | |
| chr | 381208 | C | T | TRIOBP | p.P754 | 0.021 | 0.000 | 3.86 | 2405.39 | CGAGACAACCCCAGAACATC | SEQ |
| 22 | 24 | | | | L | 08 | 01 | E− | [334.92- | CTGTGCCCAGCGGGACAATC | ID |
| | | | | | | | | 124 | 17275.56] | [C/T]CAGAGCCTCCTCTCC | NO: |
| | | | | | | | | | | TAACAGAACCATCCAACAAG | 633 |
| | | | | | | | | | | AGAAC | |
| chr | 381224 | G | T | TRIOBP | p.G129 | 0.026 | 0.000 | 4.02 | Inf | GGCCCAGAGACAGCCAGGGC | SEQ |
| 22 | 49 | | | | 6W | 23 | 00 | E− | | CCCAGGCGCAGTGCAGCAGC | ID |
| | | | | | | | | 141 | | [G/T]GGGGCCGCACCCACA | NO: |
| | | | | | | | | | | GCCCTGGCCGTGCAGAGGTG | 634 |
| | | | | | | | | | | GAGCG | |
| chr | 425646 | G | A | TCF20 | p.S195 | 0.015 | 0.000 | 1.36 | Inf | ACTGCCCCCCTCACCCCCGC | SEQ |
| 22 | 89 | | | | 1S | 44 | 00 | E−91 | | TCCGACTGCTCTGTGCTGAG | ID |
| | | | | | | | | | | [G/A]CTGCCTTTCGCGGTC | NO: |
| | | | | | | | | | | TTGTTCTGCAAGGGGGGGAG | 635 |
| | | | | | | | | | | AGGGC | |
| chr | 466578 | T | C | PKDREJ | p.R447 | 0.006 | 0.002 | 2.21 | 2.21 | ATGTGTGCTATGGCTTTTGG | SEQ |
| 22 | 81 | | | | G | 51 | 96 | E−03 | [1.33- | TCCTTGGAGCACGTGGACCC | ID |
| | | | | | | | | | 3.47] | [T/C]CTTATCAGAAAACGC | NO: |
| | | | | | | | | | | TGTCCTAGAGTCCTTCCGAA | 636 |
| | | | | | | | | | | TCACC | |
| chr | 503153 | C | A | CRELD | p.D182 | 0.035 | 0.027 | 4.33 | 1.29 | ACATGGGGTACCAGGGCCCG | SEQ |
| 22 | 63 | | | 2 | E | 54 | 77 | E−03 | [1.09- | CTGTGCACTGACTGCATGGA | ID |
| | | | | | | | | | 1.53] | [C/A]GGCTACTTCAGCTCG | NO: |
| | | | | | | | | | | CTCCGGAACGAGACCCACAG | 637 |
| | | | | | | | | | | CATCT | |
| chr | 507212 | T | C | PLXNB | p.M95 | 0.009 | 0.006 | 2.76 | 1.47 | TTGGGCACGGGGGACCCCCC | SEQ |
| 22 | 52 | | | 2 | 9V | 31 | 34 | E−02 | [1.06- | GTAGGAGACCTCCAGAAGCA | ID |
| | | | | | | | | | 2.04] | [T/C]CTGGCCCCGTGTCGC | NO: |
| | | | | | | | | | | CTGGGGGCCAGTGACACACT | 638 |
| | | | | | | | | | | GGAGC | |
| chr | 126965 | G | A | CNTN6 | p.K113 | 0.007 | 0.005 | 1.83 | 1.57 | GCCTGGCCACCAATCTTCTG | SEQ |
| 3 | 8 | | | | K | 84 | 02 | E−02 | [1.1- | GGGACAATTCTGAGTCGGAA | ID |
| | | | | | | | | | 2.24] | [G/A]GCAAAGCTCCAATTT | NO: |
| | | | | | | | | | | GCATGTGAGTTTGGGGTAAA | 639 |
| | | | | | | | | | | TTTTG | |
| chr | 109768 | C | T | SLC6A1 | p.C564 | 0.005 | 0.003 | 3.50 | 1.63 | ATGGCATTGGCTGGCTCATG | SEQ |
| 3 | 31 | | | 1 | C | 15 | 17 | E−02 | [1.05- | GCCCTGTCCTCCATGCTCTG | ID |
| | | | | | | | | | 2.53] | [C/T]ATCCCGCTCTGGATC | NO: |
| | | | | | | | | | | TGCATCACAGTGTGGAAGAC | 640 |
| | | | | | | | | | | GGAGG | |
| chr | 147246 | C | T | C3orf2 | p.L26L | 0.009 | 0.006 | 5.92 | 1.61 | ACAGGTTTCAGCAGCAGTCC | SEQ |
| 3 | 64 | | | 0 | | 80 | 11 | E−03 | [1.17- | ATCCACCTGCTGACGGAGCT | ID |
| | | | | | | | | | 2.22] | [C/T]CTCAGACTGAAGATG | NO: |
| | | | | | | | | | | AAGGCCATGGTGGAGTCTAT | 641 |
| | | | | | | | | | | GTCGG | |
| chr | 324094 | C | T | CMTM | p.A122 | 0.008 | 0.005 | 1.57 | 1.54 | TGTGCTTTAACGGCAGTGCC | SEQ |
| 3 | 08 | | | 8 | A | 82 | 74 | E−02 | [1.1- | TTCGTCTTGTACCTCTCTGC | ID |
| | | | | | | | | | 2.16] | [C/T]GCTGTTGTAGATGCA | NO: |
| | | | | | | | | | | TCTTCCGTCTCCCCTGAGAG | 642 |
| | | | | | | | | | | GGACA | |
| chr | 367800 | C | T | DCLK3 | p.R24 | 0.012 | 0.009 | 4.43 | 1.36 | TGGAGAAGGGGCACGGCTGT | SEQ |
| 3 | 80 | | | | Q | 50 | 21 | E−02 | [1.02- | GCTGGGCCAGTGTCAGGGCC | ID |
| | | | | | | | | | 1.81] | [C/T]GGGCTTTGTTGGGGT | NO: |
| | | | | | | | | | | ACAGTTCTTCTACAGCCACC | 643 |
| | | | | | | | | | | TGAAT | |
| chr | 383476 | C | T | SLC22A | p.L55F | 0.009 | 0.006 | 3.16 | 1.44 | GAGGGCTGTCCACACCAAGC | SEQ |
| 3 | 80 | | | 14 | | 56 | 64 | E−02 | [1.04- | AGGATGACAAGTTTGCCAAC | ID |
| | | | | | | | | | 1.99] | [C/T]TCCTGGATGCGGTGG | NO: |
| | | | | | | | | | | GGGAGTTTGGCACATTCCAG | 644 |
| | | | | | | | | | | CAGAG | |
| chr | 386718 | G | A | SCN5A | p.H118 | 0.005 | 0.002 | 2.12 | 2.01 | ATGAGTGAACCAGAATCTTC | SEQ |
| 3 | 40 | | | | H | 88 | 94 | E−03 | [1.33- | ACAGCCGCTCTCCGGATGGG | ID |
| | | | | | | | | | 3.05] | [G/A]TCGAAGGGACTGAGG | NO: |
| | | | | | | | | | | ACATACAAGGCGTTGGTGGC | 645 |
| | | | | | | | | | | ACTGA | |
| chr | 419493 | G | A | ULK4 | p.P391 | 0.008 | 0.005 | 2.71 | 1.5 | TAGGAAGAAAATTTCCCAAG | SEQ |
| 3 | 48 | | | | S | 58 | 74 | E−02 | [1.06- | TCTGCTCACCTTGGTCAGAG | ID |
| | | | | | | | | | 2.11] | [G/A]AGAAGTCTTCTGTGG | NO: |
| | | | | | | | | | | TGAACAGTGAGTCATATCCT | 646 |
| | | | | | | | | | | CACCA | |
| chr | 427750 | G | A | CCDC1 | p.R471 | 0.007 | 0.000 | 8.25 | 88.72 | CTGGGTCCTCCAGGAACTGG | SEQ |
| 3 | 60 | | | 3 | R | 11 | 08 | E−35 | [41.97- | GTATAGGCAGGGCTGACCTC | ID |
| | | | | | | | | | 187.53] | [G/A]CGGCCACTGGACCCC | NO: |
| | | | | | | | | | | TCACCCACTCCTTTATTCCG | 647 |
| | | | | | | | | | | AAGAT | |
| chr | 455420 | C | T | LARS2 | p.A564 | 0.006 | 0.003 | 1.03 | 2.02 | GGATGCCTGTGGATTTGTAC | SEQ |
| 3 | 03 | | | | A | 86 | 41 | E−03 | [1.38- | ATTGGAGGGAAAGAACATGC | ID |
| | | | | | | | | | 2.97] | [C/T]GTCATGCACTTGTTC | NO: |
| | | | | | | | | | | TATGCAAGATTCTTTAGTCA | 648 |
| | | | | | | | | | | TTTTT | |
| chr | 460629 | G | A | XCR1 | p.S173 | 0.009 | 0.005 | 1.02 | 1.57 | GGAGGTGAGGTACCACGTGA | SEQ |
| 3 | 22 | | | | L | 31 | 97 | E−02 | [1.13- | GTTCGGAATAATCACAGCCC | ID |
| | | | | | | | | | 2.17] | [G/A]AAGAAAGCACCTTGT | NO: |
| | | | | | | | | | | GGAAGATGGTGTCGAGGATG | 649 |
| | | | | | | | | | | GAGGA | |
| chr | 464969 | G | A | LTF | p.A174 | 0.007 | 0.004 | 2.66 | 1.55 | GGTTGGGGAACTGTCCTTTA | SEQ |
| 3 | 10 | | | | A | 11 | 61 | E−02 | [1.06- | TCTGCACCGGGAACACAGCT | ID |
| | | | | | | | | | 2.25] | [G/A]GCTGAGAAGAACCTG | NO: |
| | | | | | | | | | | GCCACAGCTGTTAAACACAG | 650 |
| | | | | | | | | | | AGAAG | |
| chr | 495691 | G | A | DAG1 | p.V411 | 0.006 | 0.002 | 7.27 | 2.16 | CTGGCCAGATTCGCCCAACG | SEQ |
| 3 | 77 | | | | V | 37 | 96 | E−04 | [1.45- | ATGACCATTCCTGGCTATGT | ID |
| | | | | | | | | | 3.22] | [G/A]GAGCCTACTGCAGTT | NO: |
| | | | | | | | | | | GCTACCCCTCCCACAACCAC | 651 |
| | | | | | | | | | | CACCA | |
| chr | 497288 | A | G | RNF12 | p.E32G | 0.009 | 0.006 | 2.22 | 1.49 | CTTTTCTCCCTTCTGACTTG | SEQ |
| 3 | 70 | | | 3 | | 56 | 43 | E−02 | [1.08- | TGGCTCAGGCATTGTGCAGG | ID |
| | | | | | | | | | 2.06] | [A/G]GAAGCTGCTGAATGA | NO: |
| | | | | | | | | | | CTACCTGAACCGCATCTTTT | 652 |
| | | | | | | | | | | CCTCT | |
| chr | 503345 | C | T | NAT6 | p.V141 | 0.008 | 0.005 | 2.17 | 1.53 | TGGTTCAGCACCCGTGACAG | SEQ |
| 3 | 40 | | | | I | 09 | 29 | E−02 | [1.07- | GCGGGCATGGCCCACCACAA | ID |
| | | | | | | | | | 2.18] | [C/T]GGGTGCTGCTTCAAG | NO: |
| | | | | | | | | | | TGTGGGGTGGGGGCTTAGCA | 653 |
| | | | | | | | | | | GCATC | |
| chr | 520056 | G | A | ABHD1 | p.R8C | 0.007 | 0.005 | 4.86 | 1.45 | AAGAAGAGGGCCTGGCCCTG | SEQ |
| 3 | 65 | | | 4B | | 60 | 26 | E−02 | [1.01- | CACCTGGATGGTGCCCTCGC | ID |
| | | | | | | | | | 2.08] | [G/A]CTGCTCCACGCTTGC | NO: |
| | | | | | | | | | | TGCCATGCCTGCTGCTGCTG | 654 |
| | | | | | | | | | | TGCTG | |
| chr | 525408 | C | T | STAB1 | p.S655 | 0.006 | 0.004 | 2.53 | 1.61 | TGCCCCCGACCATCCTGCCC | SEQ |
| 3 | 42 | | | | S | 62 | 12 | E−02 | [1.09- | ATCCTGCCCAAGCACTGCAG | ID |
| | | | | | | | | | 2.38] | [C/T]GAGGAGCAGCACAAG | NO: |
| | | | | | | | | | | ATTGTGGCGGTGAGCCTCGC | 655 |
| | | | | | | | | | | CTGCA | |
| chr | 757862 | A | G | ZNF71 | p.S855 | 0.005 | 0.000 | 1.25 | 89.92 | TTTTCTCCTGTGTGTGTTCT | SEQ |
| 3 | 11 | | | 7 | P | 15 | 06 | E-14 | [12.09- | CTGATGTATACTGAGGCCTG | ID |
| | | | | | | | | | 668.7] | [A/G]CTTCTGGGAGAAAGT | NO: |
| | | | | | | | | | | TTTCCTACATTCATTACATC | 656 |
| | | | | | | | | | | TAAAG | |
| chr | 757869 | C | A | ZNF71 | p.R611 | 0.008 | 0.000 | 1.48 | Inf | ACATTCATTACATTCATAGG | SEQ |
| 3 | 42 | | | 7 | I | 58 | 00 | E−41 | | GTCTTTCCCCTGTGTGAGTT | ID |
| | | | | | | | | | | [C/A]TCTTGTGTATCCCAA | NO: |
| | | | | | | | | | | GGTTTAACTTATTGATAAAG | 657 |
| | | | | | | | | | | GTTTT | |
| chr | 757872 | G | T | ZNF71 | p.P506 | 0.011 | 0.000 | 9.28 | Inf | TTACAGCGAAAGGTTTTCCC | SEQ |
| 3 | 58 | | | 7 | T | 52 | 00 | E−35 | | ACATTCATTGCATTCGTAGG | ID |
| | | | | | | | | | | [G/T]TTTTTCCCCTGTGTG | NO: |
| | | | | | | | | | | AGTCCATTGATGGATAGTGA | 658 |
| | | | | | | | | | | GGAAT | |
| chr | 757875 | G | C | ZNF71 | p.L410 | 0.027 | 0.000 | 3.86 | Inf | TAGGGCTTTTCCCCTGTGTG | SEQ |
| 3 | 46 | | | 7 | V | 45 | 00 | E−62 | | AGTTCTATGATGTATTGTGA | ID |
| | | | | | | | | | | [G/C]GTATGACTTCTGGCT | NO: |
| | | | | | | | | | | AAAGGTTTTTCCACATTCAC | 659 |
| | | | | | | | | | | TACAC | |
| chr | 757881 | G | C | ZNF71 | p.L206 | 0.007 | 0.000 | 3.87 | Inf | TTGAAGGTTTTCCCTTGTTC | SEQ |
| 3 | 58 | | | 7 | V | 35 | 00 | E−34 | | ATTACATTGAAAAGTCTGCA | ID |
| | | | | | | | | | | [G/C]CAGAGTTTGAATCTT | NO: |
| | | | | | | | | | | GTGATGCTGAGTAAGATGTT | 660 |
| | | | | | | | | | | CATGA | |
| chr | 757882 | T | A | ZNF71 | p.D161 | 0.006 | 0.000 | 7.04 | 14.75 | TGTCTCCCCAGGCTTAATAG | SEQ |
| 3 | 92 | | | 7 | V | 13 | 42 | E−12 | [6.04- | GGAAAAGCATGTTCTGGCAA | ID |
| | | | | | | | | | 35.97] | [T/A]CATTAAACTGCCCAG | NO: |
| | | | | | | | | | | GCTTCATTCCTGAACTGTTT | 661 |
| | | | | | | | | | | CCATT | |
| chr | 999985 | G | C | TBC1D | p.C31S | 0.008 | 0.005 | 4.33 | 1.45 | AGGGAAAAAGATCTTGAAGA | SEQ |
| 3 | 31 | | | 23 | | 09 | 59 | E−02 | [1.02- | AGCTCTGGAAGCAGGAGGTT | ID |
| | | | | | | | | | 2.06] | [G/C]TGATCTTGAAACGTT | NO: |
| | | | | | | | | | | GAGAAATATAATTCAAGGAA | 662 |
| | | | | | | | | | | GACCG | |
| chr | 113052 | G | C | WDR5 | p.P118 | 0.006 | 0.004 | 4.42 | 1.5 | TTCCTCTTCCTTCTTGGCAG | SEQ |
| 3 | 314 | | | 2 | 5R | 86 | 57 | E−02 | [1.02- | CATTTATTCTCATGTGCTCA | ID |
| | | | | | | | | | 2.23] | [G/C]GTATCTTGTAGTCTG | NO: |
| | | | | | | | | | | GGGCTGTCTTCAGATTGAAA | 663 |
| | | | | | | | | | | TCTCC | |
| chr | 124578 | C | G | ITGB5 | p.E80 | 0.009 | 0.006 | 3.42 | 1.45 | GGCAGGCTCCTCAGGACATG | SEQ |
| 3 | 212 | | | | Q | 07 | 25 | E−02 | [1.04- | GAAGCTGCTGGCTGGGCTCT | ID |
| | | | | | | | | | 2.03] | [C/G]TATCTCACCTCCACA | NO: |
| | | | | | | | | | | GCCATTTTTGACAAGGTTTG | 664 |
| | | | | | | | | | | CCCTC | |
| chr | 124646 | G | A | MUC1 | p.T66I | 0.006 | 0.004 | 3.25 | 1.59 | GGAGGAACTATGTGTACTAA | SEQ |
| 3 | 693 | | | 3 | | 37 | 03 | E−02 | [1.07- | TTATGGGGGGAGCAGGTGAA | ID |
| | | | | | | | | | 2.36] | [G/A]TAGCTGTTGGGAAAG | NO: |
| | | | | | | | | | | GTGTATTTGCTGTGGTGCTA | 665 |
| | | | | | | | | | | GCAGT | |
| chr | 129196 | C | T | IFT122 | p.R366 | 0.008 | 0.005 | 3.60 | 1.46 | CTATGAGTTGTATTCAGAGG | SEQ |
| 3 | 984 | | | | W | 33 | 73 | E−02 | [1.03- | ACTTATCAGACATGCATTAC | ID |
| | | | | | | | | | 2.06] | [C/T]GGGTAAAGGAGAAGA | NO: |
| | | | | | | | | | | TTATCAAGAAGTTTGAGTGC | 666 |
| | | | | | | | | | | AACCT | |
| chr | 132198 | G | A | DNAJC | p.R912 | 0.006 | 0.003 | 1.75 | 1.68 | ATTTATTTCAATAGTGCACA | SEQ |
| 3 | 097 | | | 13 | R | 13 | 65 | E−02 | [1.12- | GATAAACTTGAACGAGATAG | ID |
| | | | | | | | | | 2.53] | [G/A]TTGATTCTCTTCCTT | NO: |
| | | | | | | | | | | AACAAGTTGATCCTTAATAA | 667 |
| | | | | | | | | | | GGTAC | |
| chr | 132247 | T | G | DNAJC | p.L217 | 0.006 | 0.004 | 1.27 | 1.68 | GCTCAGATTGTTAAAGCTCT | SEQ |
| 3 | 160 | | | 13 | 0W | 86 | 09 | E−02 | [1.15- | CAAGGCAATGACTCGAAGTT | ID |
| | | | | | | | | | 2.47] | [T/G]GCAGTATGGAGAACA | NO: |
| | | | | | | | | | | GGTGAGTCTGCATAGAGTCA | 668 |
| | | | | | | | | | | ACTTT | |
| chr | 136664 | A | T | NCK1 | p.S139 | 0.011 | 0.008 | 4.08 | 1.38 | AAGTGTTGCATGTGGTACAG | SEQ |
| 3 | 807 | | | | S | 03 | 02 | E−02 | [1.02- | GCTCTTTACCCATTCAGCTC | ID |
| | | | | | | | | | 1.86] | [A/T]TCTAATGATGAAGAA | NO: |
| | | | | | | | | | | CTTAATTTCGAGAAAGGAGA | 669 |
| | | | | | | | | | | TGTAA | |
| chr | 137849 | G | T | A4GNT | p.P97P | 0.008 | 0.005 | 2.16 | 1.52 | TTGCTGACAGGAAGGAAAAA | SEQ |
| 3 | 808 | | | | | 82 | 83 | E−02 | [1.08- | GCTGGGTATGTGGAGTTTGA | ID |
| | | | | | | | | | 2.13] | [G/T]GGCATCGGTGTGGAA | NO: |
| | | | | | | | | | | TCAGTAAGACCCTTCATAAA | 670 |
| | | | | | | | | | | GAACA | |
| chr | 186953 | C | T | MASP1 | p.P582 | 0.009 | 0.005 | 1.70 | 1.54 | AGATGCCCCAGCCGGCCACC | SEQ |
| 3 | 913 | | | | P | 07 | 90 | E−02 | [1.11- | AGGCCCAGCATGTGGGGGGC | ID |
| | | | | | | | | | 2.15] | [C/T]GGGCCTTCAGGCTCA | NO: |
| | | | | | | | | | | AGCCTTGGCAGGCAGACAGG | 671 |
| | | | | | | | | | | CATAA | |
| chr | 192980 | C | T | HRASL | p.S160 | 0.008 | 0.005 | 7.49 | 1.64 | AATTCTACTTTATAGATGGC | SEQ |
| 3 | 784 | | | S | S | 33 | 09 | E−03 | [1.16- | AATTCCTGCGTCCTTTACAAG | ID |
| | | | | | | | | | 2.33] | [C/T]GCCAAGTCTGTATTC | NO: |
| | | | | | | | | | | AGCAGTAAGGCCCTGGTGAA | 672 |
| | | | | | | | | | | AATGC | |
| chr | 195306 | A | G | APOD | p.F1.5S | 0.009 | 0.005 | 9.46 | 1.59 | GCACTTCCCAAGATGAAATG | SEQ |
| 3 | 289 | | | | | 31 | 89 | E−03 | [1.14- | CTTGTCCCTCTGCCGCACCG | ID |
| | | | | | | | | | 2.2] | [A/G]AGAGGCCAGCCAGTG | NO: |
| | | | | | | | | | | CGGAAAGCAGCAGCAGCAGC | 673 |
| | | | | | | | | | | ATCAC | |
| chr | 195505 | C | G | MUC4 | p.V422 | 0.025 | 0.000 | 6.23 | Inf | GGGGTGGCGTGACCTGTGGA | SEQ |
| 3 | 772 | | | | 7L | 74 | 00 | E− | | TACTGAGGAAAGGCTGGTGA | ID |
| | | | | | | | | 146 | | [C/G]AGGAAGAGGGGTGGC | NO: |
| | | | | | | | | | | GTGACCTGTGGATGCTGAGG | 674 |
| | | | | | | | | | | AAGTG | |
| chr | 195508 | G | C | MUC4 | p.L342 | 0.009 | 0.000 | 2.06 | 51.16 | GCGTGACCGGTGGATGCTGA | SEQ |
| 3 | 178 | | | | 5V | 80 | 19 | E−37 | [26.23- | GGAAGTGCTGGTGACAGGAA | ID |
| | | | | | | | | | 99.79] | [G/C]AGGGGTGGCGTGACC | NO: |
| | | | | | | | | | | TGTGGATGCTGAGGAAGGGC | 675 |
| | | | | | | | | | | TAGTG | |
| chr | 195508 | G | C | MUC4 | p.T341 | 0.016 | 0.000 | 6.58 | 38.6 | CTGAGGAAGTGCTGGTGACA | SEQ |
| 3 | 194 | | | | 91 | 42 | 43 | E−58 | [24.19- | GGAAGAGGGGTGGCGTGACC | ID |
| | | | | | | | | | 61.59] | [T/G]GTGGATGCTGAGGAA | NO: |
| | | | | | | | | | | GGGCTAGTGACAGGAAGAGG | 676 |
| | | | | | | | | | | CATGG | |
| chr | 195512 | T | C | MUC4 | p.S205 | 0.015 | 0.000 | 2.79 | 68.26 | GGAAGAGGCGTGGTGTCACC | SEQ |
| 3 | 294 | | | | 3G | 20 | 23 | E−60 | [37.51- | TGTGGATACTGAGGAAAGGC | ID |
| | | | | | | | | | 124.21] | [T/C]GGTGACAGGAAGAGG | NO: |
| | | | | | | | | | | GGTGTCCTGACCTGTGGATG | 677 |
| | | | | | | | | | | CTGAG | |
| chr | 195512 | C | G | MUC4 | p.Q20 | 0.011 | 0.000 | 1.51 | 32.71 | TGGATACTGAGGAAAGGCTG | SEQ |
| 3 | 316 | | | | 45H | 27 | 35 | E−38 | [19.15- | GTGACAGGAAGAGGGGTGTC | ID |
| | | | | | | | | | 55.88] | [C/G]TGACCTGTGGATGCT | NO: |
| | | | | | | | | | | GAGGAAGTATCGGTGACAGG | 678 |
| | | | | | | | | | | AAGCG | |
| chr | 195512 | G | A | MUC4 | p.P182 | 0.011 | 0.000 | 3.64 | 352 | TCACCTGTGGATGCTGAGGA | SEQ |
| 3 | 981 | | | | 4S | 52 | 03 | E−54 | [85.47- | AGCGTCGGTGACAGGAAGAG | ID |
| | | | | | | | | | 1449.66] | [G/A]GGTGGTGTCACCTGT | NO: |
| | | | | | | | | | | GGATGCTGAGGAAGGGCTGG | 679 |
| | | | | | | | | | | TGACA | |
| chr | 196214 | C | T | RNF16 | p.R164 | 0.023 | 0.000 | 6.42 | 388.53 | GTTCCTCATCACTTTTCAGT | SEQ |
| 3 | 336 | | | 8 | R | 77 | 06 | E− | [180.3- | TGTTCTTCCATCGCTCTTCG | ID |
| | | | | | | | | 132 | 837.21] | [C/T]CTTTTTTCTGCCTGT | NO: |
| | | | | | | | | | | CTTTTTTCCTCTTCTTCCTC | 680 |
| | | | | | | | | | | CTCTG | |
| chr | 196214 | T | C | RNF16 | p.R164 | 0.009 | 0.000 | 1.81 | Inf | TCCTCATCACTTTTCAGTTG | SEQ |
| 3 | 338 | | | 8 | G | 56 | 00 | E−57 | | TTCTTCCATCGCTCTTCGCC | ID |
| | | | | | | | | | | [T/C]TTTTTCTGCCTGTCT | NO: |
| | | | | | | | | | | TTTTTCCTCTTCTTCCTCCT | 681 |
| | | | | | | | | | | CTGCC | |
| chr | 265813 | A | T | ZNF73 | p.F277 | 0.022 | 0.000 | 2.19 | 492.83 | TGAGGATGAGGTAATGATTT | SEQ |
| 4 | | | | 2 | Y | 30 | 05 | E− | [200.2- | TGCCACATTCTTCACATGTG | ID |
| | | | | | | | | 124 | 1213.19] | [A/T]AGGGTTTCTCTTCAG | NO: |
| | | | | | | | | | | CATGAATTCTCTTATGCTTA | 682 |
| | | | | | | | | | | GTAAG | |
| chr | 265825 | T | C | ZNF73 | p.E273 | 0.011 | 0.000 | 2.01 | Inf | AATGATTTTGCCACATTCTT | SEQ |
| 4 | | | | 2 | G | 52 | 00 | E−68 | | CACATGTGAAGGGTTTCTCT | ID |
| | | | | | | | | | | [T/C]CAGCATGAATTCTCT | NO: |
| | | | | | | | | | | TATGCTTAGTAAGGGTTGAG | 683 |
| | | | | | | | | | | GACCT | |
| chr | 265829 | C | T | ZNF73 | p.A272 | 0.018 | 0.000 | 1.83 | Inf | ATTTTGCCACATTCTTCACA | SEQ |
| 4 | | | | 2 | T | 14 | 00 | E− | | TGTGAAGGGTTTCTCTTCAG | ID |
| | | | | | | | | 107 | | [C/T]ATGAATTCTCTTATG | NO: |
| | | | | | | | | | | CTTAGTAAGGGTTGAGGACC | 684 |
| | | | | | | | | | | TATTA | |
| chr | 436337 | G | A | ZNF72 | p.P640 | 0.008 | 0.000 | 4.30 | Inf | TGATGGGGCAAAGGCTTTGC | SEQ |
| 4 | | | | 1 | L | 82 | 00 | E−53 | | CACACTCTTCACATTTGTAA | ID |
| | | | | | | | | | | [G/A]GTTTCTCCCCAGTGT | NO: |
| | | | | | | | | | | AAATTTTCTTCTGTTGATTC | 685 |
| | | | | | | | | | | AGGTC | |
| chr | 436390 | A | G | ZNF72 | p.F622 | 0.005 | 0.000 | 3.07 | 660.68 | TGTAAATTTTCTTCTGTTGA | SEQ |
| 4 | | | | 1 | F | 88 | 01 | E−34 | [89.36- | TTCAGGTCCGTGTACCATAC | ID |
| | | | | | | | | | 4884.86] | [A/G]AAGTCTTTGCCACAC | NO: |
| | | | | | | | | | | TCTTCACATTTGTAAAGTTT | 686 |
| | | | | | | | | | | CTCTC | |
| chr | 437293 | A | G | ZNF72 | p.Y7321 | 0.013 | 0.000 | 1.88 | 103.33 | ATGTGTAGGGTTTCTCTCCA | SEQ |
| 4 | | | | 1 | Y | 73 | 13 | E−67 | [58.4- | GTATGAATTCTCCTATGTAC | ID |
| | | | | | | | | | 182.84] | [A/G]TAAAGGTTTGCGGAC | NO: |
| | | | | | | | | | | TGTCTAAAGGCTTTGCCACA | 687 |
| | | | | | | | | | | TACTT | |
| chr | 676125 | G | C | MFSD7 | p.S434 | 0.007 | 0.004 | 9.52 | 1.71 | GGCGCCGGTATGGGGTGTGG | SEQ |
| 4 | | | | | R | 11 | 18 | E−03 | [1.16- | AAGAAGACCGCCAGGATGCA | ID |
| | | | | | | | | | 2.51] | [G/C]CTGAAGAAGGTGCAC | NO: |
| | | | | | | | | | | AGGCCGGCCATCAGCAGCAG | 688 |
| | | | | | | | | | | AGACA | |
| chr | 138836 | G | A | CRIPAK | p.A24T | 0.006 | 0.000 | 1.22 | 109.46 | GGAGTGCCCGCCTGCTCACA | SEQ |
| 4 | 9 | | | | | 86 | 06 | E−34 | [47.78- | CGTGCCCATGTGGAGTGCCC | ID |
| | | | | | | | | | 250.72] | [G/A]CCTGCTCATGTGCCC | NO: |
| | | | | | | | | | | ATGTGGAGTGCCCGCCTGCT | 689 |
| | | | | | | | | | | CACAC | |
| chr | 138941 | C | T | CRIPAK | p.P373 | 0.006 | 0.000 | 5.30 | 238.42 | GAGTGCCCGCCTGCTCACAC | SEQ |
| 4 | 7 | | | | L | 37 | 03 | E−35 | [72.13- | ACGTGCCCATGTGGAGTGCC | ID |
| | | | | | | | | | 788.02] | [C/T]GCCTGCTCACACGTG | NO: |
| | | | | | | | | | | CCCATGTGGAGTGCCTGCCT | 690 |
| | | | | | | | | | | GCTCA | |
| chr | 180550 | C | T | FGFR3 | p.T338 | 0.007 | 0.003 | 1.52 | 1.9 | CCTTGCACAACGTCACCTTT | SEQ |
| 4 | 2 | | | | T | 35 | 89 | E−03 | [1.31- | GAGGACGCCGGGGAGTACAC | ID |
| | | | | | | | | | 2.75] | [C/T]TGCCTGGCGGGCAAT | NO: |
| | | | | | | | | | | TCTATTGGGTTTTCTCATCA | 691 |
| | | | | | | | | | | CTCTG | |
| chr | 341781 | C | T | RGS12 | p.A149 | 0.010 | 0.006 | 1.19 | 1.52 | ATCGACAGCCAGGCCCAGCT | SEQ |
| 4 | 1 | | | | V | 29 | 78 | E−02 | [1.11- | AGCAGACGACGTCCTCCGCG | ID |
| | | | | | | | | | 2.08] | [C/T]ACCTCACCCAGACAT | NO: |
| | | | | | | | | | | GTTCAAGGAGCAGCAGCTGC | 692 |
| | | | | | | | | | | AGGTA | |
| chr | 351988 | C | T | LRPAP | p.D211 | 0.005 | 0.003 | 4.80 | 1.62 | AGCTCCGTGTGCCTGCTGTG | SEQ |
| 4 | 1 | | | 1 | N | 15 | 19 | E−02 | [1.04- | CAGGACGCTGCCCTTGATGT | ID |
| | | | | | | | | | 2.51] | [C/T]GCTCAGGTCCGAGGG | NO: |
| | | | | | | | | | | GCTAATGACGTTCTCGTGGA | 693 |
| | | | | | | | | | | TTTCT | |
| chr | 700663 | 6 | C | TBC1D | p.E166 | 0.006 | 0.004 | 2.71 | 1.58 | AGCCAAGGAGAGGTGGCGGT | SEQ |
| 4 | 6 | | | 14 | Q | 62 | 20 | E−02 | [1.07- | CCCTTAGCACAGGAGGCTCT | ID |
| | | | | | | | | | 2.33] | [G/C]AAGTGGAGAACGAAG | NO: |
| | | | | | | | | | | GTAGAATGTCTTCTAAAACC | 694 |
| | | | | | | | | | | AGCGG | |
| chr | 135457 | C | G | NKX3-2 | p.A113 | 0.005 | 0.000 | 8.15 | Inf | CCGAGGCTCAAGGATCCCCC | SEQ |
| 4 | 02 | | | | P | 15 | 00 | E−28 | | CGCAAGGCCGGCCCCGCTGG | ID |
| | | | | | | | | | | [C/G]CCCCCGCGCGTCCGC | NO: |
| | | | | | | | | | | GCAGCGCCGCCTGCTCTCGT | 695 |
| | | | | | | | | | | TCTCC | |
| chr | 165042 | T | G | LDB2 | p.N36 | 0.020 | 0.000 | 1.23 | 2373.68 | CTGGTGCCGATCATCTTATT | SEQ |
| 4 | 91 | | | | 6T | 83 | 01 | E− | [330.46- | GGGAAGCCTGGGGTGGGGGG | ID |
| | | | | | | | | 122 | 17050.06] | [T/G]TTTCTGATTTGGTCT | NO: |
| | | | | | | | | | | CTTGAGTGGCGGGAGGTTTA | 696 |
| | | | | | | | | | | CTGTT | |
| chr | 577972 | A | G | REST | p.I747 | 0.010 | 0.000 | 4.04 | Inf | CTCCTCCCATGGAGGTGGTC | SEQ |
| 4 | 65 | | | | M | 05 | 00 | E−60 | | CAGAAGGAGCCTGTTCAGAT | ID |
| | | | | | | | | | | [A/G]GAGCTGTCTCCTCCC | NO: |
| | | | | | | | | | | ATGGAGGTGGTCCAGAAGGA | 697 |
| | | | | | | | | | | ACCTG | |
| chr | 629360 | C | A | LPHN3 | p.N12 | 0.006 | 0.004 | 3.32 | 1.65 | GTGAACAGAACAGGAATCTG | SEQ |
| 4 | 92 | | | | 92K | 831 | 163 | E−02 | [1.01- | ATGAACAAGCTGGTGAATAA | ID |
| | | | | | | | | | 2.55] | [C/A]CTTGGCAGTGGAAGG | NO: |
| | | | | | | | | | | GAAGATGATGCCATTGTCCT | 698 |
| | | | | | | | | | | GGATG | |
| chr | 694337 | T | A | UGT2B | p.D147 | 0.009 | 0.006 | 1.90 | 1.48 | CAGCTCACCACAGGGATTAA | SEQ |
| 4 | 63 | | | 17 | V | 80 | 63 | E−02 | [1.08- | CGGCATCTGCCAGAAGGACA | ID |
| | | | | | | | | | 2.04] | [T/A]CAAATTTTGACTCTT | NO: |
| | | | | | | | | | | GTAGTTTTCTCATAAGTTTC | 699 |
| | | | | | | | | | | TTGTT | |
| chr | 698747 | T | C | UGT2B | p.T134 | 0.010 | 0.000 | 4.99 | 27.9 | AACAATGGAATGCCCACCAT | SEQ |
| 4 | 38 | | | 10 | A | 78 | 39 | E−40 | [18.31- | AGGGATCCCATGGTAGATTG | ID |
| | | | | | | | | | 42.53] | [T/C]CTCGTAGATGCCATT | NO: |
| | | | | | | | | | | GGCTCCACCATGAGTTATAA | 700 |
| | | | | | | | | | | AAGCT | |
| chr | 698747 | G | A | UGT2B | p.Y132 | 0.011 | 0.000 | 1.22 | 26.17 | ATGGAATGCCCACCATAGGG | SEQ |
| 4 | 42 | | | 10 | Y | 76 | 45 | E−42 | [17.59- | ATCCCATGGTAGATTGTCTC | ID |
| | | | | | | | | | 38.94] | [G/A]TAGATGCCATTGGCT | NO: |
| | | | | | | | | | | CCACCATGAGTTATAAAAGC | 701 |
| | | | | | | | | | | TCTGG | |
| chr | 712325 | C | A | SIMR3A | p.S79Y | 0.007 | 0.000 | 8.43 | Inf | CCCCTTTCTCCACCCTATGG | SEQ |
| 4 | 42 | | | | | 60 | 00 | E−46 | | TCCAGGGAGAATCCCACCAT | ID |
| | | | | | | | | | | [C/A]CCCTCCTCCACCCTA | NO: |
| | | | | | | | | | | TGGTCCAGGGAGAATTCAAT | 702 |
| | | | | | | | | | | CACAC | |
| chr | 723385 | A | G | SLC4A4 | p.K602 | 0.007 | 0.003 | 7.37 | 2.04 | TCCTCTCTGATTAGCTTCAT | SEQ |
| 4 | 89 | | | | R | 11 | 50 | E−04 | [1.4- | CTTTATCTATGATGCTTTCA | ID |
| | | | | | | | | | 2.98] | [A/G]GAAGATGATCAAGCT | NO: |
| | | | | | | | | | | TGCAGATTACTACCCCATCA | 703 |
| | | | | | | | | | | ACTCC | |
| chr | 772045 | C | T | FAM47 | p.R283 | 0.010 | 0.006 | 1.10 | 1.54 | TTAGTTCCTTGAGAATATGT | SEQ |
| 4 | 70 | | | E | C | 29 | 72 | E−02 | [1.12- | ATATCGGGAAGGAATGTAAA | ID |
| | | | | | | | | | 2.12] | [C/T]GTGCATGTAATAAGA | NO: |
| | | | | | | | | | | CTCCTATAAAACGAACTCAA | 704 |
| | | | | | | | | | | GCATA | |
| chr | 797921 | G | A | BMP2K | p.Q48 | 0.012 | 0.000 | 1.06 | 1376.85 | AACAGCAACAGCAGCAGCAG | SEQ |
| 4 | 48 | | | | 1Q | 75 | 01 | E−73 | [190.3- | CAACAGCAACAGCAGCAGCA | ID |
| | | | | | | | | | 9961.91] | [G/A]CAGCAGCAGCAGCAG | NO: |
| | | | | | | | | | | CACCACCACCACCACCACCA | 705 |
| | | | | | | | | | | CCACC | |
| chr | 819672 | C | T | BMP3 | p.T222 | 0.000 | 0.000 | 1.00 | 0.79 | GCCAAAGAAAATGAAGAGTT | SEQ |
| 4 | 40 | | | | M | 49 | 62 | E+00 | [0.19- | CCTCATAGGATTTAACATTA | ID |
| | | | | | | | | | 3.22] | [C/T]GTCCAAGGGACGCCA | NO: |
| | | | | | | | | | | GCTGCCAAAGAGGAGGTTAC | 706 |
| | | | | | | | | | | CTTTT | |
| chr | 876662 | A | G | PTPN1 | p.H865 | 0.009 | 0.005 | 1.49 | 1.77 | AAGATATGCCAGTACCTGCT | SEQ |
| 4 | 25 | | | 3 | R | 31 | 28 | E−03 | [1.27- | GCACCTCTGCTCTTACCAGC | ID |
| | | | | | | | | | 2.46] | [A/G]TAAGTTCCAGCTACA | NO: |
| | | | | | | | | | | GATGAGAGCAAGACAGAGCA | 707 |
| | | | | | | | | | | ACCAA | |
| chr | 876722 | G | T | PTPN1 | p.D104 | 0.008 | 0.005 | 9.90 | 1.61 | GAGTTTAAATAGAAGTCCTG | SEQ |
| 4 | 35 | | | 3 | 2Y | 82 | 50 | E−03 | [1.15- | AAAGGAGGAAACATGAATCA | ID |
| | | | | | | | | | 2.26] | [G/T]ACTCCTCATCCATTG | NO: |
| | | | | | | | | | | AAGACCCTGGGCAAGCATAT | 708 |
| | | | | | | | | | | GTTCT | |
| chr | 877491 | G | A | SLC10A | p.H249 | 0.028 | 0.000 | 2.75 | Inf | AGTTTATGGATAGTTTAACT | SEQ |
| 4 | 62 | | | 6 | Y | 68 | 00 | E− | | ATACCTTTGCCAAGACTGGT | ID |
| | | | | | | | | 171 | | [G/A]GGTAAAAAGTGCCAG | NO: |
| | | | | | | | | | | CAGAAAACCCGTGACATGGC | 709 |
| | | | | | | | | | | CAATC | |
| chr | 885375 | C | T | DSPP | p.S124 | 0.010 | 0.000 | 3.86 | Inf | AAAGCAGCGACAGCAGTGAC | SEQ |
| 4 | 52 | | | | 6S | 78 | 00 | E−52 | | AGCAGCGATAGCAGTGACAG | ID |
| | | | | | | | | | | [C/T]AGCAACAGCAGTGAC | NO: |
| | | | | | | | | | | AGCAGCGACAGCAGTGATAG | 710 |
| | | | | | | | | | | CAGTG | |
| chr | 885375 | C | T | DSPP | p.N12 | 0.011 | 0.000 | 5.36 | 54.14 | GCGACAGCAGTGACAGCAGC | SEQ |
| 4 | 58 | | | | 48N | 52 | 22 | E−43 | [28.06- | GATAGCAGTGACAGCAGCAA | ID |
| | | | | | | | | | 104.46] | [C/T]AGCAGTGACAGCAGC | NO: |
| | | | | | | | | | | GACAGCAGTGATAGCAGTGA | 711 |
| | | | | | | | | | | CAGCA | |
| chr | 113303 | A | 6 | ALPK1 | p.Q67 | 0.011 | 0.007 | 2.92 | 1.61 | GCAAAGGAAATGAAGTGGCC | SEQ |
| 4 | 632 | | | | R | 76 | 35 | E−03 | [1.2- | CTTCGTGCCTGAAAAGTGGC | ID |
| | | | | | | | | | 2.15] | [A/G]GTACAAACAAGCCGT | NO: |
| | | | | | | | | | | GGGCCCAGAGGACAAAACAA | 712 |
| | | | | | | | | | | ACCTG | |
| chr | 115997 | T | C | NDST4 | p.I283 | 0.012 | 0.009 | 3.24 | 1.37 | AGCCTCTTCCCTGACAAGAA | SEQ |
| 4 | 346 | | | | V | 75 | 37 | E−02 | [1.03- | GGAGATGGCATCTATGAAGA | ID |
| | | | | | | | | | 1.81] | [T/C]GAGCTTGTGCAGCCA | NO: |
| | | | | | | | | | | AAAGTTCAAGTTGTTGCCAA | 713 |
| | | | | | | | | | | AAAGT | |
| chr | 125592 | G | A | ANKRD | p.A521 | 0.011 | 0.008 | 3.46 | 1.39 | CATTATCTAATAATGTCCGA | SEQ |
| 4 | 869 | | | 50 | A | 27 | 16 | E−02 | [1.03- | ATGGAATCCTCTCTTTCTAA | ID |
| | | | | | | | | | 1.87] | [G/A]GCTTGTCGAACTATG | NO: |
| | | | | | | | | | | CATGATGTGCGATCGTCTTC | 714 |
| | | | | | | | | | | ACTGT | |
| chr | 153690 | G | A | TIGD4 | p.T477 | 0.005 | 0.003 | 1.38 | 1.74 | ATCTTGACTTCTGAGAAATT | SEQ |
| 4 | 727 | | | | I | 88 | 39 | E−02 | [1.15- | TTTTCAGAGTATCTAAAGCA | ID |
| | | | | | | | | | 2.63] | [G/A]TTATTGCCTCAGATT | NO: |
| | | | | | | | | | | TTGATGGTAAAGGGAGTTCA | 715 |
| | | | | | | | | | | GTTCC | |
| chr | 165962 | A | T | TRIM6 | p.E422 | 0.006 | 0.003 | 2.13 | 1.64[ | TAGTAAAACCCAGTAAAATT | SEQ |
| 4 | 490 | | | 0 | D | 37 | 89 | E−02 | 1.11- | GGTATTTTTCTGGACTATGA | ID |
| | | | | | | | | | 2.45] | [A/T]TTGGGTGATCTTTCC | NO: |
| | | | | | | | | | | TTTTATAATATGAATGATAG | 716 |
| | | | | | | | | | | GTCTA | |
| chr | 166300 | T | C | CPE | p.F51L | 0.005 | 0.000 | 4.59 | Inf | GAGGCGGCGCCGGCGGCTGC | SEQ |
| 4 | 524 | | | | | 15 | 00 | E−30 | | AGCAAGAGGACGGCATCTCC | ID |
| | | | | | | | | | | [T/C]TCGAGTACCACCGCT | NO: |
| | | | | | | | | | | ACCCCGAGCTGCGCGAGGCG | 717 |
| | | | | | | | | | | CTCGT | |
| chr | 167656 | A | T | SPOCK | p.X317 | 0.003 | not | 5.64 | Inf | TTAGAAATGTAGAATTTATT | SEQ |
| 4 | 074 | | | 3 | R | 93 | found | E−08 | | GATTTCAACTGTCATCAATC | ID |
| | | | | | | | | | | [A/T]AATGTATACATCATG | NO: |
| | | | | | | | | | | GTCATCACCACCATCATCAT | 718 |
| | | | | | | | | | | CATCC | |
| chr | 170671 | C | G | C4orf2 | p.G82 | 0.005 | 0.003 | 4.94 | 1.6 | TTCTTCGTTTTATGTTTTCC | SEQ |
| 4 | 841 | | | 7 | R | 15 | 23 | E−02 | [1.03- | AGCAAGGATATCATAAGGAC | ID |
| | | | | | | | | | 2.48] | [C/G]AACTAATTGAAGTCC | NO: |
| | | | | | | | | | | AAGGCTTGCAGAAAGTGAAT | 719 |
| | | | | | | | | | | CTATA | |
| chr | 175898 | T | C | ADAM | p.W73 | 0.006 | 0.000 | 1.43 | 33.53 | TCAGCGTCGACCTCATGAGT | SEQ |
| 4 | 879 | | | 29 | 5R | 37 | 19 | E−25 | [18.85- | TACCTCCCCAGAGTCAACCT | ID |
| | | | | | | | | | 59.63] | [T/C]GGGTGATGCCTTCCC | NO: |
| | | | | | | | | | | AGAGTCAACCTCCTGTGACG | 720 |
| | | | | | | | | | | CCTTC | |
| chr | 175898 | C | T | ADAM | p.S757 | 0.006 | 0.000 | 7.49 | 12.91 | CTGTGACGCCTTCCCAGAGT | SEQ |
| 4 | 947 | | | 29 | S | 62 | 52 | E−19 | [8.16- | CATCCTCAGGTGATGCCTTC | ID |
| | | | | | | | | | 20.42] | [C/T]CAGAGTCAACCTCCT | NO: |
| | | | | | | | | | | GTGACACCCTCCCAGAGTCA | 721 |
| | | | | | | | | | | ACCTC | |
| chr | 177083 | G | A | WDR1 | p.D933 | 0.006 | 0.004 | 1.23 | 1.7 | GCACAAAGTCAGTAAAGAAC | SEQ |
| 4 | 272 | | | 7 | N | 86 | 05 | E−02 | [1.16- | TGGCAGAATGGTATTTTCAA | ID |
| | | | | | | | | | 2.49] | [G/A]ATGGTCGAGCAGTAC | NO: |
| | | | | | | | | | | TAGCCGCATGTTGCCATCTT | 722 |
| | | | | | | | | | | GCCAT | |
| chr | 191718 | C | G | LRRC1 | p.A22 | 0.008 | 0.000 | 8.74 | Inf | TTCATTTCTGCAGAAGCTCT | SEQ |
| 5 | | | | 4B | G | 82 | 00 | E−53 | | GGTGTCCCACCCCCAGGTGG | ID |
| | | | | | | | | | | [C/G]CCGGCAGAGCCTGGA | NO: |
| | | | | | | | | | | CAGCGTGGCCCACAACCTCT | 723 |
| | | | | | | | | | | ACCCA | |
| chr | 891400 | T | C | BRD9 | p. K39R | 0.000 | 0.001 | 5.63 | 10 | CCGTGTCACAGTGCTCCCTC | SEQ |
| 5 | | | | | | 90 | 70 | E−09 | [5- | TCTCGCTTCCGCTTCTTCTC | ID |
| | | | | | | | | | 21] | [T/C]TCCTGGGCGGCAGAG | NO: |
| | | | | | | | | | | TCAAGGGAGTGAGAAAGGCA | 724 |
| | | | | | | | | | | GGAGT | |
| chr | 739660 | T | G | ADCY2 | p.F65V | 0.008 | 0.000 | 3.47 | Inf | GCTCATCGTCATGGGCTCCT | SEQ |
| 5 | 2 | | | | | 58 | 00 | E−49 | | GCCTCGCCCTGCTCGCCGTC | ID |
| | | | | | | | | | | [T/G]TCTTCGCGCTCGGGC | NO: |
| | | | | | | | | | | TGGTGAGTGGCCTCCCCGCG | 725 |
| | | | | | | | | | | GGTCC | |
| chr | 369854 | G | A | NIPBL | p.G720 | 0.005 | 0.000 | 9.80 | 628.63 | GTGAAAGCCGGCCTGAGACT | SEQ |
| 5 | 42 | | | | G | 64 | 01 | E−33 | [84.88- | CCAAAACAAAAGAGTGATGG | ID |
| | | | | | | | | | 4655.98] | [G/A]CATCCTGAAACCCCA | NO: |
| | | | | | | | | | | AAACAGAAGGGTGATGGAAG | 726 |
| | | | | | | | | | | GCCTG | |
| chr | 523473 | A | C | ITGA2 | p.T252 | 0.008 | 0.005 | 2.65 | 1.51 | CATCCCAGACATCCCAATAT | SEQ |
| 5 | 66 | | | | T | 58 | 69 | E−02 | [1.07- | GGTGGGGACCTCACAAACAC | ID |
| | | | | | | | | | 2.13] | [A/C]TTCGGAGCAATTCAA | NO: |
| | | | | | | | | | | TATGCAAGGTAAGTTTTGGT | 727 |
| | | | | | | | | | | GCTAA | |
| chr | 550836 | G | T | DDX4 | p.A199 | 0.005 | 0.000 | 2.43 | 603.79 | GCAACTTAACTTCTAGGCGG | SEQ |
| 5 | 98 | | | | S | 39 | 01 | E−31 | [81.37- | CTTTTCTCCTACCAATTTTG | ID |
| | | | | | | | | | 4480.44] | [G/T]CTCATATGATGCATG | NO: |
| | | | | | | | | | | ATGGAATAACTGCCAGTCGT | 728 |
| | | | | | | | | | | TTTAA | |
| chr | 708062 | C | A | BDP1 | p.G110 | 0.008 | 0.000 | 1.09 | Inf | TGGAAGAAACTGAAAGAGAA | SEQ |
| 5 | 31 | | | | 4G | 09 | 00 | E−48 | | ATATCCCCACAGGAAAATGG | ID |
| | | | | | | | | | | [C/A]CTAGAGGAGGTTAAG | NO: |
| | | | | | | | | | | CCTCTAGGTGAAATGCAAAC | 729 |
| | | | | | | | | | | AGATT | |
| chr | 715167 | G | C | MRPS2 | p.Q39 | 0.005 | 0.003 | 3.05 | 1.68 | GCTTTCTGAGCCTGGTACTC | SEQ |
| 5 | 95 | | | 7 | 6E | 15 | 06 | E−02 | [1.08- | CTGCTTCGCTTGCTCCCTCT | ID |
| | | | | | | | | | 2.62] | [G/C]TTGCTGTTCTCTCTG | NO: |
| | | | | | | | | | | GATCAACTGTACAAGGTCTA | 730 |
| | | | | | | | | | | GATGC | |
| chr | 762495 | G | A | CRHBP | p.P53P | 0.010 | 0.007 | 4.12 | 1.4 | TCAGCGCCAACCTGAAGCGG | SEQ |
| 5 | 03 | | | | | 29 | 39 | E−02 | [1.02- | GAGCTGGCTGGGGAGCAGCC | ID |
| | | | | | | | | | 1.91] | [G/A]TACCGCCGCGCTCTG | NO: |
| | | | | | | | | | | CGTGAGTCGAGGCTGCCCGG | 731 |
| | | | | | | | | | | CTCGC | |
| chr | 762498 | A | AC | CRH8P | NM_0 | 0.006 | not | 7.44 | 223.6 | GCTGCAGCCCGGGACTTATT | SEQ |
| 5 | 52 | | | | 01882: | 87 | found | E−12 | [46.4- | GCCCCATGCCCTCCTCCCCC | ID |
| | | | | | exon3: | | | | 1077.6] | [A/AC]GGGTGCCTGGACAT | NO: |
| | | | | | c.176- | | | | | GCTGAGCCTCCAGGGCCAGT | 732 |
| | | | | | 2- > C | | | | | TCACCT | |
| chr | 767606 | C | T | WDR4 | p.G61 | 0.005 | 0.003 | 3.47 | 1.62 | ACACACCTGGGCATTCCACA | SEQ |
| 5 | 20 | | | 1 | D | 88 | 65 | E−02 | [1.07- | CAACTACAATTCCATCATCA | ID |
| | | | | | | | | | 2.44] | [C/T]CAGCAGATGCAAATC | NO: |
| | | | | | | | | | | TGGTTAGGGAGAAAGGGTCA | 733 |
| | | | | | | | | | | AGAAA | |
| chr | 798548 | A | G | ANKRD | p.V338 | 0.005 | 0.003 | 2.71 | 1.65 | AAATATTGTCTGGTTAGAAT | SEQ |
| 5 | 26 | | | 34B | A | 39 | 28 | E−02 | [1.07- | CTGGGTCCTGGTCAACAGGG | ID |
| | | | | | | | | | 2.54] | [A/G]CTTCAATGCATTGCT | NO: |
| | | | | | | | | | | GATTTCCTTCTGAAAGATAA | 734 |
| | | | | | | | | | | GATTG | |
| chr | 899698 | A | G | GPR98 | p.I164 | 0.010 | 0.006 | 2.82 | 1.44 | GCTTAGTGCCTCTGGATATT | SEQ |
| 5 | 80 | | | | 7V | 05 | 98 | E−02 | [1.05- | TATATTTTTAGGTTCTGAAT | ID |
| | | | | | | | | | 1.98] | [A/G]TATATGTTCTTGATG | NO: |
| | | | | | | | | | | ATGATATTCCTGAACTTAAT | 735 |
| | | | | | | | | | | GAGTA | |
| chr | 899795 | G | A | GPR98 | p.D194 | 0.009 | 0.005 | 6.02 | 1.64 | TATCACTGTGGAGATATTGC | SEQ |
| 5 | 68 | | | | 4N | 31 | 69 | E−03 | [1.18- | CTGACGAAGACCCAGAACTG | ID |
| | | | | | | | | | 2.28] | [G/A]ATAAGGCATTCTCTG | NO: |
| | | | | | | | | | | TGTCAGTCCTCAGTGTTTCC | 736 |
| | | | | | | | | | | AGTGG | |
| chr | 929210 | C | T | NR2F1 | p.H97 | 0.029 | 0.000 | 1.83 | 838.44 | TCGAGTGCGTGGTGTGCGGG | SEQ |
| 5 | 20 | | | | H | 66 | 04 | E− | [309.51- | GACAAGTCGAGCGGCAAGCA | ID |
| | | | | | | | | 169 | 2271.28] | [C/T]TACGGCCAATTCACC | NO: |
| | | | | | | | | | | TGCGAGGGCTGCAAAAGTTT | 737 |
| | | | | | | | | | | CTTCA | |
| chr | 1.34002 | G | C | SEC24 | p.A223 | 0.013 | 0.000 | 5.18 | 1523.15 | TCATGGGCCCCCTCCAGCTG | SEQ |
| 5 | 614 | | | A | P | 48 | 01 | E−79 | [210.73- | GAGGCCCACCCCCAGTGAGG | ID |
| | | | | | | | | | 11009.22] | [G/C]CCCTCACGCCCCTGA | NO: |
| | | | | | | | | | | CATCATCATATAGAGATGTA | 738 |
| | | | | | | | | | | CCCCA | |
| chr | 137621 | C | T | CDC25 | p.R388 | 0.006 | 0.003 | 2.71 | 1.63 | TCATGGGCTCATGTCCTTCA | SEQ |
| 5 | 421 | | | C | Q | 13 | 76 | E−02 | [1.09- | CCAGAAGGGCAATCTGCTCC | ID |
| | | | | | | | | | 2.45] | [C/T]GCAGCTGCCGCTCCC | NO: |
| | | | | | | | | | | CTTCCTGCACTTTGCTCTGG | 739 |
| | | | | | | | | | | CTTCG | |
| chr | 140209 | G | A | PCDHA | p.R498 | 0.006 | 0.004 | 3.84 | 1.55 | AGGAGAACGCGCTGGTGTCC | SEQ |
| 5 | 170 | | | 6 | R | 62 | 29 | E−02 | [1.05- | TACTCGCTGGTGGAGCGGCG | ID |
| | | | | | | | | | 2.28] | [G/A]GTGGGCGAGCGCGCG | NO: |
| | | | | | | | | | | TTGTCGAGCTACATTTCGGT | 740 |
| | | | | | | | | | | GCACG | |
| chr | 140559 | T | C | PCDHB | p.L576 | 0.007 | 0.003 | 8.04 | 2.02 | CTGTACCCGCTGCAGAATGG | SEQ |
| 5 | 342 | | | 8 | P | 11 | 53 | E−04 | [1.38- | CTCCGCGCCCTGCACCGAGC | ID |
| | | | | | | | | | 2.95] | [T/C]GGTGCCCCGGGCGGC | NO: |
| | | | | | | | | | | CGAGCCGGGCTACCTGGTGA | 741 |
| | | | | | | | | | | CCAAG | |
| chr | 141336 | G | A | PCDH1 | p.T261 | 0.009 | 0.005 | 7.37 | 1.6 | GCCTTGGTCAGGGTCTGTGG | SEQ |
| 5 | 635 | | | 2 | M | 56 | 98 | E−03 | [1.16- | CGGTCAGTTTTATGAGAAGC | ID |
| | | | | | | | | | 2.22] | [G/A]TACCAGGTGCAGCAT | NO: |
| | | | | | | | | | | CTTCTTGGATTTCCAGTGCC | 742 |
| | | | | | | | | | | AGTGA | |
| chr | 141694 | G | T | SPRY4 | p.S218 | 0.014 | 0.001 | 2.15 | 11.5 | GCAGTTGGAGCGGGAGCAGG | SEQ |
| 5 | 021 | | | | Y | 31 | 26 | E−28 | [7.94- | AGCAGGGGTGGTCAGCGCAG | ID |
| | | | | | | | | | 16.41] | [G/T]AGCCCTCATCGTCCT | NO: |
| | | | | | | | | | | CATTCGTGCAGTGGTAGAAG | 743 |
| | | | | | | | | | | ATGCC | |
| chr | 148384 | T | A | SH3TC | p.D122 | 0.007 | 0.004 | 2.62 | 1.84 | GACCGCTGCTGCCAGGGCCA | SEQ |
| 5 | 455 | | | 2 | 9V | 35 | 02 | E−03 | [1.27- | GAAGGAAGTACTCAGTGGCA | ID |
| | | | | | | | | | 2.66] | [T/A]CATGGGCATCCTAAC | NO: |
| | | | | | | | | | | CCCGTGGTATGGGGGCAAAG | 744 |
| | | | | | | | | | | AAGAG | |
| chr | 149276 | T | G | PDE6A | 0.049 | 0.019 | 0.001 | 8.09 | 11.32 | ATTTATTAATTTCGTATTTA | SEQ |
| 5 | 063 | | | | 29 | 52 | 76 | E−37 | [8.17- | TCTGCATCTGGCAGCTCCGC | ID |
| | | | | | | | | | 15.55] | [T/G]TGCTGTATAAGGAAT | NO: |
| | | | | | | | | | | AGAGTCAGGTGATTAGGAAA | 745 |
| | | | | | | | | | | CATGA | |
| chr | 149301 | G | A | PDE6A | p.P293 | 0.007 | 0.004 | 3.83 | 1.5[ | CCTGGGACCAGAGTAAGGTG | SEQ |
| 5 | 253 | | | | L | 11 | 75 | E−02 | 1.03- | GAACTTCACCCATCAGAACC | ID |
| | | | | | | | | | 2.18] | [G/A]GCCACACATCAAAAA | NO: |
| | | | | | | | | | | ATTCCTAGGAATGAGAAAAA | 746 |
| | | | | | | | | | | CAATA | |
| chr | 149512 | C | T | PDGFR | p.V316 | 0.006 | 0.004 | 1.88 | 1.64 | TCAGCAAATTGTAGTGTGCC | SEQ |
| 5 | 494 | | | B | M | 86 | 19 | E−02 | [1.11- | CACCTCTCCCAGGAGCCGCA | ID |
| | | | | | | | | | 2.43] | [C/T]GTAGCCGCTCTCTGC | NO: |
| | | | | | | | | | | AAGGGGTGACCGTCAGGGGC | 747 |
| | | | | | | | | | | GGGGC | |
| chr | 150905 | G | T | FAT2 | p.P347 | 0.006 | 0.000 | 4.42 | Inf | CCTCCTGCTTAGGCCCTCAG | SEQ |
| 5 | 399 | | | | 9Q | 13 | 00 | E−37 | | CAGTCACCAGCCATCCATCC | ID |
| | | | | | | | | | | [G/T]GGGTCACTCGGAAGG | NO: |
| | | | | | | | | | | CAGAGCCGTTGTTCCCCTTG | 748 |
| | | | | | | | | | | GTGAT | |
| chr | 167689 | C | A | TENM2 | p.R257 | 0.005 | 0.003 | 2.93 | 1.71 | CATCATTGGCAAAGGCATCA | SEQ |
| 5 | 228 | | | | 1R | 15 | 02 | E−02 | [1.1- | TGTTTGCCATCAAAGAAGGG | ID |
| | | | | | | | | | 2.66] | [C/A]GGGTGACCACGGGCG | NO: |
| | | | | | | | | | | TGTCCAGCATCGCCAGCGAA | 749 |
| | | | | | | | | | | GATAG | |
| chr | 167881 | A | T | WWC1 | p.E862 | 0.011 | 0.000 | 5.03 | Inf | GAGAATGAGGCAGTAGCCGA | SEQ |
| 5 | 032 | | | | V | 76 | 00 | E−70 | | GGAAGAGGAGGAGGAGGTGG | ID |
| | | | | | | | | | | [A/T]GGAGGAGGAGGGAGA | NO: |
| | | | | | | | | | | AGAGGATGTTTTCACCGAGA | 750 |
| | | | | | | | | | | AAGCC | |
| chr | 168112 | G | A | SLIT3 | p.A118 | 0.005 | 0.002 | 1.12 | 2.16 | AAGGGCAGGGCAGGGCGGGA | SEQ |
| 5 | 707 | | | | 0A | 64 | 62 | E−03 | [1.41- | CACACCTGCAGGGAGATGTT | ID |
| | | | | | | | | | 3.31] | [G/A]GCCTGGGGTCGGACC | NO: |
| | | | | | | | | | | TTGGCGGAGGCCAGTTCCAC | 751 |
| | | | | | | | | | | GTAGG | |
| chr | 171661 | T | C | UBTD2 | p.489 | 0.009 | 0.006 | 3.47 | 1.45 | CATGTGGTAATGTTATGTTT | SEQ |
| 5 | 166 | | | | A | 07 | 28 | E−02 | [1.04- | GCACCATCAATGATTGCTTG | ID |
| | | | | | | | | | 2.02] | [T/C]GCCAGTTCATGATCA | NO: |
| | | | | | | | | | | TTGCTCTCAAAAGCATGTGC | 752 |
| | | | | | | | | | | AGCAG | |
| chr | 178139 | C | T | ZNF35 | p.E498 | 0.020 | 0.002 | 8.40 | 8.69 | GATTACTAAGTGATGAGTTA | SEQ |
| 5 | 385 | | | 4A | E | 34 | 38 | E−44 | [6.78- | CACCTGAATGTTTTCCCACA | ID |
| | | | | | | | | | 11.14] | [C/T]TCGTTACATTTATAG | NO: |
| | | | | | | | | | | GGTCTTTCTCCAGTATGCAT | 753 |
| | | | | | | | | | | TCTCT | |
| chr | 178139 | T | C | ZNF35 | p.K495 | 0.020 | 0.002 | 4.38 | 7.3 | GTGATGAGTTACACCTGAAT | SEQ |
| 5 | 394 | | | 4A | K | 34 | 84 | E−39 | [5.72- | GTTTTCCCACACTCGTTACA | ID |
| | | | | | | | | | 9.32] | [T/C]TTATAGGGTCTTTCT | NO: |
| | | | | | | | | | | CCAGTATGCATTCTCTGATG | 754 |
| | | | | | | | | | | TTGAA | |
| chr | 179192 | A | G | MAML | p.T110 | 0.010 | 0.007 | 4.35 | 1.4 | AAGTCATTCTTTTCAATGTT | SEQ |
| 5 | 341 | | | 1 | T | 54 | 57 | E−02 | [1.03- | TTTCAGCATCTTCATGATAC | ID |
| | | | | | | | | | 1.9] | [A/G]GTTAAGAGGAATCTT | NO: |
| | | | | | | | | | | GACAGCGCCACTTCCCCTCA | 755 |
| | | | | | | | | | | GAATG | |
| chr | 179192 | C | T | MAML | p.Y130 | 0.010 | 0.007 | 4.35 | 1.4 | CGCCACTTCCCCTCAGAATG | SEQ |
| 5 | 401 | | | 1 | Y | 54 | 56 | E−02 | [1.03- | GCGATCAACAGAATGGCTA | ID |
| | | | | | | | | | 1.9] | [C/T]GGGGACCTCTTTCCT | NO: |
| | | | | | | | | | | GGGCATAAGAAGACTCGCCG | 756 |
| | | | | | | | | | | GGAGG | |
| chr | 117684 | C | T | ADTRP | p.T96T | 0.005 | 0.002 | 6.64 | 2.12 | CACATTTCTGTTAGATTATG | SEQ |
| 6 | 82 | | | | | 53 | 61 | E−03 | [1.22- | TACACATCTTTGAAACTTAC | ID |
| | | | | | | | | | 3.46] | [C/T]GTGGATACAGGAAAA | NO: |
| | | | | | | | | | | GCCAGAGTGGTGAAAAGCAG | 757 |
| | | | | | | | | | | GTCTC | |
| chr | 260322 | C | T | HIST1H | p.K24K | 0.007 | 0.000 | 8.79 | Inf | TTTTCACGCCGCCGGTAGCC | SEQ |
| 6 | 17 | | | 3B | | 11 | 00 | E−43 | | GGCGCGCTCTTGCGAGCAGC | ID |
| | | | | | | | | | | [C/T]TTGGTAGCCAGCTGC | NO: |
| | | | | | | | | | | TTGCGTGGCGCTTTACCGCC | 758 |
| | | | | | | | | | | GGTGG | |
| chr | 294087 | T | G | OR10C | p.M31 | 0.009 | 0.005 | 7.68 | 1.63 | AAAGCTGCCCTAAAGAGAAC | SEQ |
| 5 | 21 | | | 1 | 0R | 07 | 59 | E−03 | [1.17- | CATCCAGAAAACGGTGCCTA | ID |
| | | | | | | | | | 2.27] | [T/G]GGAGATTTGAAAAGG | NO: |
| | | | | | | | | | | GGGCGATAGTGACTTCTGTG | 759 |
| | | | | | | | | | | CAGTG | |
| chr | 300389 | C | T | RNF39 | p.L337 | 0.005 | 0.003 | 3.63 | 1.59 | GTACAATGCGGAGCGGAGCA | SEQ |
| 6 | 42 | | | | L | 88 | 72 | E−02 | [1.04- | CGAGGGTCGCAGGTGCAGAA | ID |
| | | | | | | | | | 2.41] | [C/T]AGCGGGAAGATGCGC | NO: |
| | | | | | | | | | | TCCCCCAGGGGGCCAGGCGC | 760 |
| | | | | | | | | | | CTGGA | |
| chr | 306732 | G | A | MDC1 | p.A122 | 0.011 | 0.000 | 4.12 | 264.73 | AGGGGTCTTGACAGAGGATC | SEQ |
| 6 | 80 | | | | 7V | 76 | 04 | E−64 | [105.33- | TATTTTTTCTTCCCCTAGTA | ID |
| | | | | | | | | | 665.33] | [G/A]CCTGAGAGGTGGGTT | NO: |
| | | | | | | | | | | CAGAGGTGACAGGTCGGTCG | 761 |
| | | | | | | | | | | GTGGA | |
| chr | 309171 | G | A | DPCR1 | p.G290 | 0.020 | 0.000 | 2.77 | Inf | GAGCTCACACAATCTCTAGC | SEQ |
| 6 | 10 | | | | E | 59 | 00 | E− | | AGAGCCTACAGAACATGGAG | ID |
| | | | | | | | | 100 | | [G/A]AAGGACAGCCAATGA | NO: |
| | | | | | | | | | | GAACAACACACCATCCCCAG | 762 |
| | | | | | | | | | | CAGAG | |
| chr | 309174 | T | C | DPCR1 | p.T392 | 0.006 | 0.000 | 2.15 | 78.33 | AGCCTACAGAACATGGAGAA | SEQ |
| 6 | 17 | | | | T | 37 | 08 | E−25 | [27.32- | AGGACAGCCAATGAGAACAC | ID |
| | | | | | | | | | 224.54] | [T/C]ACACCATCCCCAGCA | NO: |
| | | | | | | | | | | GAGCCTACAGAACATGGAGA | 763 |
| | | | | | | | | | | AAGGA | |
| chr | 309178 | A | G | DPCR1 | p.E539 | 0.012 | 0.000 | 4.79 | Inf | ACCCCACTGGCCAATGAGAA | SEQ |
| 6 | 57 | | | | G | 25 | 00 | E−60 | | CACCACACCATCCCCAGCAG | ID |
| | | | | | | | | | | [A/G]GCCTACAGAAAATAG | NO: |
| | | | | | | | | | | AGAAAGGACAGCCAATGAGA | 764 |
| | | | | | | | | | | AGACC | |
| chr | 309181 | G | A | DPCR1 | p.G640 | 0.005 | 0.000 | 6.87 | 42.35 | GAAAGGACAGCCAATGAGAA | SEQ |
| 6 | 60 | | | | E | 64 | 13 | E−21 | [18.16- | CACCACACCATCCCCAGCAG | ID |
| | | | | | | | | | 98.74] | [G/A]GCCTACAGAAAATAG | NO: |
| | | | | | | | | | | AGAAATGACAGCCAACGAGA | 765 |
| | | | | | | | | | | AGACC | |
| chr | 309207 | A | C | DPCR1 | p.Y134 | 0.005 | 0.002 | 4.32 | 1.75 | GTTCTCATTCCTCCTTTCTC | SEQ |
| 6 | 55 | | | | 8S | 21 | 99 | E−02 | [0.98- | ATCCCAATCACAGGTCTCCT | ID |
| | | | | | | | | | 2.88] | [A/C]TATGATGCGGACACG | NO: |
| | | | | | | | | | | CCGCACACTAACCCAGAACA | 766 |
| | | | | | | | | | | CCCAG | |
| chr | 309543 | C | T | MUC2 | p.S125 | 0.013 | 0.000 | 5.07 | Inf | CAACCTCCAGTGGGGCCAGC | SEQ |
| 6 | 27 | | | 1 | S | 24 | O0 | E−73 | | ACAGCCACCAACTCTGAGTC | ID |
| | | | | | | | | | | [C/T]AGCACACCCTCCAGT | NO: |
| | | | | | | | | | | GGGGCCAGCACAGCCACCAA | 767 |
| | | | | | | | | | | CTCTG | |
| chr | 309544 | A | G | MUC2 | p.S163 | 0.019 | 0.000 | 1.38 | Inf | AGCCACCAACTCTGACTCCA | SEQ |
| 6 | 39 | | | 1 | G | 61 | 00 | E− | | GCACAACCTCCAGTGAGGCC | ID |
| | | | | | | | | 116 | | [A/G]GCACAGCCACCAACT | NO: |
| | | | | | | | | | | CTGAGTCCAGCACAACCTCC | 768 |
| | | | | | | | | | | AGTGG | |
| chr | 309956 | C | T | MUC2 | p.S809 | 0.009 | 0.000 | 5.89 | Inf | CTACAGTTTCCACCACAGGC | SEQ |
| 6 | 35 | | | 2 | S | 56 | 00 | E−43 | | TTGGAGACCACCACCACTTC | ID |
| | | | | | | | | | | [C/T]ACTGAAGGCTCTGAG | NO: |
| | | | | | | | | | | ATGACTACAGTCTCCACCAC | 769 |
| | | | | | | | | | | AGGTG | |
| chr | 316916 | C | A | C6orf2 | p.G104 | 0.005 | 0.002 | 2.87 | 2.08 | TCCGGCGGCTGGAGCTCCTC | SEQ |
| 6 | 66 | | | 5 | G | 39 | 60 | E−03 | [1.35- | TTGAGCGCGGGGGACTCGGG | ID |
| | | | | | | | | | 3.22] | [C/A]ACTTTTTTCTGCAAG | NO: |
| | | | | | | | | | | GGCCGCCACGAGGACGAGAG | 770 |
| | | | | | | | | | | CCGTA | |
| chr | 317368 | C | T | VWA7 | p.R488 | 0.005 | 0.002 | 1.64 | 2.13 | CAGGGCAGCCATGCTCTCCC | SEQ |
| 6 | 35 | | | | Q | 39 | 54 | E−03 | [1.38- | CAACAATGGCTGCCACGTCT | ID |
| | | | | | | | | | 3.29] | [C/T]GAATGTGCTGGTCTT | NO: |
| | | | | | | | | | | TGGTGAAGATCACCTCTCCT | 771 |
| | | | | | | | | | | CCTGA | |
| chr | 326342 | A | G | HLA- | p.S35P | 0.007 | 0.004 | 1.76 | 1.73 | TGGCGGCTCTGGAGAGCAGC | SEQ |
| 6 | 82 | | | DQB1 | | 48 | 33 | E−02 | [1.09- | TGCCCTGCACTTACCGGGAG | ID |
| | | | | | | | | | 2.64] | [A/G]GTCTCTGCCCTCAGC | NO: |
| | | | | | | | | | | CAGTAGGGAGCTCAGCATCG | 772 |
| | | | | | | | | | | CCAGC | |
| chr | 327136 | C | A | HLA- | p.P128 | 0.006 | 0.000 | 1.10 | Inf | GTCACAGTGTTTTCCAAGTT | SEQ |
| 6 | 19 | | | DQA2 | H | 62 | 00 | E−39 | | TCCTGTGACGCTGGGTCAGC | ID |
| | | | | | | | | | | [C/A]CAACACCCTCATCTG | NO: |
| | | | | | | | | | | TCTTGTGGACAACATCTTTC | 773 |
| | | | | | | | | | | CTCCT | |
| chr | 327140 | T | G | HLA- | p.L219 | 0.020 | 0.000 | 4.06 | 2275.46 | GCCTGAGATTCCAGCCCCTA | SEQ |
| 6 | 58 | | | DQA2 | V | 59 | 01 | E− | [316.74- | TGTCAGAGCTCACAGAGACT | ID |
| | | | | | | | | 120 | 16346.8] | [T/G]TGGTCTGCGCCCTGG | NO: |
| | | | | | | | | | | GGTTGTCTGTGGGCCTCATG | 774 |
| | | | | | | | | | | GGCAT | |
| chr | 327141 | C | G | HLA- | p.G235 | 0.012 | 0.000 | 1.19 | Inf | CCCTGGGGTTGTCTGTGGGC | SEQ |
| 6 | 08 | | | DQA2 | G | 75 | 00 | E−75 | | CTCATGGGCATTGTGGTGGG | ID |
| | | | | | | | | | | [C/G]ACTGTCTTCATCATC | NO: |
| | | | | | | | | | | CAAGGCCTGCGTTCAGTTGG | 775 |
| | | | | | | | | | | TGCTT | |
| chr | 327141 | T | C | HLA- | p.T236 | 0.012 | 0.000 | 3.37 | Inf | TGGGGTTGTCTGTGGGCCTC | SEQ |
| 6 | 11 | | | DQA2 | T | 50 | 00 | E−74 | | ATGGGCATTGTGGTGGGCAC | ID |
| | | | | | | | | | | [T/C]GTCTTCATCATCCAA | NO: |
| | | | | | | | | | | GGCCTGCGTTCAGTTGGTGC | 776 |
| | | | | | | | | | | TTCCA | |
| chr | 327141 | C | G | HLA- | p.F238 | 0.016 | 0.000 | 4.00 | Inf | TGTCTGTGGGCCTCATGGGC | SEQ |
| 6 | 17 | | | DQA2 | L | 91 | 00 | E− | | ATTGTGGTGGGCACTGTCTT | ID |
| | | | | | | | | 100 | | [C/G]ATCATCCAAGGCCTG | NO: |
| | | | | | | | | | | CGTTCAGTTGGTGCTTCCAG | 777 |
| | | | | | | | | | | ACACC | |
| chr | 328200 | C | A | TAP1 | p.V304 | 0.005 | 0.002 | 1.19 | 2.21 | TGCACGTGGCCCATGGTGTT | SEQ |
| 6 | 00 | | | | L | 39 | 45 | E−03 | [1.43- | GTTATAGATCCCGTCACCCA | ID |
| | | | | | | | | | 3.42] | [C/A]GAACTCCAGCACTGC | NO: |
| | | | | | | | | | | ACTATAAAGAACCCGGAAAA | 778 |
| | | | | | | | | | | AAAGG | |
| chr | 333658 | G | T | KIFC1 | p.R5S | 0.005 | 0.003 | 3.11 | 1.62 | CTCCTGGGTATTGTCTTAAG | SEQ |
| 6 | 08 | | | | | 64 | 49 | E−02 | [1.06- | GGTCTCTTTTCCCAACAGAG | ID |
| | | | | | | | | | 2.47] | [G/T]TCCCCCCTATTGGAA | NO: |
| | | | | | | | | | | GTAAAGGGGAACATAGAACT | 779 |
| | | | | | | | | | | GAAGA | |
| chr | 340039 | C | T | GRM4 | p.S520 | 0.005 | 0.003 | 4.08 | 1.59 | AGCTGATGCTCATCCCTAGT | SEQ |
| 6 | 28 | | | | S | 39 | 40 | E−02 | [1.03- | CCCAGGAAGATTCGGCGCAG | ID |
| | | | | | | | | | 2.45] | [C/T]GAGCAGGTGCCAAGG | NO: |
| | | | | | | | | | | TCGGGCTCAGCGATCATGAG | 780 |
| | | | | | | | | | | GAAGG | |
| chr | 357150 | C | T | ARMC1 | p.I188I | 0.005 | 0.002 | 1.08 | 1.84 | AGGAACACTCCATCAAAGTA | SEQ |
| 6 | 76 | | | 2 | | 15 | 81 | E−02 | [1.18- | CTCGAACTGATCTCCACCAT | ID |
| | | | | | | | | | 2.86] | [C/T]TGGGACACGGAACTG | NO: |
| | | | | | | | | | | CACATTGCGGGCCTCAGACT | 781 |
| | | | | | | | | | | CCTCA | |
| chr | 367100 | T | A | CPNE5 | p.I593 | 0.006 | 0.000 | 5.82 | Inf | CCCAGGCCCCAGCCACCTGC | SEQ |
| 6 | 50 | | | | F | 62 | 00 | E−39 | | CTGCTGAGACCAGGTTCAGA | ID |
| | | | | | | | | | | [T/A]GTGCGTGTGCAGGGG | NO: |
| | | | | | | | | | | GGACGCAGGGGGCGTGCGGG | 782 |
| | | | | | | | | | | CTGGG | |
| chr | 392828 | G | A | KCNK1 | p.Q25 | 0.007 | 0.004 | 4.28 | 1.75 | CCTCAGCTTCCCAGTCCTTT | SEQ |
| 6 | 16 | | | 6 | 1X | 84 | 49 | E−03 | [1.23- | CTTGGATATGGGGAAGTCCT | ID |
| | | | | | | | | | 2.51] | [G/A]GGGTGTGACTTGGAC | NO: |
| | | | | | | | | | | TCCTCTTGCTGCTGTAGAGC | 783 |
| | | | | | | | | | | CTCTC | |
| chr | 441438 | G | A | CAPN1 | p.A297 | 0.005 | 0.002 | 2.43 | 1.85 | ACTGGAATCCATGACTGACA | SEQ |
| 6 | 62 | | | 1 | T | 21 | 82 | E−02 | [1.04- | AGATGCTGGTGAGAGGGCAC | ID |
| | | | | | | | | | 3.06] | [G/A]CTTACTCTGTGACTG | NO: |
| | | | | | | | | | | GCCTTCAGGATGTGAGTCCT | 784 |
| | | | | | | | | | | GAGAA | |
| chr | 466559 | C | G | TDRD6 | p.A12 | 0.012 | 0.000 | 4.48 | Inf | TCAAGATGTGCTCGACGCCC | SEQ |
| 6 | 01 | | | | A | 01 | 00 | E−58 | | GGAATGCCGGCGCCGGGGGC | ID |
| | | | | | | | | | | [C/G]TCGCTGGCCCTGCGG | NO: |
| | | | | | | | | | | GTGTCCTTCGTGGACGTGCA | 785 |
| | | | | | | | | | | TCCCG | |
| chr | 560330 | G | A | COL21 | p.T343 | 0.067 | 0.071 | 3.19 | 0.94 | TACTAAGAGACGAATTTGGT | SEQ |
| 6 | 94 | | | A1 | M | 40 | 69 | E−01 | [0.83- | GCCAGCCTTCATCAAACAAC | ID |
| | | | | | | | | | 1.06] | [G/A]TCTACAAAAAGAAAG | NO: |
| | | | | | | | | | | TGTGGAAGATTCATAAATAA | 786 |
| | | | | | | | | | | AGCCC | |
| chr | 767318 | G | A | IMPG1 | p.N13 | 0.010 | 0.007 | 3.74 | 1.39 | AACTCTAGGAACTTCTTACT | SEQ |
| 6 | 54 | | | | 7N | 78 | 76 | E−02 | [1.03- | GTTGTAGGCATCTTGGTGTC | ID |
| | | | | | | | | | 1.89] | [G/A]TTGAGTGTATTATCG | NO: |
| | | | | | | | | | | AGAATTTCATTGAGGAGGGT | 787 |
| | | | | | | | | | | GTCAT | |
| chr | 843032 | T | C | SNAP9 | p.T553 | 0.010 | 0.007 | 1.83 | 1.49 | AAATTACCACCAAAGATATC | SEQ |
| 6 | 30 | | | 1 | A | 78 | 26 | E−02 | [1.09- | TAGAGCAGGAGGAGCAGTGG | ID |
| | | | | | | | | | 2.04] | [T/C]GGCGGTGGCAGCGGA | NO: |
| | | | | | | | | | | GGTGGTGGTAGTGGTGGTGG | 788 |
| | | | | | | | | | | CAGCG | |
| chr | 854737 | C | T | TBX18 | p.G48 | 0.414 | 0.494 | 5.64 | 0.72 | GCGCCGCCGCCGCGGCTGCA | SEQ |
| 6 | 58 | | | | R | 71 | 51 | E−23 | [0.68- | GCCTCCGTCGTCCACGGCCC | ID |
| | | | | | | | | | 0.77] | [C/T]CGCCGCCTCTTCGGC | NO: |
| | | | | | | | | | | GCCCAGTTTTCGCCGCTTCT | 789 |
| | | | | | | | | | | TCTGA | |
| chr | 861950 | G | A | NT5E | p.V278 | 0.007 | 0.004 | 1.07 | 1.64 | ATTCATAGTCACTTCTGATG | SEQ |
| 6 | 33 | | | | I | 60 | 66 | E−02 | [1.14- | ATGGGCGGAAGGTTCCTGTA | ID |
| | | | | | | | | | 2.35] | [G/A]TCCAGGCCTATGCTT | NO: |
| | | | | | | | | | | TTGGCAAATACCTAGGCTAT | 790 |
| | | | | | | | | | | CTGAA | |
| chr | 905721 | G | A | CASP8 | p.G237 | 0.005 | 0.003 | 2.39 | 1.69[ | AATGGTGTTTGGTCACGTTC | SEQ |
| 6 | 38 | | | AP2 | F | 39 | 19 | E−02 | 1.1- | TCATTATCAGGTTGGCGAGG | ID |
| | | | | | | | | | 2.61] | [G/A]TAGCTCAAATGAGGA | NO: |
| | | | | | | | | | | TAGTAGAAGAGGAAGAAAAG | 791 |
| | | | | | | | | | | ATATT | |
| chr | 108882 | A | T | FOXO3 | p.S26C | 0.005 | 0.000 | 2.37 | 20.81 | TCCGCTCGAAGTGGAGCTGG | SEQ |
| 6 | 487 | | | | | 39 | 26 | E−17 | [10.92- | ACCCGGAGTTCGAGCCCCAG | ID |
| | | | | | | | | | 39.65] | [A/T]GCCGTCCGCGATCCT | NO: |
| | | | | | | | | | | GTACGTGGCCCCTGCAAAGG | 792 |
| | | | | | | | | | | CCGGA | |
| chr | 109867 | T | C | AK9 | p.E103 | 0.023 | 0.000 | 6.49 | 295.82 | CGTTCTCAGAATCTTCCTCA | SEQ |
| 6 | 190 | | | | SE | 28 | 08 | E− | [149.23- | AATTCAGGTCCCACTTTCTT | ID |
| | | | | | | | | 127 | 586.43] | [T/C]TCAGTTTTGAGTAGT | NO: |
| | | | | | | | | | | AGTTTTTCTTGAAGAACTTC | 793 |
| | | | | | | | | | | TTCAA | |
| chr | 126073 | T | G | HEY2 | p.L74L | 0.005 | 0.003 | 3.66 | 1.58 | GGGATCGGATAAATAACAGT | SEQ |
| 6 | 212 | | | | | 88 | 72 | E−02 | [1.05- | TTATCTGAGTTGAGAAGACT | ID |
| | | | | | | | | | 2.39] | [T/G]GTGCCAACTGCTTTT | NO: |
| | | | | | | | | | | GAAAAACAAGTAAGCTATCC | 794 |
| | | | | | | | | | | CCTCC | |
| chr | 136597 | G | A | BCLAF | p.P497 | 0.005 | 0.002 | 2.13 | 2.47 | TCAAAGAGGTCTTTGAGCTT | SEQ |
| 6 | 174 | | | 1 | S | 64 | 29 | E−04 | [1.61- | TTCAGACTTTACCTGCTCAG | ID |
| | | | | | | | | | 3.78] | [G/A]TGACTGAGTTTCTTT | NO: |
| | | | | | | | | | | CTTTACTGTTATTCTTTCAG | 795 |
| | | | | | | | | | | AATTT | |
| chr | 136597 | C | A | BCLAF | p.E403 | 0.008 | 0.004 | 1.21 | 1.83 | AGGACTGACTTCCTGAACTG | SEQ |
| 6 | 456 | | | 1 | X | 58 | 71 | E−03 | [1.3- | TCTATAATCCTCTGTCTCCT | ID |
| | | | | | | | | | 2.58] | [C/A]TGTGTCATCCCCTTC | NO: |
| | | | | | | | | | | TGAATCATTAAACTTTTGTT | 796 |
| | | | | | | | | | | TTCCA | |
| chr | 137814 | G | T | OLIG3 | p.I124I | 0.024 | 0.000 | 1.52 | 2806.41 | TGAGCATGAGGATGTAGTTT | SEQ |
| 6 | 936 | | | | | 51 | 01 | E− | [391.38- | CTGGCGAGCAGGAGTGTGGC | ID |
| | | | | | | | | 144 | 20123.7] | [G/T]ATCTTGGAGAGCTTG | NO: |
| | | | | | | | | | | CGCACCGACGGCCCATGCGC | 797 |
| | | | | | | | | | | GTAGG | |
| chr | 139113 | A | T | CCDC2 | p.T271 | 0.008 | 0.002 | 7.18 | 3.81 | ACAAAAACTCCATTTGGCAG | SEQ |
| 6 | 926 | | | 8A | S | 14 | 15 | E−08 | [2.41- | ATGCACAAGATGTTCCAAAT | ID |
| | | | | | | | | | 5.78] | [A/T]CTTCTGCTAGCTAAA | NO: |
| | | | | | | | | | | ATGAAATGTAGTTTGCTTTC | 798 |
| | | | | | | | | | | TTGTG | |
| chr | 152457 | C | T | SYNE1 | p.E853 | 0.008 | 0.001 | 6.73 | 5.3 | GGCACTGCATCAGGGCATCC | SEQ |
| 6 | 795 | | | | 9E | 36 | 60 | E−08 | [3.21- | TGCAGCAGGCCCCGCCACTC | ID |
| | | | | | | | | | 8.74] | [C/T]TCCAGCAGAGAGCAC | NO: |
| | | | | | | | | | | ACTCGGTCCCAGCGCCCATT | 799 |
| | | | | | | | | | | CATCT | |
| chr | 155143 | A | G | SCAF8 | p.T629 | 0.005 | 0.003 | 3.35 | 1.59 | TCAGAGCCCAACTCCAGTTG | SEQ |
| 6 | 502 | | | | A | 64 | 54 | E−02 | [1.05- | AAAAGGAGACAGTGGTCACA | ID |
| | | | | | | | | | 2.43] | [A/G]CCCAGGCAGAGGTTT | NO: |
| | | | | | | | | | | TCCCTCCTCCTGTTGCTATG | 800 |
| | | | | | | | | | | TTGCA | |
| chr | 158487 | T | C | SYNJ2 | p.M29 | 0.009 | 0.005 | 9.88 | 1.57 | CAGTCCGAATTCACAAATTT | SEQ |
| 6 | 551 | | | | 7T | 31 | 94 | E−03 | [1.13- | CAAGCGGATCCGGATTGCTA | ID |
| | | | | | | | | | 2.18] | [T/C]GGGGACCTGGAACGT | NO: |
| | | | | | | | | | | GAACGGAGGAAAGCAGTTCC | 801 |
| | | | | | | | | | | GGAGC | |
| chr | 167728 | T | C | UNC93 | p.Y387 | 0.007 | 0.000 | 1.30 | 7.62 | CGTTCTCTTTGAGAAGAGCA | SEQ |
| 6 | 725 | | | A | H | 35 | 97 | E−15 | [5.08- | AGGAAGCTGCCTTCGCCAAT | ID |
| | | | | | | | | | 11.44] | [T/C]ACCGCCTGTGGGAGG | NO: |
| | | | | | | | | | | CCCTGGGCTTCGTCATTGCC | 802 |
| | | | | | | | | | | TTCGG | |
| chr | 331061 | A | G | WI2- | p.K103 | 0.015 | 0.000 | 1.78 | 377.58 | GCGCGCAGGTGCCGCGGTCC | SEQ |
| 7 | | | | 2373I1 | K | 69 | 04 | E−52 | [52.37- | GAGGGCCACGAGAAGGGCAA | ID |
| | | | | .2 | | | | | 2722.42] | [A/G]GGCAACTACTGGACG | NO: |
| | | | | | | | | | | TTCGCGGGCGGCTGCGAGTC | 803 |
| | | | | | | | | | | GCTGC | |
| chr | 102700 | G | A | CYP2W | p.R328 | 0.023 | 0.000 | 9.98 | 1179.78 | CCACCCTTTGCCCCAGGCCG | SEQ |
| 7 | 7 | | | 1 | H | 77 | 02 | E− | [290.79- | GGTGCAGGAGGAGCTAGACC | ID |
| | | | | | | | | 133 | 4786.53] | [G/A]CGTGCTGGGCCCTGG | NO: |
| | | | | | | | | | | GCGGACTCCCCGGCTGGAGG | 804 |
| | | | | | | | | | | ACCAG | |
| chr | 102837 | C | T | CYP2W | p.P464 | 0.010 | 0.007 | 2.76 | 1.43 | CTGCAGAGGTACCGCCTGCT | SEQ |
| 7 | 6 | | | 1 | L | 78 | 56 | E−02 | [1.05- | GCCCCCGCCTGGCGTCAGTC | ID |
| | | | | | | | | | 1.94] | [C/T]GGCCTCCCTGGACAC | NO: |
| | | | | | | | | | | CACGCCCGCCCGGGCTTTTA | 805 |
| | | | | | | | | | | CCATG | |
| chr | 178430 | C | T | ELFN1 | p.R26C | 0.006 | 0.003 | 4.27 | 1.55 | CGTGGCGGCCGCCACCCTGC | SEQ |
| 7 | 8 | | | | | 13 | 97 | E−02 | [1.02- | TGCACGCTGGCGGCCTGGCC | ID |
| | | | | | | | | | 2.34] | [C/T]GCGCAGACTGCTGGC | NO: |
| | | | | | | | | | | TGATCGAGGGCGACAAGGGC | 806 |
| | | | | | | | | | | TTCGT | |
| chr | 225589 | C | G | MAD1L | p.E236 | 0.007 | 0.004 | 1.35 | 1.62 | CCAGCTCAGACTTCATGTTC | SEQ |
| 7 | 3 | | | 1 | D | 35 | 54 | E−02 | [1.12- | TTCACAATCGCTGCATCCTG | ID |
| | | | | | | | | | 2.35] | [C/G]TCTTGCAGGGACAGC | NO: |
| | | | | | | | | | | TTCTGCTCCAGATCCTGATG | 807 |
| | | | | | | | | | | GAGGC | |
| chr | 418545 | G | A | SDK1 | p.P144 | 0.010 | 0.007 | 4.98 | 1.38 | GCGCCACAGTGAGGCAGTTC | SEQ |
| 7 | 7 | | | | 4P | 05 | 28 | E−02 | [1.01- | ACAGCCACCGACCTGGCCCC | ID |
| | | | | | | | | | 1.9] | [G/A]GAGTCCGCATACATC | NO: |
| | | | | | | | | | | TTCAGGCTGTCCGCCAAGAC | 808 |
| | | | | | | | | | | GAGGC | |
| chr | 485690 | T | C | RADIL | p.Y565 | 0.009 | 0.006 | 2.59 | 1.47 | GTGCACCTTGGAGACATAGT | SEQ |
| 7 | 4 | | | | C | 07 | 17 | E−02 | [1.06- | AGACGCACTGCTGGAAGGCG | ID |
| | | | | | | | | | 2.06] | [T/C]ACAGCACCACCTCCT | NO: |
| | | | | | | | | | | CCAGCACCGCCATGGCCTCC | 809 |
| | | | | | | | | | | TCGCT | |
| chr | 602682 | G | C | PMS2 | p.S523 | 0.006 | 0.003 | 1.88 | 1.66 | CCTGAGAGTCCACATGTTCC | SEQ |
| 7 | 7 | | | | S | 13 | 70 | E−02 | [1.11- | TGCGAGCCCCTGTCCCCTGG | ID |
| | | | | | | | | | 2.49] | [G/C]GAGCTGGCCGCATAC | NO: |
| | | | | | | | | | | TCGCTGCTGCAGTGACTGCC | 810 |
| | | | | | | | | | | CGTGT | |
| chr | 232218 | A | G | NUPL2 | p.Q36 | 0.005 | 0.000 | 4.20 | 195.45 | CCCGGTGCTAGGGGTGCAGG | SEQ |
| 7 | 11 | | | | R | 39 | 03 | E−29 | [58.48- | AGGAGGACGGCAGCAACCGC | ID |
| | | | | | | | | | 653.28] | [A/G]GCAGCAGCCTTCAGG | NO: |
| | | | | | | | | | | TGACTCTCCTCTGAATCCTC | 811 |
| | | | | | | | | | | CGCGG | |
| chr | 262176 | A | G | NFE2L | p.I233 | 0.001 | not | 4.03 | Inf | GGAGAACTCACTTCAGCAGA | SEQ |
| 7 | 89 | | | 3 | V | 47 | found | E−06 | | ATGATGATGATGAAAACAAA | ID |
| | | | | | | | | | | [A/G]TAGCAGAGAAACCTG | NO: |
| | | | | | | | | | | ACTGGGAGGCAGAAAAGACC | 812 |
| | | | | | | | | | | ACTGA | |
| chr | 309219 | G | I | FAM18 | p.R696 | 0.006 | 0.000 | 2.24 | 370.83 | GCCTGCAGCCGGGGCTCCTG | SEQ |
| 7 | 12 | | | 8B | S | 62 | 02 | E−37 | [88.15- | CGTGACTGGAGGACTGAGAG | ID |
| | | | | | | | | | 1559.92] | [G/T]CTCTTTGACTTGTAC | NO: |
| | | | | | | | | | | TACTACGATGGCCTGGCCAA | 813 |
| | | | | | | | | | | CCAGC | |
| chr | 379885 | G | T | EPDR1 | p.G79 | 0.007 | 0.000 | 1.62 | 159.69 | CATTCCTCAAAACTCCACCT | SEQ |
| 7 | 90 | | | | W | 11 | 04 | E−37 | [61.78- | TTGAAGACCAGTACTCCATC | ID |
| | | | | | | | | | 412.75] | [G/T]GGGGGCCTCAGGAGC | NO: |
| | | | | | | | | | | AGATCACCGTCCAGGAGTGG | 814 |
| | | | | | | | | | | TCGGA | |
| chr | 420072 | T | C | GLI3 | p.I808 | 0.006 | 0.002 | 4.10 | 2.47 | CTGAGCAGATGCATGGTCTG | SEQ |
| 7 | 01 | | | | M | 86 | 79 | E−05 | [1.67- | ATGTAGAACTCACCATTTCC | ID |
| | | | | | | | | | 3.63] | [T/C]ATGAGAGGAGAGACC | NO: |
| | | | | | | | | | | GCAGGGGCTTTAGGGGGTAG | 815 |
| | | | | | | | | | | AATGG | |
| chr | 441544 | G | A | POLD2 | p.C447 | 0.006 | 0.004 | 4.50 | 1.53 | CATCGTCCTCTGCCCCGAAG | SEQ |
| 7 | 53 | | | | C | 13 | 02 | E−02 | [1.02- | CCCGAGAAGCTGATGGGCTG | ID |
| | | | | | | | | | 2.29] | [G/A]CAGGCCAGGCTGCGC | NO: |
| | | | | | | | | | | AGGTTCACAAGGCAGGCGGT | 816 |
| | | | | | | | | | | CTGCG | |
| chr | 451239 | C | T | NACAD | p.K618 | 0.005 | 0.000 | 6.20 | 47.37 | CTTCAGCCTGCTGGGACACA | SEQ |
| 7 | 25 | | | | K | 15 | 11 | E−19 | [17.85- | ATCGTGGCTGCAGCCACAGG | ID |
| | | | | | | | | | 125.69] | [C/T]TTTGGGGCTGATGAG | NO: |
| | | | | | | | | | | AGATCTGTGTCTTGTAGGGG | 817 |
| | | | | | | | | | | CAGAG | |
| chr | 479255 | C | T | PKD1L | p.R990 | 0.009 | 0.006 | 3.03 | 1.46 | TGAAGTGGCAGGTTGGCCAA | SEQ |
| 7 | 20 | | | 1 | Q | 56 | 56 | E−02 | [1.06- | GGGTCACGGGTGAAGGTTCC | ID |
| | | | | | | | | | 2.02] | [C/T]GTGAGAATGGTGTGG | NO: |
| | | | | | | | | | | TCGTTGCATCAGGATCTGCA | 818 |
| | | | | | | | | | | GTGCC | |
| chr | 505717 | C | A | DDC | p.M23 | 0.005 | 0.003 | 3.84 | 1.62 | TGTCAAAGGAGCAGCATGTT | SEQ |
| 7 | 55 | | | | 9I | 39 | 34 | E−02 | [1.05- | GTGGTCCCCAGGGTGGCAAC | ID |
| | | | | | | | | | 2.49] | [C/A]ATCTAGAGGGTAAAA | NO: |
| | | | | | | | | | | AGCAGACAGCCTTTTATTCC | 819 |
| | | | | | | | | | | CCAGG | |
| chr | 506730 | C | T | GRB10 | p.P390 | 0.005 | 0.003 | 3.94 | 1.61 | AGGCGTGGCCCTCCTCCAGG | SEQ |
| 7 | 32 | | | | P | 39 | 37 | E−02 | [1.04- | TGCTGCGCTCTGGGCCTCTGC | ID |
| | | | | | | | | | 2.47] | [C/T]GGATTCTCTATCACG | NO: |
| | | | | | | | | | | CGTCCTGTTTGCCCAGAAAA | 820 |
| | | | | | | | | | | ATCCA | |
| chr | 636803 | C | A | ZNF73 | p.G303 | 0.008 | 0.000 | 1.80 | 989.53 | TTCATACTGGAGAGAGACCC | SEQ |
| 7 | 38 | | | 5P | G | 82 | 01 | E−51 | [135.64- | TACAAATGTGAAGAATGTGG | ID |
| | | | | | | | | | 7219.03] | [C/A]AAAGCCTTTAGCGTA | NO: |
| | | | | | | | | | | TCCTCAGCCCTCATTTACCA | 821 |
| | | | | | | | | | | CAAGA | |
| chr | 638092 | C | A | ZNF73 | p.I342I | 0.014 | 0.000 | 8.84 | Inf | GTAAACATAAGAGAATTCAT | SEQ |
| 7 | 67 | | | 6 | | 22 | 00 | E−84 | | ACTGGAGAGAAACCCTACAT | ID |
| | | | | | | | | | | [C/A]TGTGAAGAATGTGGC | NO: |
| | | | | | | | | | | AAAGCCTTTACCCGCTCCTC | 822 |
| | | | | | | | | | | AACCC | |
| chr | 871606 | G | A | ABCB1 | p.L884 | 0.007 | 0.005 | 4.73 | 1.46 | CTCACCTTCCCAGAACCTTC | SEQ |
| 7 | 45 | | | | L | 60 | 22 | E−02 | [1.02- | TAGTTCTTTCTTATCTTTCA | ID |
| | | | | | | | | | 2.1] | [G/A]TGCTTGTCCAGACAA | NO: |
| | | | | | | | | | | CATTTTCATTTCAACAACTC | 823 |
| | | | | | | | | | | CTGCT | |
| chr | 889655 | C | T | ZNF80 | p.T108 | 0.006 | 0.003 | 1.55 | 1.98 | TTCCCTGGTGCTTTTCCGTC | SEQ |
| 7 | 53 | | | 4B | 6I | 62 | 35 | E−03 | [1.34- | TAATAAATATACTGGTGTGA | ID |
| | | | | | | | | | 2.93] | [C/T]TGATTCAACAGAGAC | NO: |
| | | | | | | | | | | CCAAGAAGACCAAATAAATC | 824 |
| | | | | | | | | | | TAGAC | |
| chr | 916030 | C | T | AKAP9 | p.S27L | 0.005 | 0.002 | 6.73 | 1.92 | TTTTCTTAGCTTGCCCAGTT | SEQ |
| 7 | 56 | | | | | 39 | 82 | E−03 | [1.24- | TCGACAAAGAAAAGCTCAGT | ID |
| | | | | | | | | | 2.96] | [C/T]GGATGGGCAGAGTCC | NO: |
| | | | | | | | | | | TTCCAAGAAGCAGAAAAAAA | 825 |
| | | | | | | | | | | AGAGA | |
| chr | 978223 | G | A | LMTK2 | p.A862 | 0.009 | 0.005 | 9.20 | 1.6 | TGTCCCGGAGGACTGTCTCC | SEQ |
| 7 | 61 | | | | T | 31 | 85 | E−03 | [1.15- | ACCAGGACATCAGTCCAGAC | ID |
| | | | | | | | | | 2.22] | [G/A]CTGTGACTGTCCCGG | NO: |
| | | | | | | | | | | TTGAAATTCTCTCAACTGAT | 826 |
| | | | | | | | | | | GCCAG | |
| chr | 999995 | T | C | ZCWP | p.R529 | 0.005 | 0.002 | 1.11 | 1.83 | CCTGGCTGGTCAGAATCTGA | SEQ |
| 7 | 51 | | | W1 | G | 15 | 82 | E−02 | [1.17- | ATTCCCTTGGCCTTCTTTCC | ID |
| | | | | | | | | | 2.85] | [T/C]TCCCATTCTGGGTGC | NO: |
| | | | | | | | | | | AGGAGGAGCTGTGGATTTCC | 827 |
| | | | | | | | | | | TGCCT | |
| chr | 100228 | G | T | TFR2 | p.A376 | 0.006 | 0.004 | 2.67 | 1.58 | ATAAGGGGAGCCTAGGAGGC | SEQ |
| 7 | 655 | | | | D | 62 | 19 | E−02 | [1.07- | TCCCCTGCCATTCTTGGGGG | ID |
| | | | | | | | | | 2.34] | [G/T]CCACAGGGCCTTTGA | NO: |
| | | | | | | | | | | GCTTCCTGGAGAGGAGGAAG | 828 |
| | | | | | | | | | | GCAGA | |
| chr | 100633 | G | A | MUC1 | p.G32S | 0.005 | 0.006 | 9.17 | 0.95 | CTCTCAAATCACAGGCTCAA | SEQ |
| 7 | 938 | | | 2 | | 88 | 17 | E−01 | [0.63- | CAGTAAACACCAGTATTGGA | ID |
| | | | | | | | | | 1.44] | [G/A]GTAATACAACTTCTG | NO: |
| | | | | | | | | | | CATCCACACCCAGTTCAAGC | 829 |
| | | | | | | | | | | GACCC | |
| chr | 100633 | C | T | MUC1 | p.T39I | 0.000 | 0.000 | 1.85 | 7.1 | GTAAACACCAGTATTGGAGG | SEQ |
| 7 | 960 | | | 2 | | 25 | 03 | E−01 | [0.64- | TAATACAACTTCTGCATCCA | ID |
| | | | | | | | | | 78.35] | [C/T]ACCCAGTTCAAGCGA | NO: |
| | | | | | | | | | | CCCTTTTACCACCTTTAGTG | 830 |
| | | | | | | | | | | ACTAT | |
| chr | 100634 | G | A | MUC1 | p.A101 | 0.002 | 0.001 | 1.71 | 1.59 | CCCAGGTGCAACTGGAACAA | SEQ |
| 7 | 145 | | | 2 | T | 70 | 70 | E−01 | [0.85- | CACTCTTCCCTTCCCACTCT | ID |
| | | | | | | | | | 2.96] | [G/A]CAACCTCAGTTTTTG | NO: |
| | | | | | | | | | | TTGGAGAACCTAAAACCTCA | 831 |
| | | | | | | | | | | CCCAT | |
| chr | 100634 | C | T | MUC1 | p.T122 | 0.000 | 0.000 | 1.84 | 7.15 | CCTAAAACCTCACCCATCAC | SEQ |
| 7 | 209 | | | 2 | I | 25 | 03 | E−01 | [0.65- | TTCAGCCTCAATGGAAACAA | ID |
| | | | | | | | | | 78.87] | [C/T]AGCGTTACCTGGCAG | NO: |
| | | | | | | | | | | TACCACAACAGCAGGCCTGA | 832 |
| | | | | | | | | | | GTGAG | |
| chr | 100634 | C | G | MUC1 | p.P153 | 0.000 | 0.000 | 1.00 | 0.92 | TTCTACAGTAGCCCCAGATC | SEQ |
| 7 | 302 | | | 2 | R | 49 | 53 | E+00 | [0.22- | ACCAGACAGAACACTCTCAC | ID |
| | | | | | | | | | 3.85] | [C/G]TGCCCGCACGACAAG | NO: |
| | | | | | | | | | | CTCAGGCGTCAGTGAAAAAT | 833 |
| | | | | | | | | | | CAACC | |
| chr | 100634 | C | T | MUC1 | p.P172 | 0.006 | 0.006 | 7.62 | 1.06 | CTCAGGCGTCAGTGAAAAAT | SEQ |
| 7 | 358 | | | 2 | S | 86 | 48 | E−01 | [0.72- | CAACCACCTCCCACAGCCGA | ID |
| | | | | | | | | | 1.56] | [C/T]CAGGCCCAACGCACA | NO: |
| | | | | | | | | | | CAATAGCGTTCCCTGACAGT | 834 |
| | | | | | | | | | | ACCAC | |
| chr | 100634 | C | A | MUC1 | p.T177 | 0.000 | 0.000 | 1.00 | 1.02 | AAATCAACCACCTCCCACAG | SEQ |
| 7 | 374 | | | 2 | K | 25 | 24 | E+00 | [0.13- | CCGACCAGGCCCAACGCACA | ID |
| | | | | | | | | | 7.75] | [C/A]AATAGCGTTCCCTGA | NO: |
| | | | | | | | | | | CAGTACCACCATGCCAGGCG | 835 |
| | | | | | | | | | | TCAGT | |
| chr | 100634 | C | T | MUC1 | p.P181 | 0.001 | 0.002 | 7.38 | 0.83 | TCCCACAGCCGACCAGGCCC | SEQ |
| 7 | 386 | | | 2 | L | 96 | 37 | E−01 | [0.41- | AACGCACACAATAGCGTTCC | ID |
| | | | | | | | | | 1.69] | [C/T]TGACAGTACCACCAT | NO: |
| | | | | | | | | | | GCCAGGCGTCAGTCAGGAAT | 836 |
| | | | | | | | | | | CTACA | |
| chr | 100634 | T | G | MUC1 | p.I199 | 0.000 | 0.000 | 5.26 | 1.42 | ATGCCAGGCGTCAGTCAGGA | SEQ |
| 7 | 440 | | | 2 | S | 25 | 17 | E−01 | [0.18- | ATCTACAGCTTCCCACAGCA | ID |
| | | | | | | | | | 11.13] | [T/G]CCCCGGCTCCACAGA | NO: |
| | | | | | | | | | | CACAACACTGTCCCCTGGCA | 837 |
| | | | | | | | | | | CTACC | |
| chr | 100634 | G | C | MUC1 | p.D286 | 0.005 | 0.005 | 1.00 | 0.99 | GGGAGAACCTACCACCTTCC | SEQ |
| 7 | 700 | | | 2 | H | 39 | 46 | E+00 | [0.64- | AGAGCTGGCCAAGCTCAAAG | ID |
| | | | | | | | | | 1.52] | [G/C]ACACTTCGCCTGCAC | NO: |
| | | | | | | | | | | CTTCTGGTACCACATCAGCC | 838 |
| | | | | | | | | | | TTTGT | |
| chr | 100634 | C | T | MUC1 | p.T315 | 0.000 | 0.000 | 5.28 | 1.42 | TCTACAACTTATCACAGCAG | SEQ |
| 7 | 788 | | | 2 | I | 25 | 17 | E−01 | [0.18- | CCCGAGCTCAACTCCAACAA | ID |
| | | | | | | | | | 11.07] | [C/T]CCACTTTTCTGCCAG | NO: |
| | | | | | | | | | | CTCCACAACCTTGGGCCATA | 839 |
| | | | | | | | | | | GTGAG | |
| chr | 100634 | G | A | MUC1 | p.R348 | 0.013 | 0.015 | 3.63 | 0.87 | AGCAGCCCAGTTGCAACTGC | SEQ |
| 7 | 887 | | | 2 | H | 97 | 95 | E−01 | [0.67- | AACAACACCCCCACCTGCCC | ID |
| | | | | | | | | | 1.14] | [G/A]CTCCGCGACCTCAGG | NO: |
| | | | | | | | | | | CCATGTTGAAGAATCTACAG | 840 |
| | | | | | | | | | | CCTAC | |
| chr | 100635 | A | T | MUC1 | p.K397 | 0.000 | 0.000 | 6.69 | 1.3 | GAAGAATCAGCAACTTTCCA | SEQ |
| 7 | 034 | | | 2 | I | 49 | 38 | E−01 | [0.31- | CGGCAGCACAACACACACAA | ID |
| | | | | | | | | | 5.53] | [A/T]ATCTTCAACTCCTAG | NO: |
| | | | | | | | | | | CACCACAGCTGCCCTAGCAC | 841 |
| | | | | | | | | | | ATACA | |
| chr | 100635 | C | G | MUC1 | p.T403 | 0.000 | 0.000 | 6.53 | Inf | CACGGCAGCACAACACACAC | SEQ |
| 7 | 052 | | | 2 | S | 25 | 00 | E−02 | [NaN- | AAAATCTTCAACTCCTAGCA | ID |
| | | | | | | | | | Inf] | [C/G]CACAGCTGCCCTAGC | NO: |
| | | | | | | | | | | ACATACAAGCTACCACAGCA | 842 |
| | | | | | | | | | | GCCTG | |
| chr | 100635 | T | C | MUC1 | p.L416 | 0.000 | 0.000 | 1.00 | 0.89 | ACCACAGCTGCCCTAGCACA | SEQ |
| 7 | 091 | | | 2 | P | 49 | 55 | E+00 | [0.21- | TACAAGCTACCACAGCAGCC | ID |
| | | | | | | | | | 3.73] | [T/C]GGGCTCAACTGAAAC | NO: |
| | | | | | | | | | | AACACACTTCCGTGATAGCT | 843 |
| | | | | | | | | | | CCACA | |
| chr | 100635 | C | G | MUC1 | p.D464 | 0.000 | 0.001 | 6.61 | 0.7 | TCTTACCTGCCGGCTCTACA | SEQ |
| 7 | 236 | | | 2 | E | 98 | 41 | E−01 | [0.26- | CCCTCAGTTCTTGTTGGAGA | ID |
| | | | | | | | | | 1.9] | [C/G]TCGACGCCCTCACCC | NO: |
| | | | | | | | | | | ATCAGTTCAGGCTCAATGGA | 844 |
| | | | | | | | | | | AACCA | |
| chr | 100635 | C | A | MUC1 | p.P469 | 0.001 | 0.001 | 6.90 | 0.71 | TCTACACCCTCAGTTCTTGT | SEQ |
| 7 | 250 | | | 2 | H | 23 | 72 | E−01 | [0.29- | TGGAGACTCGACGCCCTCAC | ID |
| | | | | | | | | | 1.75] | [C/A]CATCAGTTCAGGCTC | NO: |
| | | | | | | | | | | AATGGAAACCACAGCGTTAC | 845 |
| | | | | | | | | | | CCGGC | |
| chr | 100635 | A | C | MUC1 | p.M47 | 0.000 | 0.000 | 3.33 | 2.86 | TGTTGGAGACTCGACGCCCT | SEQ |
| 7 | 267 | | | 2 | 5L | 25 | 09 | E−01 | [0.33- | CACCCATCAGTTCAGGCTCA | ID |
| | | | | | | | | | 24.49] | [A/C]TGGAAACCACAGCGT | NO: |
| | | | | | | | | | | TACCCGGCAGTACCACAAAA | 846 |
| | | | | | | | | | | CCAGG | |
| chr | 100635 | A | C | MUC1 | p.S498 | 0.005 | 0.005 | 8.30 | 1.03 | CACAAAACCAGGCCTCAGTG | SEQ |
| 7 | 336 | | | 2 | G | 88 | 70 | E−01 | [0.68- | AGAAATCTACCACTTTCTAC | ID |
| | | | | | | | | | 1.56] | [A/G]GTAGCCCCAGATCAC | NO: |
| | | | | | | | | | | CAGACACAACACACTTACCT | 847 |
| | | | | | | | | | | GCCAG | |
| chr | 100635 | C | G | MUC1 | p.H525 | 0.000 | 0.000 | 4.92 | 1.59 | TGACAAGCTCAGGCGTCAGT | SEQ |
| 7 | 419 | | | 2 | Q | 25 | 15 | E−01 | [0.2- | GAAGAATCCACCACCTCCCA | ID |
| | | | | | | | | | 12.54] | [C/G]AGCCGACCAGGCTCA | NO: |
| | | | | | | | | | | ACACACACAACAGCATTCCC | 848 |
| | | | | | | | | | | TGGCA | |
| chr | 100635 | C | A | MUC1 | p.T533 | 0.000 | 0.000 | 1.26 | 14.32 | GAATCCACCACCTCCCACAG | SEQ |
| 7 | 442 | | | 2 | K | 25 | 02 | E−01 | [0.9- | CCGACCAGGCTCAACACACA | ID |
| | | | | | | | | | 228.9] | [C/A]AACAGCATTCCCTGG | NO: |
| | | | | | | | | | | CAGTACCACCATGCCAGGCC | 849 |
| | | | | | | | | | | TCAGT | |
| chr | 100635 | C | G | MUC1 | p.L602 | 0.000 | 0.000 | 1.00 | 0.71 | AACAACACTCTTACCTGACA | SEQ |
| 7 | 648 | | | 2 | V | 49 | 69 | E+00 | [0.17- | ACACCACAGCCTCAGGACTC | ID |
| | | | | | | | | | 2.95] | [C/G]TTGAAGCATCTATGC | NO: |
| | | | | | | | | | | CCGTCCACAGCAGCACCAGA | 850 |
| | | | | | | | | | | TCGCC | |
| chr | 100635 | A | C | MUC1 | p.E603 | 0.001 | 0.000 | 1.73 | 1.75 | ACACTCTTACCTGACAACAC | SEQ |
| 7 | 652 | | | 2 | A | 47 | 84 | E−01 | [0.75- | CACAGCCTCAGGACTCCTTG | ID |
| | | | | | | | | | 4.09] | [A/C]AGCATCTATGCCCGT | NO: |
| | | | | | | | | | | CCACAGCAGCACCAGATCGC | 851 |
| | | | | | | | | | | CACAC | |
| chr | 100635 | G | A | MUC1 | p.S614 | 0.005 | 0.003 | 3.40 | 1.68 | TCCTTGAAGCATCTATGCCC | SEQ |
| 7 | 686 | | | 2 | S | 39 | 22 | E−02 | [1.08- | GTCCACAGCAGCACCAGATC | ID |
| | | | | | | | | | 2.61] | [G/A]CCACACACAACACTG | NO: |
| | | | | | | | | | | TCCCCTGCCGGCTCTACAAC | 852 |
| | | | | | | | | | | CCGTC | |
| chr | 100635 | C | T | MUC1 | p.P657 | 0.004 | 0.003 | 7.98 | 1.04 | AGGCCTGCACCTCCTACTAC | SEQ |
| 7 | 814 | | | 2 | L | 17 | 99 | E−01 | [0.64- | CACATCAGCCTTTGTTGAGC | ID |
| | | | | | | | | | 1.71] | [C/T]ATCTACAACCTCCCA | NO: |
| | | | | | | | | | | CGGCAGCCCGAGCTCAATTC | 853 |
| | | | | | | | | | | CAACA | |
| chr | 100635 | C | G | MUC1 | p.H672 | 0.000 | 0.000 | 4.91 | 1.59 | TACAACCTCCCACGGCAGCC | SEQ |
| 7 | 858 | | | 2 | D | 25 | 15 | E−01 | [0.2- | CGAGCTCAATTCCAACAACC | ID |
| | | | | | | | | | 12.55] | [C/G]ACATTTCTGCCCGCT | NO: |
| | | | | | | | | | | CCACAACCTCAGGCCTCGTT | 854 |
| | | | | | | | | | | GAAGA | |
| chr | 100635 | T | A | MUC1 | p.S674 | 0.000 | 0.000 | 7.03 | 3.58 | CTCCCACGGCAGCCCGAGCT | SEQ |
| 7 | 864 | | | 2 | T | 74 | 21 | E−02 | [1.01- | CAATTCCAACAACCCACATT | ID |
| | | | | | | | | | 12.69] | [T/A]CTGCCCGCTCCACAA | NO: |
| | | | | | | | | | | CCTCAGGCCTCGTTGAAGAA | 855 |
| | | | | | | | | | | TCTAC | |
| chr | 100635 | G | A | MUC1 | p.R676 | 0.000 | 0.000 | 1.84 | 7.15 | GGCAGCCCGAGCTCAATTCC | SEQ |
| 7 | 871 | | | 2 | H | 25 | 03 | E−01 | [0.65- | AACAACCCACATTTCTGCCC | ID |
| | | | | | | | | | 78.87] | [G/A]CTCCACAACCTCAGG | NO: |
| | | | | | | | | | | CCTCGTTGAAGAATCTACGA | 856 |
| | | | | | | | | | | CCTAC | |
| chr | 100635 | C | A | MUC1 | p.T679 | 0.004 | 0.003 | 2.87 | 1.28[ | AGCTCAATTCCAACAACCCA | SEQ |
| 7 | 880 | | | 2 | N | 66 | 65 | E−01 | 0.8- | CATTTCTGCCCGCTCCACAA | ID |
| | | | | | | | | | 2.04] | [C/A]CTCAGGCCTCGTTGA | NO: |
| | | | | | | | | | | AGAATCTACGACCTACCACA | 857 |
| | | | | | | | | | | GCAGC | |
| chr | 100635 | C | G | MUC1 | p.S695 | 0.000 | 0.000 | 3.71 | Inf | CTCGTTGAAGAATCTACGAC | SEQ |
| 7 | 928 | | | 2 | X | 25 | 00 | E−02 | [NaN- | CTACCACAGCAGCCCGGGCT | ID |
| | | | | | | | | | Inf] | [C/G]AACTCAAACAATGCA | NO: |
| | | | | | | | | | | CTTCCCTGAAAGCGACACAA | 858 |
| | | | | | | | | | | CTTCA | |
| chr | 100636 | C | A | MUC1 | p.S910 | 0.005 | 0.016 | 8.63 | 0.35 | AGCACCACCACCTCAGGCCC | SEQ |
| 7 | 573 | | | 2 | Y | 64 | 13 | E−09 | [0.23- | CAGTCAGGAATCAACAACTT | ID |
| | | | | | | | | | 0.53] | [C/A]CCACAGCAGCTCAGG | NO: |
| | | | | | | | | | | TTCAACTGACACAGCACTGT | 859 |
| | | | | | | | | | | CCCCT | |
| chr | 100636 | G | A | MUC1 | p.R974 | 0.000 | 0.000 | 2.64 | 18.63 | GAAGCATCTACACGCGTCCA | SEQ |
| 7 | 765 | | | 2 | H | 49 | 03 | E−02 | [1.69- | CAGCAGCACTGGCTCACCAC | ID |
| | | | | | | | | | 205.52] | [G/A]CACAACACTGTCCCG | NO: |
| | | | | | | | | | | TGCCAGCTCCACAAGCCCTG | 860 |
| | | | | | | | | | | GACTT | |
| chr | 100636 | C | G | MUC1 | p.T996 | 0.000 | 0.000 | 2.81 | 2.12 | ACAAGCCCTGGACTTCAGGG | SEQ |
| 7 | 831 | | | 2 | S | 49 | 23 | E−01 | [0.46- | AGAATCTACTGCCTTCCAGA | ID |
| | | | | | | | | | 9.8] | [C/G]CCACCCAGCCTCAAC | NO: |
| | | | | | | | | | | TCACACAACGCCTTCACCTC | 861 |
| | | | | | | | | | | CTAGC | |
| chr | 100636 | T | C | MUC1 | p.S100 | 0.005 | 0.006 | 3.48 | 0.78 | TGCCTTCCAGACCCACCCAG | SEQ |
| 7 | 860 | | | 2 | 6P | 15 | 58 | E−01 | [0.5- | CCTCAACTCACACAACGCCT | ID |
| | | | | | | | | | 1.22] | [T/C]CACCTCCTAGCACCG | NO: |
| | | | | | | | | | | CAACAGCCCCTGTTGAAGAA | 862 |
| | | | | | | | | | | TCTAC | |
| chr | 100637 | C | G | MUC1 | p.P113 | 0.006 | 0.000 | 5.46 | 250.3 | CTGGGCGTCGGTGAAGAATC | SEQ |
| 7 | 251 | | | 2 | 62 | 37 | 03 | E−26 | [33.96- | CACCACCTCCCGTAGCCAAC | ID |
| | | | | | | | | | 1844.95] | [C/G]AGGTTCTACTCACTC | NO: |
| | | | | | | | | | | AACAGTGTCACCTGCCAGCA | 863 |
| | | | | | | | | | | CCACC | |
| chr | 100637 | C | G | MUC1 | p.T118 | 0.001 | 0.001 | 4.55 | 1.37 | CACAGCACCACAACCTCAGT | SEQ |
| 7 | 407 | | | 2 | 8S | 47 | 07 | E−01 | [0.58- | TCATGGTGAAGAGCCTACAA | ID |
| | | | | | | | | | 3.23] | [C/G]CTTCCACAGCCGGCC | NO: |
| | | | | | | | | | | AGCCTCAACTCACACAACAC | 864 |
| | | | | | | | | | | TGTTC | |
| chr | 100637 | G | A | MUC1 | p.G123 | 0.008 | 0.011 | 1.98 | 0.79 | CCAAACAGGGTTACCTGCCA | SEQ |
| 7 | 556 | | | 2 | 8S | 82 | 19 | E−01 | [0.56- | CACTCACAACCGCAGACCTC | ID |
| | | | | | | | | | 1.11] | [G/A]GTGAGGAATCAACTA | NO: |
| | | | | | | | | | | CCTTTCCCAGCAGCTCAGGC | 865 |
| | | | | | | | | | | TCAAC | |
| chr | 100637 | C | T | MUC1 | p.P135 | 0.001 | 0.001 | 1.00 | 0.87 | TTCCCTGACAGCACCACCAC | SEQ |
| 7 | 902 | | | 2 | 3L | 47 | 69 | E+00 | [0.37- | CTCAGACCTCAGTCAGGAAC | ID |
| | | | | | | | | | 2.04] | [C/T]TACAACTTCCCACAG | NO: |
| | | | | | | | | | | CAGCCAAGGCTCAACAGAGG | 866 |
| | | | | | | | | | | CAACA | |
| chr | 100638 | C | G | MUC1 | p.H158 | 0.006 | 0.000 | 1.36 | Inf | CGACAAGCTCAGGCGTCAGT | SEQ |
| 7 | 584 | | | 2 | 0Q | 13 | 00 | E−29 | | GAAGAATCCACCACCTCCCA | ID |
| | | | | | | | | | | [C/G]AGCCGACCAGGCTCA | NO: |
| | | | | | | | | | | ACGCACACAACAGCATTCCC | 867 |
| | | | | | | | | | | TGGCA | |
| chr | 100638 | G | T | MUCl | p.S161 | 0.001 | 0.000 | 1.36 | 21.28 | ATGCCAGGCGTCAGTCAGGA | SEQ |
| 7 | 673 | | | 2 | 0I | 47 | 07 | E−05 | [6- | ATCTACAGCTTCCCACAGCA | ID |
| | | | | | | | | | 75.44] | [G/T]CCCAGGCTCCACAGA | NO: |
| | | | | | | | | | | CACAACATTGTCCCCTGGCA | 868 |
| | | | | | | | | | | GTACC | |
| chr | 100638 | G | A | MUC1 | p.S163 | 0.000 | 0.000 | 2.36 | 14.25 | ACAGCATCATCCCTTGGTCC | SEQ |
| 7 | 754 | | | 2 | 7N | 49 | 03 | E−02 | [2.01- | AGAATCTACTACTTTCCACA | ID |
| | | | | | | | | | 101.22] | [G/A]CAGCCCAGGCTCCAC | NO: |
| | | | | | | | | | | TGAAACAACACTCTTACCTG | 869 |
| | | | | | | | | | | ACAAC | |
| chr | 100638 | C | T | MUC1 | p.S166 | 0.000 | 0.000 | 6.13 | 1.1 | CTCCTTGAAGCATCTACGCC | SEQ |
| 7 | 850 | | | 2 | 9L | 25 | 22 | E−01 | [0.14- | CGTCCACAGCAGCACTGGAT | ID |
| | | | | | | | | | 8.39] | [C/T]GCCACACACAACACT | NO: |
| | | | | | | | | | | GTCCCCTGCCGGCTCTACAA | 870 |
| | | | | | | | | | | CACGT | |
| chr | 100638 | G | A | MUC1 | p.R168 | 0.001 | 0.000 | 2.33 | 1.7[ | TCGCCACACACAACACTGTC | SEQ |
| 7 | 889 | | | 2 | 2H | 23 | 72 | E−01 | 0.67- | CCCTGCCGGCTCTACAACAC | ID |
| | | | | | | | | | 4.31] | [G/A]TCAGGGAGAATCTAC | NO: |
| | | | | | | | | | | CACCTTCCAGAGCTGGCCAA | 871 |
| | | | | | | | | | | GCTCA | |
| chr | 100638 | G | A | MUC1 | p.W16 | 0.000 | 0.000 | 1.84 | 7.15 | TCTACAACACGTCAGGGAGA | SEQ |
| 7 | 919 | | | 2 | 92X | 25 | 03 | E−01 | [0.65- | ATCTACCACCTTCCAGAGCT | ID |
| | | | | | | | | | 78.9] | [G/A]GCCAAGCTCAAAGGA | NO: |
| | | | | | | | | | | CACTATGCCTGCACCTCCTA | 872 |
| | | | | | | | | | | CTACC | |
| chr | 100638 | C | G | MUC1 | p.P169 | 0.000 | 0.000 | 6.53 | Inf | ACAACACGTCAGGGAGAATC | SEQ |
| 7 | 922 | | | 2 | 3R | 25 | 00 | E−02 | [NaN- | TACCACCTTCCAGAGCTGGC | ID |
| | | | | | | | | | Inf] | [C/G]AAGCTCAAAGGACAC | NO: |
| | | | | | | | | | | TATGCCTGCACCTCCTACTA | 873 |
| | | | | | | | | | | CCACA | |
| chr | 100638 | G | A | MUC1 | p.S169 | 0.000 | 0.000 | 6.45 | 1.51 | ACACGTCAGGGAGAATCTAC | SEQ |
| 7 | 925 | | | 2 | 4N | 49 | 33 | E−01 | [0.35- | CACCTTCCAGAGCTGGCCAA | ID |
| | | | | | | | | | 6.47] | [G/A]CTCAAAGGACACTAT | NO: |
| | | | | | | | | | | GCCTGCACCTCCTACTACCA | 874 |
| | | | | | | | | | | CATCA | |
| chr | 100638 | C | G | MUC1 | p.S169 | 0.000 | 0.000 | 3.71 | Inf | CGTCAGGGAGAATCTACCAC | SEQ |
| 7 | 928 | | | 2 | 5X | 25 | 00 | E−02 | [NaN- | CTTCCAGAGCTGGCCAAGCT | ID |
| | | | | | | | | | Inf] | [C/G]AAAGGACACTATGCC | NO: |
| | | | | | | | | | | TGCACCTCCTACTACCACAT | 875 |
| | | | | | | | | | | CAGCC | |
| chr | 100643 | C | G | MUC1 | p.H313 | 0.017 | 0.000 | 9.03 | Inf | CGACAAGCTCAGGCGTCAGT | SEQ |
| 7 | 255 | | | 2 | 7Q | 16 | 00 | E−84 | | GAAGAATCCACCACCTCCCA | ID |
| | | | | | | | | | | [C/G]AGCCGACCAGGCTCA | NO: |
| | | | | | | | | | | ACGCACACAACAGCATTCCC | 876 |
| | | | | | | | | | | TGGCA | |
| chr | 100643 | G | A | MUC1 | p.A318 | 0.005 | 0.001 | 2.43 | 3.89 | AGGCTCCACAGACACAACAC | SEQ |
| 7 | 388 | | | 2 | 2T | 88 | 52 | E−07 | [2.47- | TGTCCCCTGGCAGTACCACA | ID |
| | | | | | | | | | 6.12] | [G/A]CATCATCCCTTGGTC | NO: |
| | | | | | | | | | | CAGAATCTACTACCTTCCAC | 877 |
| | | | | | | | | | | AGCGG | |
| chr | 100643 | G | A | MUC1 | p.R323 | 0.003 | 0.000 | 2.27 | 44.85 | TCGCCACACACAACACTGTC | SEQ |
| 7 | 560 | | | 2 | 9H | 92 | 09 | E−15 | [16.42- | CCCTGCCGGCTCTACAACCC | ID |
| | | | | | | | | | 122.48] | [G/A]TCAGGGAGAATCTAC | NO: |
| | | | | | | | | | | CACCTTCCAGAGCTGGCCTA | 878 |
| | | | | | | | | | | ACTCG | |
| chr | 100643 | A | G | MUC1 | p.T324 | 0.000 | 0.000 | 3.74 | 9.54 | ACTGTCCCCTGCCGGCTCTA | SEQ |
| 7 | 574 | | | 2 | 4A | 49 | 05 | E−02 | [1.59- | CAACCCGTCAGGGAGAATCT | ID |
| | | | | | | | | | 57.13] | [A/G]CCACCTTCCAGAGCT | NO: |
| | | | | | | | | | | GGCCTAACTCGAAGGACACT | 879 |
| | | | | | | | | | | ACCCC | |
| chr | 100643 | C | T | MUC1 | p.S329 | 0.000 | 0.000 | 6.47 | 1.48 | TTTTCTGCCAGCTCCACAAC | SEQ |
| 7 | 737 | | | 2 | 8L | 49 | 33 | E−01 | [0.34- | CTTGGGCCGTAGTGAGGAAT | ID |
| | | | | | | | | | 6.34] | [C/T]GACAACAGTCCACAG | NO: |
| | | | | | | | | | | CAGCCCAGTTGCAACTGCAA | 880 |
| | | | | | | | | | | CAACA | |
| chr | 100643 | G | A | MUC1 | p.R331 | 0.008 | 0.000 | 1.03 | 36.59 | AGCAGCCCAGTTGCAACTGC | SEQ |
| 7 | 791 | | | 2 | 6H | 09 | 22 | E−28 | [18.88- | AACAACACCCTCGCCTGCCC | ID |
| | | | | | | | | | 70.9] | [G/A]CTCCACAACCTCAGG | NO: |
| | | | | | | | | | | CCTCGTTGAAGAATCTACGA | 881 |
| | | | | | | | | | | CCTAC | |
| chr | 100646 | G | A | MUC1 | p.S424 | 0.000 | 0.000 | 4.92 | 1.36 | ACCATGCCAGGCGTCAGTCA | SEQ |
| 7 | 590 | | | 2 | 9N | 74 | 54 | E−01 | [0.42- | GGAATCTACAGCTTCCCACA | ID |
| | | | | | | | | | 4.44] | [G/A]CAGCCCAGGCTCCAC | NO: |
| | | | | | | | | | | AGACACAACACTGTCCCCTG | 882 |
| | | | | | | | | | | GCAGT | |
| chr | 100646 | A | C | MUC1 | p.N42 | 0.021 | 0.051 | 5.47 | 0.4 | CAGCAGCCCAGGCTCCACTG | SEQ |
| 7 | 712 | | | 2 | 90H | 08 | 65 | E−22 | [0.32- | AAACAACACTCTTACCTGAC | ID |
| | | | | | | | | | 0.49] | [A/C]ACACCACAGCCTCAG | NO: |
| | | | | | | | | | | GCCTCCTTGAAGCATCTACA | 883 |
| | | | | | | | | | | CCCGT | |
| chr | 100646 | C | G | MUC1 | p.P430 | 0.022 | 0.000 | 6.14 | 313.86 | GACAACACCACAGCCTCAGG | SEQ |
| 7 | 749 | | | 2 | 2R | 55 | 07 | E−92 | [99.34- | CCTCCTTGAAGCATCTACAC | ID |
| | | | | | | | | | 991.56] | [C/G]CGTCCACAGCAGCAC | NO: |
| | | | | | | | | | | TGGATCGCCACACACAACAC | 884 |
| | | | | | | | | | | TGTCC | |
| chr | 100646 | G | A | MUC1 | p.R432 | 0.000 | 0.000 | 1.00 | 0.89 | TCGCCACACACAACACTGTC | SEQ |
| 7 | 809 | | | 2 | 2H | 25 | 27 | E+00 | [0.12- | CCCTGCCGGCTCTACAACCC | ID |
| | | | | | | | | | 6.73] | [G/A]TCAGGGAGAATCTAC | NO: |
| | | | | | | | | | | CACCTTCCAGAGCTGGCCAA | 885 |
| | | | | | | | | | | ACTCG | |
| chr | 100646 | C | T | MUC1 | p.R437 | 0.002 | 0.002 | 8.75 | 0.89 | TCCAACAACCCACTTTTCTG | SEQ |
| 7 | 973 | | | 2 | 7C | 45 | 75 | E−01 | [0.47- | CCAGCTCCACAACATTGGGC | ID |
| | | | | | | | | | 1.7] | [C/T]GTAGTGAGGAATCGA | NO: |
| | | | | | | | | | | CAACAGTCCACAGCAGCCCA | 886 |
| | | | | | | | | | | GTTGC | |
| chr | 100647 | A | G | MUC1 | p.R463 | 0.000 | 0.000 | 6.60 | Inf | CCCTGAAAGCTCCACAGCTT | SEQ |
| 7 | 735 | | | 2 | 1G | 25 | 00 | E−02 | [NaN- | CAGGTCGTAGTGAAGAATCA | ID |
| | | | | | | | | | Inf] | [A/G]GAACTTCCCACAGCA | NO: |
| | | | | | | | | | | GCACAACACACACAATATCT | 887 |
| | | | | | | | | | | TCACC | |
| chr | 100647 | C | G | MUC1 | p.P464 | 0.006 | 0.006 | 9.21 | 0.95 | AAGAACTTCCCACAGCAGCA | SEQ |
| 7 | 774 | | | 2 | 4A | 37 | 73 | E−01 | [0.64- | CAACACACACAATATCTTCA | ID |
| | | | | | | | | | 1.41] | [C/G]CTCCTAGCACCACAT | NO: |
| | | | | | | | | | | CTGCCCTTGTTGAAGAACCT | 888 |
| | | | | | | | | | | ACCAG | |
| chr | 100647 | C | G | MUC1 | p.S471 | 0.005 | 0.003 | 1.60 | 1.37 | TTACCTGCCCATTTTACTAC | SEQ |
| 7 | 976 | | | 2 | 1C | 39 | 95 | E−01 | [0.88- | CTCAGGCCGCATTGCAGAAT | ID |
| | | | | | | | | | 2.12] | [C/G]TACCACCTTCTATAT | NO: |
| | | | | | | | | | | CTCTCCAGGCTCAATGGAAA | 889 |
| | | | | | | | | | | CAACA | |
| chr | 100647 | A | G | MUC1 | p.Y471 | 0.000 | 0.000 | 2.36 | 14.27 | TTTACTACCTCAGGCCGCAT | SEQ |
| 7 | 988 | | | 2 | 5C | 49 | 03 | E−02 | [2.01- | TGCAGAATCTACCACCTTCT | ID |
| | | | | | | | | | 101.32] | [A/G]TATCTCTCCAGGCTC | NO: |
| | | | | | | | | | | AATGGAAACAACATTAGCCA | 890 |
| | | | | | | | | | | GCACT | |
| chr | 100648 | C | G | MUC1 | p.L473 | 0.005 | 0.006 | 7.56 | 0.91 | AATGGAAACAACATTAGCCA | SEQ |
| 7 | 044 | | | 2 | 4V | 64 | 19 | E−01 | [0.6- | GCACTGCCACAACACCAGGC | ID |
| | | | | | | | | | 1.39] | [C/G]TCAGTGCAAAATCTA | NO: |
| | | | | | | | | | | CCATCCTTTACAGTAGCTCC | 891 |
| | | | | | | | | | | AGATC | |
| chr | 100648 | C | G | MUC1 | p.S476 | 0.000 | 0.000 | 3.78 | 2.38 | CCAGCATGACAAGCTCCAGC | SEQ |
| 7 | 148 | | | 2 | 8R | 25 | 10 | E−01 | [0.29- | ATCAGTGGAGAACCCACCAG | ID |
| | | | | | | | | | 19.75] | [C/G]TTGTATAGCCAAGCA | NO: |
| | | | | | | | | | | GAGTCAACACACACAACAGC | 892 |
| | | | | | | | | | | GTTCC | |
| chr | 100648 | C | T | MUC1 | p.A478 | 0.000 | 0.000 | 1.84 | 7.12 | ACCAGCTTGTATAGCCAAGC | SEQ |
| 7 | 183 | | | 2 | 0V | 25 | 03 | E−01 | [0.65- | AGAGTCAACACACACAACAG | ID |
| | | | | | | | | | 78.55] | [C/T]GTTCCCTGCCAGCAC | NO: |
| | | | | | | | | | | CACCACCTCAGGCCTCAGTC | 893 |
| | | | | | | | | | | AGGAA | |
| chr | 100649 | G | T | MUC1 | p.C498 | 0.000 | 0.000 | 7.00 | 1.12 | CACGGTGACTGCTGTGGATT | SEQ |
| 7 | 758 | | | 2 | 8F | 49 | 44 | E−01 | [0.27- | CTATCTCTCCACAGGGTTGT | ID |
| | | | | | | | | | 4.73] | [G/T]CCAGGAAGGACAAAT | NO: |
| | | | | | | | | | | TTGGAATGGAAAACAATGCG | 894 |
| | | | | | | | | | | TCTGT | |
| chr | 100649 | G | C | MUC2 | p.G500 | 0.000 | 0.000 | 2.41 | 4.67 | TGGAATGGAAAACAATGCGT | SEQ |
| 7 | 815 | | | 2 | 7A | 25 | 05 | E−01 | [0.49- | CTGTCCCCAAGGCTACGTTG | ID |
| | | | | | | | | | 44.94] | [G/C]TTACCAGTGCTTGTC | NO: |
| | | | | | | | | | | CCCTCTGGAATCCTTCCCTG | 895 |
| | | | | | | | | | | TAGGT | |
| chr | 100649 | C | T | MUC1 | p.P501 | 0.000 | 0.000 | 2.59 | 0.29 | CTACGTTGGTTACCAGTGCT | SEQ |
| 7 | 847 | | | 2 | 85 | 25 | 86 | E−01 | [0.04- | TGTCCCCTCTGGAATCCTTC | ID |
| | | | | | | | | | 2.07] | [C/T]CTGTAGGTAATGACC | NO: |
| | | | | | | | | | | TTTTCTGAGACCTGCAGCTC | 896 |
| | | | | | | | | | | TTTGC | |
| chr | 100649 | T | C | MUC1 | p.V501 | 0.000 | 0.000 | 9.98 | 3 | GTTGGTTACCAGTGCTTGTC | SEQ |
| 7 | 851 | | | 2 | 9A | 74 | 24 | E−02 | [0.86- | CCCTCTGGAATCCTTCCCTG | ID |
| | | | | | | | | | 10.46] | [T/C]AGGTAATGACCTTTT | NO: |
| | | | | | | | | | | CTGAGACCTGCAGCTCTTTG | 897 |
| | | | | | | | | | | CAGGC | |
| chr | 100651 | C | T | MUC1 | p.P502 | 0.000 | 0.000 | 4.29 | 1.69 | GCTGTCTCACGCATACCATG | SEQ |
| 7 | 921 | | | 2 | 2L | 74 | 43 | E−01 | [0.51- | GCCTTTTCCCACAGAAACCC | ID |
| | | | | | | | | | 5.6] | [C/T]GGAAAAACTCAACGC | NO: |
| | | | | | | | | | | CACTTTAGGTATGACAGTGA | 898 |
| | | | | | | | | | | AAGTG | |
| chr | 100656 | T | C | MUC1 | p.L520 | 0.000 | 0.000 | 1.29 | 14.02 | AAGTGCACCAAAGGAACGAA | SEQ |
| 7 | 384 | | | 2 | 0P | 25 | 02 | E−01 | [0.88- | GTCGCAAATGAACTGTAACC | ID |
| | | | | | | | | | 224.21] | [T/C]GGGCACATGTCAGCT | NO: |
| | | | | | | | | | | GCAACGCAGTGGCCCCCGCT | 899 |
| | | | | | | | | | | GCCTG | |
| chr | 100657 | T | C | MUC1 | p.I523 | 0.000 | 0.000 | 6.19 | 1.08 | AACACACACTGGTACTGGGG | SEQ |
| 7 | 247 | | | 2 | 1T | 25 | 23 | E−01 | [0.14- | AGAGACCTGTGAATTCAACA | ID |
| | | | | | | | | | 8.25] | [T/C]CGCCAAGAGCCTCGT | NO: |
| | | | | | | | | | | GTATGGGATCGTGGGGGCTG | 900 |
| | | | | | | | | | | TGATG | |
| chr | 100678 | G | A | MUC1 | p.P140 | 0.018 | 0.000 | 2.01 | 1009.33 | GAACCACTCCGTTAACAAGT | SEQ |
| 7 | 918 | | | 7 | 7P | 14 | 02 | E− | [247.69- | ATACCTGTCAGCACCACGCC | ID |
| | | | | | | | | 104 | 4112.96] | [G/A]GTAGTCAGTTCTGAG | NO: |
| | | | | | | | | | | GCTAGCACCCTTTCAGCAAC | 901 |
| | | | | | | | | | | TCCTG | |
| chr | 100681 | C | T | MUC1 | p.A217 | 0.012 | 0.000 | 8.18 | Inf | CTCCTTTAACAAGTATGCCT | SEQ |
| 7 | 219 | | | 7 | 4A | 99 | 00 | E−78 | | GTCAGCACCACAGTGGTGGC | ID |
| | | | | | | | | | | [C/T]AGTTCTGCAATCAGC | NO: |
| | | | | | | | | | | ACCCTTTCAACAACTCCTGT | 902 |
| | | | | | | | | | | TGACA | |
| chr | 100681 | T | G | MUC1 | p.S220 | 0.006 | 0.000 | 2.25 | Inf | TGTGACCAATTCTACTGAAG | SEQ |
| 7 | 310 | | | 7 | 5A | 37 | 00 | E−38 | | CCCGTTCATCTCCTACAACT | ID |
| | | | | | | | | | | [T/G]CTGAAGGTACCAGCA | NO: |
| | | | | | | | | | | TGCCAACCTCAACTCCTAGT | 903 |
| | | | | | | | | | | GAAGG | |
| chr | 100682 | T | C | MUC1 | p.S263 | 0.007 | 0.001 | 4.22 | 4.95 | TACCAGCATGCCAATCTCAA | SEQ |
| 7 | 597 | | | 7 | 4P | 11 | 44 | E−11 | [3.33- | CTCCTAGTGAAGTAAGTACT | ID |
| | | | | | | | | | 7.38] | [T/C]CATTAACAAGTATAC | NO: |
| | | | | | | | | | | TTGTCAGCACCATGCCAGTG | 904 |
| | | | | | | | | | | GCCAG | |
| chr | 100682 | T | C | MUC1 | p.L263 | 0.006 | 0.000 | 2.08 | 14.32 | TCAACTCCTAGTGAAGTAAG | SEQ |
| 7 | 613 | | | 7 | 9P | 86 | 48 | E−20 | [9.05- | TACTTCATTAACAAGTATAC | ID |
| | | | | | | | | | 22.65] | [T/C]TGTCAGCACCATGCC | NO: |
| | | | | | | | | | | AGTGGCCAGTTCTGAGGCTA | 905 |
| | | | | | | | | | | GCACC | |
| chr | 102087 | C | T | ORAI2 | p.L168 | 0.011 | 0.006 | 2.80 | 1.82 | TGCTTGGCATCCTACTCTTC | SEQ |
| 7 | 238 | | | | L | 27 | 23 | E−04 | [1.35- | CTGGCCGAGGTGGTGCTGCT | ID |
| | | | | | | | | | 2.46] | [C/T]TGCTGGATCAAGTTC | NO: |
| | | | | | | | | | | CTCCCCGTGGATGCCCGGCG | 906 |
| | | | | | | | | | | CCAGC | |
| chr | 108112 | A | G | PNPLA | p.D764 | 0.005 | 0.003 | 1.58 | 1.69 | ATGGAAGTCCTTCATACATA | SEQ |
| 7 | 902 | | | 8 | D | 88 | 48 | E−02 | [1.12- | TCAGTTTTTAATTTTATCCA | ID |
| | | | | | | | | | 2.56] | [A/G]TCATTAATTTTCTGC | NO: |
| | | | | | | | | | | AGAGTTGTTTTTTCTTGACT | 907 |
| | | | | | | | | | | TAATA | |
| chr | 111368 | G | A | DOCK4 | p.P191 | 0.009 | 0.005 | 2.33 | 1.74 | CGCGGGCGGCTCCGACGTGA | SEQ |
| 7 | 481 | | | | 7L | 31 | 38 | E−03 | [1.25- | CGGGGATGGAGAGGCTGTGA | ID |
| | | | | | | | | | 2.42] | [G/A]GTAGCGGGACGGGGC | NO: |
| | | | | | | | | | | GCCGCAGAGTCCGCTCGTAG | 908 |
| | | | | | | | | | | ACGCT | |
| chr | 117232 | A | G | CFTR | p.E695 | 0.021 | 0.000 | 3.53 | 2406.22 | ACAAAAAAACAATCTTTTAA | SEQ |
| 7 | 305 | | | | G | 32 | 01 | E− | [335.08- | ACAGACTGGAGAGTTTGGGG | ID |
| | | | | | | | | 125 | 17279.21] | [A/G]AAAAAGGAAGAATTC | NO: |
| | | | | | | | | | | TATTCTCAATCCAATCAACT | 909 |
| | | | | | | | | | | CTATA | |
| chr | 123143 | G | A | IQUB | p.P278 | 0.007 | 0.004 | 1.12 | 1.65 | ACATTACCTGCGTATCCCTA | SEQ |
| 7 | 031 | | | | P | 84 | 78 | E−02 | [1.15- | CAAAATATACTGAGTCTTTC | ID |
| | | | | | | | | | 2.36] | [G/A]GGAATCCTTTTAGGT | NO: |
| | | | | | | | | | | ACAGTTTGTGTTCCAGCATT | 910 |
| | | | | | | | | | | GTGAT | |
| chr | 141366 | A | G | KIAA11 | p.M23 | 0.006 | 0.004 | 4.89 | 1.52 | GATGAGGATCTGTTCTCCAA | SEQ |
| 7 | 203 | | | 47 | 5T | 37 | 20 | E−02 | [1.02- | AGAACTTTATAAACTGAGAC | ID |
| | | | | | | | | | 2.26] | [A/G]TGCAGCCAGCTGGGT | NO: |
| | | | | | | | | | | GTGTGATCTGAAAAAATTGA | 911 |
| | | | | | | | | | | GGGGA | |
| chr | 141763 | C | A | MGAM | p.P142 | 0.012 | 0.009 | 2.68 | 1.38 | GAGGTATGTCTGTGTTTGGC | SEQ |
| 7 | 311 | | | | 41 | 99 | 45 | E−02 | [1.05- | ATTTCTAGGATATGAATGAA | ID |
| | | | | | | | | | 1.82] | [C/A]CATCAAGCTTCGTGA | NO: |
| | | | | | | | | | | ATGGGGCAGTTTCTCCAGGC | 912 |
| | | | | | | | | | | TGCAG | |
| chr | 141794 | C | T | MGAM | p.F154 | 0.006 | 0.002 | 1.83 | 2.75 | CTGTGCTTCTCGTTGCAGGC | SEQ |
| 7 | 442 | | | | 7F | 13 | 24 | E−05 | [1.82- | ATGATGGAGTTCAGCCTCTT | ID |
| | | | | | | | | | 4.15] | [C/T]GGCATATCCTATGTG | NO: |
| | | | | | | | | | | AGTGTCCTTGGGATCCTCCT | 913 |
| | | | | | | | | | | AAGCA | |
| chr | 150069 | G | A | REPIN1 | p.K248 | 0.009 | 0.000 | 1.71 | Inf | CCTTCCAGTGTGCCTGTTGT | SEQ |
| 7 | 074 | | | | K | 56 | 00 | E−56 | | GGCAAGCGCTTCCGGCACAA | ID |
| | | | | | | | | | | [G/A]CCCAACTTGATCGCT | NO: |
| | | | | | | | | | | CACCGCCGCGTGCACACGGG | 914 |
| | | | | | | | | | | CGAGC | |
| chr | 150738 | C | T | ABCB8 | p.G405 | 0.005 | 0.002 | 5.50 | 1.9 | TGCCCCCTGGCAAGATCGTG | SEQ |
| 7 | 005 | | | | G | 64 | 97 | E−03 | [1.24- | GCCCTCGTGGGCCAGTCTGG | ID |
| | | | | | | | | | 2.91] | [C/T]GGAGGTAAGGGGAGC | NO: |
| | | | | | | | | | | CCACCACCTCTTCACCCTCT | 915 |
| | | | | | | | | | | GACTC | |
| chr | 150840 | A | T | AGAP3 | p.E431 | 0.005 | 0.002 | 1.04 | 1.85 | TCATGCCCTGATGGGCCTGT | SEQ |
| 7 | 440 | | | | D | 15 | 79 | E−02 | [1.19- | GGTTGCAGAGAGGAGAAGGA | ID |
| | | | | | | | | | 2.88] | [A/T]CGCTGGATACGGGCC | NO: |
| | | | | | | | | | | AAGTATGAACAGAAGCTCTT | 916 |
| | | | | | | | | | | CCTGG | |
| chr | 151078 | C | T | WDR8 | p.G313 | 0.006 | 0.003 | 8.14 | 1.73 | GAGGGACCTACCTGGATGCA | SEQ |
| 7 | 993 | | | 6 | S | 86 | 98 | E−03 | [1.18- | GTTGATGAATGAATGTGTGGC | ID |
| | | | | | | | | | 2.54] | [C/T]CCGGAACACCCTCCG | NO: |
| | | | | | | | | | | CAGCTCTCCAGACTGCGCGT | 917 |
| | | | | | | | | | | CGAAG | |
| chr | 151859 | G | A | KMT2C | p.S358 | 0.005 | 0.003 | 4.53 | 1.58 | TTTTTCCTCTGGGATTATAT | SEQ |
| 7 | 899 | | | | 8L | 64 | 57 | E−02 | [1.04- | CAGAATACAACTGAATGAGC | ID |
| | | | | | | | | | 2.41] | [G/A]ATTGGGTTGATCCCG | NO: |
| | | | | | | | | | | GATAACTGTGTCCATGGGTT | 918 |
| | | | | | | | | | | ATAGT | |
| chr | 623435 | G | A | ERICH1 | p.P306 | 0.027 | 0.000 | 7.40 | 1561.55 | CTCCCCGGAGTCTGCACCCT | SEQ |
| 8 | | | | | L | 21 | 02 | E− | [385.58- | CTTCCTCCCCAGCCCATGTC | ID |
| | | | | | | | | 159 | 6324.12] | [G/A]GGTCTTCCTCGCTGG | NO: |
| | | | | | | | | | | CGTCCGCACCGTCCTCCTCC | 919 |
| | | | | | | | | | | CTGGT | |
| chr | 623519 | A | C | ERICH1 | p.I278 | 0.014 | 0.000 | 1.13 | Inf | TTTACCGTCTTCCTCCCCGG | SEQ |
| 8 | | | | | S | 71 | 00 | E−87 | | CCCGTGTCAGGTCTTCCTCA | ID |
| | | | | | | | | | | [A/C]TGGTGTCCACACCGT | NO: |
| | | | | | | | | | | CCTCCTCCCTGGCGTCTTTA | 920 |
| | | | | | | | | | | ACGTC | |
| chr | 623675 | A | C | ERICH1 | p.V226 | 0.024 | 0.000 | 1.64 | Inf | CTCGCTAGCGTCCGCACCAT | SEQ |
| 8 | | | | | G | 51 | 00 | E− | | CTTCCTCCCTGGTATCTTTA | ID |
| | | | | | | | | 145 | | [A/C]CGTCTTCCTCCCCGG | NO: |
| | | | | | | | | | | CCAGTGTCGGGTCTTCCTCG | 921 |
| | | | | | | | | | | CTGGT | |
| chr | 104660 | A | C | RP1L1 | p.0185 | 0.005 | 0.000 | 1.36 | Inf | CTTCTGACTCTGGCTGGGCC | SEQ |
| 8 | 31 | | | | 9E | 15 | 00 | E−30 | | TCCCCTTCAGCCTCCTGGGC | ID |
| | | | | | | | | | | [A/C]TCCCCTTCTGCCTCT | NO: |
| | | | | | | | | | | GGGGCCTCTACACCTTCTGA | 922 |
| | | | | | | | | | | CTCTG | |
| chr | 171597 | A | G | MTMR | p.M52 | 0.005 | 0.002 | 1.04 | 1.85 | TAGTTCTTCCTCTAGCTGCT | SEQ |
| 8 | 18 | | | 7 | 2T | 15 | 79 | E−02 | [1.19- | GAGTTTCTTCCTTCACTGCC | ID |
| | | | | | | | | | 2.88] | [A/G]TTAGGTAATCTGTAA | NO: |
| | | | | | | | | | | CTGACTGTCGGGGCTGCATC | 923 |
| | | | | | | | | | | CCCTT | |
| chr | 180803 | A | T | NAT1 | p.D251 | 0.005 | 0.003 | 1.32 | 1.76 | ACCCTCACCCATAGGAGATT | SEQ |
| 8 | 08 | | | | V | 88 | 35 | E−02 | [1.16- | CAATTATAAGGACAATACAG | ID |
| | | | | | | | | | 2.66] | [A/T]TCTAATAGAGTTCAA | NO: |
| | | | | | | | | | | GACTCTGAGTGAGGAAGAAA | 924 |
| | | | | | | | | | | TAGAA | |
| chr | 234289 | C | G | SLC25A | p.T191 | 0.005 | 0.003 | 3.48 | 1.63 | ACCGGTCAGCAATCAGCTGC | SEQ |
| 8 | 24 | | | 37 | T | 15 | 17 | E−02 | [1.05- | ATCCGGACGGTGTGGAGGAC | ID |
| | | | | | | | | | 2.53] | [C/G]GAGGGGTTGGGGGCC | NO: |
| | | | | | | | | | | TTCTACCGGAGCTACACCAC | 925 |
| | | | | | | | | | | GCAGC | |
| chr | 251746 | C | T | DOCK5 | p.T469 | 0.010 | 0.007 | 4.77 | 1.38 | GACAAAGGGAAGAAGAAGAC | SEQ |
| 8 | 10 | | | | M | 78 | 86 | E−02 | [1.01- | GCCAAAGAATGTGGAGGTGA | ID |
| | | | | | | | | | 1.87] | [C/T]GATGTCTGTGCACGA | NO: |
| | | | | | | | | | | TGAGGAGGGCAAGCTCTTGG | 926 |
| | | | | | | | | | | AGGTG | |
| chr | 267219 | C | T | ADRA1 | p.R166 | 0.006 | 0.003 | 2.42 | 1.74 | GTTGATCTGGCAGATGGTCT | SEQ |
| 8 | 90 | | | A | K | 506 | 743 | E−02 | [1.05- | CGTCCTCGGGGGCCGGCTGC | ID |
| | | | | | | | | | 2.73] | [C/T]TCCAGCCGAACAGGG | NO: |
| | | | | | | | | | | GTCCAATGGATATGACCAGG | 927 |
| | | | | | | | | | | GAGAG | |
| chr | 356480 | G | A | UNC5D | p.T930 | 0.009 | 0.005 | 1.25 | 1.79 | CCCTGGCCTGTGCCCTTGAA | SEQ |
| 8 | 09 | | | | T | 07 | 08 | E−03 | [1.28- | GAGATTGGGAGGACACACAC | ID |
| | | | | | | | | | 2.5] | [G/A]AAACTCTCAAACATT | NO: |
| | | | | | | | | | | TCAGAATCCCAGCTTGATGA | 928 |
| | | | | | | | | | | AGCCG | |
| chr | 367933 | T | C | KCNU1 | p.N11 | 0.005 | 0.002 | 8.09 | 2.24 | TATCATCTCAGATACCTTTA | SEQ |
| 8 | 75 | | | | 29N | 64 | 53 | E−04 | [1.46- | GGTGACAATGCAAAAGAAAA | ID |
| | | | | | | | | | 3.43] | [T/C]GAAAGGAAAACTTCA | NO: |
| | | | | | | | | | | GATGAGGTTTATGATGAGGA | 929 |
| | | | | | | | | | | TCCCT | |
| chr | 376997 | G | A | GPR12 | p.K130 | 0.005 | 0.000 | 9.89 | Inf | CGTACCCGCTCAACGCCGCC | SEQ |
| 8 | 77 | | | 4 | 7K | 64 | 00 | E−29 | | AGCCTAAACGGCGCCCCCAA | ID |
| | | | | | | | | | | [G/A]GGGGGCAAGTACGAC | NO: |
| | | | | | | | | | | GACGTCACCCTGATGGGCGC | 930 |
| | | | | | | | | | | GGAGG | |
| chr | 382600 | C | T | LETM2 | p.A331 | 0.006 | 0.003 | 4.19 | 1.56 | AAGTTCCAACTGCATCCCTT | SEQ |
| 8 | 50 | | | | V | 13 | 94 | E−02 | [1.04- | ACATTTCTTTCAGATAATTG | ID |
| | | | | | | | | | 2.34] | [C/T]CAAGGAAGGGGTGAC | NO: |
| | | | | | | | | | | AGCATTGAGTGTATCAGAAC | 931 |
| | | | | | | | | | | TACAG | |
| chr | 382657 | C | T | LETM2 | p.T385 | 0.005 | 0.002 | 2.15 | 1.92 | GTTTTTTACGCCTAGACACT | SEQ |
| 8 | 55 | | | | M | 205 | 716 | E−02 | [1.08- | CCAGGCCAAATCACAAATGA | ID |
| | | | | | | | | | 3.18] | [C/T]GGCCCAGAACAGCAA | NO: |
| | | | | | | | | | | GGCTAGTTCAAAAGGAGCAT | 932 |
| | | | | | | | | | | AAAGG | |
| chr | 523208 | G | C | PXDNL | p.L111 | 0.007 | 0.004 | 4.90 | 1.54 | TAAGCCGCGGAGAAGAGCCT | SEQ |
| 8 | 32 | | | | 8V | 482 | 863 | E−02 | [0.97- | CTGGGTCAGCTCAGGACTGA | ID |
| | | | | | | | | | 2.34] | [G/C]AAGGTAGGAGGGTGC | NO: |
| | | | | | | | | | | CCGCCATTTAGCAGCCACGC | 933 |
| | | | | | | | | | | CAAAC | |
| chr | 550491 | A | G | MRPL1 | p.R57 | 0.012 | 0.008 | 4.23 | 1.36 | GAGAAGAGGTAGAAAATGTG | SEQ |
| 8 | 31 | | | 5 | G | 01 | 88 | E−02 | [1.02- | GCAGAGGCCATAAAGGAGAA | ID |
| | | | | | | | | | 1.81] | [A/G]GGCAAAGAGGAACCC | NO: |
| | | | | | | | | | | GGCCCCGCTTGGGCTTTGAG | 934 |
| | | | | | | | | | | GGAGG | |
| chr | 813991 | C | T | ZBTB1 | p.S36L | 0.018 | 0.014 | 3.39 | 1.31 | GGCGGCGGCTCCACGAACAA | SEQ |
| 8 | 52 | | | 0 | | 87 | 49 | E−02 | [1.03- | TAACGCTGGCGGGGAGGCCT | ID |
| | | | | | | | | | 1.67] | [C/T]AGCTTGGCCTCCGCA | NO: |
| | | | | | | | | | | GCCCCAGCCGAGACAGCCCC | 935 |
| | | | | | | | | | | CGCCG | |
| chr | 919530 | G | A | NECAB | p.A271 | 0.007 | 0.004 | 1.54 | 1.74 | GATGTCTGTGATAGAAGAGG | SEQ |
| 8 | 77 | | | 1 | T | 16 | 12 | E−02 | [1.08- | ACCTGGAAGAATTCCAGCTC | ID |
| | | | | | | | | | 2.68] | [G/A]CTCTGAAACACTACG | NO: |
| | | | | | | | | | | TGGAGAGTGCTTCCTCCCAA | 936 |
| | | | | | | | | | | AGTGG | |
| chr | 947463 | C | G | RBM12 | p.E777 | 0.005 | 0.000 | 7.19 | Inf | GGCCGCCTGAAATGCTCCTG | SEQ |
| 8 | 10 | | | B | Q | 88 | 00 | E−34 | | GGGCGGTCTCCGGAAGTGCT | ID |
| | | | | | | | | | | [C/G]CGGGGGCGGGCGCCT | NO: |
| | | | | | | | | | | GAAATGCTCTGGGGGTGGCC | 937 |
| | | | | | | | | | | GCCTG | |
| chr | 978921 | G | A | CPQ | p.M24 | 0.008 | 0.004 | 1.07 | 1.75 | CCTGTATTACGGTGGAAGAT | SEQ |
| 8 | 19 | | | | 5I | 133 | 667 | E−02 | [1.12- | GCAGAAATGATGTCAAGAAT | ID |
| | | | | | | | | | 2.62] | [G/A]GCTTCTCATGGGATC | NO: |
| | | | | | | | | | | AAAATTGTCATTCAGCTAAA | 938 |
| | | | | | | | | | | GATGG | |
| chr | 989912 | A | 3 | MATN | p.K356 | 0.006 | 0.000 | 2.40 | Inf | CTTTGCCAGTGCCATGAAGG | SEQ |
| 8 | 22 | | | 2 | R | 86 | 00 | E−41 | | ATTTGCTCTTAACCCAGATA | ID |
| | | | | | | | | | | [A/G]AAAAACGTGCACAAG | NO: |
| | | | | | | | | | | TAAGTTACACACACATGCAC | 939 |
| | | | | | | | | | | ACACA | |
| chr | 100832 | A | G | VPS13 | p.N29 | 0.008 | 0.005 | 7.31 | 1.65 | ACTTTGTTGATAGAACTTCT | SEQ |
| 8 | 259 | | | B | 68S | 33 | 07 | E−03 | [1.17- | GCCCTGGGCCCTGCTTATCA | ID |
| | | | | | | | | | 2.34] | [A/G]TGAATCCAAATGGGA | NO: |
| | | | | | | | | | | CCTCTGGCTATTTGAAGGAG | 940 |
| | | | | | | | | | | AGAAA | |
| chr | 103573 | G | A | ODF1 | p.S228 | 0.005 | 0.000 | 4.61 | Inf | TGCAGCCCCTGCAACCCCTG | SEQ |
| 8 | 042 | | | | N | 64 | 00 | E−34 | | CAGCCCCTGCAACCCGTGCA | ID |
| | | | | | | | | | | [G/A]GCCATATGATCCTTG | NO: |
| | | | | | | | | | | CAACCCGTGTTATCCCTGTG | 941 |
| | | | | | | | | | | GAAGC | |
| chr | 104897 | G | A | RIMS2 | p.R175 | 0.005 | 0.003 | 4.50 | 1.59 | GGATCCATGCTGAAGTGTCC | SEQ |
| 8 | 928 | | | | R | 64 | 56 | E−02 | [1.04- | CGAGCACGGCATGAGAGAAG | ID |
| | | | | | | | | | 2.42] | [G/A]CATAGTGATGTTTCT | NO: |
| | | | | | | | | | | TTGGCAAATGCTGATCTGGA | 942 |
| | | | | | | | | | | AGATT | |
| chr | 125711 | A | G | MTSS1 | p.A62 | 0.009 | 0.006 | 1.95 | 1.52 | CAGCCTCCATCTGCTTACCA | SEQ |
| 8 | 789 | | | | A | 31 | 16 | E−02 | [1.09- | CGTGTGTTGGTGGCCATGTC | ID |
| | | | | | | | | | 2.1] | [A/G]GCCACTTTCTGAAAG | NO: |
| | | | | | | | | | | GCGTCCAAGAAGGCAGCTGC | 943 |
| | | | | | | | | | | TGCTA | |
| chr | 144297 | G | A | GPIHB | p.G159 | 0.005 | 0.000 | 1.65 | Inf | GTCCAGGACCCAACAGGCAA | SEQ |
| 8 | 314 | | | P1 | D | 39 | 00 | E−32 | | GGGGGCAGGCGGCCCCCGGG | ID |
| | | | | | | | | | | [G/A]CAGCTCCGAAACTGT | NO: |
| | | | | | | | | | | GGGCGCAGCCCTCCTGCTCA | 944 |
| | | | | | | | | | | ACCTC | |
| chr | 144874 | G | C | SCRIB | p.P145 | 0.013 | 0.000 | 9.10 | 229.41 | AGCTTTGGCCGTCCGCACCG | SEQ |
| 8 | 555 | | | | 0R | 97 | 06 | E−60 | [71.81- | GGGCGCCACCTCCCAGGGGT | ID |
| | | | | | | | | | 732.85] | [G/C]GGGGGGACGCCGGGC | NO: |
| | | | | | | | | | | TCTGCCTGGGGAAGGGACAG | 945 |
| | | | | | | | | | | GACGT | |
| chr | 144940 | C | T | EPPK1 | p.A226 | 0.008 | 0.001 | 2.32 | 7.88 | GCCTCAGGTTGCGCACGGGG | SEQ |
| 8 | 621 | | | | 7A | 09 | 03 | E−17 | [5.34- | TCGATGACGAAGCCGGTGGC | ID |
| | | | | | | | | | 11.63] | [C/T]GCCTGCGCCTCCAGC | NO: |
| | | | | | | | | | | AGCACCAGGGCCGTGCCGGG | 946 |
| | | | | | | | | | | CCGCA | |
| chr | 144941 | A | T | EPPK1 | p.Y206 | 0.006 | 0.003 | 2.84 | 1.61 | GTGTCCTCTTGTGGGCGGCA | SEQ |
| 8 | 229 | | | | 5N | 13 | 82 | E−02 | [1.07- | CCTCTCCTGCAGCTCTCGGT | ID |
| | | | | | | | | | 2.41] | [A/T]CGAGACCTTCTCTTG | NO: |
| | | | | | | | | | | CGTGTTCGGGTCCACAAACC | 947 |
| | | | | | | | | | | GTTTC | |
| chr | 144993 | G | A | PLEC | p.L359 | 0.008 | 0.006 | 3.15 | 1.46 | TGCTCCTCGGGGATCAGGTC | SEQ |
| 8 | 230 | | | | 1L | 82 | 04 | E−02 | [1.05- | CGACTGCATCACCTCCCACA | ID |
| | | | | | | | | | 2.05] | [G/A]GGACATGGTGGAGCC | NO: |
| | | | | | | | | | | GCCGTGGCTGCCGCCGCCGG | 948 |
| | | | | | | | | | | GAATG | |
| chr | 145736 | C | G | RECQL | p.V119 | 0.011 | 0.000 | 2.26 | 1295.85 | GTCAGCGGGCCACCTGCAGG | SEQ |
| 8 | 853 | | | 4 | 6V | 52 | 01 | E−67 | [178.75- | AGCTCTTCCGTGGCCAGGCC | ID |
| | | | | | | | | | 9394.47] | [C/G]ACCAGGGCATGGAAG | NO: |
| | | | | | | | | | | CTCAGGTGCAGGTATTTTCT | 949 |
| | | | | | | | | | | CCAGA | |
| chr | 146157 | C | T | ZNF16 | p.S303 | 0.005 | 0.003 | 4.01 | 1.6 | CATGTGAGACTTTTGGTGCT | SEQ |
| 8 | 265 | | | | N | 39 | 38 | E−02 | [1.04- | TTTTAAGGCTCGAGTTCTGG | ID |
| | | | | | | | | | 2.46] | [C/T]TGAAGGCTTTTCCAC | NO: |
| | | | | | | | | | | ATTCATTACACATATAAGGC | 950 |
| | | | | | | | | | | CTCTC | |
| chr | 411793 | C | G | GLIS3 | p.E360 | 0.008 | 0.004 | 6.65 | 1.8 | GCTGGTCGATGTGGACCTTC | SEQ |
| 9 | 3 | | | | D | 133 | 527 | E−03 | [1.15- | TCGATGTGCCGCACGAGCTC | ID |
| | | | | | | | | | 2.7] | [C/G]TCCTGCTGGTCGTAC | NO: |
| | | | | | | | | | | AGGGCGCTGCAGTCGATCCA | 951 |
| | | | | | | | | | | GCGGC | |
| chr | 601362 | C | T | RANBP | p.D662 | 0.006 | 0.002 | 5.81 | 2.36 | CTCTGCTGGTCTCCAAGATT | SEQ |
| 9 | 4 | | | 6 | N | 831 | 903 | E−04 | [1.44- | TACAAATTGCCAGCCATCAT | ID |
| | | | | | | | | | 3.68] | [C/T]GTCACTCATATTTTC | NO: |
| | | | | | | | | | | CACATCCTGTGTGTCTAAGA | 952 |
| | | | | | | | | | | GAGCA | |
| chr | 154230 | C | T | SNAPC | p.H43Y | 0.013 | 0.000 | 1.53 | 117.22 | TCCAGAGTATGAGCTTCCCG | SEQ |
| 9 | 04 | | | 3 | | 73 | 12 | E−64 | [59.77- | AGCTAAATACGCGCGCTTTC | ID |
| | | | | | | | | | 229.91] | [C/T]ATGTGGGCGCCTTTG | NO: |
| | | | | | | | | | | GGGAGCTGTGGCGGGGCCGT | 953 |
| | | | | | | | | | | CTGCG | |
| chr | 190503 | G | A | RRAGA | p.Q22 | 0.007 | 0.004 | 1.55 | 1.64 | CTACATTCTTGGTTATTTCC | SEQ |
| 9 | 23 | | | | 2Q | 11 | 34 | E−02 | [1.13- | CACTACCAGTGCAAAGAGCA | ID |
| | | | | | | | | | 2.39] | [G/A]CGCGACGTCCACCGG | NO: |
| | | | | | | | | | | TTTGAGAAGATCAGCAACAT | 954 |
| | | | | | | | | | | CATCA | |
| chr | 337948 | A | C | PRSS3 | p.K12T | 0.007 | 0.004 | 6.12 | 1.78 | GACAGGATGCACATGAGAGA | SEQ |
| 9 | 24 | | | | | 35 | 13 | E−03 | [1.22- | GACAAGTGGCTTCACATTGA | ID |
| | | | | | | | | | 2.62] | [A/C]GAAGGGGAGGAGTGC | NO: |
| | | | | | | | | | | GCCATTGGTTTTCCATCCTC | 955 |
| | | | | | | | | | | CAGAT | |
| chr | 337967 | G | T | PRSS3 | p.G106 | 0.005 | 0.000 | 3.09 | Inf | CCCTACCAGGTGTCCCTGAA | SEQ |
| 9 | 46 | | | | V | 15 | 00 | E−31 | | TTCTGGCTCCCACTTCTGCG | ID |
| | | | | | | | | | | [G/T]TGGCTCCCTCATCAG | NO: |
| | | | | | | | | | | CGAACAGTGGGTGGTATCAG | 956 |
| | | | | | | | | | | CAGCT | |
| chr | 356741 | G | A | CA9 | p.G79 | 0.014 | 0.001 | 1.18 | 10.67 | GCCCAGTGAAGAGGATTCAC | SEQ |
| 9 | 91 | | | | R | 46 | 37 | E−35 | [7.89- | CCAGAGAGGAGGATCCACCC | ID |
| | | | | | | | | | 14.43] | [G/A]GAGAGGAGGATCTAC | NO: |
| | | | | | | | | | | CTGGAGAGGAGGATCTACCT | 957 |
| | | | | | | | | | | GGAGA | |
| chr | 358100 | G | A | SPAG8 | p.F433 | 0.005 | 0.003 | 2.25 | 1.67 | GAGACAAGGGTACTGGTGTT | SEQ |
| 9 | 94 | | | | F | 88 | 53 | E−02 | [1.1- | GAGAAGCTGCAGTTCTTCCG | ID |
| | | | | | | | | | 2.52] | [G/A]AATGGTGTGTCCAAT | NO: |
| | | | | | | | | | | GTCCTGATGTTACTGACACC | 958 |
| | | | | | | | | | | CTGGA | |
| chr | 391092 | C | T | CNTNA | p.A769 | 0.022 | 0.000 | 4.31 | 1284.01 | GGCCCCAGTGTATAAGCTGC | SEQ |
| 9 | 17 | | | P3 | T | 55 | 02 | E− | [316.24- | TTCGGAATGTGGTCGGCCTG | ID |
| | | | | | | | | 131 | 5213.39] | [C/T]GTCTGTCATCACAAT | NO: |
| | | | | | | | | | | CTGAGTGACTGGCAGGTGCT | 959 |
| | | | | | | | | | | CCTTT | |
| chr | 776135 | A | G | C9orf4 | p.D295 | 0.009 | 0.006 | 3.33 | 1.44 | TTGATTTGGACTTACTGCAT | SEQ |
| 9 | 39 | | | 1 | D | 80 | 81 | E−02 | [1.05- | TCTGAATAAATCTCTTGAAA | ID |
| | | | | | | | | | 1.99] | [A/G]TCTCCTGCTGTCATA | NO: |
| | | | | | | | | | | GAAAAGTTAGAACCAGGAGG | 960 |
| | | | | | | | | | | AAGAC | |
| chr | 845625 | A | G | SPATA | p.K779 | 0.012 | 0.000 | 2.19 | Inf | GTGGGGAATTATCAGGGATG | SEQ |
| 9 | 04 | | | 31D3 | R | 25 | 00 | E−72 | | CAGCCAGGAGACTGCCCCAA | ID |
| | | | | | | | | | | [A/G]AAACCATCTCTTGCA | NO: |
| | | | | | | | | | | TGATCCGGAGACATCTTCAG | 961 |
| | | | | | | | | | | AGGAG | |
| chr | 941725 | C | T | NFIL3 | p.1M17 | 0.005 | 0.003 | 3.57 | 1.6 | GTGGAGAGTGTTTAATGACA | SEQ |
| 9 | 07 | | | | 0I | 88 | 69 | E−02 | [1.06- | GAAATACAACTACTTGACAC | ID |
| | | | | | | | | | 2.41] | [C/T]ATCGAGGGTTCGTGC | NO: |
| | | | | | | | | | | TCGTCCACAAATGAACTCAC | 962 |
| | | | | | | | | | | ATTGG | |
| chr | 960518 | G | A | WNK2 | p.A164 | 0.005 | 0.000 | 1.02 | 56.07 | GCGGGGGGGACCTGGCCCTG | SEQ |
| 9 | 69 | | | | 8A | 39 | 10 | E−22 | [23.94- | CCCCCAGTGCCTAAGGAGGC | ID |
| | | | | | | | | | 131.32] | [G/A]GTCTCAGGGCGTGTC | NO: |
| | | | | | | | | | | CAGCTGCCCCAGCCCTTGGT | 963 |
| | | | | | | | | | | GAGTA | |
| chr | 960814 | C | T | C9orf1 | p.R130 | 0.010 | 0.007 | 2.68 | 1.43 | TGCCTGTGAATCCCTTCCTT | SEQ |
| 9 | 33 | | | 29 | H | 54 | 41 | E−02 | [1.05- | GTACATGGTGGTCAGTGGCA | ID |
| | | | | | | | | | 1.94] | [C/T]GGAATCCCCAATAGA | NO: |
| | | | | | | | | | | TTGTATATCTGAAGGAGAAA | 964 |
| | | | | | | | | | | AATAA | |
| chr | 964390 | C | A | PHF2 | p.T992 | 0.022 | 0.001 | 8.06 | 20.02 | CCTCCACCACGCCAGCCTCT | SEQ |
| 9 | 19 | | | | T | 30 | 14 | E−65 | [14.5- | ACCACCCCGGCCTCCACCAC | ID |
| | | | | | | | | | 27.65] | [C/A]CCGGCCTCCACCAGC | NO: |
| | | | | | | | | | | ACGGCCAGCAGCCAGGCCTC | 965 |
| | | | | | | | | | | GCAGG | |
| chr | 970809 | A | C | NUTM | p.S689 | 0.007 | 0.000 | 5.91 | 16.69 | AAGAGAGGTCGCTTCTTGGA | SEQ |
| 9 | 53 | | | 2F | A | 84 | 47 | E−24 | [10.5- | CTTGCTGGCAGGAGAAGGTG | ID |
| | | | | | | | | | 26.52] | [A/C]TGGGCTGAGGCCTCT | NO: |
| | | | | | | | | | | TTTCTGAGCACATGGAGACT | 966 |
| | | | | | | | | | | CAAGA | |
| chr | 106889 | C | T | SMC2 | p.S867 | 0.005 | 0.003 | 3.42 | 1.63 | CCTCACCACATATTTTCTTT | SEQ |
| 9 | 571 | | | | L | 15 | 16 | E−02 | [1.05- | AATTTTTTTGTTTTAGGAGT | ID |
| | | | | | | | | | 2.54] | [C/T]AGTAAATAAAGCTCA | NO: |
| | | | | | | | | | | AGAAGAGGTGACCAAGCAAA | 967 |
| | | | | | | | | | | AAGAG | |
| chr | 113562 | T | C | MUSK | p.V558 | 0.006 | 0.004 | 2.64 | 1.59 | GAAACTGAGACTAACAGGGA | SEQ |
| 9 | 589 | | | | A | 62 | 17 | E−02 | [1.08- | TGGTCTTTTGGTTCCAGGAG | ID |
| | | | | | | | | | 2.35] | [T/C]GTGTGCTGTCGGGAA | NO: |
| | | | | | | | | | | GCCAATGTGCCTGCTCTTTG | 968 |
| | | | | | | | | | | AATAC | |
| chr | 117170 | G | C | DFNB3 | p.P562 | 0.119 | 0.117 | 6.55 | 1.02 | AACCAAAGGGCCAGCCAGGG | SEQ |
| 9 | 241 | | | 1 | A | 36 | 07 | E−01 | [0.93- | CCTTACCACGGACACATCTG | ID |
| | | | | | | | | | 1.13] | [G/C]GAGGGCGTTGATATT | NO: |
| | | | | | | | | | | GCCCTGGACAGCCTCGCCAG | 969 |
| | | | | | | | | | | TTTCC | |
| chr | 127623 | G | A | RPL35 | p.R32R | 0.011 | 0.008 | 3.12 | 1.39 | TAGAGAGCTTGGAGGCCGCA | SEQ |
| 9 | 742 | | | | | 76 | 52 | E−02 | [1.03- | CCGCCTGTCACTTTGGCGAC | ID |
| | | | | | | | | | 1.85] | [G/A]CGCAGCTGGGACAGC | NO: |
| | | | | | | | | | | TCCACCTTCAGGTCGTCCAG | 970 |
| | | | | | | | | | | CTGTT | |
| chr | 131094 | G | C | COQ4 | p.E161 | 0.012 | 0.008 | 1.55 | 1.44 | ATGATGAGGAGCTAGCGTAT | SEQ |
| 9 | 512 | | | | D | 25 | 51 | E−02 | [1.09- | GTGATTCAGCGGTACCGGGA | ID |
| | | | | | | | | | 1.92] | [G/C]GTGCACGACATGCTT | NO: |
| | | | | | | | | | | CACACCCTGCTGGGGATGCC | 971 |
| | | | | | | | | | | CACCA | |
| chr | 131258 | G | C | ODF2 | p.Q61 | 0.007 | 0.000 | 2.84 | Inf | TAAACCAGTCTGTGTTCCTG | SEQ |
| 9 | 331 | | | | 7H | 84 | 00 | E−47 | | TCATTTTAGATCGAACACCA | ID |
| | | | | | | | | | | [G/C]GGGGACAAGCTGGAG | NO: |
| | | | | | | | | | | ATGGCGAGAGAGAAACATCA | 972 |
| | | | | | | | | | | GGCTT | |
| chr | 132630 | G | A | USP20 | p.S288 | 0.005 | 0.003 | 9.34 | 1.85 | ACCGGAGCCCATCAGAAGAT | SEQ |
| 9 | 457 | | | | S | 64 | 05 | E−03 | [1.21- | GAGTTCTTGTCCTGTGACTC | ID |
| | | | | | | | | | 2.83] | [G/A]AGCAGTGACCGGGGT | NO: |
| | | | | | | | | | | GAGGGTGACGGGCAGGGGCG | 973 |
| | | | | | | | | | | TGGCG | |
| chr | 134353 | G | A | PRRC2 | p.E147 | 0.005 | 0.003 | 2.96 | 1.71 | CTGGTTAACAAGATCCTCTT | SEQ |
| 9 | 141 | | | B | 3K | 15 | 03 | E−02 | [1.1- | TCCCTTACAGATCCCCAGAC | ID |
| | | | | | | | | | 2.65] | [G/A]AGGCCTTGCCTGGAG | NO: |
| | | | | | | | | | | GTCTTAGTGGCTGCAGCAGT | 974 |
| | | | | | | | | | | GGGAG | |
| chr | 135140 | A | G | SETX | p.I254 | 0.008 | 0.005 | 2.68 | 1.5 | GGGTTGTGGATCCCAAAGGA | SEQ |
| 9 | 020 | | | | 7T | 58 | 72 | E−02 | [1.07- | ATATTCCTCCTTTGACCTCA | ID |
| | | | | | | | | | 2.12] | [A/G]TGCCCATCCTCTTCA | NO: |
| | | | | | | | | | | GCAGTCGTGGGTCCTGAAGT | 975 |
| | | | | | | | | | | TGGTC | |
| chr | 136419 | G | A | ADAM | p.G421 | 0.023 | 0.000 | 1.28 | Inf | CGAGCAGGCCGGCGGCGGGG | SEQ |
| 9 | 800 | | | TSL2 | S | 28 | 00 | E−12 | | CCTGCGAGGGGCCCCCCAGG | ID |
| | | | | | | | | | | [G/A]GCAAGGGCTTCCGAG | NO: |
| | | | | | | | | | | GTAACCAGGAGGAGGGAGGC | 976 |
| | | | | | | | | | | ATGAG | |
| chr | 137309 | G | A | RXRA | p.M25 | 0.006 | 0.003 | 2.76 | 1.62 | CCGTGGAGCCCAAGACCGAG | SEQ |
| 9 | 155 | | | | 4I | 13 | 79 | E−02 | [1.08- | ACCTACGTGGAGGCAAACAT | ID |
| | | | | | | | | | 2.43] | [G/A]GGGCTGAACCCCAGC | NO: |
| | | | | | | | | | | TCGGTGAGTTGCAGCCTGTG | 977 |
| | | | | | | | | | | CAGGG | |
| chr | 139333 | G | C | INPP5E | p.G120 | 0.007 | 0.000 | 1.78 | 447.13 | TCAGGCAGGGCGGGGAGCAG | SEQ |
| 9 | 512 | | | | G | 11 | 02 | E−34 | [60.89- | CTGTGGGCGGGGGCCCCGGG | ID |
| | | | | | | | | | 3283.17] | [G/C]CCCTCGCTCTGCACT | NO: |
| | | | | | | | | | | GAGCCCCTGGAGGGACTGGT | 978 |
| | | | | | | | | | | CCCAT | |
| chr | 139701 | G | T | CCDC1 | p.M45 | 0.005 | 0.003 | 4.82 | 1.63 | GCGAGGGGAAGCTCACGTAC | SEQ |
| 9 | 301 | 83 | 7I | 856 | 603 | | | E−02 | [0.95- | CTGGCTGACAGAGTGCAGAT | ID |
| | | | | | | | | | 2.61] | [G/T]GTGTCCAGGACCGAG | NO: |
| | | | | | | | | | | GAGGTAGCCCCGGGCTGGGA | 979 |
| | | | | | | | | | | GGAAC | |
| chr | 139752 | A | T | MAMD | p.T771 | 0.009 | 0.006 | 4.61 | 1.42 | CTCGGGCCATGCTGCCTGGG | SEQ |
| 9 | 023 | | | C4 | S | 07 | 39 | E−02 | [1.02- | GCCCCCCAACAGACCATACC | ID |
| | | | | | | | | | 1.98] | [A/T]CTGAGACAGCCCAAG | NO: |
| | | | | | | | | | | GTATGGGGGCCTGGCAGGGG | 980 |
| | | | | | | | | | | CAGGG | |
| chr | 140008 | G | A | DPP7 | p.Q38 | 0.005 | 0.000 | 4.86 | Inf | TTGTTGCCGAAGCGCTCGAA | SEQ |
| 9 | 984 | | | | X | 15 | 00 | E−28 | | GTTGAAGTGGTCCAGACGCT | ID |
| | | | | | | | | | | [G/A]CTGGAAGAAGCGCTC | NO: |
| | | | | | | | | | | CTGGAAGCCGGGGTCCGGGG | 981 |
| | | | | | | | | | | CCCTG | |
| chr | 140120 | G | T | CYSRT1 | p.A148 | 0.011 | 0.000 | 2.82 | Inf | AGCGCCAGGCCGGACTGACC | SEQ |
| 9 | 397 | | | | A | 03 | 00 | E−52 | | TACGCTGGCCCTCCGCCCGC | ID |
| | | | | | | | | | | [G/T]GGGCGCGGGGATGAC | NO: |
| | | | | | | | | | | ATCGCCCACCACTGCTGCTG | 982 |
| | | | | | | | | | | CTGCC | |
| chr | 986397 | C | CT | SHROO | p.L676 | 0.005 | 0.000 | 2.57 | 61.9 | CTGGAGGGCCGGGTTGGGAG | SEQ |
| X | 4 | | | M2 | fs | 89 | 10 | E−07 | [12.5- | GTGGCACCCAGGAAGGACCC | ID |
| | | | | | | | | | 307.1] | [C/CT]TCGCTGGCACCTAT | NO: |
| | | | | | | | | | | AAAGACCACCTGAAAGAGGC | 983 |
| | | | | | | | | | | CCAAGC | |
| chr | 100856 | C | T | WWC3 | p.H520 | 0.006 | 0.003 | 4.13 | 1.56 | GGGACGAAGACTTACCAGGC | SEQ |
| X | 59 | | | | H | 13 | 94 | E−02 | [1.03- | ATGGCGGCCCTTCAGCCACA | ID |
| | | | | | | | | | 2.36] | [C/T]GGGGTCCCCGGGGAT | NO: |
| | | | | | | | | | | GGGGAAGGGCCGCACGAGCG | 984 |
| | | | | | | | | | | AGGAC | |
| chr | 349618 | G | A | FAM47 | p.P297 | 0.005 | 0.000 | 6.33 | 473.89 | GCCCGGAGCCTCCCGAGACT | SEQ |
| X | 39 | | | B | P | 88 | 01 | E−31 | [64.09- | CGCGTATCTCATCTCCACCC | ID |
| | | | | | | | | | 3503.83] | [G/A]GAGCCTCCTGAGACT | NO: |
| | | | | | | | | | | GGAGTGTCCCATCTCCGCCC | 985 |
| | | | | | | | | | | AGAGC | |
| chr | 370279 | C | G | FAM47 | p.D492 | 0.006 | 0.000 | 5.71 | Inf | CAGAGAAGGACGTATCTCAT | SEQ |
| X | 59 | | | C | E | 86 | 00 | E−37 | | CTCCGCCCAGAGCCTCCCGA | ID |
| | | | | | | | | | | [C/G]ACTGGAGTGTCCCAT | NO: |
| | | | | | | | | | | CTCTGCCCAGAGCCCCCCAA | 986 |
| | | | | | | | | | | GACAC | |
| chr | 370287 | C | T | FAM47 | p.R763 | 0.008 | 0.000 | 2.98 | 692.67 | TCTCCGCCCAGAGCCTCTTG | SEQ |
| X | 70 | | | C | C | 58 | 01 | E−45 | [94.87- | AGACTCGCGTATCTCATCTC | ID |
| | | | | | | | | | 5057.22] | [C/T]GCCCGGAGCCTCCTG | NO: |
| | | | | | | | | | | AGACTGGAGTGTCCCATCTC | 987 |
| | | | | | | | | | | CACCC | |
| chr | 436286 | G | A | MAOB | p.T426 | 0.008 | 0.000 | 6.54 | Inf | CAGCCCCCTCCATGTAGCCG | SEQ |
| X | 23 | | | | T | 82 | 00 | E−48 | | CTCCAGTGTGTGGCAGTCTC | ID |
| | | | | | | | | | | [G/A]GTGCCTGCAAAGTAA | NO: |
| | | | | | | | | | | ATCCTGTCCACTGGCTGGCG | 988 |
| | | | | | | | | | | TAGAA | |
| chr | 474267 | C | T | ARAF | p.A337 | 0.010 | 0.007 | 3.68 | 1.42 | TTGGCACCGTGTTTCGAGGG | SEQ |
| X | 57 | | | | A | 05 | 11 | E−02 | [1.03- | CGGTGGCATGGCGATGTGGC | ID |
| | | | | | | | | | 1.95] | [C/T]GTGAAGGTGCTCAAG | NO: |
| | | | | | | | | | | GTGTCCCAGCCCACAGCTGA | 989 |
| | | | | | | | | | | GCAGG | |
| chr | 486648 | C | T | HDAC6 | p.Y171 | 0.005 | 0.002 | 1.98 | 2.04 | ACATGAATGAGGGAGAACTC | SEQ |
| X | 50 | | | | Y | 88 | 90 | E−03 | [1.34- | CGTGTCCTAGCAGACACCTA | ID |
| | | | | | | | | | 3.1] | [C/T]GACTCAGTTTATCTG | NO: |
| | | | | | | | | | | CATCCGGTATGGATGAGAAC | 990 |
| | | | | | | | | | | TCTGC | |
| chr | 491059 | G | A | CCDC2 | p.D546 | 0.008 | 0.005 | 3.56 | 1.48 | GCAGCCCACTGATACCTTTG | SEQ |
| X | 70 | | | 2 | N | 58 | 80 | E−02 | [1.05- | AGGTCCCTGTGTCTGGTCAG | ID |
| | | | | | | | | | 2.09] | [G/A]ATGCCAAGAAGGACG | NO: |
| | | | | | | | | | | ATGCTGTTCGGAAGGCCTAT | 991 |
| | | | | | | | | | | AAGTA | |
| chr | 494559 | C | T | PAGE1 | p.G56 | 0.008 | 0.005 | 2.89 | 1.49 | TTGGCTGAACCAGTTCCTGG | SEQ |
| X | 76 | | | | G | 82 | 92 | E−02 | [1.06- | CTATCAGCTTCAGGCTCCTG | ID |
| | | | | | | | | | 2.1] | [C/T]CCTTAAAGATAAAAC | NO: |
| | | | | | | | | | | AAAATTATCATTTTAAGCAG | 992 |
| | | | | | | | | | | CAACA | |
| chr | 531153 | G | A | TSPYL2 | p.E607 | 0.009 | 0.006 | 2.37 | 1.5 | AAGGCAGCGATGATGACGAC | SEQ |
| X | 95 | | | | E | 07 | 06 | E−02 | [1.07- | AGAGACATTGAGTACTATGA | ID |
| | | | | | | | | | 2.1] | [G/A]AAAGTTATTGAAGAC | NO: |
| | | | | | | | | | | TTTGACAAGGATCAGGCTGA | 993 |
| | | | | | | | | | | CTACG | |
| chr | 562918 | A | G | KLF8 | p.I108 | 0.009 | 0.006 | 4.00 | 1.43 | CAAGGCTCCTCTCCAGCCTG | SEQ |
| X | 53 | | | | V | 56 | 71 | E−02 | [1.03- | CTAGCATGCTACAAGCTCCA | ID |
| | | | | | | | | | 1.98] | [A/G]TACGTCCCCCCAAGC | NO: |
| | | | | | | | | | | CACAGTCTTCTCCCCAGACC | 994 |
| | | | | | | | | | | CTTGT | |
| chr | 708237 | G | C | ACRC | p.K218 | 0.005 | 0.000 | 1.40 | 33.76 | CCGACGACAACAGTGATGAT | SEQ |
| X | 81 | | | | N | 88 | 18 | E−22 | [17.45- | TCGGATGTTCCCGACGACAA | ID |
| | | | | | | | | | 65.31] | [G/C]AGTGATGATTCGGAT | NO: |
| | | | | | | | | | | GTTCCCGACGACAGCAGTGA | 995 |
| | | | | | | | | | | TGATT | |
| chr | 738116 | G | A | RLIM | p.S501 | 0.009 | 0.000 | 1.61 | Inf | ATGTCGACCCTCTCGCCTGG | SEQ |
| X | 48 | | | | L | 80 | 00 | E−52 | | CACCTGATGAGCCTGATGAT | ID |
| | | | | | | | | | | [G/A]AGCTTCCTTCATTAC | NO: |
| | | | | | | | | | | TGCCTTCAAATAAATCTGAG | 996 |
| | | | | | | | | | | CTAGT | |
| chr | 738116 | A | G | RLIM | p.S485 | 0.010 | 0.000 | 6.36 | 46.16 | CTTCATTACTGCCTTCAAAT | SEQ |
| X | 95 | | | | S | 29 | 23 | E−41 | [26.25- | AAATCTGAGCTAGTTTCTGA | ID |
| | | | | | | | | | 81.16] | [A/G]CTTTCACCACCGGAA | NO: |
| | | | | | | | | | | CTGGAACTAGGACTGGAACT | 997 |
| | | | | | | | | | | GGAAC | |
| chr | 738117 | C | T | RLIM | p.S453 | 0.010 | 0.000 | 2.96 | 825.58 | ACTCGAACTGGAACTGGAAC | SEQ |
| X | 92 | | | | N | 29 | 01 | E−54 | [113.6- | TCGAACTGGAACCAGAACTA | ID |
| | | | | | | | | | 5999.93] | [C/T]TACCACCACCAGAAC | NO: |
| | | | | | | | | | | CTCCTCTTCCACTCCGTGAC | 998 |
| | | | | | | | | | | TCTGC | |
| chr | 100507 | G | T | DRP2 | p.L571 | 0.011 | 0.008 | 3.77 | 1.38 | CCTGCTTCTTGACAGGCAGG | SEQ |
| X | 675 | | | | L | 76 | 56 | E−02 | [1.03- | GCCAGCAAAGGCAATAAGCT | ID |
| | | | | | | | | | 1.85] | [G/T]CACTACCCCATCATG | NO: |
| | | | | | | | | | | GAGTATTACACACCGGTATG | 999 |
| | | | | | | | | | | AAGCC | |
| chr | 100524 | C | T | TAF7L | p.R372 | 0.011 | 0.007 | 2.26 | 1.44 | TGTGGGCCACGCCAATGGCT | SEQ |
| X | 197 | | | | H | 03 | 69 | E−02 | [1.06- | CTCCTCACTTCTTCAGAAAA | ID |
| | | | | | | | | | 1.95] | [C/T]GCTGCAACTGTTCCT | NO: |
| | | | | | | | | | | GTAGGGAAATGAGCTGTAGG | 1000 |
| | | | | | | | | | | GAGAG | |
| chr | 100745 | C | G | ARMC | p.A770 | 0.008 | 0.000 | 8.99 | Inf | CAGGGTGAGGTCTTGCCTGG | SEQ |
| X | 885 | | | X4 | G | 33 | 00 | E−34 | | TGCCAAAAATAAGGTCAAGG | ID |
| | | | | | | | | | | [C/G]CAATCTTAATGCTGT | NO: |
| | | | | | | | | | | GTCTAAGGCAGAAGCTGGGA | 1001 |
| | | | | | | | | | | TGGGT | |
| chr | 100746 | G | C | ARMC | p.Q94 | 0.009 | 0.000 | 1.04 | Inf | CTAAGGCAGAGGCTGGGGCA | SEQ |
| X | 423 | | | X4 | 9H | 31 | 00 | E−38 | | GGCATAATGGGCTCTGTCCA | ID |
| | | | | | | | | | | [G/C]GTCCAGGTTGTGGCC | NO: |
| | | | | | | | | | | AGTTTTCAGGGTGAGGTCTT | 1002 |
| | | | | | | | | | | GCCTG | |
| chr | 101971 | C | T | ARMC | p.S721 | 0.011 | 0.007 | 5.08 | 1.58 | TGACTATTGACTATCACACA | SEQ |
| X | 960 | | | X5- | S | 52 | 33 | E−03 | [1.17- | CTGATTGCCAACTATATGTC | ID |
| | | | | GPRAS | | | | | 2.13] | [C/T]GGGTTTCTCTCCTTA | NO: |
| | | | | P2 | | | | | | TTAACCACAGCCAATGCGAG | 1003 |
| | | | | | | | | | | AACGA | |
| chr | 102754 | C | T | RAB40 | p.E257 | 0.008 | 0.001 | 5.24 | 4.28 | GTGCAGTTTTTGGGTGGGCT | SEQ |
| X | 916 | | | A | K | 33 | 96 | E−11 | [2.95- | CTGGGGTGGGCAGACGATCT | ID |
| | | | | | | | | | 6.22] | [C/T]CACTTTGCAGAGGCT | NO: |
| | | | | | | | | | | GCTCTTGTGAGTGGAGCTGG | 1004 |
| | | | | | | | | | | TGGTG | |
| chr | 114425 | G | A | RBMXL | p.R514 | 0.007 | 0.000 | 5.32 | 323.05 | AGCGACCGCTACGGAGTAGG | SEQ |
| X | 545 | | | 3 | Q | 60 | 02 | E−32 | [44.09- | AGGCCACTATGAGGAGAACC | ID |
| | | | | | | | | | 2367.01] | [G/A]AGGCCACTCTCTGGA | NO: |
| | | | | | | | | | | TGCCAACAGCGGAGGCCGTT | 1005 |
| | | | | | | | | | | CACCC | |
| chr | 114426 | C | T | RBMXL | p.Y849 | 0.012 | 0.000 | 4.17 | 101.99 | ACGCCTACAGTGGGGGCCGT | SEQ |
| X | 551 | | | 3 | Y | 01 | 12 | E−46 | [40.62- | GACAGTTCCAGCAACAGTTA | ID |
| | | | | | | | | | 256.12] | [C/T]GACCGGAGCCACCGC | NO: |
| | | | | | | | | | | TATGGAGGAGGAGGCCACTA | 1006 |
| | | | | | | | | | | CGAAG | |
| chr | 120008 | G | C | CT478 | p.P182 | 0.012 | 0.000 | 1.16 | 1046.3 | CGACGCAGCCTCCTGGATCA | SEQ |
| X | 980 | | | 1 | R | 99 | 01 | E−68 | [144.66- | GGCCGAGGCCCTCGCCTTCT | ID |
| | | | | | | | | | 7567.63] | [G/C]GGGCTGCAGCCCCTG | NO: |
| | | | | | | | | | | CACCCAGCCTCTGGGACAGC | 1007 |
| | | | | | | | | | | AGCAG | |
| chr | 124455 | G | C | LOC10 | p.K430 | 0.017 | 0.000 | 8.76 | Inf | ACAGCCACAGCATGAAGAAA | SEQ |
| X | 258 | | | 01295 | N | 40 | 00 | E−72 | | GATCCAGTGATGCCCCAGAA | ID |
| | | | | 20 | | | | | | [G/C]ATGGTCCCCCTGGGG | NO: |
| | | | | | | | | | | GACAGCAACAGCCACAGTCT | 1008 |
| | | | | | | | | | | GAAGA | |
| chr | 140993 | A | G | MACE | p.Q18 | 0.013 | 0.002 | 4.36 | 6.11 | CTTTAGTGAGTATTTTCCAG | SEQ |
| X | 751 | | | C1 | 7Q | 24 | 19 | E−16 | [3.92- | AGTTCCCCTGAGAGTACTCA | ID |
| | | | | | | | | | 9.52] | [A/G]AGTCCTTTTCAGGGT | NO: |
| | | | | | | | | | | TTTCCCCAGTCTCCACTCCA | 1009 |
| | | | | | | | | | | GATTC | |
| chr | 140994 | T | A | MAGE | p.C501 | 0.014 | 0.000 | 9.16 | Inf | CTCCTCCACTTTATTGAGTC | SEQ |
| X | 691 | | | C1 | S | 71 | 00 | E−80 | | TTTTCCAGAGTTCCCCTGAG | ID |
| | | | | | | | | | | [T/A]GTACTCAAAGTACTT | NO: |
| | | | | | | | | | | TTGAGGGTTTTCCCCAGTCT | 1010 |
| | | | | | | | | | | CCTCT | |
| chr | 149100 | C | T | CXorf4 | p.G155 | 0.009 | 0.005 | 1.69 | 1.54 | AACATTCCTTTCAGGAGCCC | SEQ |
| X | 775 | | | 0B | E | 07 | 92 | E−02 | [1.1- | ACACTTGTCACACTTCATGC | ID |
| | | | | | | | | | 2.15] | [C/T]CCAAAGGGATCAGGT | NO: |
| | | | | | | | | | | GCTCTGGGATGTCTACCTGG | 1011 |
| | | | | | | | | | | AATAC | |
| chr | 150908 | G | T | CNGA2 | p.G113 | 0.010 | 0.007 | 4.45 | 1.38 | GGGCCTGAACTCCAGACTGT | SEQ |
| X | 168 | | | | V | 54 | 65 | E−02 | [1.01- | GACCACACAGGAGGGGGATG | ID |
| | | | | | | | | | 1.88] | [G/T]CAAAGGCGACAAGGA | NO: |
| | | | | | | | | | | TGGCGAGGACAAAGGCACCA | 1012 |
| | | | | | | | | | | AGTAC | |
| chr | 153295 | C | T | MECP2 | p.K443 | 0.018 | 0.000 | 3.45 | Inf | TGGCGGCGGTGGCAACCGCG | SEQ |
| X | 986 | | | | K | 87 | 00 | E− | | GGCTGAGTCTTAGCTGGCTC | ID |
| | | | | | | | | 102 | | [C/T]TTGGGGCAGCCGTCG | NO: |
| | | | | | | | | | | CTCTCCAGTGAGCCTCCTCT | 1013 |
| | | | | | | | | | | GGGCA | |

TABLE 2
Variants associated with infertility symptom of endometriosis

| | | | Alter- | | | Chron- | | | | | |
|---|---|---|---|---|---|---|---|---|---|---|---|
| | | | nate | | Amino | ic | | | | | |
| | | Refer- | Allele/ | | Acid | Pelvic | Infer- | | OR | | |
| | Posi- | ence | Minor | | posi- | Pain | tility | p | [L95- | | SEQ ID |
| Chr | tion | Allele | Allele | Gene | tion | MAF | MAF | value | U95] | Context Sequence | NO |
| chr | 544404 | C | T | OR51Q | p.L204 | 0.008 | 0.028 | 2.59 | 0.30 | CTGTGCTGACATCAGGCTCA | SEQ ID |
| 11 | 0 | | | 1 | F | 94 | 99 | E−02 | | ACAGCTGGTATGGATTTGCT | NO: 129 |
| | | | | | | | | | | [C/T]TTGCCTTGCTCATTA | |
| | | | | | | | | | | TTATCGTGGATCCTCTGCTC | |
| | | | | | | | | | | ATTGT | |
| chr | 537931 | C | T | BIRC8 | p.A156 | 0.000 | 0.007 | 1.16 | 0.00 | GAAGTCTGATTCAATTCATT | SEQ ID |
| 19 | 62 | | | | T | 00 | 25 | E−03 | | TTCTGTAGTGTCTTTCTGAG | NO: 531 |
| | | | | | | | | | | [C/T]GCTCACTAGATCTGC | |
| | | | | | | | | | | AACAAGAACCTCAAGCGTTT | |
| | | | | | | | | | | TATAG | |
| chr | 238973 | A | G | SCLY | p.K60E | 0.000 | 0.007 | 1.11 | 0.00 | AACGACTCCCCTGGAGCCAG | SEQ ID |
| 2 | 062 | | | | | 00 | 30 | E−03 | | AAGTTATCCAGGCCATGACC | NO: 592 |
| | | | | | | | | | | [A/G]AGGCCATGTGGGAAG | |
| | | | | | | | | | | CCTGGGGAAATCCCAGCAGC | |
| | | | | | | | | | | CCGTA | |
| chr | 503153 | C | A | CRELD | p.D182 | 0.028 | 0.061 | 4.03 | 0.44 | ACATGGGGTACCAGGGCCCG | SEQ ID |
| 22 | 63 | | | 2 | E | 20 | 59 | E−03 | | CTGTGCACTGACTGCATGGA | NO: 637 |
| | | | | | | | | | | [C/A]GGCTACTTCAGCTCG | |
| | | | | | | | | | | CTCCGGAACGAGACCCACAG | |
| | | | | | | | | | | CATCT | |
| chr | 819672 | C | T | BMP3 | p.T222 | 0.000 | 0.007 | 1.16 | 0.00 | GCCAAAGAAAATGAAGAGTT | SEQ ID |
| 4 | 40 | | | | M | 00 | 25 | E−03 | | CCTCATAGGATTTAACATTA | NO: 706 |
| | | | | | | | | | | [C/T]GTCCAAGGGACGCCA | |
| | | | | | | | | | | GCTGCCAAAGAGGAGGTTAC | |
| | | | | | | | | | | CTTTT | |

TABLE 3
Variants associated with pelvic pain symptom of endometriosis

| | | | Alter- | | | Chron- | | | | | |
|---|---|---|---|---|---|---|---|---|---|---|---|
| | | | nate | | Amino | ic | | | | | |
| | | Refer- | Allele/ | | Acid | Pelvic | Infer- | | OR | | |
| | Posi- | ence | Minor | | posi- | Pain | tility | p | [L95- | | SEQ ID |
| Chr | tion | Allele | Allele | Gene | tion | MAF | MAF | value | U95] | Context Sequence | NO |
| chr | 141232 | C | T | LRP1B | p.A317 | 0.000 | 0.010 | 7.31 | 0.00 | GCCCAGTAGAGTCTACGATT | SEQ ID |
| 2 | 800 | | | | 8T | 00 | 87 | E−05 | | AACATAATCTATTGTTAGTG | NO: 577 |
| | | | | | | | | | | [C/T]CATAGGTCTAGAAAT | |
| | | | | | | | | | | CTTGGTTTCTATGACAACAC | |
| | | | | | | | | | | TCTGA | |
| chr | 560330 | G | A | COL21 | p.T343 | 0.063 | 0.115 | 2.12 | 0.52 | TACTAAGAGACGAATTTGGT | SEQ ID |
| 6 | 94 | | | A1 | M | 89 | 90 | E−03 | | GCCAGCCTTCATCAAACAAC | NO: 786 |
| | | | | | | | | | | [G/A]TCTACAAAAAGAAAGT | |
| | | | | | | | | | | GTGGAAGATTCATAAATAAA | |
| | | | | | | | | | | GCCC | |
| chr | 854737 | C | T | TBX18 | p.G48 | 0.480 | 0.576 | 2.41 | 0.68 | GCGCCGCCGCCGCGGCTGCA | SEQ ID |
| 6 | 58 | | | | R | 50 | 60 | E−03 | | GCCTCCGTCGTCCACGGCCC | NO: 789 |
| | | | | | | | | | | [C/T]CGCCGCCTCTTCGGC | |
| | | | | | | | | | | GCCCAGTTTTCGCCGCTTCT | |
| | | | | | | | | | | TCTGA | |
| chr | 117170 | 0 | C | DFNB3 | p.P562 | 0.100 | 0.160 | 4.01 | 0.59 | AACCAAAGGGCCAGCCAGGG | SEQ ID |
| 9 | 241 | | | 1 | A | 70 | 60 | E−03 | | CCTTACCACGGACACATCTG | NO: 969 |
| | | | | | | | | | | [G/C]GAGGGCGTTGATATT | |
| | | | | | | | | | | GCCCTGGACAGCCTCGCCAG | |
| | | | | | | | | | | TTTCC | |

TABLE 4
Additional variants associated with endometriosis.

| | | | | | L95 | U95 | | | | | | | |
|---|---|---|---|---|---|---|---|---|---|---|---|---|---|
| | | | | | (low- | (up- | | | | | | | |
| | | | | | er | per | | | | | | | |
| | Local | | | | lim- | lim- | | | | | | | |
| | popu- | | | | it | it | | | | | | | |
| Endo- | lat- | | | | 95% | 95% | | | | | | | |
| metri- | tion | | | | con- | Con- | | | | | | | |
| osis | Con- | gnomA | | | fi- | fi- | | | | | | | |
| pa- | trol | D | | OR | dence | dence | | | Base | mi- | ma- | | |
| tient | Fre- | Fre- | P | (odds | In- | In- | | | Pair | nor | jor | | SEQ |
| Fre- | quen- | quen- | (Chisq | Ra- | ter- | ter- | | | Posi- | Al- | Al- | | ID |
| quency | cy | cy | test) | tio) | val) | val) | CHR | SNP | tion | lele | lele | Context Sequence | NO |
| 0.3055 | 0.28 | 0.288 | 4.49 | 1.13 | 1.07 | 1.20 | 1 | rs3410 | 16,08 | C | T | GCATCAGGTATTTTTACCCA | SEQ |
| | | 3 | E−05 | | | | | 8989 | 2,127 | | | CATTTACCCCACCAGATTCT | ID |
| | | | | | | | | | | | | [T/C]GCTATGAAGCCACAA | NO: |
| | | | | | | | | | | | | GGGACAAACCTGGGTTGGCA | 1014 |
| | | | | | | | | | | | | ACCCC | |
| 0.1844 | 0.149 | 0.159 | 1.75 | 1.29 | 1.20 | 1.38 | 1 | rs2235 | 22,45 | T | C | AAGCATCTGTGCCCCTAAAG | SEQ |
| | 4 | 1 | E−12 | | | | | 529 | 0,487 | | | CTGATGGCGGCTCCTCCAG | ID |
| | | | | | | | | | | | | [C/T]TTCTCTACCTGGTTC | NO: |
| | | | | | | | | | | | | TGGTGTCCAGCCCTTGGACT | 1015 |
| | | | | | | | | | | | | CCAGG | |
| 0.2294 | 0.199 | 0.208 | 5.07 | 1.20 | 1.12 | 1.28 | 1 | rs1204 | 22,47 | A | G | CATGAGCCACCTTGCCTGGC | SEQ |
| | 2 | 6 | E−08 | | | | | 2083 | 2,732 | | | CGGAAATTCTTAATGAGAAA | ID |
| | | | | | | | | | | | | [G/A]TCTCTTGGAGGAAAT | NO: |
| | | | | | | | | | | | | GCTCTTCTAACTTTCAAGAA | 1016 |
| | | | | | | | | | | | | CAGCC | |
| 0.4374 | 0.404 | 0.420 | 1.07 | 1.15 | 1.09 | 1.21 | 1 | rs4623 | 22,48 | G | A | ATCTTCAGCCTCCTACCAGC | SEQ |
| | 2 | 5 | E−06 | | | | | 666 | 0,312 | | | AACTATGCACACAGAAGCCC | ID |
| | | | | | | | | | | | | [A/G]GCCGGTATCCCCACA | NO: |
| | | | | | | | | | | | | GAGGCAGACGCCCCGGCACT | 1017 |
| | | | | | | | | | | | | GCCTT | |
| 0.1126 | 0.096 | 0.099 | 9.43 | 1.19 | 1.09 | 1.30 | 1 | rs1206 | 97,98 | T | C | AGTTGAAACTCACAAACTGC | SEQ |
| | 37 | 15 | E−05 | | | | | 1124 | 9,751 | | | AGGAATATAGTCATTGGGGT | ID |
| | | | | | | | | | | | | [C/T]CCTTAGATGCAGAAA | NO: |
| | | | | | | | | | | | | AGAAAATTAACTACAGCGAG | 1018 |
| | | | | | | | | | | | | TTATG | |
| 0.3216 | 0.348 | 0.338 | 3.65 | 0.89 | 0.84 | 0.94 | 2 | rs2349 | 49,24 | T | C | AAAACTTTATTCATAAAAAC | SEQ |
| | 7 | 8 | E−05 | | | | | 415 | 7,832 | | | AGGTGTCAGGCTGGATTTGA | ID |
| | | | | | | | | | | | | [T/C]CCATTGGCTGTAGTT | NO: |
| | | | | | | | | | | | | CAGTGACACTGTCCTAGATC | 1019 |
| | | | | | | | | | | | | GTGGA | |
| 0.0955 | 0.077 | 0.086 | 1.24 | 1.26 | 1.15 | 1.38 | 2 | rs1702 | 98,63 | G | A | TCCGGGGAACACGATTCCAC | SEQ |
| 9 | 47 | 25 | E−06 | | | | | 5778 | 7,504 | | | CCATCACTGGGTGCTAGGTC | ID |
| | | | | | | | | | | | | [A/G]AGGGTTCAGTTCTAT | NO: |
| | | | | | | | | | | | | GTCCTTCAGCACTTATGAAA | 1020 |
| | | | | | | | | | | | | CTGAG | |
| 0.1044 | 0.087 | 0.090 | 2,55 | 1.21 | 1.11 | 1.32 | 2 | rs1702 | 98,67 | A | G | GGATGAATGGAAACTTGATT | SEQ |
| | 78 | 62 | E−05 | | | | | 6292 | 7,164 | | | CTCTTAATACAGTCCACTTG | ID |
| | | | | | | | | | | | | [G/A]GCTCCATTTGTCTTC | NO: |
| | | | | | | | | | | | | ACAGCAACCATTTGCTGGAT | 1021 |
| | | | | | | | | | | | | TTATT | |
| 0.4036 | 0.374 | 0.382 | 1.47 | 1.13 | 1.07 | 1.20 | 2 | rs7555 | 135,1 | A | G | TATGCTTAGGAAATATGTAT | SEQ |
| | 4 | 7 | E−05 | | | | | 03 | 44,45 | | | ATATGGGATATCTCAAAATA | ID |
| | | | | | | | | | 4 | | | [A/G]GGAAAAGTTGGAGTG | NO: |
| | | | | | | | | | | | | AAGATTAAAATAGAAAATAA | 1022 |
| | | | | | | | | | | | | CAAAA | |
| 0.1662 | 0.188 | 0.182 | 4.81 | 0.86 | 0.80 | 0.93 | 2 | rs1017 | 219,7 | C | T | CTATGTGAATGTGACTGAAA | SEQ |
| | | 2 | E−05 | | | | | 7996 | 46,56 | | | CATATCTGTGGGAGTGGGCT | ID |
| | | | | | | | | | 1 | | | [T/C]GTGGGGAACCCTGTG | NO: |
| | | | | | | | | | | | | TGTATGGGCATCTATTCCTG | 1023 |
| | | | | | | | | | | | | GGGAT | |
| 0.2852 | 0.259 | 0.263 | 1.47 | 1.14 | 1.08 | 1.21 | 2 | rs3882 | 225,9 | T | C | ACAGTTAATATTGACTGCTT | SEQ |
| | | | E−05 | | | | | 08 | 38,99 | | | TGTTCATTGATACATTCCCT | ID |
| | | | | | | | | | 6 | | | [T/C]GACCTAGACCATTGC | NO: |
| | | | | | | | | | | | | TGGGCACATAGTAGGCTCTC | 1024 |
| | | | | | | | | | | | | AGTAA | |
| 0.1818 | 0.161 | 0.169 | 5.28 | 1.16 | 1.08 | 1.24 | 3 | rs6792 | 6,10 | A | G | CTATTGATTTTTGAGGTAGA | SEQ |
| | 3 | 5 | E−05 | | | | | 001 | 6,251 | | | TATTGATGCAATTAGAGATA | ID |
| | | | | | | | | | | | | [A/G]GCTTTAGGAAGATCT | NO: |
| | | | | | | | | | | | | TCCTGGAAGTGGTATATAAA | 1025 |
| | | | | | | | | | | | | TAGTT | |
| 0.2338 | 0.258 | 0.258 | 6.26 | 0.88 | 0.82 | 0.94 | 3 | rs6777 | 8,78 | G | A | CACCCTTCAGATCATAAAAC | SEQ |
| | | 4 | E−05 | | | | | 088 | 6,487 | | | AATAGAATTTGAGAGCTGCG | ID |
| | | | | | | | | | | | | [A/G]CTATAGCACTGCCAC | NO: |
| | | | | | | | | | | | | TAAGTCACTGTTGGCTTAAG | 1026 |
| | | | | | | | | | | | | CAAG | |
| 0.1513 | 0.174 | 0.168 | 1.05 | 0.84 | 0.78 | 0.91 | 3 | rs4293 | 25,91 | T | C | AATTGACACACTACTGAAAA | SEQ |
| | 4 | 2 | E−05 | | | | | 672 | 3,415 | | | GAAAAGAGAATTAGAACAAC | ID |
| | | | | | | | | | | | | [T/C]TGCCTGGAGTTAAAG | NO: |
| | | | | | | | | | | | | TCCCTTAGTTAATGGATAAG | 1027 |
| | | | | | | | | | | | | TCACC | |
| 0.1244 | 0.146 | 0.134 | 9.21 | 0.83 | 0.77 | 0.90 | 3 | rs1684 | 100,8 | G | T | TCTGGTGTCATTAAGGAAGC | SEQ |
| | | 4 | E−06 | | | | | 3225 | 01,25 | | | AGGTTACAGGCCAGCATATC | ID |
| | | | | | | | | | 7 | | | [T/G]TCAAATAGCTACACA | NO: |
| | | | | | | | | | | | | GGTGTTAGAACTGCATGGTC | 1028 |
| | | | | | | | | | | | | TTATA | |
| 0.1405 | 0.122 | 0.126 | 8.98 | 1.17 | 1.08 | 1.27 | 3 | rs4680 | 156,2 | A | G | GTGCTAATTATCCAGAATCA | SEQ |
| | 6 | | E−05 | | | | | 277 | 45,78 | | | GCTGCAGTTGCTACCATGGA | ID |
| | | | | | | | | | 1 | | | [A/G]GTAACCAGCTCTGCC | NO: |
| | | | | | | | | | | | | CAGTGGGTTCTCCIGTGCCC | 1029 |
| | | | | | | | | | | | | TACAG | |
| 0.1399 | 0.120 | 0.125 | 2.78E | 1.18 | 1.09 | 1.28 | 3 | rs6795 | 156,2 | T | C | TAGTGAAGAAAACATCATGC | SEQ |
| | 8 | 9 | E−05 | | | | | 731 | 62,46 | | | TGGTTATGTTACCATTTTTC | ID |
| | | | | | | | | | 0 | | | [C/T]CAGGCAACCAGGGTT | NO: |
| | | | | | | | | | | | | ATGGAAGAAAGGACTCATTA | 1030 |
| | | | | | | | | | | | | ATGGC | |
| 0.2683 | 0.298 | 0.288 | 1.43 | 0.86 | 0.81 | 0.92 | 4 | rs1250 | 56,00 | A | C | GATGTGGTCATATGAAGGCT | SEQ |
| | 8 | | E−06 | | | | | 5096 | 6,102 | | | TGACTGGGGCTGAAGAATAC | ID |
| | | | | | | | | | | | | [C/A]TTTCTGGTGTGACTC | NO: |
| | | | | | | | | | | | | ACTCACATGACTATTGGCAA | 1031 |
| | | | | | | | | | | | | GAGAA | |
| 0.2068 | 0.182 | 0.190 | 6.96 | 1.17 | 1.09 | 1.25 | 4 | rs1001 | 161,3 | A | G | CCTTGGAGAGTTCCTCCACT | SEQ |
| | 6 | 7 | E−06 | | | | | 4285 | 07,97 | | | TCTCTCTGACAATTAAAATC | ID |
| | | | | | | | | | 2 | | | [G/A]GTGTTTGCTGAGATT | NO: |
| | | | | | | | | | | | | AGACATTTTTTTCTTCTCTG | 1032 |
| | | | | | | | | | | | | TTTAG | |
| 0.0461 | 0.035 | 0.032 | 5.50 | 1.31 | 1.15 | 1.49 | 4 | rs1265 | 186,3 | A | G | TGGTGGTAGGGAGACCTTTT | SEQ |
| 1 | 63 | 3 | E−05 | | | | | 0364 | 65,99 | | | GGTGGTATTTGAATTAAACA | ID |
| | | | | | | | | | 8 | | | [G/A]TATCATTTTCTTTAA | NO: |
| | | | | | | | | | | | | AACCAACTCCACAGACTACA | 1033 |
| | | | | | | | | | | | | AAAAT | |
| 0.0548 | 0.040 | 0.047 | 1.06 | 1.39 | 1.23 | 1.57 | 4 | rs4611 | 188,9 | G | T | GTGTTGGTCGGTACAGTTCT | SEQ |
| 1 | 1 | 9 | E−07 | | | | | 976 | 90,95 | | | AGAAGGAAAGCTCTGAGCTG | ID |
| | | | | | | | | | 5 | | | [T/G]GCCCCTCTCTCCAGG | NO: |
| | | | | | | | | | | | | TGGAATTAGATTTTATATAT | 1034 |
| | | | | | | | | | | | | TCACT | |
| 0.3727 | 0.346 | 0.343 | 7.34 | 1.12 | 1.06 | 1.19 | 5 | rs4128 | 76,42 | T | C | ATTCCCCATTCCTTTACAAT | SEQ |
| | 6 | 7 | E−05 | | | | | 741 | 3,967 | | | TATAATTGCCTCCATATTGT | ID |
| | | | | | | | | | | | | [C/T]CAAGGACCATAGTTA | NO: |
| | | | | | | | | | | | | CCACTTGACCCAGAGCCTCT | 1035 |
| | | | | | | | | | | | | CCCTT | |
| 0.4173 | 0.383 | 0.393 | 6.02 | 1.15 | 1.09 | 1.22 | 5 | rs1252 | 76,42 | A | C | AGCTGTTCTCAGATACCAGA | SEQ |
| | 7 | 9 | E−07 | | | | | 1058 | 6,987 | | | CTGGAATAAACGAGAGACAT | ID |
| | | | | | | | | | | | | [C/A]TGGAGAAAGGAGACC | NO: |
| | | | | | | | | | | | | TCTTCCTATCCCAACAGGAC | 1036 |
| | | | | | | | | | | | | TGTGT | |
| 0.1807 | 0.156 | 0.164 | 1.77 | 1.19 | 1.11 | 1.28 | 6 | rs6456 | 19,76 | G | A | GCTCACCAAGCAAGATTCCT | SEQ |
| | 6 | 5 | E−06 | | | | | 259 | 1,718 | | | CTCATCCCCTGCCACTCCCT | ID |
| | | | | | | | | | | | | [A/G]TTTAATGCCTTTGTA | NO: |
| | | | | | | | | | | | | AAAACTGTAATTTGGTGAAT | 1037 |
| | | | | | | | | | | | | CCCAA | |
| 0.1874 | 0.165 | 0.161 | 2.88 | 1.16 | 1.08 | 1.24 | 6 | rs5634 | 151,2 | C | T | GCTACTCTTTTCTTCCAAAA | SEQ |
| | 9 | 5 | E−05 | | | | | 40 | 88,99 | | | TACTCTCTCCTCAGCAGCCA | ID |
| | | | | | | | | | 1 | | | [T/C]AGAGACTGAAACCTA | NO: |
| | | | | | | | | | | | | ATGAAGCCCTGTTGCCTTCC | 1038 |
| | | | | | | | | | | | | TACTT | |
| 0.1003 | 0.118 | 0.126 | 6.95 | 0.83 | 0.76 | 0.91 | 6 | rs9347 | 166,3 | T | G | TCATTGGGAGTTATGAGCAC | SEQ |
| | | 2 | E−05 | | | | | 099 | 27,88 | | | ATTTCATAAACATAATTCCA | ID |
| | | | | | | | | | 6 | | | [G/T]GGGTTCGCCTGTGAT | NO: |
| | | | | | | | | | | | | GACATCATTCCTTTTCACAA | 1039 |
| | | | | | | | | | | | | GGTTT | |
| 0.4488 | 0.410 | 0.415 | 2.01 | 1.17 | 1.11 | 1.23 | 7 | rs1177 | 27,20 | G | T | CTCCCCCTGCCCCCAATTCC | SEQ |
| | 7 | 2 | E−08 | | | | | 3804 | 6,688 | | | TAACAGAAAGCAGCGACTCC | ID |
| | | | | | | | | | | | | [T/G]AGAACAGGGGTAATC | NO: |
| | | | | | | | | | | | | AAATTCACGTGTGGATACTG | 1040 |
| | | | | | | | | | | | | TGCCT | |
| 0.1704 | 0.191 | 0.182 | 9.23 | 0.87 | 0.81 | 0.93 | 7 | rs1153 | 37,74 | G | A | AGGAAAATAAATTATGGAGA | SEQ |
| | 6 | 9 | E−05 | | | | | 5191 | 7,276 | | | CATTAAGTAAATTGCCCAAG | ID |
| | | | | | | | | | | | | [A/G]TGGCCCAGCTAGTAA | NO: |
| | | | | | | | | | | | | ATAATAAAGGCAAGATTTTA | 1041 |
| | | | | | | | | | | | | GAGCC | |
| 0.2479 | 0.224 | 0.198 | 5.67 | 1.14 | 1.07 | 1.21 | 8 | rs1734 | 60,82 | G | A | TAATGAATCTGAGTGGGATA | SEQ |
| | 6 | 5 | E−05 | | | | | 2242 | 8,697 | | | GTGATCAGAATAAGGAAGTA | ID |
| | | | | | | | | | | | | [A/G]GGCCAATAACATTTC | NO: |
| | | | | | | | | | | | | TGGGTAACTTGCCATGAGCC | 1042 |
| | | | | | | | | | | | | AAGCA | |
| 0.0619 | 0.079 | 0.08 | 2.88 | 0.77 | 0.69 | 0.86 | 9 | rs9695 | 106,1 | A | C | TTATAGTCCCAAGTAGTCAG | SEQ |
| 9 | 25 | | E−06 | | | | | 167 | 69,26 | | | AGATGGACTGTATAATATGC | ID |
| | | | | | | | | | 8 | | | [C/A]GGGCACAGGGCAAAA | NO: |
| | | | | | | | | | | | | CAAGAATGAGGGAAGTTGTT | 1043 |
| | | | | | | | | | | | | GACAG | |
| 0.3579 | 0.391 | 0.386 | 4.64 | 0.87 | 0.82 | 0.92 | 10 | rs1125 | 5,422 | C | A | AGCTATCATTCCCCAGTGTG | SEQ |
| | 9 | 1 | E−07 | | | | | 3141 | 2,196 | | | AACCTCAAGTCATCAGATTG | ID |
| | | | | | | | | | | | | [A/C]ATCTCCCCACCTGCC | NO: |
| | | | | | | | | | | | | ATTGTTTTTATCACCTACCA | 1044 |
| | | | | | | | | | | | | ACACC | |
| 0.1681 | 0.142 | 0.132 | 1.62 | 1.22 | 1.13 | 1.31 | 10 | rs1125 | 9,22 | C | A | TGAAATTGAAGTGGTGTTTA | SEQ |
| | 5 | 7 | E−07 | | | | | 6106 | 2,228 | | | TGAATCACATATGATAGATT | ID |
| | | | | | | | | | | | | [A/C]GGCAATTGAGTTATA | NO: |
| | | | | | | | | | | | | TTTTTATATCTGCTTATCTC | 1045 |
| | | | | | | | | | | | | TCTAA | |
| 0.4008 | 0.373 | 0.369 | 4.37 | 1.12 | 1.06 | 1.19 | 13 | rs7997 | 46,36 | A | G | GGCTGGAGGTCGAAAGACTC | SEQ |
| | 4 | 4 | E−05 | | | | | 707 | 0,678 | | | TAATCTGTTTCACTGTTTAC | ID |
| | | | | | | | | | | | | [G/A]TGTTCAGTCAGTTCT | NO: |
| | | | | | | | | | | | | CTCATTGGCAAAATATTTAT | 1046 |
| | | | | | | | | | | | | CTCAA | |
| 0.1636 | 0.184 | 0.172 | 7.49 | 0.86 | 0.80 | 0.93 | 13 | rs9317 | 66,13 | C | T | TGTTAAGTTATTCCAATAAT | SEQ |
| | 8 | 6 | E−05 | | | | | 519 | 7,562 | | | AAAATGTCATCCATAGGTTA | ID |
| | | | | | | | | | | | | [T/C]TGTCACGTTTTAATA | NO: |
| | | | | | | | | | | | | TAAGACTTCTAATCAAATTC | 1047 |
| | | | | | | | | | | | | CTGGG | |
| 0.1589 | 0.139 | 0.130 | 5.40 | 1.17 | 1.08 | 1.26 | 13 | rs3362 | 110,4 | T | C | TGGCTTCTTCGCAACTTGCA | SEQ |
| | 5 | 5 | E−05 | | | | | 37 | 96,41 | | | TAGAGGCTACCTCTGTGTCC | ID |
| | | | | | | | | | 0 | | | [C/T]CTTATGGCTCGATAG | NO: |
| | | | | | | | | | | | | CTCATTTCTTTTTATCCCCA | 1048 |
| | | | | | | | | | | | | AATAA | |
| 0.3534 | 0.326 | 0.32 | 3.80 | 1.13 | 1.07 | 1.19 | 14 | rs1049 | 52,54 | G | A | ATAAACATAGTTATGCTTCA | SEQ |
| | 6 | | E−05 | | | | | 8441 | 4,224 | | | TTACTCTGGTACAGAAACCC | ID |
| | | | | | | | | | | | | [G/A]GTTCATTAGCCATTC | NO: |
| | | | | | | | | | | | | AGAATGATTGTGATATCCAA | 1049 |
| | | | | | | | | | | | | AATGA | |
| 0.3145 | 0.287 | 0.285 | 1.36 | 1.14 | 1.07 | 1.21 | 14 | rs7157 | 52,57 | T | C | TGTATCCAACCATGGGAAAA | SEQ |
| | 1 | 5 | E−05 | | | | | 151 | 1,583 | | | AGACTTAGCTACATTGTATA | ID |
| | | | | | | | | | | | | [T/C]ATTTGATGAGTAACG | NO: |
| | | | | | | | | | | | | TGTTTATAATACAACAAAAA | 1050 |
| | | | | | | | | | | | | GTGAA | |
| 0.1256 | 0.108 | 0.113 | 9.94 | 1.18 | 1.08 | 1.28 | 14 | rs1258 | 71,18 | T | C | TTGTGCTGCCTGAGAGGAGA | SEQ |
| | 7 | 1 | E−05 | | | | | 6828 | 6,513 | | | GGGAGCATCTCACCATCTCC | ID |
| | | | | | | | | | | | | [C/T]GCCTTGGTATCTTTT | NO: |
| | | | | | | | | | | | | ATTCTTTAGGACTCAGCTCA | 1051 |
| | | | | | | | | | | | | GGTTC | |
| 0.4297 | 0.460 | 0.457 | 5.73 | 0.88 | 0.83 | 0.93 | 14 | rs1951 | 100,7 | G | A | AATAAGTGAAAGAACTAGCA | SEQ |
| | 9 | 2 | E−06 | | | | | 521 | 43,42 | | | GTGCAGCTAGTAAATCTAAC | ID |
| | | | | | | | | | 1 | | | [G/A]TGGTTCTTTTTTGAC | NO: |
| | | | | | | | | | | | | AACTGACACCAGAACCCTTA | 1052 |
| | | | | | | | | | | | | ATCAT | |
| 0.3167 | 0.343 | 0.337 | 3.97 | 0.89 | 0.84 | 0.94 | 15 | rs7181 | 40,36 | G | A | AAAAAACCCTTACATTAGCA | SEQ |
| | 6 | 8 | E−05 | | | | | 230 | 0,741 | | | TAAAATCTGTAACAGGAGTG | ID |
| | | | | | | | | | | | | [A/G]AATGGAAATACAAGT | NO: |
| | | | | | | | | | | | | TCTTGGAGAGAACGAAATAA | 1053 |
| | | | | | | | | | | | | TGTAA | |
| 0.5069 | 0.479 | 0.474 | 7.28 | 1.12 | 1.06 | 1.18 | 15 | rs1244 | 47,14 | C | T | TTGCCTTTAGGACAGGACTG | SEQ |
| | 4 | 6 | E−05 | | | | | 2708 | 4,386 | | | TTCTTAGTCCTCTCCAGTTC | ID |
| | | | | | | | | | | | | [T/C]ACTCTATTGTAAAGT | NO: |
| | | | | | | | | | | | | TTCTGAAAGTGCCTCAGGTA | 1054 |
| | | | | | | | | | | | | TTTCA | |
| 0.4955 | 0.466 | 0.471 | 1.79 | 1.13 | 1.07 | 1.19 | 16 | rs1085 | 66,40 | C | T | AGAATCTTAGGCTCATTTTG | SEQ |
| | | 2 | E−05 | | | | | 2432 | 2,515 | | | CCCACATGGACCCATGACTG | ID |
| | | | | | | | | | | | | [T/C]TCCCTGTATCCTCTC | NO: |
| | | | | | | | | | | | | TCTGCACCCCCTCAGTCACA | 1055 |
| | | | | | | | | | | | | CTGAA | |
| 0.1229 | 0.104 | 0.105 | 2.60 | 1.20 | 1.10 | 1.30 | 16 | rs1528 | 72,12 | T | C | CAGTGTCTACATCACTGACC | SEQ |
| | 9 | 6 | E−05 | | | | | 28 | 3,886 | | | TCTGTGGTATTTCCTCCTGC | ID |
| | | | | | | | | | | | | [T/C]TATGACTGAGGGTAG | NO: |
| | | | | | | | | | | | | AATCCTCTGGTCCTTTTTTC | 1056 |
| | | | | | | | | | | | | CCCAA | |
| 0.3705 | 0.343 | 0.348 | 2.69 | 1.13 | 1.07 | 1.19 | 17 | rs8076 | 66,51 | A | G | GAGCCAGGTCATAGATGTAG | SEQ |
| | | 8 | E−05 | | | | | 465 | 3,025 | | | CTTGTTTTGAAGTCAAGTGC | ID |
| | | | | | | | | | | | | [A/G]TTCCTGGAGATCCGG | NO: |
| | | | | | | | | | | | | TTTTGAAATGGGTCACTGTA | 1057 |
| | | | | | | | | | | | | AGGTG | |
| 0.3709 | 0.343 | 0.347 | 2.45 | 1.13 | 1.07 | 1.19 | 17 | rs2907 | 66,53 | A | G | CCCTTAGCTTGTCAAGTTAG | SEQ |
| | 2 | 5 | E−05 | | | | | 373 | 3,655 | | | CCTGGCCAGAGTCTGGGGCC | ID |
| | | | | | | | | | | | | [A/G]ACTGTTCCACTGGGC | NO: |
| | | | | | | | | | | | | CGTCGACTATGACACTCTGC | 1058 |
| | | | | | | | | | | | | TGTCC | |
| 0.2337 | 0.210 | 0.207 | 6.31 | 1.14 | 1.07 | 1.22 | 18 | rs2175 | 46,07 | G | A | GACGGTGAGGAGCGGGTGAT | SEQ |
| | 9 | | E−05 | | | | | 565 | 9,852 | | | GGGGTAATTCCCGGAATGCA | ID |
| | | | | | | | | | | | | [G/A]ACTGTAACCAGGGCA | NO: |
| | | | | | | | | | | | | GTCAGAACAAGGATTGTTAA | 1059 |
| | | | | | | | | | | | | CCTGC | |
| 0.3788 | 0.352 | 0.361 | 7.47 | 1.12 | 1.06 | 1.18 | 18 | rs3900 | 74,73 | T | C | GTGAGTCGCCACTGTTGGCT | SEQ |
| | 5 | 7 | E−05 | | | | | 176 | 9,022 | | | TATTTTATGTATTTGCATCG | ID |
| | | | | | | | | | | | | [T/C]TCCCATCTAAATGGG | NO: |
| | | | | | | | | | | | | GATTCCCAGACTTCATAGGC | 1060 |
| | | | | | | | | | | | | CAGTA | |
| 0.0717 | 0.057 | 0.061 | 2.35 | 1.26 | 1.13 | 1.40 | 20 | rs6110 | 15,69 | G | A | GTACTTATAAAGCAGCGGAA | SEQ |
| 2 | 86 | 64 | E−05 | | | | | 759 | 3,977 | | | TCTCCTGCTTTATGAACTTT | ID |
| | | | | | | | | | | | | [A/G]GTTCTGGGCTTCAGC | NO: |
| | | | | | | | | | | | | TCTGTATTAGTCTGTTCTCA | 1061 |
| | | | | | | | | | | | | CACTG | |
| 0.2432 | 0.22 | 0.230 | 5.67 | 1.14 | 1.07 | 1.21 | 20 | rs6043 | 16,45 | C | A | AATTCTCAGATCCACCAGTG | SEQ |
| | | | E−05 | | | | | 979 | 1,642 | | | AGACAGAAAACATAGGAGAC | ID |
| | | | | | | | | | | | | [A/C]GGAAAAGAAGAATCA | NO: |
| | | | | | | | | | | | | AATGGGAAGTGGAAAAAAGA | 1062 |
| | | | | | | | | | | | | CAGGG | |
| 0.0277 | 0.019 | 0.016 | 8.72 | 1.46 | 1.24 | 1.73 | 21 | rs1170 | 41,90 | T | C | AAATGCTCCTAGAACTGCAA | SEQ |
| 7 | 14 | 63 | E−06 | | | | | 2826 | 8,935 | | | AACACCTAACTTATTCCAAA | ID |
| | | | | | | | | | | | | [C/T]TTTCCGGATGAAAAG | NO: |
| | | | | | | | | | | | | GCAGAGGATTTTCTACTCCC | 1063 |
| | | | | | | | | | | | | ATTTC | |
| 0.2375 | 0.262 | 0.248 | 4.68 | 0.88 | 0.82 | 0.93 | 22 | rs1296 | 18,02 | G | A | TCTCTTTCCAGGTTAAATGT | SEQ |
| | 3 | | E−05 | | | | | 795 | 1,760 | | | TGTTCATTGCGTCCTTTCCC | ID |
| | | | | | | | | | | | | [A/G]AAGAGTCTGTTCCCA | NO: |
| | | | | | | | | | | | | TAGAGAAGCATGGCACAAAG | 1064 |
| | | | | | | | | | | | | TGTGC | |
| 0.077 | 0.094 | 0.090 | 1.61 | 0.80 | 0.73 | 0.89 | 22 | rs7364 | 45,33 | T | C | CAGCCGATGGGCTCTGCCAG | SEQ |
| | 21 | 76 | E−05 | | | | | 90 | 8,213 | | | ATTCCTGATCCACAGTAGGA | ID |
| | | | | | | | | | | | | [C/T]CCTGGGGGCACCCTC | NO: |
| | | | | | | | | | | | | TGCCCGAGGACCCTGGAACA | 1065 |
| | | | | | | | | | | | | CACAG | |

While exemplary embodiments of the present disclosure have been shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed:

1. A method comprising:
(a) hybridizing a nucleic acid probe to a nucleic acid sample from a human subject suspected of having or developing endometriosis; and
(b) detecting a genetic variant in a panel comprising two or more genetic variants defining a minor allele listed in Table 1.

2. The method of claim 1, wherein the nucleic acid sample comprises mRNA, cDNA, genomic DNA, or PCR amplified products produced therefrom, or any combination thereof.

3. The method of claim 1, wherein the nucleic acid sample comprises PCR amplified nucleic acids produced from cDNA or mRNA.

4. The method of claim 1, wherein the nucleic acid sample comprises PCR amplified nucleic acids produced from genomic DNA.

5. The method of claim 1, wherein the nucleic acid probe is a sequencing primer.

6. The method of claim 1, wherein the nucleic acid probe is an allele specific probe.

7. The method of claim 1, wherein the detecting comprises DNA sequencing, hybridization with a complementary probe, an oligonucleotide ligation assay, a PCR-based assay, or any combination thereof.

8. The method of claim 1, wherein the panel comprises at least: 5, 10, 15, 20, 25, 50, 75, 100, 150, 200, 250, 500, or more genetic variants defining minor alleles listed in Table 1.

9. The method of claim 1, wherein the genetic variant has an odds ratio (OR) of at least: 1.5, 2, 5, 10, 20, 50, 100, or more.

10. The method of claim 1, wherein the genetic variant comprises a synonymous mutation, a non-synonymous mutation, a nonsense mutation, an insertion, a deletion, a splice-site variant, a frameshift mutation, or any combination thereof.

11. The method of claim 1, wherein the genetic variant comprises a protein damaging mutation.

12. The method of claim 1, wherein the panel further comprises one or more protein damaging or loss of function variants in one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof.

13. The method of claim 12, further comprising sequencing the one or more genes to identify the one or more protein damaging or loss of function variants.

14. The method of claim 13, wherein the one or more protein damaging or loss of function variants are identified based on a predictive computer algorithm.

15. The method of claim 13, wherein the one or more protein damaging or loss of function variants are identified based on reference to a database.

16. The method of claim 12, wherein the one or more protein damaging or loss of function variants comprise a stop-gain mutation, a spice-site mutation, a frameshift mutation, a missense mutation, or any combination thereof.

17. The method of claim 1, wherein the panel further comprises one or more additional variants defining a minor allele listed in Table 4.

18. The method of claim 1, wherein the panel is capable of identifying human subjects as having or being at risk of developing endometriosis with a specificity of at least: 80%, 85%, 90%, 95%, 96%, 97%, 98%, or 99%.

19. The method of claim 1, wherein the panel is capable of identifying human subjects as having or being at risk of developing endometriosis with a sensitivity of at least: 80%, 85%, 90%, 95%, 96%, 97%, 98%, or 99%.

20. The method of claim 1, wherein the panel is capable of identifying human subjects as having or being at risk of developing endometriosis with an accuracy of at least: 80%, 85%, 90%, 95%, 96%, 97%, 98%, or 99%.

21. The method of claim 1, further comprising administering a therapeutic to the human subject.

22. The method of claim 21, wherein the therapeutic comprises hormonal therapy, an advanced reproductive therapy, a pain managing medication, or any combination thereof.

23. The method of claim 21, wherein the therapeutic comprises hormonal contraceptives, gonadotropin-releasing hormone (Gn-RH) agonists, gonadotropin-releasing hormone (Gn-RH) antagonists, progestin, danazol, or any combination thereof.

24. The method of claim 1, wherein the human subject is asymptomatic for endometriosis.

25. The method of claim 1, wherein the human subject is a teenager.

26. A method comprising detecting one or more genetic variants defining a minor allele listed in Table 1 in genetic material from a human subject suspected of having or developing endometriosis.

27. The method of claim 26, wherein the genetic material comprises mRNA, cDNA, genomic DNA, or PCR amplified products produced therefrom, or any combination thereof.

28. The method of claim 26, wherein the detecting comprises DNA sequencing, hybridization with a complementary probe, an oligonucleotide ligation assay, a PCR-based assay, of any combination thereof.

29. The method of claim 26, wherein the detecting comprises hybridizing a nucleic acid probe to the genetic material.

30. The method of claim 26, wherein the detecting comprises testing for the presence or absence of at least: 2, 3, 4, 5, 6, 7, 8, 9, 10, 15, 20, 25, 50, 100, 150, 250, or 500 genetic variants defining a minor allele listed in Table 1.

31. The method of claim 26, wherein the one or more genetic variants have an odds ratio (OR) of at least: 1.5, 2, 5, 10, 20, 50, 100, or more.

32. The method of claim 26, further comprising administering a therapeutic to the human subject.

33. A method comprising:
(a) sequencing one or more genes selected from the group consisting of GAT2, CCDC169, CASP8AP2, POU2F3, CD19, IGSF3, GLI3, PEX26, OLIG3, CIB4, NKX3-2, CFTR, and any combinations thereof to identify one or more protein damaging or loss of function variants in a human subject suspected of having or developing endometriosis; and
(b) administering an endometriosis therapy to the human subject.

34. The method of claim 33, wherein the one or more protein damaging or loss of function variants are identified based on a predictive computer algorithm, reference to a database, or a combination thereof.

35. The method of claim 33, wherein the one or more protein damaging or loss of function variants comprise a stop-gain mutation, a spice-site mutation, a frameshift mutation, a missense mutation, or any combination thereof.

36. The method of claim 33, wherein the endometriosis therapy comprises a hormonal therapy, an assisted reproductive therapy, a pain medication, or any combination thereof.

37. A method of preventing endometriosis comprising administering a hormonal therapy to a human subject having at least one genetic variant defining a minor allele listed in Table 1.

38. The method of claim 37, wherein the hormonal therapy comprises administration of hormonal contraceptives, gonadotropin-releasing hormone (Gn-RH) agonists, gonadotropin-releasing hormone (Gn-RH) antagonists, progestin, danazol, or any combination thereof.

39. The method of claim 37, further comprising detecting the at least one genetic variant in a genetic material from the human subject.

40. The method of claim 39, wherein the detecting comprises DNA sequencing, hybridization with a complementary probe, an oligonucleotide ligation assay, a PCR-based assay, or any combination thereof.

41. The method of claim 39, wherein the detecting comprises hybridizing a nucleic acid probe to the genetic material.

42. The method of claim 41, wherein the nucleic acid probe is a sequencing primer or an allele-specific probe.

43. The method of claim 37, wherein the at least one genetic variant has an odds ratio (OR) of at least: 1.5, 2, 5, 10, 20, 50, 100, or more.

44. The method of claim 37, wherein the at least one genetic variant comprises a synonymous mutation, a non-synonymous mutation, a nonsense mutation, an insertion, a deletion, a splice-site variant, a frameshift mutation, or any combination thereof.

45. A method of treating endometriosis associated infertility comprising administering an assisted reproductive therapy to a human subject having at least one genetic variant defining a minor allele listed in Table 2.

46. The method of claim 45, wherein the assisted reproductive therapy comprises in vitro fertilization, intrauterine insemination, ovulation induction, gamete intrafallopian transfer, or any combination thereof.

47. The method of claim 45, further comprising detecting the at least one genetic variant in a genetic material from the human subject.

48. The method of claim 47, wherein the detecting comprises DNA sequencing, hybridization with a complementary probe, an oligonucleotide ligation assay, a PCR-based assay, or any combination thereof.

49. The method of claim 47, wherein the detecting comprises hybridizing a nucleic acid probe to the genetic material.

50. The method of claim 49, wherein the nucleic acid probe is a sequencing primer or an allele-specific probe.

51. The method of claim 45, wherein the at least one genetic variant has an odds ratio (OR) of at least: 1.5, 2, 5, 10, 20, 50, 100, or more.

52. The method of claim 45, wherein the at least one genetic variant comprises a synonymous mutation, a non-synonymous mutation, a nonsense mutation, an insertion, a deletion, a splice-site variant, a frameshift mutation, or any combination thereof.

53. A method comprising administering a pain medication to a human subject having at least one genetic variant defining a minor allele listed in Table 3.

54. The method of claim 53, wherein the pain medication comprises a nonsteroidal anti-inflammatory drug (NSAID), ibuprofen, naproxen, an opioid, a cannabis-based therapeutic, or any combination thereof.

55. The method of claim 53, further comprising detecting the at least one genetic variant in a genetic material from the human subject.

56. The method of claim 55, wherein the detecting comprises DNA sequencing, hybridization with a complementary probe, an oligonucleotide ligation assay, a PCR-based assay, or any combination thereof.

57. The method of claim 55, wherein the detecting comprises hybridizing a nucleic acid probe to the genetic material.

58. The method of claim 57, wherein the nucleic acid probe is a sequencing primer or an allele-specific probe.

59. The method of claim 53, wherein the at least one genetic variant has an odds ratio (OR) of at least: 1.5, 2, 5, 10, 20, 50, 100, or more.

60. The method of claim 53, wherein the at least one genetic variant comprises a synonymous mutation, a non-synonymous mutation, a nonsense mutation, an insertion, a deletion, a splice-site variant, a frameshift mutation, or any combination thereof.
